SEQUENCE LISTING (1511 sequences)

SEQ ID NO: 1-562 - skipped (562 entries)

SEQ ID NO: 563 - DNA, 290 bp, Artificial Sequence
  Description of Artificial Sequence: Synthetic polynucleotide
  FNR Responsive Promoter
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 564 - DNA, 173 bp, Artificial Sequence
  Description of Artificial Sequence: Synthetic polynucleotide
  FNR Responsive Promoter
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 565 - DNA, 305 bp, Artificial Sequence
  Description of Artificial Sequence: Synthetic polynucleotide
  FNR Responsive Promoter
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 566 - DNA, 180 bp, Artificial Sequence
  Description of Artificial Sequence: Synthetic polynucleotide
  FNR Responsive Promoter
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 567 - DNA, 199 bp, Artificial Sequence
  Description of Artificial Sequence: Synthetic polynucleotide
  FNR Responsive Promoter
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 568 - DNA, 117 bp, Artificial Sequence
  Description of Artificial Sequence: Synthetic polynucleotide
  FNR-responsive regulatory region
atccccatcactcttgatggagatcaattccccaagctgctagagcgttaccttgcccttaaacattagcaatgtcgatttatcagagggccgacaggctcccacaggagaaaaccg

SEQ ID NO: 569 - DNA, 108 bp, Artificial Sequence
  Description of Artificial Sequence: Synthetic polynucleotide
  FNR-responsive regulatory region
ctcttgatcgttatcaattcccacgctgtttcagagcgttaccttgcccttaaacattagcaatgtcgatttatcagagggccgacaggctcccacaggagaaaaccg

SEQ ID NO: 570 - DNA, 290 bp, Artificial Sequence; identical to SEQ ID NO: 563
  Description of Artificial Sequence: Synthetic polynucleotide
  nirB1 , FNR-responsive regulatory region

SEQ ID NO: 571 - DNA, 433 bp, Artificial Sequence
  Description of Artificial Sequence: Synthetic polynucleotide
  nirB2 , FNR-responsive regulatory region
cggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgtttgtttaactttaagaaggagatatacat

SEQ ID NO: 572 - DNA, 290 bp, Artificial Sequence
  Description of Artificial Sequence: Synthetic polynucleotide
  nirB3 , FNR-responsive regulatory region
gtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaa

SEQ ID NO: 573 - DNA, 173 bp, Artificial Sequence; identical to SEQ ID NO: 564
  Description of Artificial Sequence: Synthetic polynucleotide
  ydfZ , FNR-responsive regulatory region

SEQ ID NO: 574 - DNA, 305 bp, Artificial Sequence; identical to SEQ ID NO: 565
  Description of Artificial Sequence: Synthetic polynucleotide
  nirB+RBS , FNR-responsive regulatory region

SEQ ID NO: 575 - DNA, 180 bp, Artificial Sequence; identical to SEQ ID NO: 566
  Description of Artificial Sequence: Synthetic polynucleotide
  ydfZ+RBS , FNR-responsive regulatory region

SEQ ID NO: 576 - DNA, 199 bp, Artificial Sequence; identical to SEQ ID NO: 567
  Description of Artificial Sequence: Synthetic polynucleotide
  FNRS1 , FNR-responsive regulatory region

SEQ ID NO: 577 - DNA, 207 bp, Artificial Sequence
  Description of Artificial Sequence: Synthetic polynucleotide
  FNRS2 , FNR-responsive regulatory region
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 578 - DNA, 390 bp, Artificial Sequence
  Description of Artificial Sequence: Synthetic polynucleotide
  nirB+crp , FNR-responsive regulatory region
tcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtagaaatgtgatctagttcacatttgcggtaatagaaaagaaatcgaggcaaaaatgtttgtttaactttaagaaggagatatacat

SEQ ID NO: 579 - DNA, 200 bp, Artificial Sequence
  Description of Artificial Sequence: Synthetic polynucleotide
  FNRS+crp , FNR-responsive regulatory region
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcaaatgtgatctagttcacattttttgtttaactttaagaaggagatatacat

SEQ ID NO: 580-597 - skipped (18 entries)

SEQ ID NO: 598 - DNA, 30 bp, Artificial Sequence
  Description of Artificial Sequence: Synthetic oligonucleotide
  BBa_I14018
gtttatacataggcgagtactctgttatgg

SEQ ID NO: 599 - DNA, 30 bp, Artificial Sequence
  Description of Artificial Sequence: Synthetic oligonucleotide
  BBa_I14033
agaggttccaactttcaccataatgaaaca

SEQ ID NO: 600 - DNA, 30 bp, Artificial Sequence
  Description of Artificial Sequence: Synthetic oligonucleotide
  BBa_I14034
taaacaactaacggacaattctacctaaca

SEQ ID NO: 601 - DNA, 30 bp, Artificial Sequence
  Description of Artificial Sequence: Synthetic oligonucleotide
  BBa_I732021
acatcaagccaaattaaacaggattaacac

SEQ ID NO: 602 - DNA, 30 bp, Artificial Sequence
  Description of Artificial Sequence: Synthetic oligonucleotide
  BBa_I742126
gaggtaaaatagtcaacacgcacggtgtta

SEQ ID NO: 603 - DNA, 30 bp, Artificial Sequence
  Description of Artificial Sequence: Synthetic oligonucleotide
  BBa_J01006
caggccggaataactccctataatgcgcca

SEQ ID NO: 604 - DNA, 30 bp, Artificial Sequence
  Description of Artificial Sequence: Synthetic oligonucleotide
  BBa_J23100
ggctagctcagtcctaggtacagtgctagc

SEQ ID NO: 605 - DNA, 30 bp, Artificial Sequence
  Description of Artificial Sequence: Synthetic oligonucleotide
  BBa_J23101
agctagctcagtcctaggtattatgctagc

SEQ ID NO: 606 - DNA, 30 bp, Artificial Sequence
  Description of Artificial Sequence: Synthetic oligonucleotide
  BBa_J23102
agctagctcagtcctaggtactgtgctagc

SEQ ID NO: 607 - DNA, 30 bp, Artificial Sequence
  Description of Artificial Sequence: Synthetic oligonucleotide
  BBa_J23103
agctagctcagtcctagggattatgctagc

SEQ ID NO: 608 - DNA, 30 bp, Artificial Sequence
  Description of Artificial Sequence: Synthetic oligonucleotide
  BBa_J23104
agctagctcagtcctaggtattgtgctagc

SEQ ID NO: 609 - DNA, 30 bp, Artificial Sequence
  Description of Artificial Sequence: Synthetic oligonucleotide
  BBa_J23105
ggctagctcagtcctaggtactatgctagc

SEQ ID NO: 610 - DNA, 30 bp, Artificial Sequence
  Description of Artificial Sequence: Synthetic oligonucleotide
  BBa_J23106
ggctagctcagtcctaggtatagtgctagc

SEQ ID NO: 611 - DNA, 30 bp, Artificial Sequence
  Description of Artificial Sequence: Synthetic oligonucleotide
  BBa_J23107
ggctagctcagccctaggtattatgctagc

SEQ ID NO: 612 - DNA, 30 bp, Artificial Sequence
  Description of Artificial Sequence: Synthetic oligonucleotide
  BBa_J23108
agctagctcagtcctaggtataatgctagc

SEQ ID NO: 613 - DNA, 30 bp, Artificial Sequence
  Description of Artificial Sequence: Synthetic oligonucleotide
  BBa_J23109
agctagctcagtcctagggactgtgctagc

SEQ ID NO: 614 - DNA, 30 bp, Artificial Sequence
  Description of Artificial Sequence: Synthetic oligonucleotide
  BBa_J23110
ggctagctcagtcctaggtacaatgctagc

SEQ ID NO: 615 - DNA, 30 bp, Artificial Sequence; identical to SEQ ID NO: 610
  Description of Artificial Sequence: Synthetic oligonucleotide
  BBa_J23111

SEQ ID NO: 616 - DNA, 30 bp, Artificial Sequence; identical to SEQ ID NO: 607
  Description of Artificial Sequence: Synthetic oligonucleotide
  BBa_J23112

SEQ ID NO: 617 - DNA, 30 bp, Artificial Sequence
  Description of Artificial Sequence: Synthetic oligonucleotide
  BBa_J23113
ggctagctcagtcctagggattatgctagc

SEQ ID NO: 618 - DNA, 30 bp, Artificial Sequence; identical to SEQ ID NO: 614
  Description of Artificial Sequence: Synthetic oligonucleotide
  BBa_J23114

SEQ ID NO: 619 - DNA, 30 bp, Artificial Sequence
  Description of Artificial Sequence: Synthetic oligonucleotide
  BBa_J23115
agctagctcagcccttggtacaatgctagc

SEQ ID NO: 620 - DNA, 30 bp, Artificial Sequence
  Description of Artificial Sequence: Synthetic oligonucleotide
  BBa_J23116
agctagctcagtcctagggactatgctagc

SEQ ID NO: 621 - DNA, 30 bp, Artificial Sequence
  Description of Artificial Sequence: Synthetic oligonucleotide
  BBa_J23117
agctagctcagtcctagggattgtgctagc

SEQ ID NO: 622 - DNA, 30 bp, Artificial Sequence
  Description of Artificial Sequence: Synthetic oligonucleotide
  BBa_J23118
ggctagctcagtcctaggtattgtgctagc

SEQ ID NO: 623 - DNA, 30 bp, Artificial Sequence; identical to SEQ ID NO: 612
  Description of Artificial Sequence: Synthetic oligonucleotide
  BBa_J23119

SEQ ID NO: 624 - DNA, 30 bp, Artificial Sequence
  Description of Artificial Sequence: Synthetic oligonucleotide
  BBa_J23150
ggctagctcagtcctaggtattatgctagc

SEQ ID NO: 625 - DNA, 30 bp, Artificial Sequence; identical to SEQ ID NO: 614
  Description of Artificial Sequence: Synthetic oligonucleotide
  BBa_J23151

SEQ ID NO: 626 - DNA, 30 bp, Artificial Sequence
  Description of Artificial Sequence: Synthetic oligonucleotide
  BBa_J44002
aaagtgtgacgccgtgcaaataatcaatgt

SEQ ID NO: 627 - DNA, 30 bp, Artificial Sequence
  Description of Artificial Sequence: Synthetic oligonucleotide
  BBa_J48104
gacgaatacttaaaatcgtcatacttattt

SEQ ID NO: 628 - DNA, 30 bp, Artificial Sequence
  Description of Artificial Sequence: Synthetic oligonucleotide
  BBa_J54200
aaacctttcgcggtatggcatgatagcgcc

SEQ ID NO: 629 - DNA, 30 bp, Artificial Sequence
  Description of Artificial Sequence: Synthetic oligonucleotide
  BBa_J56015
tgatagcgcccggaagagagtcaattcagg

SEQ ID NO: 630 - DNA, 30 bp, Artificial Sequence
  Description of Artificial Sequence: Synthetic oligonucleotide
  BBa_J64951
ttatttaccgtgacgaactaattgctcgtg

SEQ ID NO: 631 - DNA, 30 bp, Artificial Sequence
  Description of Artificial Sequence: Synthetic oligonucleotide
  BBa_K088007
catacgccgttatacgttgtttacgctttg

SEQ ID NO: 632 - DNA, 30 bp, Artificial Sequence
  Description of Artificial Sequence: Synthetic oligonucleotide
  BBa_K119000
ttatgcttccggctcgtatgttgtgtggac

SEQ ID NO: 633 - DNA, 30 bp, Artificial Sequence
  Description of Artificial Sequence: Synthetic oligonucleotide
  BBa_K119001
ttatgcttccggctcgtatggtgtgtggac

SEQ ID NO: 634 - DNA, 30 bp, Artificial Sequence; identical to SEQ ID NO: 609
  Description of Artificial Sequence: Synthetic oligonucleotide
  BBa_K1330002

SEQ ID NO: 635 - DNA, 30 bp, Artificial Sequence
  Description of Artificial Sequence: Synthetic oligonucleotide
  BBa_K137029
atatatatatatatataatggaagcgtttt

SEQ ID NO: 636 - DNA, 30 bp, Artificial Sequence; identical to SEQ ID NO: 635
  Description of Artificial Sequence: Synthetic oligonucleotide
  BBa_K137030

SEQ ID NO: 637 - DNA, 30 bp, Artificial Sequence
  Description of Artificial Sequence: Synthetic oligonucleotide
  BBa_K137031
ccccgaaagcttaagaatataattgtaagc

SEQ ID NO: 638 - DNA, 30 bp, Artificial Sequence; identical to SEQ ID NO: 637
  Description of Artificial Sequence: Synthetic oligonucleotide
  BBa_K137032

SEQ ID NO: 639 - DNA, 30 bp, Artificial Sequence
  Description of Artificial Sequence: Synthetic oligonucleotide
  BBa_K137085
tgacaatatatatatatatataatgctagc

SEQ ID NO: 640 - DNA, 30 bp, Artificial Sequence
  Description of Artificial Sequence: Synthetic oligonucleotide
  BBa_K137086
acaatatatatatatatatataatgctagc

SEQ ID NO: 641 - DNA, 30 bp, Artificial Sequence
  Description of Artificial Sequence: Synthetic oligonucleotide
  BBa_K137087
aatatatatatatatatatataatgctagc

SEQ ID NO: 642 - DNA, 30 bp, Artificial Sequence
  Description of Artificial Sequence: Synthetic oligonucleotide
  BBa_K137088
tatatatatatatatatatataatgctagc

SEQ ID NO: 643 - DNA, 30 bp, Artificial Sequence; identical to SEQ ID NO: 642
  Description of Artificial Sequence: Synthetic oligonucleotide
  BBa_K137089

SEQ ID NO: 644 - DNA, 30 bp, Artificial Sequence
  Description of Artificial Sequence: Synthetic oligonucleotide
  BBa_K137090
aaaaaaaaaaaaaaaaaatataatgctagc

SEQ ID NO: 645 - DNA, 30 bp, Artificial Sequence; identical to SEQ ID NO: 644
  Description of Artificial Sequence: Synthetic oligonucleotide
  BBa_K137091

SEQ ID NO: 646 - DNA, 30 bp, Artificial Sequence
  Description of Artificial Sequence: Synthetic oligonucleotide
  BBa_K1585100
ggaattgtgagcggataacaatttcacaca

SEQ ID NO: 647 - DNA, 30 bp, Artificial Sequence; identical to SEQ ID NO: 646
  Description of Artificial Sequence: Synthetic oligonucleotide
  BBa_K1585101

SEQ ID NO: 648 - DNA, 30 bp, Artificial Sequence; identical to SEQ ID NO: 646
  Description of Artificial Sequence: Synthetic oligonucleotide
  BBa_K1585102

SEQ ID NO: 649 - DNA, 30 bp, Artificial Sequence; identical to SEQ ID NO: 646
  Description of Artificial Sequence: Synthetic oligonucleotide
  BBa_K1585103

SEQ ID NO: 650 - DNA, 30 bp, Artificial Sequence; identical to SEQ ID NO: 646
  Description of Artificial Sequence: Synthetic oligonucleotide
  BBa_K1585104

SEQ ID NO: 651 - DNA, 30 bp, Artificial Sequence; identical to SEQ ID NO: 646
  Description of Artificial Sequence: Synthetic oligonucleotide
  BBa_K1585105

SEQ ID NO: 652 - DNA, 30 bp, Artificial Sequence; identical to SEQ ID NO: 646
  Description of Artificial Sequence: Synthetic oligonucleotide
  BBa_K1585106

SEQ ID NO: 653 - DNA, 30 bp, Artificial Sequence; identical to SEQ ID NO: 646
  Description of Artificial Sequence: Synthetic oligonucleotide
  BBa_K1585110

SEQ ID NO: 654 - DNA, 30 bp, Artificial Sequence; identical to SEQ ID NO: 646
  Description of Artificial Sequence: Synthetic oligonucleotide
  BBa_K1585113

SEQ ID NO: 655 - DNA, 30 bp, Artificial Sequence; identical to SEQ ID NO: 646
  Description of Artificial Sequence: Synthetic oligonucleotide
  BBa_K1585115

SEQ ID NO: 656 - DNA, 30 bp, Artificial Sequence; identical to SEQ ID NO: 646
  Description of Artificial Sequence: Synthetic oligonucleotide
  BBa_K1585116

SEQ ID NO: 657 - DNA, 30 bp, Artificial Sequence; identical to SEQ ID NO: 646
  Description of Artificial Sequence: Synthetic oligonucleotide
  BBa_K1585117

SEQ ID NO: 658 - DNA, 30 bp, Artificial Sequence; identical to SEQ ID NO: 646
  Description of Artificial Sequence: Synthetic oligonucleotide
  BBa_K1585118

SEQ ID NO: 659 - DNA, 30 bp, Artificial Sequence; identical to SEQ ID NO: 646
  Description of Artificial Sequence: Synthetic oligonucleotide
  BBa_K1585119

SEQ ID NO: 660 - DNA, 30 bp, Artificial Sequence
  Description of Artificial Sequence: Synthetic oligonucleotide
  BBa_K1824896
gattaaagaggagaaatactagagtactag

SEQ ID NO: 661 - DNA, 30 bp, Artificial Sequence
  Description of Artificial Sequence: Synthetic oligonucleotide
  BBa_K256002
caccttcgggtgggcctttctgcgtttata

SEQ ID NO: 662 - DNA, 30 bp, Artificial Sequence; identical to SEQ ID NO: 661
  Description of Artificial Sequence: Synthetic oligonucleotide
  BBa_K256018

SEQ ID NO: 663 - DNA, 30 bp, Artificial Sequence; identical to SEQ ID NO: 661
  Description of Artificial Sequence: Synthetic oligonucleotide
  BBa_K256020

SEQ ID NO: 664 - DNA, 30 bp, Artificial Sequence; identical to SEQ ID NO: 661
  Description of Artificial Sequence: Synthetic oligonucleotide
  BBa_K256033

SEQ ID NO: 665 - DNA, 30 bp, Artificial Sequence; identical to SEQ ID NO: 604
  Description of Artificial Sequence: Synthetic oligonucleotide
  BBa_K292000

SEQ ID NO: 666 - DNA, 30 bp, Artificial Sequence
  Description of Artificial Sequence: Synthetic oligonucleotide
  BBa_K292001
tgctagctactagagattaaagaggagaaa

SEQ ID NO: 667 - DNA, 30 bp, Artificial Sequence
  Description of Artificial Sequence: Synthetic oligonucleotide
  BBa_K418000
ttgtgagcggataacaagatactgagcaca

SEQ ID NO: 668 - DNA, 30 bp, Artificial Sequence; identical to SEQ ID NO: 667
  Description of Artificial Sequence: Synthetic oligonucleotide
  BBa_K418002

SEQ ID NO: 669 - DNA, 30 bp, Artificial Sequence; identical to SEQ ID NO: 667
  Description of Artificial Sequence: Synthetic oligonucleotide
  BBa_K418003

SEQ ID NO: 670 - DNA, 30 bp, Artificial Sequence; identical to SEQ ID NO: 604
  Description of Artificial Sequence: Synthetic oligonucleotide
  BBa_K823004

SEQ ID NO: 671 - DNA, 30 bp, Artificial Sequence; identical to SEQ ID NO: 605
  Description of Artificial Sequence: Synthetic oligonucleotide
  BBa_K823005

SEQ ID NO: 672 - DNA, 30 bp, Artificial Sequence; identical to SEQ ID NO: 606
  Description of Artificial Sequence: Synthetic oligonucleotide
  BBa_K823006

SEQ ID NO: 673 - DNA, 30 bp, Artificial Sequence; identical to SEQ ID NO: 607
  Description of Artificial Sequence: Synthetic oligonucleotide
  BBa_K823007

SEQ ID NO: 674 - DNA, 30 bp, Artificial Sequence; identical to SEQ ID NO: 610
  Description of Artificial Sequence: Synthetic oligonucleotide
  BBa_K823008

SEQ ID NO: 675 - DNA, 30 bp, Artificial Sequence; identical to SEQ ID NO: 617
  Description of Artificial Sequence: Synthetic oligonucleotide
  BBa_K823010

SEQ ID NO: 676 - DNA, 30 bp, Artificial Sequence; identical to SEQ ID NO: 614
  Description of Artificial Sequence: Synthetic oligonucleotide
  BBa_K823011

SEQ ID NO: 677 - DNA, 30 bp, Artificial Sequence; identical to SEQ ID NO: 621
  Description of Artificial Sequence: Synthetic oligonucleotide
  BBa_K823013

SEQ ID NO: 678 - DNA, 30 bp, Artificial Sequence; identical to SEQ ID NO: 622
  Description of Artificial Sequence: Synthetic oligonucleotide
  BBa_K823014

SEQ ID NO: 679 - DNA, 30 bp, Artificial Sequence
  Description of Artificial Sequence: Synthetic oligonucleotide
  BBa_M13101
cctgtttttatgttattctctctgtaaagg

SEQ ID NO: 680 - DNA, 30 bp, Artificial Sequence
  Description of Artificial Sequence: Synthetic oligonucleotide
  BBa_M13102
aaatatttgcttatacaatcttcctgtttt

SEQ ID NO: 681 - DNA, 30 bp, Artificial Sequence
  Description of Artificial Sequence: Synthetic oligonucleotide
  BBa_M13103
gctgataaaccgatacaattaaaggctcct

SEQ ID NO: 682 - DNA, 30 bp, Artificial Sequence
  Description of Artificial Sequence: Synthetic oligonucleotide
  BBa_M13104
ctcttctcagcgtcttaatctaagctatcg

SEQ ID NO: 683 - DNA, 30 bp, Artificial Sequence
  Description of Artificial Sequence: Synthetic oligonucleotide
  BBa_M13105
atgagccagttcttaaaatcgcataaggta

SEQ ID NO: 684 - DNA, 30 bp, Artificial Sequence
  Description of Artificial Sequence: Synthetic oligonucleotide
  BBa_M13106
ctattgattgtgacaaaataaacttattcc

SEQ ID NO: 685 - DNA, 30 bp, Artificial Sequence
  Description of Artificial Sequence: Synthetic oligonucleotide
  BBa_M13108
gtttcgcgcttggtataatcgctgggggtc

SEQ ID NO: 686 - DNA, 30 bp, Artificial Sequence
  Description of Artificial Sequence: Synthetic oligonucleotide
  BBa_M13110
ctttgcttctgactataatagtcagggtaa

SEQ ID NO: 687 - DNA, 30 bp, Artificial Sequence
  Description of Artificial Sequence: Synthetic oligonucleotide
  BBa_M31519
aaaccgatacaattaaaggctcctgctagc

SEQ ID NO: 688 - DNA, 30 bp, Artificial Sequence
  Description of Artificial Sequence: Synthetic oligonucleotide
  BBa_R1074
caccacactgatagtgctagtgtagatcac

SEQ ID NO: 689 - DNA, 30 bp, Artificial Sequence
  Description of Artificial Sequence: Synthetic oligonucleotide
  BBa_R1075
gccggaataactccctataatgcgccacca

SEQ ID NO: 690 - DNA, 30 bp, Artificial Sequence
  Description of Artificial Sequence: Synthetic oligonucleotide
  BBa_S03331
ttgacaagcttttcctcagctccgtaaact

SEQ ID NO: 691 - DNA, 30 bp, Artificial Sequence
  Description of Artificial Sequence: Synthetic oligonucleotide
  BBa_J45992
ggtttcaaaattgtgatctatatttaacaa

SEQ ID NO: 692 - DNA, 30 bp, Artificial Sequence; identical to SEQ ID NO: 691
  Description of Artificial Sequence: Synthetic oligonucleotide
  BBa_J45993

SEQ ID NO: 693 - DNA, 30 bp, Artificial Sequence
  Description of Artificial Sequence: Synthetic oligonucleotide
  BBa_J45504
tctattccaataaagaaatcttcctgcgtg

SEQ ID NO: 694 - DNA, 30 bp, Artificial Sequence
  Description of Artificial Sequence: Synthetic oligonucleotide
  BBa_K1895002
gaccgaatatatagtggaaacgtttagatg

SEQ ID NO: 695 - DNA, 30 bp, Artificial Sequence
  Description of Artificial Sequence: Synthetic oligonucleotide
  BBa_K1895003
ccacatcctgtttttaaccttaaaatggca

SEQ ID NO: 696 - DNA, 30 bp, Artificial Sequence
  Description of Artificial Sequence: Synthetic oligonucleotide
  BBa_K143012
aaaaatgggctcgtgttgtacaataaatgt

SEQ ID NO: 697 - DNA, 30 bp, Artificial Sequence
  Description of Artificial Sequence: Synthetic oligonucleotide
  BBa_K143013
aaaaaaagcgcgcgattatgtaaaatataa

SEQ ID NO: 698 - DNA, 30 bp, Artificial Sequence
  Description of Artificial Sequence: Synthetic oligonucleotide
  BBa_K780003
aattgcagtaggcatgacaaaatggactca

SEQ ID NO: 699 - DNA, 30 bp, Artificial Sequence
  Description of Artificial Sequence: Synthetic oligonucleotide
  BBa_K823000
caagcttttcctttataatagaatgaatga

SEQ ID NO: 700 - DNA, 30 bp, Artificial Sequence
  Description of Artificial Sequence: Synthetic oligonucleotide
  BBa_K823002
tctaagctagtgtattttgcgtttaatagt

SEQ ID NO: 701 - DNA, 30 bp, Artificial Sequence
  Description of Artificial Sequence: Synthetic oligonucleotide
  BBa_K823003
aatgggctcgtgttgtacaataaatgtagt

SEQ ID NO: 702 - DNA, 30 bp, Artificial Sequence
  Description of Artificial Sequence: Synthetic oligonucleotide
  BBa_K143010
atccttatcgttatgggtattgtttgtaat

SEQ ID NO: 703 - DNA, 30 bp, Artificial Sequence
  Description of Artificial Sequence: Synthetic oligonucleotide
  BBa_K143011
taaaagaattgtgagcgggaatacaacaac

SEQ ID NO: 704 - DNA, 30 bp, Artificial Sequence; identical to SEQ ID NO: 697
  Description of Artificial Sequence: Synthetic oligonucleotide
  BBa_K143013

SEQ ID NO: 705 - DNA, 30 bp, Artificial Sequence
  Description of Artificial Sequence: Synthetic oligonucleotide
  BBa_K112706
tacaaaataattcccctgcaaacattatca

SEQ ID NO: 706 - DNA, 30 bp, Artificial Sequence
  Description of Artificial Sequence: Synthetic oligonucleotide
  BBa_K112707
tacaaaataattcccctgcaaacattatcg

SEQ ID NO: 707 - DNA, 30 bp, Artificial Sequence
  Description of Artificial Sequence: Synthetic oligonucleotide
  BBa_I712074
agggaatacaagctacttgttctttttgca

SEQ ID NO: 708 - DNA, 23 bp, Artificial Sequence
  Description of Artificial Sequence: Synthetic oligonucleotide
  BBa_I719005
taatacgactcactatagggaga

SEQ ID NO: 709 - DNA, 28 bp, Artificial Sequence
  Description of Artificial Sequence: Synthetic oligonucleotide
  BBa_J34814
gaatttaatacgactcactatagggaga

SEQ ID NO: 710 - DNA, 19 bp, Artificial Sequence
  Description of Artificial Sequence: Synthetic oligonucleotide
  BBa_J64997
taatacgactcactatagg

SEQ ID NO: 711 - DNA, 30 bp, Artificial Sequence
  Description of Artificial Sequence: Synthetic oligonucleotide
  BBa_K113010
gagtcgtattaatacgactcactatagggg

SEQ ID NO: 712 - DNA, 30 bp, Artificial Sequence
  Description of Artificial Sequence: Synthetic oligonucleotide
  BBa_K113011
agtgagtcgtactacgactcactatagggg

SEQ ID NO: 713 - DNA, 30 bp, Artificial Sequence
  Description of Artificial Sequence: Synthetic oligonucleotide
  BBa_K113012
gagtcgtattaatacgactctctatagggg

SEQ ID NO: 714 - DNA, 18 bp, Artificial Sequence
  Description of Artificial Sequence: Synthetic oligonucleotide
  BBa_K1614000
taatacgactcactatag

SEQ ID NO: 715 - DNA, 23 bp, Artificial Sequence
  Description of Artificial Sequence: Synthetic oligonucleotide
  BBa_R0085
taatacgactcactatagggaga

SEQ ID NO: 716 - DNA, 23 bp, Artificial Sequence
  Description of Artificial Sequence: Synthetic oligonucleotide
  BBa_R0180
ttatacgactcactatagggaga

SEQ ID NO: 717 - DNA, 23 bp, Artificial Sequence
  Description of Artificial Sequence: Synthetic oligonucleotide
  BBa_R0181
gaatacgactcactatagggaga

SEQ ID NO: 718 - DNA, 23 bp, Artificial Sequence
  Description of Artificial Sequence: Synthetic oligonucleotide
  BBa_R0182
taatacgtctcactatagggaga

SEQ ID NO: 719 - DNA, 23 bp, Artificial Sequence
  Description of Artificial Sequence: Synthetic oligonucleotide
  BBa_R0183
tcatacgactcactatagggaga

SEQ ID NO: 720 - DNA, 30 bp, Artificial Sequence
  Description of Artificial Sequence: Synthetic oligonucleotide
  BBa_Z0251
taatacgactcactatagggagaccacaac

SEQ ID NO: 721 - DNA, 30 bp, Artificial Sequence
  Description of Artificial Sequence: Synthetic oligonucleotide
  BBa_Z0252
taattgaactcactaaagggagaccacagc

SEQ ID NO: 722 - DNA, 30 bp, Artificial Sequence
  Description of Artificial Sequence: Synthetic oligonucleotide
  BBa_Z0253
cgaagtaatacgactcactattagggaaga

SEQ ID NO: 723 - DNA, 19 bp, Artificial Sequence
  Description of Artificial Sequence: Synthetic oligonucleotide
atttaggtgacactataga

SEQ ID NO: 724 - DNA, 30 bp, Artificial Sequence
  Description of Artificial Sequence: Synthetic oligonucleotide
  BBa_I766555
acaaacacaaatacacacactaaattaata

SEQ ID NO: 725 - DNA, 30 bp, Artificial Sequence
  Description of Artificial Sequence: Synthetic oligonucleotide
  BBa_I766556
ccaagcatacaatcaactatctcatataca

SEQ ID NO: 726 - DNA, 30 bp, Artificial Sequence
  Description of Artificial Sequence: Synthetic oligonucleotide
  BBa_I766557
gatacaggatacagcggaaacaacttttaa

SEQ ID NO: 727 - DNA, 30 bp, Artificial Sequence
  Description of Artificial Sequence: Synthetic oligonucleotide
  BBa_J63005
tttcaagctataccaagcatacaatcaact

SEQ ID NO: 728 - DNA, 30 bp, Artificial Sequence
  Description of Artificial Sequence: Synthetic oligonucleotide
  BBa_K105027
cctttgcagcataaattactatacttctat

SEQ ID NO: 729 - DNA, 30 bp, Artificial Sequence; identical to SEQ ID NO: 728
  Description of Artificial Sequence: Synthetic oligonucleotide
  BBa_K105028

SEQ ID NO: 730 - DNA, 30 bp, Artificial Sequence; identical to SEQ ID NO: 728
  Description of Artificial Sequence: Synthetic oligonucleotide
  BBa_K105029

SEQ ID NO: 731 - DNA, 30 bp, Artificial Sequence; identical to SEQ ID NO: 728
  Description of Artificial Sequence: Synthetic oligonucleotide
  BBa_K105030

SEQ ID NO: 732 - DNA, 30 bp, Artificial Sequence; identical to SEQ ID NO: 728
  Description of Artificial Sequence: Synthetic oligonucleotide
  BBa_K105031

SEQ ID NO: 733 - DNA, 30 bp, Artificial Sequence
  Description of Artificial Sequence: Synthetic oligonucleotide
  BBa_K122000
ttatctactttttacaacaaatataaaaca

SEQ ID NO: 734 - DNA, 30 bp, Artificial Sequence; identical to SEQ ID NO: 724
  Description of Artificial Sequence: Synthetic oligonucleotide
  BBa_K124000

SEQ ID NO: 735 - DNA, 30 bp, Artificial Sequence
  Description of Artificial Sequence: Synthetic oligonucleotide
  BBa_K124002
gtttcgaataaacacacataaacaaacaaa

SEQ ID NO: 736 - DNA, 30 bp, Artificial Sequence; identical to SEQ ID NO: 725
  Description of Artificial Sequence: Synthetic oligonucleotide
  BBa_K319005

SEQ ID NO: 737 - DNA, 30 bp, Artificial Sequence
  Description of Artificial Sequence: Synthetic oligonucleotide
  BBa_M31201
accatcaaaggaagctttaatcttctcata

SEQ ID NO: 738 - DNA, 30 bp, Artificial Sequence
  Description of Artificial Sequence: Synthetic oligonucleotide
  BBa_I712004
agaacccactgcttactggcttatcgaaat

SEQ ID NO: 739 - DNA, 30 bp, Artificial Sequence
  Description of Artificial Sequence: Synthetic oligonucleotide
  BBa_K076017
ggccgtttttggcttttttgttagacgaag

SEQ ID NO: 740 - DNA, 66 bp, Artificial Sequence
  Description of Artificial Sequence: Synthetic oligonucleotide
  Plpp
ataagtgccttcccatcaaaaaaatattctcaacataaaaaactttgtgtaatacttgtaacgcta

SEQ ID NO: 741 - DNA, 52 bp, Artificial Sequence
  Description of Artificial Sequence: Synthetic oligonucleotide
  PapFAB46
aaaaagagtattgacttcgcatctttttgtacctataatagattcattgcta

SEQ ID NO: 742 - DNA, 59 bp, Artificial Sequence
  Description of Artificial Sequence: Synthetic oligonucleotide
  PJ23101+UP element
ggaaaatttttttaaaaaaaaaactttacagctagctcagtcctaggtattatgctagc

SEQ ID NO: 743 - DNA, 59 bp, Artificial Sequence
  Description of Artificial Sequence: Synthetic oligonucleotide
  PJ23107+UP element
ggaaaatttttttaaaaaaaaaactttacggctagctcagccctaggtattatgctagc

SEQ ID NO: 744 - DNA, 64 bp, Artificial Sequence
  Description of Artificial Sequence: Synthetic oligonucleotide
  PSYN23119
ggaaaatttttttaaaaaaaaaacttgacagctagctcagtccttggtataatgctagcacgaa

SEQ ID NO: 745 - protein, 21 aa, Artificial Sequence
  Description of Artificial Sequence: Synthetic peptide
  PhoA
MKQSTIALALLPLLFTPVTKA

SEQ ID NO: 746 - protein, 20 aa, Artificial Sequence
  Description of Artificial Sequence: Synthetic peptide
  PhoA
KQSTIALALLPLLFTPVTKA

SEQ ID NO: 747 - protein, 22 aa, Artificial Sequence
  Description of Artificial Sequence: Synthetic peptide
  OmpF
MMKRNILAVIVPALLVAGTANA

SEQ ID NO: 748 - protein, 15 aa, Artificial Sequence
  Description of Artificial Sequence: Synthetic peptide
  cvaC
MRTLTLNELDSVSGG

SEQ ID NO: 749 - protein, 43 aa, Artificial Sequence
  Description of Artificial Sequence: Synthetic polypeptide
  TorA
MNNNDLFQASRRRFLAQLGGLTVAGMLGTSLLTPRRATAAQAA

SEQ ID NO: 750 - protein, 33 aa, Artificial Sequence
  Description of Artificial Sequence: Synthetic polypeptide
  fdnG
MDVSRRQFFKICAGGMAGTTVAALGFAPKQALA

SEQ ID NO: 751 - protein, 45 aa, Artificial Sequence
  Description of Artificial Sequence: Synthetic polypeptide
  dmsA
MKTKIPDAVLAAEVSRRGLVKTTAIGGLAMASSALTLPFSRIAHA

SEQ ID NO: 752 - protein, 21 aa, Artificial Sequence
  Description of Artificial Sequence: Synthetic peptide
  PelB
KYLLPTAAAGLLLLAAQPAMA

SEQ ID NO: 753 - protein, 52 aa, Artificial Sequence
  Description of Artificial Sequence: Synthetic polypeptide
  HlyA secretion signal
LNPLINEISKIISAAGNFDVKEERAAASLLQLSGNASDFSYGRNSITLTASA

SEQ ID NO: 754 - DNA, 159 bp, Artificial Sequence
  Description of Artificial Sequence: Synthetic polynucleotide
  HlyA secretion signal
cttaatccattaattaatgaaatcagcaaaatcatttcagctgcaggtaattttgatgttaaagaggaaagagctgcagcttctttattgcagttgtccggtaatgccagtgatttttcatatggacggaactcaataactttgacagcatcagcataa

SEQ ID NO: 755-868 - skipped (114 entries)

SEQ ID NO: 869 - DNA, 28 bp, Artificial Sequence
  Description of Artificial Sequence: Synthetic oligonucleotide
  BBa_J61133
tctagagaaagacccgagacactagatg

SEQ ID NO: 870 - DNA, 28 bp, Artificial Sequence
  Description of Artificial Sequence: Synthetic oligonucleotide
  BBa_J61134
tctagagaaagaccggaaatactagatg

SEQ ID NO: 871 - DNA, 28 bp, Artificial Sequence
  Description of Artificial Sequence: Synthetic oligonucleotide
  BBa_J61135
tctagagaaagaccggagacactagatg

SEQ ID NO: 872 - skipped

SEQ ID NO: 873 - DNA, 28 bp, Artificial Sequence
  Description of Artificial Sequence: Synthetic oligonucleotide
  BBa_J61132
tctagagaaagacaggattaactagatg

SEQ ID NO: 874 - DNA, 28 bp, Artificial Sequence
  Description of Artificial Sequence: Synthetic oligonucleotide
  BBa_J61136
tctagagaaagagctgagcaactagatg

SEQ ID NO: 875 - DNA, 28 bp, Artificial Sequence
  Description of Artificial Sequence: Synthetic oligonucleotide
  BBa_J61137
tctagagaaagagtagatcaactagatg

SEQ ID NO: 876 - DNA, 28 bp, Artificial Sequence
  Description of Artificial Sequence: Synthetic oligonucleotide
  BBa_J61138
tctagagaaagatatgaataactagatg

SEQ ID NO: 877 - DNA, 28 bp, Artificial Sequence
  Description of Artificial Sequence: Synthetic oligonucleotide
  BBa_J61139
tctagagaaagattagagtcactagatg

SEQ ID NO: 878-879 - skipped (2 entries)

SEQ ID NO: 880 - DNA, 31 bp, Artificial Sequence
  Description of Artificial Sequence: Synthetic oligonucleotide
  BBa_B0029
tctagagttcacacaggaaacctactagatg

SEQ ID NO: 881 - DNA, 31 bp, Artificial Sequence
  Description of Artificial Sequence: Synthetic oligonucleotide
  BBa_B0030
tctagagattaaagaggagaaatactagatg

SEQ ID NO: 882 - DNA, 30 bp, Artificial Sequence
  Description of Artificial Sequence: Synthetic oligonucleotide
  BBa_B0031
tctagagtcacacaggaaacctactagatg

SEQ ID NO: 883 - DNA, 29 bp, Artificial Sequence
  Description of Artificial Sequence: Synthetic oligonucleotide
  BBa_B0032
tctagagtcacacaggaaagtactagatg

SEQ ID NO: 884 - DNA, 27 bp, Artificial Sequence
  Description of Artificial Sequence: Synthetic oligonucleotide
  BBa_B0033
tctagagtcacacaggactactagatg

SEQ ID NO: 885 - DNA, 28 bp, Artificial Sequence
  Description of Artificial Sequence: Synthetic oligonucleotide
  BBa_B0034
tctagagaaagaggagaaatactagatg

SEQ ID NO: 886 - DNA, 30 bp, Artificial Sequence
  Description of Artificial Sequence: Synthetic oligonucleotide
  BBa_B0035
tctagagattaaagaggagaatactagatg

SEQ ID NO: 887 - DNA, 28 bp, Artificial Sequence
  Description of Artificial Sequence: Synthetic oligonucleotide
  BBa_B0064
tctagagaaagaggggaaatactagatg

SEQ ID NO: 888-978 - skipped (91 entries)

SEQ ID NO: 979 - DNA, 45 bp, Artificial Sequence
  Description of Artificial Sequence: Synthetic oligonucleotide
  scFvLinker
ggtggaggcggctctggcggtggtgggagtggaggcggtggcagt

SEQ ID NO: 980-989 - skipped (10 entries)

SEQ ID NO: 990 - protein, 607 aa, Artificial Sequence
  Description of Artificial Sequence: Synthetic polypeptide
  Invasin display tag
MVFQPISEFLLIRNAGMSMYFNKIISFNIISRIVICIFLICGMFMAGASEKYDANAPQQVQPYSVSSSAFENLHPNNEMESSINPFSASDTERNAAIIDRANKEQETEAVNKMISTGARLAASGRASDVAHSMVGDAVNQEIKQWLNRFGTAQVNLNFDKNFSLKESSLDWLAPWYDSASFLFFSQLGIRNKDSRNTLNLGVGIRTLENGWLYGLNTFYDNDLTGHNHRIGLGAEAWTDYLQLAANGYFRLNGWHSSRDFSDYKERPATGGDLRANAYLPALPQLGGKLMYEQYTGERVALFGKDNLQRNPYAVTAGINYTPVPLLTVGVDQRMGKSSKHETQWNLQMNYRLGESFQSQLSPSAVAGTRLLAESRYNLVDRNNNIVLEYQKQQVVKLTLSPATISGLPGQVYQVNAQVQGASAVREIVWSDAELIAAGGTLTPLSTTQFNLVLPPYKRTAQVSRVTDDLTANFYSLSALAVDHQGNRSNSFTLSVTVQQPQLTLTAAVIGDGAPANGKTAITVEFTVADFEGKPLAGQEVVITTNNGALPNKITEKTDANGVARIALTNTTDGVTVVTAEVEGQRQSVDTHFVKGTIAADKSTLAAV

SEQ ID NO: 991 - protein, 148 aa, Artificial Sequence
  Description of Artificial Sequence: Synthetic polypeptide
  LppOmpA display tag
KATKLVLGAVILGSTLLAGCSSNAKIDQGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGIPG

SEQ ID NO: 992 - protein, 658 aa, Artificial Sequence
  Description of Artificial Sequence: Synthetic polypeptide
  IntiminN display tag
ITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYYRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDQTKAS

SEQ ID NO: 993-1017 - skipped (25 entries)

SEQ ID NO: 1018 - DNA, 28 bp, Artificial Sequence
  Description of Artificial Sequence: Synthetic oligonucleotide
  Master Sequence
  modified_base (13)..(15): a, c, g or t
  modified_base (18)..(20): a, c, g or t
tctagagaaagannngannnactagatg

SEQ ID NO: 1019 - DNA, 28 bp, Artificial Sequence
  Description of Artificial Sequence: Synthetic oligonucleotide
  BBa_J61100
tctagagaaagaggggacaaactagatg

SEQ ID NO: 1020 - DNA, 28 bp, Artificial Sequence
  Description of Artificial Sequence: Synthetic oligonucleotide
  BBa_J61101
tctagagaaagacaggacccactagatg

SEQ ID NO: 1021 - DNA, 28 bp, Artificial Sequence
  Description of Artificial Sequence: Synthetic oligonucleotide
  BBa_J61102
tctagagaaagatccgatgtactagatg

SEQ ID NO: 1022 - DNA, 28 bp, Artificial Sequence
  Description of Artificial Sequence: Synthetic oligonucleotide
  BBa_J61103
tctagagaaagattagacaaactagatg

SEQ ID NO: 1023 - DNA, 28 bp, Artificial Sequence
  Description of Artificial Sequence: Synthetic oligonucleotide
  BBa_J61104
tctagagaaagaagggacagactagatg

SEQ ID NO: 1024 - DNA, 28 bp, Artificial Sequence
  Description of Artificial Sequence: Synthetic oligonucleotide
  BBa_J61105
tctagagaaagacatgacgtactagatg

SEQ ID NO: 1025 - DNA, 28 bp, Artificial Sequence
  Description of Artificial Sequence: Synthetic oligonucleotide
  BBa_J61106
tctagagaaagataggagacactagatg

SEQ ID NO: 1026 - DNA, 28 bp, Artificial Sequence
  Description of Artificial Sequence: Synthetic oligonucleotide
  BBa_J61107
tctagagaaagaagagactcactagatg

SEQ ID NO: 1027 - DNA, 28 bp, Artificial Sequence
  Description of Artificial Sequence: Synthetic oligonucleotide
  BBa_J61108
tctagagaaagacgagatatactagatg

SEQ ID NO: 1028 - DNA, 28 bp, Artificial Sequence
  Description of Artificial Sequence: Synthetic oligonucleotide
  BBa_J61109
tctagagaaagactggagacactagatg

SEQ ID NO: 1029 - DNA, 28 bp, Artificial Sequence
  Description of Artificial Sequence: Synthetic oligonucleotide
  BBa_J61110
tctagagaaagaggcgaattactagatg

SEQ ID NO: 1030 - DNA, 28 bp, Artificial Sequence
  Description of Artificial Sequence: Synthetic oligonucleotide
  BBa_J61111
tctagagaaagaggcgatacactagatg

SEQ ID NO: 1031 - DNA, 28 bp, Artificial Sequence
  Description of Artificial Sequence: Synthetic oligonucleotide
  BBa_J61112
tctagagaaagaggtgacatactagatg

SEQ ID NO: 1032 - DNA, 28 bp, Artificial Sequence
  Description of Artificial Sequence: Synthetic oligonucleotide
  BBa_J61113
tctagagaaagagtggaaaaactagatg

SEQ ID NO: 1033 - DNA, 28 bp, Artificial Sequence
  Description of Artificial Sequence: Synthetic oligonucleotide
  BBa_J61114
tctagagaaagatgagaagaactagatg

SEQ ID NO: 1034 - DNA, 28 bp, Artificial Sequence
  Description of Artificial Sequence: Synthetic oligonucleotide
  BBa_J61115
tctagagaaagaagggatacactagatg

SEQ ID NO: 1035 - DNA, 28 bp, Artificial Sequence
  Description of Artificial Sequence: Synthetic oligonucleotide
  BBa_J61116
tctagagaaagacatgaggcactagatg

SEQ ID NO: 1036 - DNA, 28 bp, Artificial Sequence
  Description of Artificial Sequence: Synthetic oligonucleotide
  BBa_J61117
tctagagaaagacatgagttactagatg

SEQ ID NO: 1037 - DNA, 28 bp, Artificial Sequence
  Description of Artificial Sequence: Synthetic oligonucleotide
  BBa_J61118
tctagagaaagagacgaatcactagatg

SEQ ID NO: 1038 - DNA, 28 bp, Artificial Sequence
  Description of Artificial Sequence: Synthetic oligonucleotide
  BBa_J61119
tctagagaaagatttgatatactagatg

SEQ ID NO: 1039 - DNA, 28 bp, Artificial Sequence
  Description of Artificial Sequence: Synthetic oligonucleotide
  BBa_J61120
tctagagaaagacgcgagaaactagatg

SEQ ID NO: 1040 - DNA, 28 bp, Artificial Sequence
  Description of Artificial Sequence: Synthetic oligonucleotide
  BBa_J61121
tctagagaaagagacgagtcactagatg

SEQ ID NO: 1041 - DNA, 28 bp, Artificial Sequence
  Description of Artificial Sequence: Synthetic oligonucleotide
  BBa_J61122
tctagagaaagagaggagccactagatg

SEQ ID NO: 1042 - DNA, 28 bp, Artificial Sequence
  Description of Artificial Sequence: Synthetic oligonucleotide
  BBa_J61123
tctagagaaagagatgactaactagatg

SEQ ID NO: 1043 - DNA, 28 bp, Artificial Sequence
  Description of Artificial Sequence: Synthetic oligonucleotide
  BBa_J61124
tctagagaaagagccgacatactagatg

SEQ ID NO: 1044 - DNA, 28 bp, Artificial Sequence
  Description of Artificial Sequence: Synthetic oligonucleotide
  BBa_J61125
tctagagaaagagccgagttactagatg

SEQ ID NO: 1045 - DNA, 28 bp, Artificial Sequence
  Description of Artificial Sequence: Synthetic oligonucleotide
  BBa_J61126
tctagagaaagaggtgactcactagatg

SEQ ID NO: 1046 - DNA, 28 bp, Artificial Sequence
  Description of Artificial Sequence: Synthetic oligonucleotide
  BBa_J61127
tctagagaaagagtggaactactagatg

SEQ ID NO: 1047 - DNA, 28 bp, Artificial Sequence
  Description of Artificial Sequence: Synthetic oligonucleotide
  BBa_J61128
tctagagaaagataggactcactagatg

SEQ ID NO: 1048 - DNA, 28 bp, Artificial Sequence
  Description of Artificial Sequence: Synthetic oligonucleotide
  BBa_J61129
tctagagaaagattggacgtactagatg

SEQ ID NO: 1049 - DNA, 28 bp, Artificial Sequence
  Description of Artificial Sequence: Synthetic oligonucleotide
  BBa_J61130
tctagagaaagaaacgacatactagatg

SEQ ID NO: 1050 - DNA, 28 bp, Artificial Sequence
  Description of Artificial Sequence: Synthetic oligonucleotide
  BBa_J61131
tctagagaaagaaccgaattactagatg

SEQ ID NO: 1051-1134 - skipped (84 entries)

SEQ ID NO: 1135 - protein, 24 aa, Artificial Sequence
  Description of Artificial Sequence: Synthetic peptide
MFKSTLAAMAAVFALSALSPAAMA

SEQ ID NO: 1136-1140 - skipped (5 entries)

SEQ ID NO: 1141 - DNA, 72 bp, Artificial Sequence
  Description of Artificial Sequence: Synthetic oligonucleotide
atgtttaagtctacacttgcagccatggccgcagtcttcgcactgtcagccttgagtcctgctgcaatggca

SEQ ID NO: 1142 - DNA, 63 bp, Artificial Sequence
  Description of Artificial Sequence: Synthetic oligonucleotide
atgaagcaggcgcttcgcgttgcgtttgggttccttatcctgtgggcatccgtacttcacgcc

SEQ ID NO: 1143 - DNA, 54 bp, Artificial Sequence
  Description of Artificial Sequence: Synthetic oligonucleotide
atgcgcgtactgttattccttctgttgtctctttttatgttgcccgctttcagt

SEQ ID NO: 1144 - DNA, 63 bp, Artificial Sequence
  Description of Artificial Sequence: Synthetic oligonucleotide
atgaagaagactgccatcgctattgccgtcgcccttgcgggtttcgcaaccgtggcgcaagca

SEQ ID NO: 1145 - DNA, 66 bp, Artificial Sequence
  Description of Artificial Sequence: Synthetic oligonucleotide
atgaaatatctgcttccaacggctgctgctggcctgttgcttcttgccgcccagcctgcgatggct

SEQ ID NO: 1146-1156 - skipped (11 entries)

SEQ ID NO: 1157 - DNA, 99 bp, Artificial Sequence
  Description of Artificial Sequence: Synthetic oligonucleotide
atggggtacaaaatgaacattagctcgcttcgcaaagcattcatttttatgggggctgttgcagctttaagccttgtcaatgcccagtcagcgcttgcc

SEQ ID NO: 1158 - DNA, 57 bp, Artificial Sequence
  Description of Artificial Sequence: Synthetic oligonucleotide
atgaaaaagatttggctggctcttgccggtttagtcctggcattcagcgcaagcgcg

SEQ ID NO: 1159 - DNA, 63 bp, Artificial Sequence
  Description of Artificial Sequence: Synthetic oligonucleotide
atgaaacagagcacaatcgctttagccttgctgcctcttcttttcacgcctgtcacgaaggca

SEQ ID NO: 1160 - DNA, 63 bp, Artificial Sequence
  Description of Artificial Sequence: Synthetic oligonucleotide
atgaagcaggctctgcgcgtggcatttgggttcctgatcttatgggcgtccgtcttacacgca

SEQ ID NO: 1161 - DNA, 78 bp, Artificial Sequence
  Description of Artificial Sequence: Synthetic oligonucleotide
atgaaaatcaagacgggggcacgcattcttgccttgagcgccttgacaacgatgatgttcagtgcaagtgcattagcg

SEQ ID NO: 1162 - DNA, 69 bp, Artificial Sequence
  Description of Artificial Sequence: Synthetic oligonucleotide
atgaataagaaagtcttgacattgtcggcggtcatggcctcgatgttgttcggggcagcagcccacgca

SEQ ID NO: 1163 - DNA, 66 bp, Artificial Sequence
  Description of Artificial Sequence: Synthetic oligonucleotide
atgatgaagcgtaacatcttagccgttattgtccccgcattgcttgtggccgggacggctaacgca

SEQ ID NO: 1164 - DNA, 63 bp, Artificial Sequence; identical to SEQ ID NO: 1144
  Description of Artificial Sequence: Synthetic oligonucleotide

SEQ ID NO: 1165 - DNA, 54 bp, Artificial Sequence
  Description of Artificial Sequence: Synthetic oligonucleotide
atgcgcgtgttattgttcttgctgctgagcttgtttatgttaccagctttcagt

SEQ ID NO: 1166 - DNA, 75 bp, Artificial Sequence
  Description of Artificial Sequence: Synthetic oligonucleotide
atgatgattaccttacgcaagttgccccttgcggtagccgttgctgctggtgtgatgtccgcgcaagcaatggct

SEQ ID NO: 1167 - DNA, 66 bp, Artificial Sequence
  Description of Artificial Sequence: Synthetic oligonucleotide
atgaaatatcttcttccaacggctgctgctggtttattgcttcttgccgcccagcctgcgatggct

SEQ ID NO: 1168-1179 - skipped (12 entries)

SEQ ID NO: 1180 - protein, 33 aa, Artificial Sequence
  Description of Artificial Sequence: Synthetic polypeptide
MGYKMNISSLRKAFIFMGAVAALSLVNAQSALA

SEQ ID NO: 1181 - protein, 19 aa, Artificial Sequence
  Description of Artificial Sequence: Synthetic peptide
MKKIWLALAGLVLAFSASA

SEQ ID NO: 1182 - protein, 21 aa, Artificial Sequence
  Description of Artificial Sequence: Synthetic peptide
MKQSTIALALLPLLFTPVTKA

SEQ ID NO: 1183 - protein, 21 aa, Artificial Sequence
  Description of Artificial Sequence: Synthetic peptide
MKQALRVAFGFLILWASVLHA

SEQ ID NO: 1184 - protein, 26 aa, Artificial Sequence
  Description of Artificial Sequence: Synthetic peptide
MKIKTGARILALSALTTMMFSASALA

SEQ ID NO: 1185 - protein, 23 aa, Artificial Sequence
  Description of Artificial Sequence: Synthetic peptide
MNKKVLTLSAVMASMLFGAAAHA

SEQ ID NO: 1186 - protein, 26 aa, Artificial Sequence
  Description of Artificial Sequence: Synthetic peptide
MTNITKRSLVAAGVLAALMAGNVALA

SEQ ID NO: 1187 - protein, 21 aa, Artificial Sequence
  Description of Artificial Sequence: Synthetic peptide
MKKTAIAIAVALAGFATVAQA

SEQ ID NO: 1188 - protein, 18 aa, Artificial Sequence
  Description of Artificial Sequence: Synthetic peptide
MRVLLFLLLSLFMLPAFS

SEQ ID NO: 1189 - protein, 25 aa, Artificial Sequence
  Description of Artificial Sequence: Synthetic peptide
MMITLRKLPLAVAVAAGVMSAQAMA

SEQ ID NO: 1190 - protein, 22 aa, Artificial Sequence
  Description of Artificial Sequence: Synthetic peptide
MKYLLPTAAAGLLLLAAQPAMA

SEQ ID NO: 1191-1217 - skipped (27 entries)

SEQ ID NO: 1218 - protein, 52 aa, Artificial Sequence
  Description of Artificial Sequence: Synthetic polypeptide
MKRHLNTSYRLVWNHITGAFVVASELARARGKRAGVAVALSLAAATSLPALA

SEQ ID NO: 1219 - protein, 53 aa, Artificial Sequence
  Description of Artificial Sequence: Synthetic polypeptide
MNKIFKVIWNPATGSYTVASETAKSRGKKSGRSKLLISALVAGGLLSSFGASA

SEQ ID NO: 1220 - protein, 22 aa, Artificial Sequence
  Description of Artificial Sequence: Synthetic peptide
MQLRKPATAILALALSAGLAQA

SEQ ID NO: 1221 - protein, 40 aa, Artificial Sequence
  Description of Artificial Sequence: Synthetic polypeptide
MFWRDMTLSVWRKKTTGLKTKKRLLALVLAAALCSSPVWA

SEQ ID NO: 1222 - DNA, 159 bp, Artificial Sequence
  Description of Artificial Sequence: Synthetic polynucleotide
atgaacaagattttcaaggttatctggaaccccgcgacaggcagctacactgtggcctccgaaaccgctaagtcgcgcggcaagaagtcaggacgttcaaagctgcttatttctgctttagtagccggaggattactttctagctttggagcctcggca

SEQ ID NO: 1223 - DNA, 57 bp, Artificial Sequence
  Description of Artificial Sequence: Synthetic oligonucleotide
atgaaaaaaatctggctggcattagcaggcttagtcctggcctttagtgcctcggca

SEQ ID NO: 1224 - DNA, 66 bp, Artificial Sequence
  Description of Artificial Sequence: Synthetic oligonucleotide
atgcagttgcgcaagcctgccacggcaattttagcattagctctttctgcgggcttggcccaggca

SEQ ID NO: 1225 - DNA, 120 bp, Artificial Sequence
  Description of Artificial Sequence: Synthetic polynucleotide
atgttctggcgcgacatgacactgagcgtttggcgtaaaaaaacaacgggactgaaaaccaaaaagcgtttgttagcccttgtcctggcggcagccctgtgctcgtccccggtatgggca

SEQ ID NO: 1226 - DNA, 99 bp, Artificial Sequence
  Description of Artificial Sequence: Synthetic oligonucleotide
atgggatacaagatgaacatttcatcacttcgcaaagcttttatcttcatgggtgccgttgctgctctgtcactggtcaatgctcaatcggccctggca

SEQ ID NO: 1227 - DNA, 78 bp, Artificial Sequence
  Description of Artificial Sequence: Synthetic oligonucleotide
atgaagattaaaacaggcgctcgtattttggccctgtctgcgcttacgactatgatgtttagtgcctcggccctggca

SEQ ID NO: 1228 - DNA, 78 bp, Artificial Sequence
  Description of Artificial Sequence: Synthetic oligonucleotide
atgacgaatatcactaaacgtagccttgtggccgcgggggtcttggctgcattgatggctggcaatgttgctctggca

SEQ ID NO: 1229 - DNA, 66 bp, Artificial Sequence
  Description of Artificial Sequence: Synthetic oligonucleotide
atgaaatatctgttgcccacggctgccgcgggtctgctgctgctggcagcgcaaccggctatggca

SEQ ID NO: 1230 - DNA, 63 bp, Artificial Sequence
  Description of Artificial Sequence: Synthetic oligonucleotide
atgaagcaaagtacgatcgcccttgcgctgttgccccttcttttcacgcccgtcaccaaggca

SEQ ID NO: 1231-1249 - skipped (19 entries)

SEQ ID NO: 1250 - DNA, 24 bp, Artificial Sequence
  Description of Artificial Sequence: Synthetic oligonucleotide
ggaaaatttttttaaaaaaaaaac

SEQ ID NO: 1251-1252 - skipped (2 entries)

SEQ ID NO: 1253 - DNA, 57 bp, Artificial Sequence
  Description of Artificial Sequence: Synthetic oligonucleotide
atgaagaaaatttggctggcacttgccggtttggtattagcgttttccgcctcggca

SEQ ID NO: 1254 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 1255 - DNA, 1569 bp, Homo sapiens
atgcaaccatggcatggtaaggcaatgcaacgtgcatctgaggctggagctaccgcgcccaaagcttcagcacggaacgcgcgcggtgctccgatggatccgacggagagccctgctgcaccggaagccgctctgccaaaagcgggtaaattcgggccggcgcggaaatcaggctcacgccagaaaaagagtgctccggatacacaggaacgtccgcctgtaagagctaccggcgcgcgcgctaaaaaagcccctcagcgtgcgcaggatacccaaccatcggatgcaacgagcgcaccgggggcggagggccttgagcctcctgctgcgcgtgagccggccctctctcgcgcagggagttgccgccaacgaggcgcacgctgctcgacgaaaccgcggccccctccgggtccgtgggatgttccatcgccaggcctgccggtttctgcgcctattctcgttcgccgcgacgcggctcctggcgcttcgaaactgagagctgtcttagagaaactgaaactctcgcgggatgatatatctactgcggcagggatggtcaaaggtgttgtggatcatctcctgttacgtcttaagtgcgattcggccttccgaggtgttggccttttaaacactgggtcatactatgagcacgttaagatcagcgcgccgaacgagttcgacgttatgtttaagcttgaagtccctcgcatacagctggaagaatactccaatacacgagcatattattttgttaaattcaaacgaaaccccaaagagaaccctctttcacagtttctggagggagaaattctgtctgcgagtaagatgctctcgaagttccgaaaaatcattaaagaggaaatcaacgacatcaaagacactgatgttatcatgaagaggaaacgtgggggctcaccggcggtaacactgctgatttcggaaaagatctcagttgatattactttggcgctcgaatcaaaatcatcgtggccggcgagtacacaggaagggctccgtattcagaattggctgtctgcgaaagtccgcaaacaactgcgtttgaaaccattttatttagtgccaaaacacgcgaaagaaggcaacggtttccaggaggagacgtggcgactgtcatttagccacatagaaaaggaaatattaaataatcacgggaaaagtaagacgtgctgcgagaataaagaagagaagtgctgcaggaaagattgtctgaaactgatgaaatacctgttggagcagctgaaagagaggtttaaagataagaagcatctggacaaattttcttcatatcacgttaaaaccgcattcttccacgtatgcacacagaatccgcaggactcacagtgggatcggaaagatctgggcctgtgctttgacaactgcgtcacatatttcttgcagtgcttgaggactgaaaaattggaaaactacttcattcccgagttcaatttattcagtagtaacctgatcgacaagcgtagcaaagaattcctgacgaagcagatcgaatacgaaagaaataacgaattcccggtgtttgatgaattttaa

SEQ ID NO: 1256 - skipped

SEQ ID NO: 1257 - protein, 273 aa, Listeria monocytogenes
MDFSNMSILHYLANIVDILVVWFVIYKVIMLIRGTKAVQLLKGIFIIIAVKLLSGFFGLQTVEWITDQMLTWGFLAIIIIFQPELRRALETLGRGNIFTRYGSRIEREQHHLIESIEKSTQYMAKRRIGALISVARDTGMDDYIETGIPLNAKISSQLLINIFIPNTPLHDGAVIIKGNEIASAASYLPLSDSPFLSKELGTRHRAALGISEVTDSITIVVSEETGGISLTKGGELFRDVSEEELHKILLKELVTVTAKKPSIFSKWKGGKSE

SEQ ID NO: 1258 - DNA, 822 bp, Listeria monocytogenes
atggacttttccaacatgagtatccttcactatttagcgaatattgtagacattttggtggtttggttcgtaatttacaaggttatcatgcttatccgcggcacgaaagccgtccagctgttgaaggggattttcattattattgccgtcaagttacttagcggcttcttcggattgcagacagtggaatggattactgatcaaatgctgacttggggtttcttagccattatcattatctttcaaccggaattgcgtcgtgccctggagactttggggcgtggcaatatctttacccgctatggatcacgcattgagcgtgaacaacaccaccttattgagtctattgaaaagtccacgcaatacatggcgaagcgtcgcattggagctttgatctctgtggctcgtgatacaggcatggacgactacatcgagactggcattccgcttaatgcgaaaatctcatcgcaattattgattaatatcttcatccccaatacccctcttcacgatggcgcagtgatcattaaaggtaacgagatcgcgagcgctgccagttatctgccattgtccgactcgccgtttctttctaaggagctgggaactcgccatcgtgccgcattagggatttccgaggtgacagattcaatcacaatcgttgtcagcgaggaaacaggtgggatttcccttacgaagggaggcgaactgttccgtgatgtatccgaagaagaattgcataagattttgcttaaagagctggtgactgtcacagctaaaaaaccaagcattttctccaaatggaaaggaggtaagtccgagtga

SEQ ID NO: 1259 - skipped

SEQ ID NO: 1260 - protein, 403 aa, Kingella denitrificans
MSDYTNNLHRVFVDQKIGFKKKITPTEQDLNFFNQVKKDVKSHLKTKIKEFLEQQGIANIAPKFRIQGSWAYGTCNLPAKQDQEMDFDYGVYLPVRAFEGFNPDAGASEQAQNYFEQVELMVEDLCHQHKWQLDTSAPSSCIRIKIRSNAHMDIPLYAVPDDMFDSLEERNELQVSLGTTTAIDKSLNYSEWALEDFNIRSFAEESLMDKNIRMIHMARRDGTWQKSDCELIRKWFADKLKSLENNGQQLRAICRYLKAWRDWQFADKSFQPSSILLMIIACKYYQYHQHRDDLALLSVLEKLPNALNDNVYENIEEHESEDFNRMEEYERVEASQYADALYQSFMASLNNLDKTKVLNFITNEWGDRIPQDENLIETSPQSIFDTPPLAQSNITQQVPLRQG

SEQ ID NO: 1261 - protein, 403 aa, Neisseria bacilliformis
MSHYTNNLHRIFVDREIGFKKEITPTELDLDFFNNVKKVVKSHLKTKIKEFLEQQGLASITPKFRIQGSWAYGTCNLPAKQGQEMDFDYGVYLPVRAFDGFNPDAGASEQAKNYFEQVELMMGDLCEQHDWLLDTSAPSSCIRIKIRNNAHMDIPLYAVPDDMFDSLEERNELQVSLGSATAIHESLNYSKWVFEDFNIRSFAEESLMDKNIRMIHMARRDGTWQESDCELIRKWFADKLKSLENNGQQLRAICRYLKAWRDWQFADKSFQPSSILLMIIACKYYQYYQHRDDLALLSVLEKLPNALSGNVYENIEEHESEDFNRMKEGERVEAMQYADALYQNFMASLNNFDKTKALKFITNEWGNRIPQDEDLIETSRQAIFDTPPLVQPNITPQAPLRQG

SEQ ID NO: 1262 - protein, 468 aa, Verminephrobacter eiseniae
MGQASKLFNGNTDQTLIGRVTPTTEQREFLQQQWNDLADHLKQALAKHGYTISTWLQGSYKYATLIKPVHLGEEYDVDVGLYFEWNDDQDAEPTPKQLRDWVQAELLEYEKACEELKKVEVPPKERCSRASYIQQFHIDTPVYHLNTDSDVRRLACLSGKWEHSDPKKLYKWFKEAVSGDDRDQLRRLVRYLKAWAAVSFDDAPGSRPSSIFLTVIATEAYQDLWAQRLLGLADDDALLAVIKKMHDRLFDDRKVENPVDKNEDLNRMTAEAWDGFLPRLAALQDIAERAGDAKDEASAALIWSEAFSFLMPLPETDQVEIVDEGSGRAVMQLPEIEVKVCTGTPPRPVATYRNEVPGVAKDCMLSFAIVNPHVVPEFATVEWTVRNEGQEADQRSDLGHRRVGMRLLSVEEHTAYVGRHFMDCVVRLNGQVYAVRRVPVTIRDVQHVARNPPRPPYTKLRSLFRRRR

SEQ ID NO: 1263 - DNA, 1213 bp, Kingella denitrificans
atgtcagactacactaataacttgcatcgcgtctttgttgaccaaaaaattgggttcaagaagaaaatcactcctaccgagcaagatctgaactttttcaatcaggtcaagaaagatgtaaagtcccaccttaagaccaaaatcaaggaatttcttgagcaacagggaatcgcgaacattgctccgaagttccgtattcagggatcgtgggcttacgggacatgtaatttgcccgcaaaacaggaccaggagatggatttcgattacggggtatatctgccagtacgtgccttcgaggggttcaacccagacgcaggcgcgtctgagcaggctcaaaactattttgaacaggtcgagttgatggttgaagatctgtgccaccagcataaatggcagctggacacgtctgccccttcgtcctgtatccgcattaaaattcgcagcaacgctcatatggacattcctttatatgccgtcccggatgatatgttcgattctcttgaagagcgcaatgagttacaagtatcactgggcacgacaacggctatcgataagtcactgaattacagcgagtgggcactggaggactttaatatccgctcattcgccgaagaaagtttaatggataagaacattcgcatgatccacatggcgcgtcgtgacgggacttggcaaaagtcggattgcgagctgattcgcaaatggttcgcagacaaactgaagagcttggagaataacgggcaacagctgcgtgcgatctgtcgttaccttaaggcgtggcgcgattggcaattcgctgacaagtcgtttcaaccatcctctatcttgttaatgattatcgcgtgcaaatattatcaataccatcaacaccgtgacgatttagcacttttatccgtgcttgaaaagttgccaaacgctttaaacgataatgtttatgagaatattgaagaacacgaatccgaagactttaaccgtatggaagagtatgaacgcgttgaggcgtcgcagtatgccgacgcgttgtaccagagtttcatggcctccttaaacaatttggacaaaactaaggttttaaattttatcactaatgagtggggggatcgcattccacaagacgagaacttaatcgagacatctcctcaatcaattttcgatactccccccttggcacaaagcaatatcacgcaacaagtgcccttgcgccagggttaas

SEQ ID NO: 1264 - DNA, 1212 bp, Neisseria bacilliformis
atgtctcactacaccaataatcttcatcgtatcttcgtagatcgtgaaattggttttaagaaagagattactccgacagagctggatcttgacttcttcaacaacgtcaaaaaagtagttaagagccaccttaaaaccaaaatcaaggagtttcttgaacagcaagggctggcgagtattactcctaagtttcgtatccagggaagctgggcgtacggaacatgtaaccttccagccaagcagggtcaggaaatggacttcgattatggcgtttatcttcctgtccgcgcctttgatggatttaatcctgatgcaggtgcctcagaacaagctaagaattacttcgaacaggtggagcttatgatgggtgacctgtgcgagcagcatgactggttgttggacacatccgcgccctcttcgtgtattcgcatcaaaatccgcaataatgcccatatggacattcctttatatgcagttccagacgatatgttcgatagcctggaggaacgtaacgaattacaggtttccttgggatcagcgacagctattcacgaatcgctgaattattcaaagtgggtttttgaggattttaacatccgctcttttgctgaggaatccctgatggacaaaaatatccgcatgatccacatggcccgccgtgacggaacttggcaggagtctgattgtgagttgattcgcaaatggtttgctgacaagttgaaatcccttgagaataatggtcagcagcttcgtgcaatttgtcgctatcttaaagcctggcgcgattggcaattcgcagataagtcttttcaaccctccagtatcctgttgatgatcattgcgtgtaagtactatcagtactaccaacatcgtgacgacctggcactgctgtccgtgctggagaaattgcccaacgctttgtctgggaacgtctatgagaatattgaggaacatgagtctgaggactttaaccgtatgaaagagggcgagcgtgtagaagcaatgcaatatgcggacgcgttataccaaaacttcatggcgtctcttaacaattttgataaaacgaaggcgctgaaattcatcactaatgaatggggaaatcgtatcccacaagatgaagacttaatcgaaacgtctcgtcaggcaatttttgacacaccacccttagttcaacccaacatcactccacaggccccacttcgccaggggtaa

SEQ ID NO: 1265 - DNA, 1407 bp, Verminephrobacter eiseniae
atgggacaggcgtctaagttatttaatggtaatacagatcaaactttaattggccgcgtgacgcccactaccgaacaacgcgagtttttacaacagcagtggaatgatctggcagatcacttgaagcaagccctggcaaaacacggttatacaatttcgacatggcttcagggttcttacaaatacgcgactcttattaagcctgtgcatctgggtgaagagtatgacgtagatgtagggttgtactttgagtggaacgatgaccaggatgcggagcccacgcctaagcagctgcgtgattgggtgcaagcggagttgttagagtatgagaaggcttgtgaggaacttaagaaagttgaggtgccgccgaaagaacgctgttctcgcgcatcatacatccaacaatttcacattgacacaccagtttatcatctgaacacggactccgacgtgcgccgtcttgcttgccttagtggaaagtgggagcattctgatccgaaaaagttatacaagtggtttaaagaagcagtatccggggatgatcgtgaccaattacgccgtcttgtacgttacttaaaggcctgggctgcggtttcctttgatgacgcaccgggatcccgcccatctagcatttttcttacggttattgctactgaagcctatcaggacttgtgggcacaacgtttactgggattagctgacgatgacgctttgctggcagtgattaaaaagatgcacgaccgccttttcgatgaccgcaaggtcgagaatcccgtagataaaaacgaagacttaaatcgcatgacggctgaagcgtgggacggttttctgccccgcttggcggcacttcaagacattgcggaacgcgccggagatgctaaggatgaggcgtcagccgcattgatctggagcgaagcgtttagttttttgatgcccttgccggaaactgatcaggtagaaatcgtagacgaaggctcaggccgtgccgtcatgcaattgccagaaattgaggtaaaggtctgcactggcacgccaccccgcccagtcgcaacgtatcgtaatgaggtcccaggagtagcaaaagactgcatgttgtcctttgcgattgtcaatccacatgtggttccggagtttgccacggtggagtggactgtgcgtaatgagggacaggaggcggaccagcgtagtgatttagggcaccgtcgcgtcgggatgcgtttgttgagtgtagaggaacatacggcatatgtcggtcgtcactttatggattgtgtagtccgtctgaacgggcaagtttacgctgtccgtcgcgtacccgttaccattcgcgacgttcagcacgtcgcccgcaatcccccacgtccaccttacacaaaactgcgcagtttattccgccgccgtcgctaa

SEQ ID NO: 1266-1267 - skipped (2 entries)

SEQ ID NO: 1268 - DNA, 621 bp, Artificial Sequence
  Description of Artificial Sequence: Synthetic polynucleotide
ctaacgtttgaattttgcgtaacgttcgcgagcaatctctaaagtgctattacgtacacgctcaaaacgttccttatccttctgccataacgaacgcaccgcaagaccacgcacgctgttaaaaatcaaccacaaaatgtcctcggcatcgtcacgggataatccacgcgacaccagaacgcccaaccacatatcctcgaccacaaaacggttgcgctctaccgtgcgttggataccctcacgcagagctggatcacgatcagcggccacaatcagatcaaggctaattgaaaagtcgtcatccaagaaaaactcagcggcatcatcaagcatttgctgaatcacatcatcttccggttttaacttagccaggcgagcacggctgcgctcggtgatctgctcatacagccactcaaacgtagcaagcaacagttctaatttggtgggaaaatggtgtgactgggcgccacgagacacccccgcggcgcccggtacatccgcaatacgaaaacccgcgtatcccttctcgcgtaatacaccaagcgccgccgcgatcagtttaccttgtgtttccatcgcacgttcggcttgagtacggcgcttgggcgacatgattaccat

SEQ ID NO: 1269 - protein, 206 aa, Artificial Sequence
  Description of Artificial Sequence: Synthetic polypeptide
MVIMSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSISLDLIVAADRDPALREGIQRTVERNRFVVEDMWLGVLVSRGLSRDDAEDILWLIFNSVRGLAVRSLWQKDKERFERVRNSTLEIARERYAKFKR

SEQ ID NO: 1270 - DNA, 123 bp, Escherichia coli
taacaccgtgcgtgttgactattttacctctggcggtgataatggttgcaacaaacagacaatctggtctgtttgtattatgttaaccagcacactggcggccgcttactagaaataattttg

SEQ ID NO: 1271 - DNA, 161 bp, Artificial Sequence
  Description of Artificial Sequence: Synthetic polynucleotide
taacaccgtgcgtgttgactattttacctctggcggtgataatggttgcaacaaacagacaatctggtctgtttgtattatgttaaccagcacactggcggccgcttactagaaataattttggccgcatcccacgctaactaatataaaggaggttttaa

SEQ ID NO: 1272 - skipped

SEQ ID NO: 1273 - DNA, 79 bp, Artificial Sequence
  Description of Artificial Sequence: Synthetic oligonucleotide
tgtatttatcaatattgtttgctccgttatcgttattaacaagtcatcaataaagccatcacgagtaccatagaggatc

SEQ ID NO: 1274 - DNA, 117 bp, Artificial Sequence
  Description of Artificial Sequence: Synthetic polynucleotide
tgtatttatcaatattgtttgctccgttatcgttattaacaagtcatcaataaagccatcacgagtaccatagaggatcgccgcatcccacgctaactaatataaaggaggttttaa

SEQ ID NO: 1275-1277 - skipped (3 entries)

SEQ ID NO: 1278 - DNA, 903 bp, Pseudomonas sp.
tcaatctgtaaacaaatcgaacatcagttgacgaagccagatattggccaaatccttatggtatttagcgtgccagaacatattaatggcaatctcgggaagcactaccggatgaggcagagcggacaagccgaaaggttcgacacagcaatccgctaagcggatcggcactgttgccagcaggtcagtacgttgaagaatatggccaacggctgcgaaatggggcacttccagacgaatatcacgacggatgcctacacgggtcatatacgtgtccacttcaccgtggcctgtgcctgctgcaataacacgtacatgtccataactacaaaagcgttcaagcgtcaatggctcacgggtaacggggtggtccttgcggcacaaacaaacgtagtggttttgcagtaaacggcgttggaagaagcctgtttgaaggttgggcagaagccccaccgccaaatcgacagttccattttgcaacgcctgcattaatgacatagacgaatcgcggacagtagaaatgacgcagttcggtgcttgatgggcaagtacgtccatcaggcgaggcatgaaataaatctccccaatatccgtcattgccagggtgaaagtacgctccgaagtaagggggtcaaagctctcatgatgttgtaaagcgttacgaagggcgtgcatggcactggtgacaggctcggccaagtgtgcggcgtatggcgttggttccatcccttggtgagtacggacgaacaacggatcttgtaacgatgtacgaaggcgttttaatgcgttagaaactgcgggttgagtaagcccaagattttctgcggtgatggagacgcgacgatctaccagcagctgattaaagacaactaacaggttaaggtccagatcacgcaattccat

SEQ ID NO: 1279 - skipped

SEQ ID NO: 1280 - protein, 300 aa, Pseudomonas sp.
MELRDLDLNLLVVFNQLLVDRRVSITAENLGLTQPAVSNALKRLRTSLQDPLFVRTHQGMEPTPYAAHLAEPVTSAMHALRNALQHHESFDPLTSERTFTLAMTDIGEIYFMPRLMDVLAHQAPNCVISTVRDSSMSLMQALQNGTVDLAVGLLPNLQTGFFQRRLLQNHYVCLCRKDHPVTREPLTLERFCSYGHVRVIAAGTGHGEVDTYMTRVGIRRDIRLEVPHFAAVGHILQRTDLLATVPIRLADCCVEPFGLSALPHPVVLPEIAINMFWHAKYHKDLANIWLRQLMFDLFTD

SEQ ID NO: 1281 - DNA, 164 bp, Artificial Sequence
  Description of Artificial Sequence: Synthetic polynucleotide
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaa

SEQ ID NO: 1282 - DNA, 201 bp, Artificial Sequence
  Description of Artificial Sequence: Synthetic polynucleotide
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaacccgcatcccacgctaactaatataaggggggcaaga

SEQ ID NO: 1283 - skipped

SEQ ID NO: 1284 - DNA, 1023 bp, Artificial Sequence
  Description of Artificial Sequence: Synthetic polynucleotide
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaacccgcatcccacgctaactaatataaggggggcaagaatggacttttccaacatgagtatccttcactatttagcgaatattgtagacattttggtggtttggttcgtaatttacaaggttatcatgcttatccgcggcacgaaagccgtccagctgttgaaggggattttcattattattgccgtcaagttacttagcggcttcttcggattgcagacagtggaatggattactgatcaaatgctgacttggggtttcttagccattatcattatctttcaaccggaattgcgtcgtgccctggagactttggggcgtggcaatatctttacccgctatggatcacgcattgagcgtgaacaacaccaccttattgagtctattgaaaagtccacgcaatacatggcgaagcgtcgcattggagctttgatctctgtggctcgtgatacaggcatggacgactacatcgagactggcattccgcttaatgcgaaaatctcatcgcaattattgattaatatcttcatccccaatacccctcttcacgatggcgcagtgatcattaaaggtaacgagatcgcgagcgctgccagttatctgccattgtccgactcgccgtttctttctaaggagctgggaactcgccatcgtgccgcattagggatttccgaggtgacagattcaatcacaatcgttgtcagcgaggaaacaggtgggatttcccttacgaagggaggcgaactgttccgtgatgtatccgaagaagaattgcataagattttgcttaaagagctggtgactgtcacagctaaaaaaccaagcattttctccaaatggaaaggaggtaagtccgagtga

SEQ ID NO: 1285-1442 - skipped (158 entries)

SEQ ID NO: 1443 - DNA, 42 bp, Artificial Sequence
  Description of Artificial Sequence: Synthetic oligonucleotide
tcgacatttatcccttgcggcgaatacttacagccatagcaa

SEQ ID NO: 1444 - DNA, 49 bp, Artificial Sequence
  Description of Artificial Sequence: Synthetic oligonucleotide
ggcgcgccttgacaattaatcatccggctcctaggatgtgtggagggac

SEQ ID NO: 1445 - DNA, 50 bp, Artificial Sequence
  Description of Artificial Sequence: Synthetic oligonucleotide
ggcgcgccttgacatcaggaaaatttttctgtataatagattcatctcaa

SEQ ID NO: 1446 - DNA, 51 bp, Artificial Sequence
  Description of Artificial Sequence: Synthetic oligonucleotide
ggcgcgccttgacataaagtctaacctataggatacttacagccatacaag

SEQ ID NO: 1447-1448 - skipped (2 entries)

SEQ ID NO: 1449 - DNA, 915 bp, Artificial Sequence
  Description of Artificial Sequence: Synthetic polynucleotide
ccgcgtgctgcacaaccgcgtacacaagctgcagcccaggccgacgcagtatccacgaacacaaattcagcgttgagtgatgctatggcttcaactcaatcaattcttttggacaccggcgcatatttaactcgccatatcgcccaaaagtctcgtgcggatgcagaaaaaaatagcgtgtggatgtcgaatactgggtatgggcgcgactacgcctctgcgcagtaccgccgtttcagcagcaaacgcacccagactcaaatcgggatcgatcgtagtcttagtgagaatatgcagattggcggagttctgacttattccgactctcaacatacgttcgaccaggcagggggcaagaatacgtttgtacaggccaatctttacggaaagtactacttgaatgatgcctggtatgtcgctggggacattggcgcagggagcttgcgttcccgccttcaaacgcaacaaaaggcgaatttcaatcgcaccagcattcagactggccttacacttgggaatacacttaagattaatcaatttgagattgtgcccagtgctgggattcgttatagtcgtttatccagcgcagactataagttaggcgacgactctgtgaaagttagctcaatggccgtgaagacgttgacggctggtttggatttcgcctatcgctttaaagttggaaatctgaccgtgaagccgctgctttcggccgcttattttgcaaactacggcaagggcggcgttaatgtcggtggtaagagtttcgcctataaagctgacaatcagcagcaatattcggccggcgtcgcgttgttgtatcgcaacgtgactttaaatgtaaacggctccatcacaaagggcaagcagttggagaagcaaaagtcaggccaaatcaagattcaaatccgtttc

SEQ ID NO: 1450 - DNA, 696 bp, Artificial Sequence
  Description of Artificial Sequence: Synthetic polynucleotide
atgttaatcaatagaaacattgtcgccctttttgccctgccattcatggcgagtgcgaccgcttccgagttaagcataggggccggcgctgcgtataatgagagtccttaccgtggctataatgaaaacacaaaggcaatcccccttatctcgtatgaaggcgacaccttctatgtacgtcagactacattaggtttcatcttaagccagtccgagaaaaacgagttatcgctgacagcttcatggatgccattggaattcgacccgacagataatgatgattatgcgatgcagcagctggacaaacgggattcgacagctatggccggcgtggcttggtatcaccacgagcggtggggaactgtcaaagcttccgccgccgctgacgtgttagacaacagcaatggctgggtgggggaattaagcgtgttccataagatgcagataggtagattatcccttacaccggctttgggagtcctgtattatgatgagaacttttccgactattattatgggatttctgagtctgaaagtcgtcggagcggtcttgcgtcgtactcggcgcaagacgcgtgggtgccttatgtatctttgacagcgaagtatccaataggcgagcacgtggtattgatggcaagtgctggttattccgagttgcccgaagaaattactgacagtcctatgatagaccgt

SEQ ID NO: 1451-1463 - skipped (13 entries)

SEQ ID NO: 1464 - protein, 305 aa, Artificial Sequence
  Description of Artificial Sequence: Synthetic polypeptide
PRAAQPRTQAAAQADAVSTNTNSALSDAMASTQSILLDTGAYLTRHIAQKSRADAEKNSVWMSNTGYGRDYASAQYRRFSSKRTQTQIGIDRSLSENMQIGGVLTYSDSQHTFDQAGGKNTFVQANLYGKYYLNDAWYVAGDIGAGSLRSRLQTQQKANFNRTSIQTGLTLGNTLKINQFEIVPSAGIRYSRLSSADYKLGDDSVKVSSMAVKTLTAGLDFAYRFKVGNLTVKPLLSAAYFANYGKGGVNVGGKSFAYKADNQQQYSAGVALLYRNVTLNVNGSITKGKQLEKQKSGQIKIQIRF

SEQ ID NO: 1465 - protein, 232 aa, Artificial Sequence
  Description of Artificial Sequence: Synthetic polypeptide
MLINRNIVALFALPFMASATASELSIGAGAAYNESPYRGYNENTKAIPLISYEGDTFYVRQTTLGFILSQSEKNELSLTASWMPLEFDPTDNDDYAMQQLDKRDSTAMAGVAWYHHERWGTVKASAAADVLDNSNGWVGELSVFHKMQIGRLSLTPALGVLYYDENFSDYYYGISESESRRSGLASYSAQDAWVPYVSLTAKYPIGEHVVLMASAGYSELPEEITDSPMIDR

SEQ ID NO: 1466-1476 - skipped (11 entries)

SEQ ID NO: 1477 - protein, 5 aa, Artificial Sequence
  Description of Artificial Sequence: Synthetic peptide
GGGGS

SEQ ID NO: 1478 - protein, 10 aa, Artificial Sequence
  Description of Artificial Sequence: Synthetic peptide
GGGGSGGGGS

SEQ ID NO: 1479 - protein, 15 aa, Artificial Sequence
  Description of Artificial Sequence: Synthetic peptide
GGGGSGGGGSGGGGS

SEQ ID NO: 1480 - protein, 5 aa, Artificial Sequence
  Description of Artificial Sequence: Synthetic peptide
EAAAK

SEQ ID NO: 1481 - protein, 10 aa, Artificial Sequence
  Description of Artificial Sequence: Synthetic peptide
EAAAKEAAAK

SEQ ID NO: 1482 - protein, 15 aa, Artificial Sequence
  Description of Artificial Sequence: Synthetic peptide
EAAAKEAAAKEAAAK

SEQ ID NO: 1483 - protein, 173 aa, Escherichia coli
MQLNKVLKGLMIALPVMAIAACSSNKNASNDGSEGMLGAGTGMDANGGNGNMSSEEQARLQMQQLQQNNIVYFDLDKYDIRSDFAQMLDAHANFLRSNPSYKVTVEGHADERGTPEYNISLGERRANAVKMYLQGKGVSADQISIVSYGKEKPAVLGHDEAAYAKNRRAVLVY

SEQ ID NO: 1484 - DNA, 4307 bp, Artificial Sequence
  Description of Artificial Sequence: Synthetic polynucleotide
tagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaaggaggtaaattatgaaatatcttcttccaacggctgctgctggtttattgcttcttgccgcccagcctgcgatggctactagtgactataaggatgacgacgacaagagatctacggcattgacggaaggtgcaaaactgtttgagaaagagatcccgtatatcaccgaactggaaggcgacgtcgaaggtatgaaatttatcattaaaggcgagggtaccggtgacgcgaccacgggtaccattaaagcgaaatacatctgcactacgggcgacctgccggtcccgtgggcaaccctggtgagcaccctgagctacggtgttcagtgtttcgccaagtacccgagccacatcaaggatttctttaagagcgccatgccggaaggttatacccaagagcgtaccatcagcttcgaaggcgacggcgtgtacaagacgcgtgctatggttacctacgaacgcggttctatctacaatcgtgtcacgctgactggtgagaactttaagaaagacggtcacattctgcgtaagaacgttgcattccaatgcccgccaagcattctgtatattctgcctgacaccgttaacaatggcatccgcgttgagttcaaccaggcgtacgatattgaaggtgtgaccgaaaaactggttaccaaatgcagccaaatgaatcgtccgttggcgggctccgcggcagtgcatatcccgcgttatcatcacattacctaccacaccaaactgagcaaagaccgcgacgagcgccgtgatcacatgtgtctggtagaggtcgtgaaagcggttgatctggacacgtatcagggatccggccaccaccatcatcaccactgcagtagcaataaaaatgcttcgaatgacggatcggaaggaatgttaggtgctggtactggcatggacgccaacggtggcaacggaaatatgtctagcgaagaacaagctcgtttacaaatgcagcaattacagcaaaacaacatagtctacttcgacttagacaagtacgacatccgtagcgattttgcacaaatgttggatgcccatgccaacttccttcgttctaatccaagctacaaagtcactgttgagggccacgcggatgagagaggcactcctgaatataacatatccctgggggagcgcagagcaaacgccgtaaaaatgtacctgcaaggcaagggcgtctcggccgatcaaatctcgatcgtgagctatggcaaagagaaaccggctgtattgggacatgacgaagctgcttacagcaagaatcggcgggccgtcttggtctactaataagcatgctaatcagccgtggaattcgcaacgtaaaaaaacccgccccggcgggtttttttataccggtctcaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgc

SEQ ID NO: 1485 - DNA, 3811 bp, Artificial Sequence
  Description of Artificial Sequence: Synthetic polynucleotide
tagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaaggaggtaaattatgactagtgcttttgaccccaatctggttgggcctacgttacctccaattccgcctttcactctgcctacggactataaggatgacgacgacaagagatctacggcattgacggaaggtgcaaaactgtttgagaaagagatcccgtatatcaccgaactggaaggcgacgtcgaaggtatgaaatttatcattaaaggcgagggtaccggtgacgcgaccacgggtaccattaaagcgaaatacatctgcactacgggcgacctgccggtcccgtgggcaaccctggtgagcaccctgagctacggtgttcagtgtttcgccaagtacccgagccacatcaaggatttctttaagagcgccatgccggaaggttatacccaagagcgtaccatcagcttcgaaggcgacggcgtgtacaagacgcgtgctatggttacctacgaacgcggttctatctacaatcgtgtcacgctgactggtgagaactttaagaaagacggtcacattctgcgtaagaacgttgcattccaatgcccgccaagcattctgtatattctgcctgacaccgttaacaatggcatccgcgttgagttcaaccaggcgtacgatattgaaggtgtgaccgaaaaactggttaccaaatgcagccaaatgaatcgtccgttggcgggctccgcggcagtgcatatcccgcgttatcatcacattacctaccacaccaaactgagcaaagaccgcgacgagcgccgtgatcacatgtgtctggtagaggtcgtgaaagcggttgatctggacacgtatcagggatccggccaccaccatcatcaccactaataagcatgctaatcagccgtggaattcggtctcaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgc

SEQ ID NO: 1486 - DNA, 4195 bp, Artificial Sequence
  Description of Artificial Sequence: Synthetic polynucleotide
tggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaaggaggtaaattatgactagtaaagcaacaaaacttgtgttaggcgcggttatacttggctccaccctgcttgcaggttgctcgtctaacgcgaagatcgaccagggtatcaatccttacgtcgggtttgaaatgggatacgattggttgggacgtatgccttataagggaagtgttgaaaacggcgcttataaggcgcagggagtacagttaacggccaagcttgggtaccccataacagacgatttagatatttatacccgtttaggaggaatggtttggagagccgacacgaagtctaatgtatatggtaagaaccacgacacgggagtatcccccgtctttgcagggggagtggaatatgctatcacaccagagatcgctacccgtttggaatatcaatggacgaataatataggcgacgcccatacgataggaacgcggcccgacaacggcatccctggggtcgactataaggatgacgacgacaagcaattgacggcattgacggaaggtgcaaaactgtttgagaaagagatcccgtatatcaccgaactggaaggcgacgtcgaaggtatgaaatttatcattaaaggcgagggtaccggtgacgcgaccacgggtaccattaaagcgaaatacatctgcactacgggcgacctgccggtcccgtgggcaaccctggtgagcaccctgagctacggtgttcagtgtttcgccaagtacccgagccacatcaaggatttctttaagagcgccatgccggaaggttatacccaagagcgtaccatcagcttcgaaggcgacggcgtgtacaagacgcgtgctatggttacctacgaacgcggttctatctacaatcgtgtcacgctgactggtgagaactttaagaaagacggtcacattctgcgtaagaacgttgcattccaatgcccgccaagcattctgtatattctgcctgacaccgttaacaatggcatccgcgttgagttcaaccaggcgtacgatattgaaggtgtgaccgaaaaactggttaccaaatgcagccaaatgaatcgtccgttggcgggctccgcggcagtgcatatcccgcgttatcatcacattacctaccacaccaaactgagcaaagaccgcgacgagcgccgtgatcacatgtgtctggtagaggtcgtgaaagcggttgatctggacacgtatcagggatccggccaccaccatcatcaccactaataagcatgctaatcagccgtggaattcggtctcaggaggaacgattggtaaacccggtgaacgcatgagaaagcccccggaagatcaccttccgggggcttttttattgcgcggaccaaaacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgt

SEQ ID NO: 1487 - DNA, 4744 bp, Artificial Sequence
  Description of Artificial Sequence: Synthetic polynucleotide
aacgaaaaaagacgctcgaaagcgtctcttttctggaatttggtaccgaggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacggtgcaccctgcagggctagctgataaagcgttcgcgctgcattcggcagtttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaaggaggtaaattatgactagtaaggcaaaacgcttcaagattaacgctatcagtttatccatctttttagcgtatgcgttgactccgtactcagaggcagtcgactataaggatgacgacgacaagcaattgacggcattgacggaaggtgcaaaactgtttgagaaagagatcccgtatatcaccgaactggaaggcgacgtcgaaggtatgaaatttatcattaaaggcgagggtaccggtgacgcgaccacgggtaccattaaagcgaaatacatctgcactacgggcgacctgccggtcccgtgggcaaccctggtgagcaccctgagctacggtgttcagtgtttcgccaagtacccgagccacatcaaggatttctttaagagcgccatgccggaaggttatacccaagagcgtaccatcagcttcgaaggcgacggcgtgtacaagacgcgtgctatggttacctacgaacgcggttctatctacaatcgtgtcacgctgactggtgagaactttaagaaagacggtcacattctgcgtaagaacgttgcattccaatgcccgccaagcattctgtatattctgcctgacaccgttaacaatggcatccgcgttgagttcaaccaggcgtacgatattgaaggtgtgaccgaaaaactggttaccaaatgcagccaaatgaatcgtccgttggcgggctccgcggcagtgcatatcccgcgttatcatcacattacctaccacaccaaactgagcaaagaccgcgacgagcgccgtgatcacatgtgtctggtagaggtcgtgaaagcggttgatctggacacgtatcagggatccggccaccaccatcatcaccacccgcgtgctgcacaaccgcgtacacaagctgcagcccaggccgacgcagtatccacgaacacaaattcagcgttgagtgatgctatggcttcaactcaatcaattcttttggacaccggcgcatatttaactcgccatatcgcccaaaagtctcgtgcggatgcagaaaaaaatagcgtgtggatgtcgaatactgggtatgggcgcgactacgcctctgcgcagtaccgccgtttcagcagcaaacgcacccagactcaaatcgggatcgatcgtagtcttagtgagaatatgcagattggcggagttctgacttattccgactctcaacatacgttcgaccaggcagggggcaagaatacgtttgtacaggccaatctttacggaaagtactacttgaatgatgcctggtatgtcgctggggacattggcgcagggagcttgcgttcccgccttcaaacgcaacaaaaggcgaatttcaatcgcaccagcattcagactggccttacacttgggaatacacttaagattaatcaatttgagattgtgcccagtgctgggattcgttatagtcgtttatccagcgcagactataagttaggcgacgactctgtgaaagttagctcaatggccgtgaagacgttgacggctggtttggatttcgcctatcgctttaaagttggaaatctgaccgtgaagccgctgctttcggccgcttattttgcaaactacggcaagggcggcgttaatgtcggtggtaagagtttcgcctataaagctgacaatcagcagcaatattcggccggcgtcgcgttgttgtatcgcaacgtgactttaaatgtaaacggctccatcacaaagggcaagcagttggagaagcaaaagtcaggccaaatcaagattcaaatccgtttctaataagcatgctaatcagccgtggaattcggtctcaggaggaacgattggtaaacccggtgaacgcatgagaaagcccccggaagatcaccttccgggggcttttttattgcgcggaccaa

SEQ ID NO: 1488 - DNA, 56 bp, Artificial Sequence
  Description of Artificial Sequence: Synthetic oligonucleotide
gttgacactctatcattgatagagttattttaccactccctatcagtgatagagaa

SEQ ID NO: 1489 - DNA, 456 bp, Artificial Sequence
  Description of Artificial Sequence: Synthetic polynucleotide
tgcagtagcaataaaaatgcttcgaatgacggatcggaaggaatgttaggtgctggtactggcatggacgccaacggtggcaacggaaatatgtctagcgaagaacaagctcgtttacaaatgcagcaattacagcaaaacaacatagtctacttcgacttagacaagtacgacatccgtagcgattttgcacaaatgttggatgcccatgccaacttccttcgttctaatccaagctacaaagtcactgttgagggccacgcggatgagagaggcactcctgaatataacatatccctgggggagcgcagagcaaacgccgtaaaaatgtacctgcaaggcaagggcgtctcggccgatcaaatctcgatcgtgagctatggcaaagagaaaccggctgtattgggacatgacgaagctgcttacagcaagaatcggcgggccgtcttggtctac

SEQ ID NO: 1490 - DNA, 63 bp, Artificial Sequence
  Description of Artificial Sequence: Synthetic oligonucleotide
gcttttgaccccaatctggttgggcctacgttacctccaattccgcctttcactctgcctacg

SEQ ID NO: 1491 - DNA, 442 bp, Artificial Sequence
  Description of Artificial Sequence: Synthetic polynucleotide
atgactagttaaagcaacaaaacttgtgttaggcgcggttatacttggctccaccctgcttgcaggttgctcgtctaacgcgaagatcgaccagggtatcaatccttacgtcgggtttgaaatgggatacgattggttgggacgtatgccttataagggaagtgttgaaaacggcgcttataaggcgcagggagtacagttaacggccaagcttgggtaccccataacagacgatttagatatttatacccgtttaggaggaatggtttggagagccgacacgaagtctaatgtatatggtaagaaccacgacacgggagtatcccccgtctttgcagggggagtggaatatgctatcacaccagagatcgctacccgtttggaatatcaatggacgaataatataggcgacgcccatacgataggaacgcggcccgacaac

SEQ ID NO: 1492 - DNA, 603 bp, Artificial Sequence
  Description of Artificial Sequence: Synthetic polynucleotide
atgacaatgactcgtttgaaaatttccaaaaccctgctggccgtaatgcttacgtccgcagtcgccactggttccgcatacgcagagaataatgcgcagacaactaacgagtcagcagggcaaaaagtagactccagtatgaataaggtgggcaattttatggacgactcagctatcaccgctaaggtgaaagcggcgctggtggaccacgacaacatcaagtccacggacatctcagttaaaacggaccaaaaggtagtaaccttaagcgggttcgtagaaagccaggcgcaagcagaggaagcggtaaaagtcgctaaaggcgtagaaggggtcacttcggtgtcagacaagttgcatgtgcgtgacgcaaaggaaggatcagtaaagggttatgccggagatacggcaacgacctctgagatcaaagcgaaattactggcagacgacattgttccctcgcgtcatgttaaagtcgaaacgactgatggcgtagtccagctttcgggtacagttgactcccaagcacaaagtgatcgcgcggaatctattgcaaaggcggtggacggagtgaaatccgtcaaaaacgatcttaaaacgaaa

SEQ ID NO: 1493 - DNA, 1820 bp, Artificial Sequence
  Description of Artificial Sequence: Synthetic polynucleotide
atggttttccaacccatcagcgaatttttgctgattcgtaacgctgggatgtccatgtattttaacaagatcatttcttttaacatcatttcacgtatcgttatttgcatttttcttatctgtggtatgttcatggccggtgcatctgaaaagtatgatgcaaacgcaccccaacaggtgcagccatactcggtttcatcatcagcgttcgagaatctgcaccccaataacgagatggagtcgagtatcaacccttttagtgcttcggacaccgagcgtaatgcagctatcatcgatcgtgctaacaaggaacaagaaacggaagcagtcaacaaaatgatctccactggcgctcgtttagctgccagcggtcgcgcgtccgatgtggcgcacagtatggtaggggatgcggtcaaccaggagattaaacaatggctgaatcgcttcggcactgctcaagtgaatttaaattttgacaagaacttctcgttaaaggagtcttcgcttgactggttggccccatggtacgattcggcgtcattccttttcttttctcagttgggcatccgtaacaaggacagtcgtaatacacttaaccttggtgttggcattcgcacattagaaaatggttggttgtatggcctgaacaccttttacgacaatgacttaacgggacacaatcaccgtatcgggctgggcgccgaggcgtggactgactacttgcagttagccgcgaatgggtacttccgtcttaatggttggcactcttcccgtgacttcagcgactacaaagaacgccctgctaccgggggagatttgcgtgcgaatgcgtacctgcccgctcttccgcaacttggcgggaagttaatgtatgagcagtatactggggaacgcgtggctctgttcggaaaggacaacctgcagcgcaacccatacgctgtcactgcgggtatcaactatacgccagttccgttgctgacggtcggcgtggatcaacgtatggggaagtcgagtaaacatgaaacgcaatggaatttacaaatgaactatcgcttaggggagagtttccaaagtcagcttagcccttcggcggtcgcagggactcgtttgcttgctgagtcccgctacaacctggttgatcgcaataacaatatcgtactggaataccagaaacaacaagtggttaagctgacgttgagccctgcgaccatcagtggattgcccggacaagtttaccaggtaaatgcccaggtccagggggcctctgcggttcgcgaaattgtctggtcagacgcagaattaatcgctgcaggaggcaccttaacgccactttccactacacaattcaatttagtccttcccccatacaaacgtaccgcccaggtatcgcgcgtaactgatgacttaactgctaatttttattcactgtcggcgttagcagttgaccatcaaggcaaccgtagtaattccttcacattatctgtaacggtgcagcagccgcaactgacgcttaccgcagcggtcattggtgatggggccccagctaatgggaaaaccgcaatcactgtcgagttcacagttgcagattttgaaggcaagccgctggcgggtcaggaggttgtgattacgactaataacggtgctcttcctaataagattactgaaaagactgacgctaacggcgttgcccgcattgcccttacgaacacaaccgatggggtcacggtagttaccgcagaggtcgaggggcaacgccaatccgttgacacgcacttcgttaagggtactatcgcggccgataaaagcacgctggccgcggt

SEQ ID NO: 1494 - DNA, 1977 bp, Artificial Sequence
  Description of Artificial Sequence: Synthetic polynucleotide
atgattacgcatggctgttatacccgtacgcgtcataaacacaagttgaagaaaactctgatcatgttatccgctggacttggactttttttttacgtgaatcagaactctttcgctaatggggaaaattattttaaactgggatcagacagcaaattacttacgcatgactcataccagaatcgtctgttttatacgctgaaaactggtgaaaccgttgcagatttaagtaaaagtcaggacattaacctgtcaactatttggtcacttaataaacacttatattcgagcgaatcggaaatgatgaaagctgcaccggggcaacaaatcatcttgcccctgaagaaattgccctttgaatactccgctttgcccttgctgggctcggctcctctggtagccgccggaggcgttgccggtcacactaataagctgacaaaaatgtcacccgacgtgacgaagagcaacatgacggatgataaggctttaaattacgcagctcagcaagcggcctcgttgggaagtcagttacagagtcgttcgttaaatggtgattatgctaaggataccgcattgggtattgccggcaaccaagcgtcgagccaacttcaggcatggttgcaacattacggcactgctgaagtaaatctgcaatcaggtaataattttgacggtagttccctggatttccttttacctttttacgattcagaaaagatgttggctttcggacaggtgggggcgcgttacatcgattcacgttttaccgctaacttgggggccggtcaacgcttcttcttacctgccaatatgttgggctataatgtatttatcgaccaggacttcagtggtgacaatacacgtctgggaattggtggagagtattggcgcgattactttaagtcatctgtaaatggctattttcgcatgagcggttggcatgaaagttacaacaagaaagactacgatgagcgccccgcgaacgggtttgacatccgttttaatggttatttgccatcttatcccgccttgggagctaaattaatctacgagcaatactatggagataacgtagctttgtttaatagcgacaagttacagtctaatccaggagcggctacagtgggagttaattataccccaatcccactggtcacaatgggaatcgattatcgccacgggactggtaatgaaaacgatttattatactccatgcagtttcgttatcagttcgataagagttggtcgcagcagattgagcctcaatatgttaacgaattacgtaccttgtccggcagtcgctacgatctggtacaacgcaataacaatatcatccttgagtataagaaacaggacattctgtctttgaacattccacatgatattaatggtaccgagcactcaacacaaaaaattcagctgattgtgaaatcaaagtatggactggaccgtatcgtgtgggatgatagcgctctgcgcagtcagggtggacagatccagcactcgggtagccagtctgcccaagactaccaggctatcctgccagcgtatgtccaagggggaagtaacatctacaaagttacagctcgcgcctatgaccgcaacggtaattctagtaataatgtgcagttgacaattacggtgctgtccaatgggcaggtcgtcgatcaggtaggtgtgacggattttacagccgataaaacctctgcgaaggcagataacgcggataccatcacatacactgccactgtaaaaaaaaacggtgtcgcgcaggcaaacgttcctgttagcttcaacatcgtgtcgggtacagccacccttggggccaactcggcaaagactgacgcgaatggcaaggctacagtcacgttgaaatcctcgacaccaggacaggtcgttgtgtctgccaagacagcagagatgacctccgcccttaatgcatctgctgttatcttcttcgatcaaacgaaggcatct

SEQ ID NO: 1495 - DNA, 705 bp, Artificial Sequence
  Description of Artificial Sequence: Synthetic polynucleotide
tcggcattgacggaaggtgcaaaactgtttgagaaagagatcccgtatatcaccgaactggaaggcgacgtcgaaggtatgaaatttatcattaaaggcgagggtaccggtgacgcgaccacgggtaccattaaagcgaaatacatctgcactacgggcgacctgccggtcccgtgggcaaccctggtgagcaccctgagctacggtgttcagtgtttcgccaagtacccgagccacatcaaggatttctttaagagcgccatgccggaaggttatacccaagagcgtaccatcagcttcgaaggcgacggcgtgtacaagacgcgtgctatggttacctacgaacgcggttctatctacaatcgtgtcacgctgactggtgagaactttaagaaagacggtcacattctgcgtaagaacgttgcattccaatgcccgccaagcattctgtatattctgcctgacaccgttaacaatggcatccgcgttgagttcaaccaggcgtacgatattgaaggtgtgaccgaaaaactggttaccaaatgcagccaaatgaatcgtccgttggcgggctccgcggcagtgcatatcccgcgttatcatcacattacctaccacaccaaactgagcaaagaccgcgacgagcgccgtgatcacatgtgtctggtagaggtcgtgaaagcggttgatctggacacgtatcag

SEQ ID NO: 1496 - DNA, 705 bp, Artificial Sequence
  Description of Artificial Sequence: Synthetic polynucleotide
acggcattgacggaaggtgcaaaactgtttgagaaagagatcccgtatatcaccgaactggaaggcgacgtcgaaggtatgaaatttatcattaaaggcgagggtaccggtgacgcgaccacgggtaccattaaagcgaaatacatctgcactacgggcgacctgccggtcccgtgggcaaccctggtgagcaccctgagctacggtgttcagtgtttcgccaagtacccgagccacatcaaggatttctttaagagcgccatgccggaaggttatacccaagagcgtaccatcagcttcgaaggcgacggcgtgtacaagacgcgtgctatggttacctacgaacgcggttctatctacaatcgtgtcacgctgactggtgagaactttaagaaagacggtcacattctgcgtaagaacgttgcattccaatgcccgccaagcattctgtatattctgcctgacaccgttaacaatggcatccgcgttgagttcaaccaggcgtacgatattgaaggtgtgaccgaaaaactggttaccaaatgcagccaaatgaatcgtccgttggcgggctccgcggcagtgcatatcccgcgttatcatcacattacctaccacaccaaactgagcaaagaccgcgacgagcgccgtgatcacatgtgtctggtagaggtcgtgaaagcggttgatctggacacgtatcag

SEQ ID NO: 1497 - protein, 152 aa, Artificial Sequence
  Description of Artificial Sequence: Synthetic polypeptide
CSSNKNASNDGSEGMLGAGTGMDANGGNGNMSSEEQARLQMQQLQQNNIVYFDLDKYDIRSDFAQMLDAHANFLRSNPSYKVTVEGHADERGTPEYNISLGERRANAVKMYLQGKGVSADQISIVSYGKEKPAVLGHDEAAYSKNRRAVLVY

SEQ ID NO: 1498 - protein, 24 aa, Artificial Sequence
  Description of Artificial Sequence: Synthetic peptide
MTSAFDPNLVGPTLPPIPPFTLPT

SEQ ID NO: 1499 - protein, 147 aa, Artificial Sequence
  Description of Artificial Sequence: Synthetic polypeptide
MTSKATKLVLGAVILGSTLLAGCSSNAKIDQGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDN

SEQ ID NO: 1500 - protein, 201 aa, Artificial Sequence
  Description of Artificial Sequence: Synthetic polypeptide
MTMTRLKISKTLLAVMLTSAVATGSAYAENNAQTTNESAGQKVDSSMNKVGNFMDDSAITAKVKAALVDHDNIKSTDISVKTDQKVVTLSGFVESQAQAEEAVKVAKGVEGVTSVSDKLHVRDAKEGSVKGYAGDTATTSEIKAKLLADDIVPSRHVKVETTDGVVQLSGTVDSQAQSDRAESIAKAVDGVKSVKNDLKTK

SEQ ID NO: 1501 - protein, 659 aa, Artificial Sequence
  Description of Artificial Sequence: Synthetic polypeptide
MITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYDRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDQTKAS

SEQ ID NO: 1502 - protein, 235 aa, Artificial Sequence
  Description of Artificial Sequence: Synthetic polypeptide
TALTEGAKLFEKEIPYITELEGDVEGMKFIIKGEGTGDATTGTIKAKYICTTGDLPVPWATLVSTLSYGVQCFAKYPSHIKDFFKSAMPEGYTQERTISFEGDGVYKTRAMVTYERGSIYNRVTLTGENFKKDGHILRKNVAFQCPPSILYILPDTVNNGIRVEFNQAYDIEGVTEKLVTKCSQMNRPLAGSAAVHIPRYHHITYHTKLSKDRDERRDHMCLVEVVKAVDLDTYQ

SEQ ID NO: 1503 - protein, 470 aa, Artificial Sequence
  Description of Artificial Sequence: Synthetic polypeptide
TALTEGAKLFEKEIPYITELEGDVEGMKFIIKGEGTGDATTGTIKAKYICTTGDLPVPWATLVSTLSYGVQCFAKYPSHIKDFFKSAMPEGYTQERTISFEGDGVYKTRAMVTYERGSIYNRVTLTGENFKKDGHILRKNVAFQCPPSILYILPDTVNNGIRVEFNQAYDIEGVTEKLVTKCSQMNRPLAGSAAVHIPRYHHITYHTKLSKDRDERRDHMCLVEVVKAVDLDTYQTALTEGAKLFEKEIPYITELEGDVEGMKFIIKGEGTGDATTGTIKAKYICTTGDLPVPWATLVSTLSYGVQCFAKYPSHIKDFFKSAMPEGYTQERTISFEGDGVYKTRAMVTYERGSIYNRVTLTGENFKKDGHILRKNVAFQCPPSILYILPDTVNNGIRVEFNQAYDIEGVTEKLVTKCSQMNRPLAGSAAVHIPRYHHITYHTKLSKDRDERRDHMCLVEVVKAVDLDTYQ

SEQ ID NO: 1504 - protein, 156 aa, Artificial Sequence
  Description of Artificial Sequence: Synthetic polypeptide
AQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSPSTPPTPSPSTPPGLNDIFEAQKIEWHGSS

SEQ ID NO: 1505 - protein, 149 aa, Artificial Sequence
  Description of Artificial Sequence: Synthetic polypeptide
MKYLLPTAAAGLLLLAAQPAMAQVQLVESGGGLVEPGGSLRLSCAASGIIFKINDMGWYRQAPGKRREWVAASTGGDEAIYRDSVKDRFTISRDAKNSVFLQMNSLKPEDTAVYYCTAVISTDRDGTEWRRYWGQGTQVTVSSGGLPET

SEQ ID NO: 1506 - DNA, 70 bp, Artificial Sequence
  Description of Artificial Sequence: Synthetic oligonucleotide
tcataaaaaatttatttgctttcaggaaaatttttctgtataatagattcataaatttgagagaggagtt

SEQ ID NO: 1507 - protein, 5 aa, Artificial Sequence
  Description of Artificial Sequence: Synthetic peptide
  See specification as filed for detailed description of substitutions and preferred embodiments
GGGGS

SEQ ID NO: 1508 - protein, 656 aa, Artificial Sequence
  Description of Artificial Sequence: Synthetic polypeptide
MITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYDRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDGA

SEQ ID NO: 1509 - protein, 131 aa, Artificial Sequence
  Description of Artificial Sequence: Synthetic polypeptide
MAQVQLQESGGGLVQAGGSLLLSCAASGRTFSSYAMGWFRQAPGKEREFVAAINWSGGSTSYADSVKGRFTISRDNTKNTVYLQMNSLKPEDTAAFYCAATYNPYSRDHYFPRMTTEYDYWGQGTQVTVSS

SEQ ID NO: 1510 - protein, 139 aa, Artificial Sequence
  Description of Artificial Sequence: Synthetic polypeptide
AQVQLQESGGGLVQAGGSLLLSCAASGRTFSSYAMGWFRQAPGKEREFVAAINWSGGSTSYADSVKGRFTISRDNTKNTVYLQMNSLKPEDTAAFYCAATYNPYSRDHYFPRMTTEYDYWGQGTQVTVSSEPKTPKPQP

SEQ ID NO: 1511 - protein, 124 aa, Artificial Sequence
  Description of Artificial Sequence: Synthetic polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

(19) United States
(12) Patent Application Publication
(10) Pub. No.: US 2021/0369836 A1
(43) Pub. Date: Dec. 2, 2021

(54) SURFACE DISPLAY OF PROTEINS ON RECOMBINANT BACTERIA AND USES THEREOF

(71) Applicant: Synlogic Operating Company, Inc., Cambridge, MA (US)

(72) Inventor: Ning Li, Winchester, MA (US)

(21) Appl. No.: 17/330,563

(22) Filed: May 26, 2021

Related U.S. Application Data
(60) Provisional application No. 63/030,157, filed on May 26, 2020.

(51) Int. Cl. A61K 39/215; A61K 39/00; C07K 16/00; C07K 14/71; C07K 14/005

(52) U.S. Cl. CPC A61K 39/215; A61K 39/0005; C07K 16/00; C07K 14/71; A61K 2039/523; C07K 2319/00; C07K 2317/569; C12N 2770/20022; C12N 2770/20034; C07K 14/005

(57) ABSTRACT

Recombinant microorganisms, pharmaceutical compositions thereof, and methods of protein display on the cell surface of the microorganisms are disclosed.

### RELATED APPLICATIONS

This application claims priority to U.S. Provisional Application No. 63/030,157, filed on May 26, 2020, the entire contents of which are expressly incorporated by reference herein in their entirety.

### SEQUENCE LISTING

The instant application contains a Sequence Listing which has been submitted electronically in ASCII format and is hereby incorporated by reference in its entirety. Said ASCII copy, created on May 24, 2021, is named 126046_05802_SL.txt and is 245,493 bytes in size.

### BACKGROUND

Viral, bacterial, and fungal infections cause disease such as pneumonia, diarrhea, tuberculosis, and hepatitis among others. Several diseases caused by infections and cancers do not have available treatment and/or prevention therapies, or the available therapies are insufficient. Accordingly, there is a need to provide vaccines to prevent and/or treat infections, e.g., viral, bacterial, and fungal infections, and cancers.

### SUMMARY

The present disclosure provides compositions, methods, and uses of microorganisms that can prevent and/or treat infections, e.g., bacterial, viral, or fungal infections. In certain aspects, the present disclosure provides recombinant microorganisms that are engineered to express one or more proteins, e.g., antigens, on their surface using, for example, a display protein. In one embodiment, the display protein is a fusion protein comprising, e.g., an anchor domain, a linker, and a displayed protein. In some embodiments, the displayed protein is viral, bacterial, or fungal, or a cancer or tumor protein. In certain aspects, the recombinant microorganism is a bacteria, e.g., Salmonella typhimurium, Escherichia coli Nissle, Clostridium novyi NT, and Clostridium butyricum miyairi, as well as other exemplary bacterial strains provided herein. Thus, in certain embodiments, the recombinant microorganisms are administered, e.g., via oral administration, intravenous injection, subcutaneous injection, intranasal delivery, or other means, and are able to generate an immune response in a host, e.g., a human, against the displayed protein of the display protein.

In one aspect, disclosed here is a recombinant microorganism capable of displaying a protein, i.e., a displayed protein. In one aspect, the displaying protein comprises an anchor domain, e.g., intimin, peptidoglycan-associated lipoprotein (PAL), PelB-PAL, YiaT, LppOmpA, BAN, OmsY, Invasin, IgA, PgsA, ice nucleation protein (INP), and NGIgAsig-NGIgAb, a linker, and the displayed protein.

In some embodiments, the nucleotide sequence of the anchor domain has at least 90%, 91%, 92%, 93%, 94%, 95%, 96%, 97%, 98%, 99% identity to, comprises, or consists of SEQ ID NO: 1489.

In some embodiments, the nucleotide sequence of the anchor domain has at least 90%, 91%, 92%, 93%, 94%, 95%, 96%, 97%, 98%, 99% identity to, comprises, or consists of SEQ ID NO: 1490.

In some embodiments, the nucleotide sequence of the anchor domain has at least 90%, 91%, 92%, 93%, 94%, 95%, 96%, 97%, 98%, 99% identity to, comprises, or consists of SEQ ID NO: 1491.

In some embodiments, the nucleotide sequence of the anchor domain has at least 90%, 91%, 92%, 93%, 94%, 95%, 96%, 97%, 98%, 99% identity to, comprises, or consists of SEQ ID NO: 1449.

In some embodiments, the nucleotide sequence of the anchor domain has at least 90%, 91%, 92%, 93%, 94%, 95%, 96%, 97%, 98%, 99% identity to, comprises, or consists of SEQ ID NO: 1492.

In some embodiments, the nucleotide sequence of the anchor domain has at least 90%, 91%, 92%, 93%, 94%, 95%, 96%, 97%, 98%, 99% identity to, comprises, or consists of SEQ ID NO: 1492.

In some embodiments, the nucleotide sequence of the anchor domain has at least 90%, 91%, 92%, 93%, 94%, 95%, 96%, 97%, 98%, 99% identity to, comprises, or consists of SEQ ID NO: 1493.

In some embodiments, the nucleotide sequence of the anchor domain has at least 90%, 91%, 92%, 93%, 94%, 95%, 96%, 97%, 98%, 99% identity to, comprises, or consists of SEQ ID NO: 1450.

In some embodiments, the nucleotide sequence of the anchor domain has at least 90%, 91%, 92%, 93%, 94%, 95%, 96%, 97%, 98%, 99% identity to, comprises, or consists of SEQ ID NO: 1494.

In some embodiments, the amino acid sequence of the anchor domain has at least 90%, 91%, 92%, 93%, 94%, 95%, 96%, 97%, 98%, 99% identity to, comprises, or consists of SEQ ID NO: 1497.

In some embodiments, the amino acid sequence of the anchor domain has at least 90%, 91%, 92%, 93%, 94%, 95%, 96%, 97%, 98%, 99% identity to, comprises, or consists of SEQ ID NO: 1498.

In some embodiments, the amino acid sequence of the anchor domain has at least 90%, 91%, 92%, 93%, 94%, 95%, 96%, 97%, 98%, 99% identity to, comprises, or consists of SEQ ID NO: 1499.

In some embodiments, the amino acid sequence of the anchor domain has at least 90%, 91%, 92%, 93%, 94%, 95%, 96%, 97%, 98%, 99% identity to, comprises, or consists of SEQ ID NO: 1464.

In some embodiments, the amino acid sequence of the anchor domain has at least 90%, 91%, 92%, 93%, 94%, 95%, 96%, 97%, 98%, 99% identity to, comprises, or consists of SEQ ID NO: 1469.

In some embodiments, the amino acid sequence of the anchor domain has at least 90%, 91%, 92%, 93%, 94%, 95%, 96%, 97%, 98%, 99% identity to, comprises, or consists of SEQ ID NO: 1500.

In some embodiments, the amino acid sequence of the anchor domain has at least 90%, 91%, 92%, 93%, 94%, 95%, 96%, 97%, 98%, 99% identity to, comprises, or consists of SEQ ID NO: 990.

In some embodiments, the amino acid sequence of the anchor domain has at least 90%, 91%, 92%, 93%, 94%, 95%, 96%, 97%, 98%, 99% identity to, comprises, or consists of SEQ ID NO: 1465.

In some embodiments, the amino acid sequence of the anchor domain has at least 90%, 91%, 92%, 93%, 94%, 95%, 96%, 97%, 98%, 99% identity to, comprises, or consists of SEQ ID NO: 1501.

In some embodiments, the amino acid sequence of the anchor domain has at least 90%, 91%, 92%, 93%, 94%, 95%, 96%, 97%, 98%, 99% identity to, comprises, or consists of SEQ ID NO: 1508.

In some embodiments, the nucleotide sequence of the displayed protein has at least 90%, 91%, 92%, 93%, 94%, 95%, 96%, 97%, 98%, 99% identity to, comprises, or consists of SEQ ID NO: 1495. In some embodiments, the nucleotide sequence of the displayed protein has at least 90%, 91%, 92%, 93%, 94%, 95%, 96%, 97%, 98%, 99% identity to, comprises, or consists of SEQ ID NO: 1496. In some embodiments, the amino acid sequence of the displayed protein has at least 90%, 91%, 92%, 93%, 94%, 95%, 96%, 97%, 98%, 99% identity to, comprises, or consists of SEQ ID NO: 1502. In some embodiments, the amino acid sequence of the displayed protein has at least 90%, 91%, 92%, 93%, 94%, 95%, 96%, 97%, 98%, 99% identity to, comprises, or consists of SEQ ID NO: 1503. In some embodiments, the amino acid sequence of the displayed protein has at least 90%, 91%, 92%, 93%, 94%, 95%, 96%, 97%, 98%, 99% identity to, comprises, or consists of SEQ ID NO: 1505. In some embodiments, the amino acid sequence of the displayed protein has at least 90%, 91%, 92%, 93%, 94%, 95%, 96%, 97%, 98%, 99% identity to, comprises, or consists of SEQ ID NO: 1509. In some embodiments, the amino acid sequence of the displayed protein has at least 90%, 91%, 92%, 93%, 94%, 95%, 96%, 97%, 98%, 99% identity to, comprises, or consists of SEQ ID NO: 1510. In some embodiments, the amino acid sequence of the displayed protein has at least 90%, 91%, 92%, 93%, 94%, 95%, 96%, 97%, 98%, 99% identity to, comprises, or consists of SEQ ID NO: 1511. In some embodiments, the amino acid sequence of the displayed protein has at least 90%, 91%, 92%, 93%, 94%, 95%, 96%, 97%, 98%, 99% identity to, comprises, or consists of SEQ ID NO: 1504.

In one aspect, disclosed herein is a recombinant microorganism capable of expressing at least one displayed protein. In one aspect, disclosed herein is a recombinant microorganism capable of producing at least one immune modulator. In one aspect, disclosed herein is a recombinant microorganism capable of expressing at least one displayed protein and at least one immune modulator.

In another aspect, disclosed herein is a composition comprising a viral protein, e.g., a viral spike protein from a coronavirus, e.g., a viral spike protein receptor binding domain (RBD) from severe acute respiratory syndrome coronavirus 2 (SARS-CoV2).

In another aspect, disclosed herein is a composition comprising an immune modulator. In some embodiments, the immune modulator comprises an immune initiator, e.g., a cytokine, chemokine, single chain antibody, ligand, metabolic converter, T cell co-stimulatory receptor, T cell co-stimulatory receptor ligand, or lytic peptide. In some embodiments, the immune modulator comprises an immune modulator, e.g., a chemokine, a cytokine, a single chain antibody, a ligand, a metabolic converter, a T cell co-stimulatory receptor, or a T cell co-stimulatory receptor ligand.

In another aspect, disclosed herein is a composition comprising a first recombinant microorganism capable of expressing at least one display protein and at least a second recombinant microorganism capable of producing at least one immune modulator.

In one embodiment, the immune initiator is capable of enhancing oncolysis, activating antigen presenting cells (APCs), and/or priming and activating T cells. In another embodiment, the immune initiator is capable of enhancing oncolysis. In another embodiment, the immune initiator is capable of activating APCs. In yet another embodiment, the immune initiator is capable of priming and activating T cells.

In one embodiment, the microorganism induces a CTL response to a virus. In one embodiment, the microorganism produces a CTL response against epitopes in the viral nucleocapsid (N) and/or M protein. Such proteins and epitopes are well known in the art and described at least in Liu et al., Antiviral Research 137 (2017), 82-92; Huang et al., Vaccine 25 (2007):6981-6991; Ahmed et al., Viruses (2020) 12:254; Grifoni et al., Cell Host & Microbiome (2020) 27:1-10; and Chen et al., J. Immunol (2005) 175:591-598, the entire contents of each of which are expressly incorporated by reference herein in their entireties.

In one embodiment, the immune initiator is a therapeutic molecule encoded by at least one gene. In one embodiment, the immune initiator is a therapeutic molecule produced by an enzyme encoded by at least one gene. In one embodiment, the immune imitator is at least one enzyme of a biosynthetic pathway or a catabolic pathway encoded by at least one gene. In one embodiment, the immune imitator is at least one therapeutic molecule produced by at least one enzyme of a biosynthetic pathway or a catabolic pathway encoded by at least one gene. In one embodiment, the immune imitator is a nucleic acid molecule that mediates RNA interference, microRNA response or inhibition, TLR response, antisense gene regulation, target protein binding, or gene editing.

In one embodiment, the immune imitator is a cytokine, a chemokine, a single chain antibody, a ligand, a metabolic converter, a T cell co-stimulatory receptor, a T cell co-stimulatory receptor ligand, or a lytic peptide. In one embodiment, the immune initiator is a secreted peptide or a displayed peptide.

In one embodiment, the immune initiator is a STING agonist, arginine, 5-FU, TNFα, IFNγ, IFNβ1, agonistic anti-CD40 antibody, CD40L, SIRPα, GMCSF, agonistic anti-OX040 antibody, OXO40L, agonistic anti-4-1BB antibody, 4-1BBL, agonistic anti-GITR antibody, GITRL, anti-PD1 antibody, anti-PDL1 antibody, or azurin. In one embodiment, the immune initiator is a STING agonist. In one embodiment, the immune initiator is at least one enzyme of an arginine biosynthetic pathway. In one embodiment, the immune initiator is arginine. In one embodiment, the immune initiator is 5-FU. In one embodiment, the immune initiator is TNFα. In one embodiment, the immune initiator is IFNγ. In one embodiment, the immune initiator is IFNβ1. In one embodiment, the immune initiator is an agonistic anti-CD40 antibody. In one embodiment, the immune initiator is SIRPα. In one embodiment, the immune initiator is CD40L. In one embodiment, the immune initiator is GMCSF. In one embodiment, the immune initiator is an agonistic anti-OX040 antibody. In another embodiment, the immune initiator is OXO40L. In one embodiment, the immune initiator is an agonistic anti-4-1BB antibody. In one embodiment, the immune initiator is 4-1BBL. In one embodiment, the immune initiator is an agonistic anti-GITR antibody. In another embodiment, the immune initiator is GITRL. In one embodiment, the immune initiator is an anti-PD1 antibody. In one embodiment, the immune initiator is an anti-PDL1 antibody. In one embodiment, the immune initiator is azurin.

In one embodiment, the immune initiator is a STING agonist. In one embodiment, the STING agonist is c-diAMP. In one embodiment, the STING agonist is c-GAMP. In one embodiment, the STING agonist is c-diGMP.

In one embodiment, the recombinant microorganism comprises at least one gene sequence encoding an enzyme which produces the immune initiator. In one embodiment, the at least one gene sequence encoding the immune initiator is a dacA gene sequence. In one embodiment, the at least one gene sequence encoding the immune initiator is a cGAS gene sequence. In one embodiment, the cGAS gene sequence is a human cGAS gene sequence. In one embodiment, the cGAS gene sequence is selected from a human cGAS gene sequence a Verminephrobacter eiseniae cGAS gene sequence, Kingella denitrificans cGAS gene sequence, and a Neisseria bacilliformis cGAS gene sequence.

In one embodiment, the at least one gene sequence encoding the immune initiator is integrated into a chromosome of the recombinant microorganism. In one embodiment, the at least one gene sequence encoding the immune initiator is present on a plasmid. In one embodiment, the at least one gene sequence encoding the immune initiator is operably linked to an inducible promoter. In one embodiment, the inducible promoter is induced by low oxygen, anaerobic, or hypoxic conditions.

In one embodiment, the immune initiator is arginine. In another embodiment, the immune initiator is at least one enzyme of an arginine biosynthetic pathway.

In one embodiment, the microorganism comprises at least one gene sequence encoding the at least one enzyme of the arginine biosynthetic pathway. In one embodiment, the at least one gene sequence encoding the at least one enzyme of the arginine biosynthetic pathway comprises feedback resistant argA. In one embodiment, the at least one gene sequence encoding the at least one enzyme of the arginine biosynthetic pathway is selected from the group consisting of: argA, argB, argC, argD, argE, argF, argG, argH, argI, argJ, carA, and carB. In one embodiment, the microorganism further comprises a deletion or a mutation in an arginine repressor gene (argR). In one embodiment, the at least one gene sequence for the production of arginine is integrated into a chromosome of the recombinant microorganism. In one embodiment, the at least one gene sequence for the production of arginine is present on a plasmid. In one embodiment, the at least one gene sequence for the production of arginine is operably linked to an inducible promoter. In one embodiment, the inducible promoter is induced by low oxygen, anaerobic, or hypoxic conditions.

In one embodiment, the immune initiator is 5-FU.

In one embodiment, the microorganism comprises at least one gene sequence encoding an enzyme capable of converting 5-FC to 5-FU. In one embodiment, the at least one gene sequence is codA. In one embodiment, the at least one gene sequence is integrated into a chromosome of the recombinant microorganism. In another embodiment, the at least one gene sequence is present on a plasmid. In one embodiment, the at least one gene sequence encoding the immune initiator is operably linked to an inducible promoter. In one embodiment, the inducible promoter is an FNR promoter.

In one embodiment, the immune sustainer is capable of enhancing trafficking and infiltration of T cells, enhancing recognition of target cells by T cells, enhancing effector T cell response, and/or overcoming immune suppression. In one embodiment, the immune sustainer is capable of enhancing trafficking and infiltration of T cells. In one embodiment, the immune sustainer is capable of enhancing recognition of target cells by T cells. In one embodiment, the immune sustainer is capable of enhancing effector T cell response. In one embodiment, the immune sustainer is capable of overcoming immune suppression.

In one embodiment, the immune sustainer is a therapeutic molecule encoded by at least one gene. In one embodiment, the immune sustainer is a therapeutic molecule produced by an enzyme encoded by at least one gene. In one embodiment, the immune sustainer is at least one enzyme of a biosynthetic or catabolic pathway encoded by at least one gene. In one embodiment, the immune sustainer is at least one therapeutic molecule produced by at least one enzyme of a biosynthetic or catabolic pathway encoded by at least one gene. In one embodiment, the immune sustainer is a nucleic acid molecule that mediates RNA interference, microRNA response or inhibition, TLR response, antisense gene regulation, target protein binding, or gene editing.

In one embodiment, the immune sustainer is a cytokine, a chemokine, a single chain antibody, a ligand, a metabolic converter, a T cell co-stimulatory receptor, a T cell co-stimulatory receptor ligand, or a secreted or displayed peptide.

In one embodiment, the immune sustainer is a metabolic converter, arginine, a STING agonist, CXCL9, CXCL10, anti-PD1 antibody, anti-PDL1 antibody, anti-CTLA4 antibody, agonistic anti-GITR antibody or GITRL, agonistic anti-OX40 antibody or OX40L, agonistic anti-4-1BB antibody or 4-1BBL, IL-15, IL-15 sushi, IFNγ, or IL-12. In one embodiment, the immune sustainer is a secreted peptide or a displayed peptide.

In one embodiment, the immune sustainer is a metabolic converter. In one embodiment, the metabolic converter is at least one enzyme of a kynurenine consumption pathway. In another embodiment, the metabolic converter is at least one enzyme of an adenosine consumption pathway. In another embodiment, the metabolic converter is at least one enzyme of an arginine biosynthetic pathway.

In one embodiment, the microorganism comprises at least one gene sequence encoding the at least one enzyme of the kynurenine consumption pathway. In one embodiment, the at least one gene sequence encoding the at least one enzyme of the kynurenine consumption pathway is a kynureninase gene sequence. In one embodiment, he at least one gene sequence is kynU. In one embodiment, the at least one gene sequence is operably linked to a constitutive promoter. In one embodiment, the at least one gene sequence encoding the at least one enzyme of the kynurenine consumption pathway is integrated into a chromosome of the microorganism. In another embodiment, the at least one gene sequence encoding the at least one enzyme of the kynurenine consumption pathway is present on a plasmid. In one embodiment, the microorganism comprises a deletion or a mutation in trpE.

In one embodiment, the microorganism comprises at least one gene sequence encoding at least one enzyme of an adenosine consumption pathway. In one embodiment, the at least one gene sequence encoding the at least one enzyme of the adenosine consumption pathway is selected from add, xapA, deoD, xdhA, xdhB, and xdhC. In one embodiment, the at least one gene sequence encoding the at least one enzyme of the adenosine consumption pathway is operably linked to a promoter induced by low oxygen, anaerobic, or hypoxic conditions. In one embodiment, the at least one gene sequence encoding the at least one enzyme of the adenosine consumption pathway is integrated into a chromosome of the microorganism. In another embodiment, the at least one gene sequence is present on a plasmid. In one embodiment, the recombinant microorganism comprises at least one gene sequence encoding an enzyme for importing adenosine into the microorganism. In one embodiment, the at least one gene sequence encoding the enzyme for importing adenosine into the microorganism is nupC or nupG.

In one embodiment, the immune sustainer is arginine. In one embodiment, the microorganism comprises at least one gene sequence encoding at least one enzyme of the arginine biosynthetic pathway. In one embodiment, the at least one gene sequence encoding at least one enzyme of the arginine biosynthetic pathway comprises feedback resistant argA. In one embodiment, the at least one gene sequence encoding the at least one enzyme of the arginine biosynthetic pathway is selected from the group consisting of: argA, argB, argC, argD, argE, argF, argG, argH, argI, argJ, carA, and carB. In one embodiment, the at least one gene sequence encoding the at least one enzyme of the arginine biosynthetic pathway is operably linked to a promoter induced by low oxygen, anaerobic, or hypoxic conditions. In one embodiment, the at least one gene sequence encoding the at least one enzyme of the arginine biosynthetic pathway is integrated into a chromosome of the recombinant microorganism or is present on a plasmid. In one embodiment, the microorganism further comprises a deletion or a mutation in an arginine repressor gene (argR).

In one embodiment, the immune sustainer is a STING agonist. In one embodiment, the STING agonist is c-diAMP, c-GAMP, or c-diGMP. In another embodiment, the recombinant microorganism comprises at least one gene sequence encoding an enzyme which produces the STING agonist. In one embodiment, the at least one gene sequence encoding the immune sustainer is a dacA gene sequence. In one embodiment, the at least one gene sequence encoding the immune sustainer is a cGAS gene sequence. In one embodiment, the cGAS gene sequence is selected from a human cGAS gene sequence, a Verminephrobacter eiseniae cGAS gene sequence, Kingella denitrificans cGAS gene sequence, and a Neisseria bacilliformis cGAS gene sequence.

In one embodiment, the immune initiator is not the same as the immune sustainer. In one embodiment, the immune initiator is different than the immune sustainer.

In one embodiment, the recombinant microorganism comprises at least one gene sequence encoding an enzyme capable of producing the STING agonist. In one embodiment, the at least one gene sequence encoding the STING agonist is a dacA gene. In one embodiment, the at least one gene sequence encoding the STING agonist is a cGAS gene. In one embodiment, the STING agonist is c-diAMP. In one embodiment, the STING agonist is c-GAMP. In one embodiment, the STING agonist is c-diGMP.

In one embodiment, the bacterium is an auxotroph in a gene that is not complemented when the bacterium is present in a host. In one embodiment, the gene that is not complemented when the bacterium is present in a host is a dapA gene. In one embodiment, expression of the dapA gene fine-tunes the expression of the one or more immune initiators. In one embodiment, the bacterium is an auxotroph in a gene that is complemented when the bacterium is present in a host. In one embodiment, the gene that is complemented when the bacterium is present in a host is a thyA gene.

In one embodiment, the bacterium further comprises a mutation or deletion in an endogenous prophage.

In one embodiment, the at least one gene sequence is operably linked to an inducible promoter. In one embodiment, the inducible promoter is induced by low-oxygen or anaerobic conditions. In one embodiment, the inducible promoter is induced by a hypoxic environment. In one embodiment, the promoter is an FNR promoter.

In one embodiment, the at least one gene sequence is integrated into a chromosome in the bacterium. In one embodiment, the at least one gene sequence is located on a plasmid in the bacterium.

In one embodiment, the bacterium is non-pathogenic. In one embodiment, he bacterium is Escherichia coli Nissle.

In one aspect, disclosed herein is a recombinant microorganism capable of producing an effector molecule, wherein the effector molecule is selected from the group consisting of CXCL9, CXCL10, hyaluronidase, and SIRPα.

In one embodiment, the recombinant microorganism comprises at least one gene sequence encoding CXCL9. In one embodiment, the at least one gene sequence encoding CXCL9 is linked to an inducible promoter.

In one embodiment, the recombinant microorganism comprises at least one gene sequence encoding CXCL10. In one embodiment, the at least one gene sequence encoding CXCL10 is linked to an inducible promoter.

In one embodiment, the recombinant microorganism comprises at least one gene sequence encoding hyaluronidase. In one embodiment, the at least one gene sequence encoding hyaluronidase is linked to an inducible promoter.

In one embodiment, the recombinant microorganism comprises at least one gene sequence encoding the SIRPα. In one embodiment, the at least one gene sequence encoding the SIRPα is linked to an inducible promoter.

In one embodiment, the effector molecule is secreted. In another embodiment, the effector molecule is displayed on the cell surface.

In one aspect, disclosed herein is a recombinant microorganism capable of converting 5-FC to 5-FU. In another aspect, disclosed herein is a recombinant microorganism capable of converting 5-FC to 5-FU, wherein the recombinant microorganism is further capable of producing a STING agonist.

In one embodiment, the microorganism comprises at least one gene sequence encoding an enzyme capable of converting 5-FC to 5-FU. In one embodiment, the at least one gene sequence is codA. In one embodiment, the at least one gene sequence is a codA::upp fusion. In one embodiment, the at least one gene sequence is operably linked to an inducible promoter or a constitutive promoter. In one embodiment, the inducible promoter is a FNR promoter. In one embodiment, the at least one gene sequence is integrated into the chromosome of the microorganism or is present on a plasmid.

In one embodiment, the microorganism capable of converting 5-FC to 5-FU is further capable of producing a STING agonist. In one embodiment, the STING agonist is c-diAMP, c-GAMP, or c-diGMP. In one embodiment, the recombinant microorganism comprises at least one gene sequence encoding an enzyme which produces the STING agonist. In one embodiment, the at least one gene sequence encoding the enzyme which produces the STING agonist is a dacA gene sequence. In one embodiment, the at least one gene sequence encoding the enzyme which produces the STING agonist is a cGAS gene sequence. In one embodiment, the cGAS gene sequence is a human cGAS gene sequence. In one embodiment, the at least one gene sequence encoding the enzyme which produces the STING agonist is operably linked to an inducible promoter. In one embodiment, the inducible promoter is an FNR promoter. In one embodiment, the at least one gene sequence encoding the enzyme which produces the STING agonist is integrated into a chromosome of the microorganism or is present on a plasmid.

In one embodiment, the recombinant microorganism disclosed herein is a bacterium. In one embodiment, the recombinant microorganism disclosed herein is a yeast. In one embodiment, the recombinant microorganism is an E. coli bacterium. In one embodiment, the recombinant microorganism is an E. coli Nissle bacterium. In one embodiment, the recombinant microorganism is E. coli Nissle strain SYN1557 (delta PAL::CmR).

In one embodiment, the recombinant microorganism disclosed herein comprises at least one mutation or deletion in a gene which results in one or more auxotrophies. In one embodiment, the at least one deletion or mutation is in a dapA gene and/or a thyA gene.

In one embodiment, the recombinant microorganism disclosed herein comprises a phage deletion.

In one aspect, disclosed herein is a composition comprising at least a first recombinant microorganism capable of expressing at least one display protein comprising a viral antigen, and at least a second recombinant microorganism capable of producing an immune modulator.

In one aspect, disclosed herein is a composition comprising at least a first recombinant microorganism capable of expressing at least one display protein comprising a cancer antigen, and at least a second recombinant microorganism capable of producing an immune modulator.

In one aspect, disclosed herein is a composition comprising a viral protein and at least one recombinant microorganism capable of producing an immune modulator. In one embodiment, the at least one recombinant microorganism is capable of producing both the immune initiator and the immune sustainer. In another embodiment, the at least one recombinant microorganism is capable of producing the immune initiator, and at least a second recombinant microorganism is capable of producing the immune sustainer. In yet another embodiment, the immune sustainer is not produced by a recombinant microorganism in the composition. In another embodiment, the at least one recombinant microorganism is capable of producing the immune sustainer, and at least a second recombinant microorganism is capable of producing the immune initiator. In yet another embodiment, the immune initiator is not produced by a recombinant microorganism in the composition.

In one embodiment, the immune initiator is not arginine, TNFα, IFNγ, IFNβ1, GMCSF, anti-CD40 antibody, CD40L, agonistic anti-OX40 antibody, OX040L, agonistic anti-41BB antibody, 41BBL, agonistic anti-GITR antibody, GITRL, anti-PD1 antibody, anti-PDL1 antibody, and/or azurin. In one embodiment, the immune initiator is not arginine. In one embodiment, the immune initiator is not TNFα. In one embodiment, the immune initiator is not IFNγ. In one embodiment, the immune initiator is not IFNβ1. In one embodiment, the immune initiator is not an anti-CD40 antibody. In one embodiment, the immune initiator is not CD40L. In one embodiment, the immune initiator is not GMCSF. In one embodiment, the immune initiator is not an agonistic anti-OX040 antibody. In one embodiment, the immune initiator is not OX040L. In one embodiment, the immune initiator is not an agonistic anti-4-1BB antibody. In one embodiment, the immune initiator is not 4-1BBL. In one embodiment, the immune initiator is not an agonistic anti-GITR antibody. In one embodiment, the immune initiator is not GITRL. In one embodiment, the immune initiator is not an anti-PD1 antibody. In one embodiment, the immune initiator is not an anti-PDL1 antibody. In one embodiment, the immune initiator is not azurin.

In one embodiment, the immune sustainer is not at least one enzyme of a kynurenine consumption pathway, at least one enzyme of an adenosine consumption pathway, anti-PD1 antibody, anti-PDL1 antibody, anti-CTLA4 antibody, IL-15, IL-15 sushi, IFNγ, agonistic anti-GITR antibody, GITRL, an agonistic anti-OX40 antibody, OX40L, an agonistic anti-4-1BB antibody, 4-1BBL, or IL-12. In one embodiment, the immune sustainer is not at least one enzyme of a kynurenine consumption pathway. In one embodiment, the immune sustainer is not at least one enzyme of an adenosine consumption pathway. In one embodiment, the immune sustainer is not arginine. In one embodiment, the immune sustainer is not at least one enzyme of an arginine biosynthetic pathway. In one embodiment, the immune sustainer is not an anti-PD1 antibody. In one embodiment, the immune sustainer is not an anti-PDL1 antibody. In one embodiment, the immune sustainer is not an anti-CTLA4 antibody. In one embodiment, the immune sustainer is not an agonistic anti-GITR antibody. In one embodiment, the immune sustainer is not GITRL. In one embodiment, the immune sustainer is not IL-15. In one embodiment, the immune sustainer is not IL-15 sushi. In one embodiment, the immune sustainer is not IFNγ. In one embodiment, the immune sustainer is not an agonistic anti-OX40 antibody. In one embodiment, the immune sustainer is not OX40L. In one embodiment, the immune sustainer is not an agonistic anti-4-1BB antibody. In one embodiment, the immune sustainer is not 4-1BBL. In one embodiment, the immune sustainer is not IL-12.

In one aspect, disclosed herein is a pharmaceutically acceptable composition comprising a recombinant microorganism disclosed herein, and a pharmaceutically acceptable carrier. In one aspect, disclosed herein is a pharmaceutically acceptable composition comprising a composition disclosed herein, and a pharmaceutically acceptable carrier. In one embodiment, the composition is formulated for intranasal delivery. In one embodiment, the pharmaceutically acceptable composition is for use in treating a subject having a bacterial, viral, or fungal infection. In one embodiment, the pharmaceutically acceptable composition is for preventing a bacterial, viral, or fungal infection in a subject. In another embodiment, the pharmaceutically acceptable composition is for use in treating a subject having an coronavirus infection. In another embodiment, the pharmaceutically acceptable composition is for use in treating a subject having the coronavirus disease 2019 (COVID-19). In one embodiment, the pharmaceutically acceptable composition is for treating cancer in a subject. In another embodiment, the pharmaceutically acceptable composition is for use in inducing and modulating an immune response in a subject.

In one aspect, disclosed herein is a kit comprising a pharmaceutically acceptable composition disclosed herein, and instructions for use thereof.

In one aspect, disclosed herein is a method of treating a bacterial, viral, or fungal infection in a subject, the method comprising administering to the subject a pharmaceutically acceptable composition disclosed herein, thereby treating the bacterial, viral, or fungal infection in the subject.

In one aspect, disclosed herein is a method of treating the coronavirus disease 2019 (COVID-19) in a subject, the method comprising administering to the subject a pharmaceutically acceptable composition disclosed herein, thereby treating the coronavirus disease 2019 (COVID-19) in the subject.

In one aspect, disclosed herein is a method of treating cancer in a subject, the method comprising administering to the subject a pharmaceutically acceptable composition disclosed herein, thereby treating the cancer in the subject.

In one aspect, disclosed herein is a method of inducing and sustaining an immune response in a subject, the method comprising administering to the subject a pharmaceutically acceptable composition disclosed herein, thereby inducing and sustaining the immune response in the subject.

In one aspect, disclosed herein is a method of inducing and sustaining an immune response in a subject, the method comprising administering to the subject a pharmaceutically acceptable composition described herein, thereby inducing and sustaining the immune response in the subject.

In one aspect, disclosed herein is a method of treating a microbial infection in a subject, the method comprising administering a first recombinant microorganism to the subject, wherein the first recombinant microorganism is capable of expressing a display protein; and administering a second recombinant microorganism to the subject, wherein the second recombinant microorganism is capable of producing an immune modulator, thereby treating the microbial infection in the subject.

In one aspect, disclosed herein is a method of treating cancer in a subject, the method comprising administering a first recombinant microorganism to the subject, wherein the first recombinant microorganism is capable of expressing a cancer protein on its cell surface; and administering a second recombinant microorganism to the subject, wherein the second recombinant microorganism is capable of producing an immune modulator, thereby treating cancer in the subject.

In one aspect, disclosed herein is a method of treating the coronavirus disease 2019 (COVID-19) in a subject, the method comprising administering a first recombinant microorganism to the subject, wherein the first recombinant microorganism is capable of producing a viral protein; and administering a second recombinant microorganism to the subject, wherein the second recombinant microorganism is capable of producing an immune modulator, thereby treating the coronavirus disease 2019 (COVID-19) in the subject.

In one aspect, disclosed herein is a method of inducing and sustaining an immune response in a subject, the method comprising administering a first recombinant microorganism to the subject, wherein the first recombinant microorganism is capable of producing a viral protein; and administering a second recombinant microorganism to the subject, wherein the second recombinant microorganism is capable of producing an immune modulator, thereby inducing and sustaining the immune response in the subject.

In one embodiment, the administering steps are performed at the same time. In one embodiment, the administering of the first recombinant microorganism to the subject occurs before the administering of the second recombinant microorganism to the subject. In one embodiment, the administering of the second recombinant microorganism to the subject occurs before the administering of the first recombinant microorganism to the subject.

In one aspect, disclosed herein is a method of treating a microbial infection in a subject, the method comprising administering a first recombinant microorganism to the subject, wherein the first recombinant microorganism is capable of displaying a display protein on its surface; and administering an immune modulator to the subject, thereby treating the microbial infection in the subject.

In one aspect, disclosed herein is a method of treating cancer in a subject, the method comprising administering a first recombinant microorganism to the subject, wherein the first recombinant microorganism is capable of expressing a protein on its surface via a display protein; and administering an immune modulator to the subject, thereby treating cancer in the subject.

In one aspect, disclosed herein is a method of treating the coronavirus disease 2019 (COVID-19) in a subject, the method comprising administering a first recombinant microorganism to the subject, wherein the first recombinant microorganism is capable of displaying a viral protein; and administering an immune modulator to the subject, thereby treating the coronavirus disease 2019 (COVID-19) in the subject.

In one aspect, disclosed herein is a method of inducing and sustaining an immune response in a subject, the method comprising administering a first recombinant microorganism to the subject, wherein the first recombinant microorganism is capable of displaying a viral protein; and administering an immune modulator to the subject, thereby inducing and sustaining the immune response in the subject.

In one embodiment, the administering steps are performed at the same time. In one embodiment, the administering of the first recombinant microorganism to the subject occurs before the administering of the immune sustainer to the subject. In another embodiment, the administering of the immune sustainer to the subject occurs before the administering of the first recombinant microorganism to the subject.

In one aspect, disclosed herein is a method of treating a microbial infection in a subject, the method comprising administering a protein to the subject; and administering a first recombinant microorganism to the subject, wherein the first recombinant microorganism is capable of producing an immune modulator, thereby treating the microbial infection in the subject.

In one aspect, disclosed herein is a method of treating the coronavirus disease 2019 (COVID-19) in a subject, the method comprising administering a viral protein to the subject; and administering a first recombinant microorganism to the subject, wherein the first recombinant microorganism is capable of producing an immune modulator, thereby treating the coronavirus disease 2019 (COVID-19) in the subject.

In one aspect, disclosed herein is a method of inducing and sustaining an immune response in a subject, the method comprising administering a viral protein to the subject; and administering a first recombinant microorganism to the subject, wherein the first recombinant microorganism is capable of producing an immune modulator, thereby inducing and sustaining the immune response in the subject.

In one embodiment, the administering steps are performed at the same time. In one embodiment, the administering of the first recombinant microorganism to the subject occurs before the administering of the immune initiator to the subject. In one embodiment, the administering of the immune initiator to the subject occurs before the administering of the first recombinant microorganism to the subject.

In one embodiment, the administering is intranasal injection.

Accordingly, the disclosure provides compositions comprising one or more modified bacteria comprising gene sequence(s) encoding one or more immune modulators. In some embodiments, the immune modulator is an immune initiator, which may for example modulate, e.g., promote cell lysis, antigen presentation by dendritic cells or macrophages, or T cell activation or priming. Examples of such immune initiators include cytokines or chemokines, such as TNFα, IFN-gamma and IFN-beta1, a single chain antibodies, such as anti-CD40 antibodies, or (3) ligands such as SIRPα or CD40L, a metabolic enzymes (biosynthetic or catabolic), such as a STING agonist producing enzyme, or (5) cytotoxic chemotherapies. The immune modulators, e.g., immune initiators, may be operably linked to a promoter not associated with the gene sequence(s) in nature.

In some embodiments, the genetically engineered bacteria are capable of producing one or more STING agonist(s), such as c-di-AMP, 3′3′-cGAMP and/or c-2′3′-cGAMP. In some embodiments, the genetically engineered bacteria comprise gene sequences encoding a diadenylate cyclase, such as DacA, e.g., from Listeria monocytogenes. In some embodiments, the genetically engineered bacteria comprise gene sequences encoding a 3′3′-cGAMP synthase. Non-limiting examples of 3′3′-cGAMP synthases described in the instant disclosure include 3′3′-cGAMP synthase Verminephrobacter eiseniae (EF01-2 Earthworm symbiont), 3′3′-cGAMP synthase from Kingella denitrificans (ATCC 33394), and 3′3′-cGAMP synthase from Neisseria bacilliformis (ATCC BAA-1200). In some embodiments, the genetically engineered bacteria comprise gene sequences encoding a 2′3′-cGAMP synthase, such as human cGAS.

In some embodiments, the genetically engineered bacteria comprise gene sequences encoding agonists of co-stimulatory receptors, including but not limited to OX40, GITR, 41BB.

In some embodiments, the composition further comprises one or more genetically engineered microorganism(s) comprising gene sequence(s) for producing an immune sustainer. Such a sustainer may be selected from a cytokine or chemokine, a single chain antibody antagonistic peptide or ligand, and a metabolic enzyme pathways.

Examples of immune sustaining cytokines which may be produced by the genetically engineered bacteria include IL-15 and CXCL10, which may be secreted into the microenvironment. Non-limiting examples of single chain antibodies include anti-PD-1, anti-PD-L1, or anti-CTLA-4, which may be secreted into the microenvironment or displayed on the microorganism cell surface.

In some embodiments, the genetically engineered bacteria comprise gene sequences encoding circuitry for one or more metabolic conversions, i.e., the bacteria are capable performing one or more enzyme-catalyzed reactions, which can be either biosynthetic or catabolic in nature. Accordingly, in some embodiments, the genetically engineered bacteria are capable of producing metabolites which modulate, e.g., promote or contribute to immune initiation and/or immune sustenance or are capable of consuming metabolites which modulate, e.g., inhibit viral infection.

In any of these compositions, the promoter operably linked to the gene sequences(s) for producing the immune modulator, e.g., the immune initiator and/or immune sustainer may an inducible promoter. In some embodiments, the promoter is induced by low-oxygen or anaerobic conditions, such as by a hypoxic environment. Non-limiting examples of such low oxygen inducible promoters of the disclosure include FNR-inducible promoters, ANR-inducible promoters, and DNR-inducible promoters. In some embodiments, the promoter operably linked to the gene sequence(s) for producing the immune modulator, e.g., the immune initiator or immune sustainer, is directly or indirectly induced by a chemical inducer that is not normally present. In some embodiments, the promoter is induced in vitro during fermentation in a suitable growth vessel. In some embodiments, the chemical inducer is selected from tetracycline, IPTG, arabinose, cumate, and salicylate.

In some embodiments, the composition comprises bacteria that are auxotrophs for a particular metabolite, e.g., the bacterium is an auxotroph in a gene that is not complemented when the microorganism(s) is present in the host. In some embodiments, the bacterium is an auxotroph in the DapA gene. In some embodiments, the composition comprises bacteria that are auxotrophs for a particular metabolite, e.g., the bacterium is an auxotroph in a gene that is complemented when the microorganism(s) is present in the host. In some embodiments, the bacterium is an auxotroph in the ThyA gene. In some embodiments, the bacterium is an auxotroph in the TrpE gene.

In some embodiments, the bacterium is a Gram-positive bacterium. In some embodiments, the bacterium is a Gram-negative bacterium. In some embodiments, the bacterium is an obligate anaerobic bacterium. In some embodiments, the bacterium is a facultative anaerobic bacterium. Non-limiting examples of bacteria contemplated in the disclosure include Clostridium novyi NT, and Clostridium butyricum, and Bifidobacterium longum. In some embodiments, the bacterium is selected from E. coli Nissle, and E. coli K-12.

In some embodiments, the bacterium comprises an antibiotic resistance gene sequence. In some embodiments, the one or more of the gene sequence(s) encoding the immune modulator(s) are present on a chromosome. In some embodiments, the one or more of the gene sequence(s) encoding the immune modulator(s) are present on a plasmid.

Additionally, pharmaceutical compositions are provided, further comprising one or more immune checkpoint inhibitors, such as CTLA-4 inhibitor, a PD-1 inhibitor, and a PD-L1 inhibitor. Such checkpoint inhibitors may be administered in combination, sequentially or concurrently with the genetically engineered bacteria.

Additionally, pharmaceutical compositions are provided, further comprising one or more agonists of co-stimulatory receptors, such as OX40, GITR, and/or 41BB, including but not limited to agonistic molecules, such as ligands or agonistic antibodies which are capable of binding to co-stimulatory receptors, such as OX40, GITR, and/or 41BB. Such agonistic molecules may be administered in combination, sequentially or concurrently with the genetically engineered bacteria.

In any of these embodiments, a combination of engineered bacteria can be used in conjunction with conventional anti-viral therapies. In any of these embodiments, the engineered bacteria can produce one or more cytotoxins or lytic peptides. In any of these embodiments, the engineered bacteria can be used in conjunction with a viral vaccine.

In one embodiment, disclosed herein is a modified bacterium comprising at least one an immune initiator, wherein the immune initiator is capable of producing a stimulator of interferon gene (STING) agonist.

### BRIEF DESCRIPTION OF THE FIGURES

FIG. 1 depicts a schematic showing surface display on the outer membrane including an anchor domain, a linker, and a displayed reporter.

FIG. 2 depicts surface display of GFP and FLAG tag analyzed by flow cytometry. E. coli Nissle cells containing a negative control or one of three constructs each containing a different anchor domain, and FLAG tag, and GFP were analyzed.

FIG. 3 depicts surface display of GFP and FLAG tag analyzed by flow cytometry. A negative control was compared to three constructs each containing a different anchor domain, a FLAG tag, and GFP. Invasin N, yiaT, and intimin N were compared as anchor domains.

FIGS. 4A, 4B, and 5A, 5B, and 5C depict additional flow cytometry analysis of constructs including different anchor domains, a FLAG tag, and GFP.

FIG. 6 depicts a schematic showing Nissle surface display of nanobody A4 binding to CD47. In vitro staining shows binding of A4 protein by recombinant CD47 protein using E. coli Nissle cells displaying A4 on the membrane.

FIG. 7 depicts surface display of A4 protein incubated with or without CD47 and analyzed by flow cytometry.

FIG. 8 depicts histogram plots of surface display of EGFR analyzed by flow cytometry.

FIG. 9 depicts a schematic showing the product concept for engineered E. coli Nissle vaccine design and mechanism of action.

FIG. 10 depicts a schematic showing the STING Pathway in Antigen Presenting Cells.

FIG. 11 depicts the design of S protein antigen variants for Nissle surface display.

FIGS. 12, 13, and 14 depict additional exemplary designs of S protein antigen variants for Nissle surface display.

### DETAILED DESCRIPTION

The disclosure relates to recombinant microorganisms, e.g., recombinant bacteria, pharmaceutical compositions thereof, and methods of preventing or treating infections, e.g., viral, bacterial, or fungal infections. In certain aspects, the compositions and methods disclosed herein may be used to display and deliver one or more viral, bacterial, fungal, or cancer protein and/or immune modulators to a host/host cells to prevent and/or treat viral, bacterial, or fungal infections. In one embodiment, the microorganism is a vaccine.

This disclosure relates to compositions and therapeutic methods for the local and target-specific display and/or delivery of one or more viral, bacterial, fungal, or cancer protein and/or immune modulators in order to prevent and/or treat viral, bacterial, or fungal infection and/or diseases, e.g., COVID-19. In certain aspects, the disclosure relates to genetically engineered microorganisms that are capable of producing one or more effector molecules e.g., immune modulators, such as any of the effector molecules provided herein. In certain aspects, the disclosure relates to genetically engineered bacteria that are capable of producing one or more effector molecules, e.g., immune modulators(s).

Specifically, in some embodiments, the genetically engineered bacteria are capable of producing one or more viral proteins. In some embodiments the genetically engineered bacteria are capable of producing one or more immune modulators in combination with one or more viral proteins. In one embodiment, the subject to which the bacteria are delivered generate and sustain an immune response against the one or more viral proteins, thereby preventing and/or treating COVID-19 in the subject.

Specifically, in some embodiments, the genetically engineered bacteria are capable of producing one or more viral, bacterial, fungal, or cancer protein. In some embodiments the genetically engineered bacteria are capable of producing one or more immune modulators in combination with one or more viral, bacterial, fungal, or cancer protein. In one embodiment, the subject to which the bacteria are delivered generate and sustain an immune response against the one or more viral, bacterial, fungal, or cancer protein, thereby preventing and/or treating the viral, bacterial, or fungal infection or cancer in the subject.

In some aspects, the disclosure provides a genetically engineered microorganism that is capable of delivering one or more effector molecules, e.g., immune modulators, such as immune initiators and/or immune sustainers. In some aspects, the disclosure relates to a genetically engineered microorganism that is delivered systemically, e.g., via any of the delivery means described in the present disclosure, and are capable of producing one or more effector molecules, e.g., immune initiators and/or immune sustainers, as described herein. In some aspects, the disclosure relates to a genetically engineered microorganism that is delivered locally, and are capable of producing one or more effector molecules, e.g., immune initiators and/or immune sustainers. In some aspects, the compositions and methods disclosed herein may be used to deliver one or more effector molecules, e.g., immune initiators and/or immune sustainers selectively, thereby reducing systemic cytotoxicity or systemic immune dysfunction, e.g., the onset of an autoimmune event or other immune-related adverse event.

In some aspects, disclosed here is a recombinant microorganism capable of displaying a protein, e.g., an antigen. In one embodiment, the recombinant microorganism express a display protein. In one aspect, the display protein comprises an anchor domain, e.g., intimin, PelB-PAL, YiaT, LppOmpA, BAN, OmsY, Invasin, IgA, PgsA, Ice nucleation protein, and NGIgAsig-NGIgAb, a linker, and a displayed protein, e.g., antigen.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “microbial,” refers to or related to a microorganism, e.g., a virus, a bacterium, and a fungi.

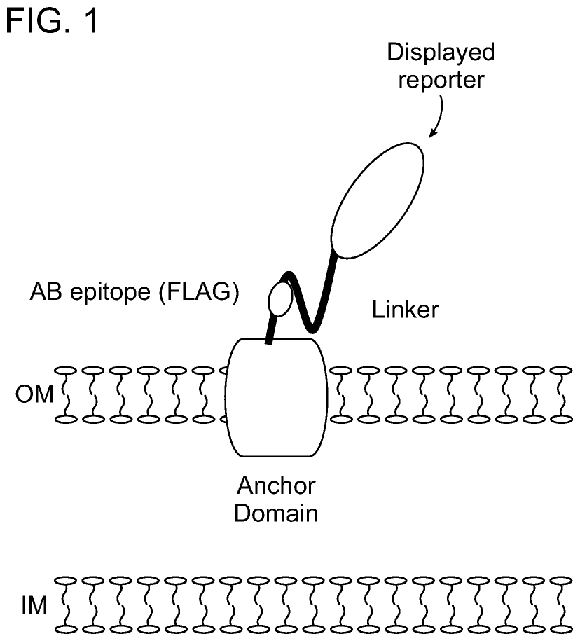

As used herein, the term “display protein” refers to a protein, e.g., a fusion protein, which comprises an anchor domain, a linker, and a displayed protein. As a non-limiting example of a display protein, see, for example, FIG. 1.

As used herein, the term “anchor domain” refers to a protein capable of anchoring a displayed protein to the outer membrane of a microorganism, e.g., recombinant bacterium. Anchor domains are well known to those of ordinary skill in the art and include, for example, Intimin, PAL, PelB-PAL, YiaT, BclA, LppOmpA, BAN, OmsY, Invasin, IgA, PgsA, Ice nucleation protein (INP), and NGIgAsig-NGIgAb.

LppOmpA is described, for example, in Francisco et al., Proc. Natl. Acad. Sci. USA 89:2713-2717, 1992; and Francisco et al., Proc. Natl. Acad. Sci. USA, 90:10444-10448, 1993; the entire contents of each of which are expressly incorporated herein by reference. LppOmpA comprises the Lpp signal peptide and first 9 amino acids-Gly-Ile-OmpA. Peptidoglycan-associated lipoprotein (PAL) is described at least in Dhillon et al., Letters in Applied Microbiology, 28:350-354, 1999, the entire contents of which are expressly incorporated herein by reference. NGIgA is described a least in Pyo et al., Vaccine, 27(14):2030-2036, 2009, the entire contents of which are expressly incorporated herein by reference. Ice nucleation protein (INP) is described in Li et al., FEMS Microbiology Letters, 299(1):44-52, 2009, the entire contents of which are expressly incorporated herein by reference. BclA is described at least in Park et al., Microbial Cell Factories, 12, article 81, 2013, the entire contents of which are expressly incorporated herein by reference. Intimin is described at least in Wentzel et al., J. Bacteriol., 183(24):7273-7284, 2001, the entire contents of which are expressly incorporated herein by reference.

As used herein the term “linker” refers to a protein used to fuse the anchor domain to the displayed protein. In one embodiment, a linker protein comprises an AB epitope, such as FLAG or HIS tags, Linker proteins are well known to those of ordinary skill in the art and include, for example, GGGGS (SEQ ID NO: 1477), (GGGGS)x2 (SEQ ID NO: 1478), (GGGGS)x3 (SEQ ID NO: 1479), EAAAK (SEQ ID NO: 1480), (EAAAK)x2 (SEQ ID NO: 1481), (EAAAK)x3 (SEQ ID NO: 1482), etc.

As used herein, the term “displayed protein” refers to a protein which can be displayed on the surface of a recombinant bacterium. In one embodiment, a displayed protein can be a reporter protein, e.g., GFP. In another embodiment, a displayed protein can be a protein which is capable of inducing an immune response in a subject, e.g., a human subject. In one embodiment, the displayed protein can be a microbial protein, e.g., a viral protein, a bacterial protein, or a fungal protein. In one embodiment, the displayed protein can be a cancer protein. In one embodiment, the viral protein can be a coronavirus protein. In another embodiment, the protein can be the nanobody A4, which is recognized by CD47-IgG. In one embodiment, the displayed protein is an antigen.

As used herein, the term “antigen” refers to molecular structure, e.g., a protein, which is recognized by host B-cell receptor or host T cell-receptor and capable of inducing immune response in a subject.

As used herein, the term “coronavirus,” (“CoV”; subfamily Coronavirinae, family Coronaviridae, order Nidovirales), refers to a group of highly diverse, enveloped, positive-sense, single-stranded RNA viruses that cause respiratory, enteric, hepatic and neurological diseases of varying severity in a broad range of animal species, including humans. Coronaviruses are subdivided into four genera: Alphacoronavirus, Betacoronavirus (13CoV), Gammacoronavirus and Deltacoronavirus.

Any coronavirus that infects humans and animals is encompassed by the term “coronavirus” as used herein. Exemplary coronaviruses encompassed by the term include the coronaviruses that cause a common cold-like respiratory illness, e.g., human coronavirus 229E (HCoV-229E), human coronavirus NL63 (HCoV-NL63), human coronavirus OC43 (HCoV-OC43), and human coronavirus HKU1 (HCoV-HKU1); the coronavirus that causes avian infectious bronchitis virus (IBV); the coronavirus that causes murine hepatitis virus (MHV); the coronavirus that causes porcine transmissible gastroenteritis virus PRCoV; the coronavirus that causes porcine respiratory coronavirus and bovine coronavirus; the coronavirus that causes Severe Acute Respiratory Syndrome (SARS), the coronavirus that causes the Middle East respiratory syndrome (MERS), and the coronavirus that causes Severe Acute Respiratory Syndrome 2 (SARS-CoV-2; COVID-19).

The coronavirus (CoV) genome is a single-stranded, non-segmented RNA genome, which is approximately 26-32 kb. It contains 5-methylated caps and 3′-polyadenylated tails and is arranged in the order of 5′, replicase genes, genes encoding structural proteins (spike glycoprotein (S), envelope protein (E), membrane protein (M) and nucleocapsid protein (N)), polyadenylated tail and then the 3′ end. The partially overlapping 5-terminal open reading frame 1a/b (ORF1a/b) is within the 5′ two-thirds of the CoV genome and encodes the large replicase polyprotein 1a (pp 1a) and pp lab. These polyproteins are cleaved by papain-like cysteine protease (PLpro) and 3C-like serine protease (3CLpro) to produce non-structural proteins, including RNA-dependent RNA polymerase (RdRp) and helicase (Hel), which are important enzymes involved in the transcription and replication of CoVs. The 3′ one-third of the CoV genome encodes the structural proteins (S, E, M and N), which are essential for virus-cell-receptor binding and virion assembly, and other non-structural proteins and accessory proteins that may have immunomodulatory effects. (Peiris J S., et al., 2003, Nat. Med. 10 (Suppl. 12): 88-97).

As a coronavirus is a positive-sense, single-stranded RNA virus having a 5′ methylated cap and a 3′ polyadenylated tail, once the virus enters the cell and is uncoated, the viral RNA genome attaches to the host cell's ribosome for direct translation. The host ribosome translates the initial overlapping open reading frame of the virus genome and forms a long polyprotein. The polyprotein has its own proteases which cleave the polyprotein into multiple nonstructural proteins.

A number of the nonstructural proteins coalesce to form a multi-protein replicase-transcriptase complex (RTC). The main replicase-transcriptase protein is the RNA-dependent RNA polymerase (RdRp). It is directly involved in the replication and transcription of RNA from an RNA strand. The other nonstructural proteins in the complex assist in the replication and transcription process. The exoribonuclease non-structural protein for instance provides extra fidelity to replication by providing a proofreading function which the RNA-dependent RNA polymerase lacks.

One of the main functions of the complex is to replicate the viral genome. RdRp directly mediates the synthesis of negative-sense genomic RNA from the positive-sense genomic RNA. This is followed by the replication of positive-sense genomic RNA from the negative-sense genomic RNA. The other important function of the complex is to transcribe the viral genome. RdRp directly mediates the synthesis of negative-sense subgenomic RNA molecules from the positive-sense genomic RNA. This is followed by the transcription of these negative-sense subgenomic RNA molecules to their corresponding positive-sense mRNAs

The replicated positive-sense genomic RNA becomes the genome of the progeny viruses.

As use herein, the terms “severe acute respiratory syndrome coronavirus 2,” “SARS-CoV-2,” “2019-nCoV,” refer to the novel coronavirus that caused a pneumonia outbreak first reported in Wuhan, China in December 2019 (“COVID-19”). Phylogenetic analysis of the complete viral genome (29,903 nucleotides) revealed that SARS-CoV-2 was most closely related (89.1% nucleotide similarity similarity) to SARS-CoV.

The term “SARS-CoV-2,” as used herein, also refers to naturally occurring RNA sequence variations of the SARS-CoV-2 genome.

Additional examples of coronavirus genomes and mRNA sequences are readily available using publicly available databases, e.g., GenBank, UniProt, and OMIM.

As used herein the term “immune initiation” or “initiating the immune response” refers to advancement through the steps which lead to the generation and establishment of an immune response.

As used herein the term “immune sustenance” or “sustaining the immune response” refers to the advancement through steps which ensure the immune response is broadened and strengthened over time and which prevent dampening or suppression of the immune response. For example, these steps could include i.e., T cell trafficking, recognition of target cells though TCRs, and overcoming immune suppression, i.e., depletion or inhibition of T regulatory cells and preventing the establishment of other active suppression of the effector response.

Accordingly, in some embodiments, the genetically engineered bacteria are capable of producing one or more effector molecules, e.g., immune modulators, which modulate, e.g., intensify the initiation of the immune response. Accordingly, in some embodiments, the genetically engineered bacteria are capable of producing one or more effector molecules, e.g., immune modulators, which modulate, e.g., enhance, sustenance of the immune response. Accordingly, in some embodiments, the genetically engineered bacteria are capable of producing one or more effector molecules, e.g., immune modulators, which modulate, e.g., intensify, the initiation of the immune response and one or more one or more effector molecules, e.g., immune modulators, which modulate, e.g., enhance, sustenance of the immune response.

Accordingly, in some embodiments, the genetically engineered bacteria comprise gene sequences encoding one or more effector molecules, e.g., immune modulators, which modulate, e.g., intensify the initiation of the immune response. Accordingly, in some embodiments, the genetically engineered bacteria comprise gene sequences encoding one or more effector molecules, e.g., immune modulators, which modulate, e.g., enhance, sustenance of the immune response. Accordingly, in some embodiments, the genetically engineered bacteria comprise gene sequences encoding one or more effector molecules, e.g., immune modulators, which modulate, e.g., intensify, the initiation of the immune response and one or more one or more effector molecules, e.g., immune modulators, which modulate, e.g., enhance, sustenance of the immune response.

An “effector”, “effector substance” or “effector molecule” refers to one or more molecules, therapeutic substances, or drugs of interest. In one embodiment, the “effector” is produced by a modified microorganism, e.g., bacteria. In another embodiment, a modified microorganism capable of producing a first effector described herein is administered in combination with a second effector, e.g., a second effector not produced by a modified microorganism but administered before, at the same time as, or after, the administration of the modified microorganism producing the first effector.

A non-limiting example of such effector or effector molecules are “immune modulators,” which include immune sustainers and/or immune initiators as described herein. In some embodiments, the modified microorganism is capable of producing two or more effector molecules or immune modulators. In some embodiments, the modified microorganism is capable of producing three, four, five, six, seven, eight, nine, or ten effector molecules or immune modulators. In some embodiments, the effector molecule or immune modulator is a therapeutic molecule that is useful for preventing and/or treating a viral disease, e.g., the coronavirus disease 2019 (COVID-19). In another embodiment, a modified microorganism capable of producing a first immune modulator described herein is administered in combination with a second immune modulator, e.g., a second immune modulator not produced by a modified microorganism but administered before, at the same time as, or after, the administration of the modified microorganism producing the first immune modulator.

In some embodiments, the effector or immune modulator is a therapeutic molecule encoded by at least one gene. In other embodiments, the effector or immune modulator is a therapeutic molecule produced by an enzyme encoded by at least one gene. In alternate embodiments, the effector molecule or immune modulator is a therapeutic molecule produced by a biochemical or biosynthetic pathway encoded by at least one gene. In another embodiment, the effector molecule or immune modulator is at least one enzyme of a biochemical, biosynthetic, or catabolic pathway encoded by at least one gene. In some embodiments, the effector molecule or immune modulator may be a nucleic acid molecule that mediates RNA interference, microRNA response or inhibition, TLR response, antisense gene regulation, target protein binding (aptamer or decoy oligos), or gene editing, such as CRISPR interference. Other types of effectors and immune modulators are described and listed herein.

Non-limiting examples of effector molecules and/or immune modulators include immune checkpoint inhibitors (e.g., CTLA-4 antibodies, PD-1 antibodies, PDL-1 antibodies), cytotoxic agents (e.g., Cly A, FASL, TRAIL, TNFα), immunostimulatory cytokines and co-stimulatory molecules (e.g., OX40 antibody or OX40L, CD28, ICOS, CCL21, IL-2, IL-18, IL-15, IL-12, IFN-gamma, IL-21, TNFs, GM-CSF), antigens and antibodies (e.g., viral antigens, tumor antigens, neoantigens, CtxB-PSA fusion protein, CPV-OmpA fusion protein, NY-ESO-1 tumor antigen, RAF1, antibodies against immune suppressor molecules, anti-VEGF, Anti-CXR4/CXCL12, anti-GLP1, anti-GLP2, anti-galectin1, anti-galectin3, anti-Tie2, anti-CD47, antibodies against immune checkpoints, antibodies against immunosuppressive cytokines and chemokines), DNA transfer vectors (e.g., endostatin, thrombospondin-1, TRAIL, SMAC, Stat3, Bcl2, FLT3L, GM-CSF, IL-12, AFP, VEGFR2), and enzymes (e.g., E. coli CD, HSV-TK), immune stimulatory metabolites and biosynthetic pathway enzymes that produce them (STING agonists, e.g., c-di-AMP, 3′3′-cGAMP, and 2′3′-cGAMP; arginine, tryptophan).

Immune modulators include, inter alia, immune initiators and immune sustainers.

As used herein, the term “immune initiator” or “initiator” refers to a class of effectors or molecules, e.g., immune modulators, or substances. In one embodiment, an immune initiator may be produced by a modified microorganism, e.g., bacterium, described herein, or may be administered in combination with a modified microorganism of the disclosure. For example, a modified microorganism capable of producing a first immune initiator or immune sustainer described herein is administered in combination with a second immune initiator, e.g., a second immune initiator not produced by a modified microorganism but administered before, at the same time as, or after, the administration of the modified microorganism producing the first immune initiator or immune sustainer. Non-limiting examples of such immune initiators are described in further detail herein.

In some embodiments, an immune initiator is a therapeutic molecule encoded by at least one gene. Non-limiting examples of such therapeutic molecules are described herein and include, but are not limited to, cytokines, chemokines, single chain antibodies (agonistic or antagonistic), ligands (agonistic or antagonistic), co-stimulatory receptors/ligands and the like. In another embodiment, an immune initiator is a therapeutic molecule produced by an enzyme encoded by at least one gene. Non-limiting examples of such enzymes are described herein and include, but are not limited to, DacA and cGAS, which produce a STING agonist. In another embodiment, an immune initiator is at least one enzyme of a biosynthetic pathway encoded by at least one gene. Non-limiting examples of such biosynthetic pathways are described herein and include, but are not limited to, enzymes involved in the production of arginine. In another embodiment, an immune initiator is at least one enzyme of a catabolic pathway encoded by at least one gene. Non-limiting examples of such catabolic pathways are described herein and include, but are not limited to, enzymes involved in the catabolism of a harmful metabolite. In another embodiment, an immune initiator is at least one molecule produced by at least one enzyme of a biosynthetic pathway encoded by at least one gene. In another embodiment, an immune initiator is a therapeutic molecule produced by metabolic conversion, i.e., the immune initiator is a metabolic converter. In other embodiments, the immune initiator may be a nucleic acid molecule that mediates RNA interference, microRNA response or inhibition, TLR response, antisense gene regulation, target protein binding (aptamer or decoy oligos), gene editing, such as CRISPR interference.

The term “immune initiator” may also refer to any modifications, such as mutations or deletions, in endogenous genes. In some embodiments, the bacterium is engineered to express the biochemical, biosynthetic, or catabolic pathway. In some embodiments, the bacterium is engineered to produce a second messenger molecule.

As used herein, the term “low oxygen” is meant to refer to a level, amount, or concentration of oxygen (O₂) that is lower than the level, amount, or concentration of oxygen that is present in the atmosphere (e.g., <21% O₂; <160 torr O₂)). Thus, the term “low oxygen condition or conditions” or “low oxygen environment” refers to conditions or environments containing lower levels of oxygen than are present in the atmosphere.

In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg 02, 3.75 mmHg 02, 4.5 mmHg 02, 6.8 mmHg 02, 11.35 mmHg 02, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorporated by reference herewith in their entireties.

In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nonaerobic, microoxic, hypoxic, anoxic, and/or anaerobic conditions. For example, Table 1 summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov. 2013, www.fondriest.com/environmental-measurements/parameters/water-quality/dissolved-oxygen/>.

In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated.

In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.).

The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂, etc.). The exemplary fractions and ranges listed here are for illustrative purposes and not meant to be limiting in any way.

TABLE 1
Oxygen present in various organs and tissues

| Compartment | Oxygen Tension |
|---|---|
| stomach | ~60 torr (e.g., 58 +/− 15 torr) |
| duodenum and first | ~30 torr (e.g., 32 +/− 8 torr);~20% |
| part of jejunum | oxygen in ambient air |
| Ileum (mid-small | ~10 torr;~6% oxygen in ambient |
| intestine) | air (e.g., 11 +/− 3 torr) |
| Distal sigmoid colon | ~3 torr (e.g., 3 +/− 1 torr) |
| colon | <2 torr |
| Lumen of cecum | <1 torr |
| tumor | <32 torr (most tumors are < |
| | 15 torr) |

As used herein, the term “gene” or “gene sequence” refers to any sequence expressing a polypeptide or protein, including genomic sequences, cDNA sequences, naturally occurring sequences, artificial sequences, and codon optimized sequences. The term “gene” or “gene sequence” inter alia includes modification of endogenous genes, such as deletions, mutations, and expression of native and non-native genes under the control of a promoter that that they are not normally associated with in nature.

As used herein the terms “gene cassette” and “circuit” or “circuitry” inter alia refers to any sequence expressing a polypeptide or protein, including genomic sequences, cDNA sequences, naturally occurring sequences, artificial sequences, and codon optimized sequences includes modification of endogenous genes, such as deletions, mutations, and expression of native and non-native genes under the control of a promoter that that they are not normally associated with in nature.

An antibody generally refers to a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer composed of two identical pairs of polypeptide chains, each pair having one “light” (about 25 kD) and one “heavy” chain (about 50-70 kD), connected through a disulfide bond.

As used herein, the term “antibody” or “antibodies” is meant to encompasses all variations of antibody and fragments thereof that possess one or more particular binding specificities. Thus, the term “antibody” or “antibodies” is meant to include full length antibodies, chimeric antibodies, humanized antibodies, single chain antibodies (ScFv, camelids), Fab, Fab′, multimeric versions of these fragments (e.g., F(ab′)2), single domain antibodies (sdAB, V_{H}H framents), heavy chain antibodies (HCAb), nanobodies, diabodies, and minibodies. Antibodies can have more than one binding specificity, e.g. be bispecific. The term “antibody” is also meant to include so-called antibody mimetics, i.e., which can specifically bind antigens but do not have an antibody-related structure.

A “single-chain antibody” or “single-chain antibodies” typically refers to a peptide comprising a heavy chain of an immunoglobulin, a light chain of an immunoglobulin, and optionally a linker or bond, such as a disulfide bond. The single-chain antibody lacks the constant Fc region found in traditional antibodies. In some embodiments, the single-chain antibody is a naturally occurring single-chain antibody, e.g., a camelid antibody. In some embodiments, the single-chain antibody is a synthetic, engineered, or modified single-chain antibody. In some embodiments, the single-chain antibody is capable of retaining substantially the same antigen specificity as compared to the original immunoglobulin despite the addition of a linker and the removal of the constant regions. In some aspects, the single chain antibody can be a “scFv antibody”, which refers to a fusion protein of the variable regions of the heavy (VH) and light chains (VL) of immunoglobulins (without any constant regions), optionally connected with a short linker peptide of ten to about 25 amino acids, as described, for example, in U.S. Pat. No. 4,946,778, the contents of which is herein incorporated by reference in its entirety. The Fv fragment is the smallest fragment that holds a binding site of an antibody, which binding site may, in some aspects, maintain the specificity of the original antibody. Techniques for the production of single chain antibodies are described in U.S. Pat. No. 4,946,778.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. In some embodiments, the polypeptide is produced by the genetically engineered bacteria of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: Ala, Pro, Gly, Gln, Asn, Ser, Thr; Cys, Ser, Tyr, Thr; Val, Ile, Leu, Met, Ala, Phe; Lys, Arg, His; Phe, Tyr, Trp, His; and Asp, Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety. In some embodiments, the linker is a glycine rich linker. In some embodiments, the linker is (Gly-Gly-Gly-Gly-Ser)n (SEQ ID NO: 1507). In some embodiments, the linker comprises SEQ ID NO: 979.

As used herein the term “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism.

Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting the immune modulator from the microbial, e.g., bacterial cytoplasm. Non-limiting examples of secretion systems for gram negative bacteria include the modified type III flagellar, type I (e.g., hemolysin secretion system), type II, type IV, type V, type VI, and type VII secretion systems, resistance-nodulation-division (RND) multi-drug efflux pumps, various single membrane secretion systems. Non-limiting examples of secretion systems are described herein.

As used herein, the term “transporter” is meant to refer to a mechanism, e.g., protein or proteins, for importing a molecule into the microorganism from the extracellular milieu.

The immune system is typically most broadly divided into two categories—innate immunity and adaptive immunity—although the immune responses associated with these immunities are not mutually exclusive. “Innate immunity” refers to non-specific defense mechanisms that are activated immediately or within hours of a foreign agent's or antigen's appearance in the body. These mechanisms include physical barriers such as skin, chemicals in the blood, and immune system cells, such as dendritic cells (DCs), leukocytes, phagocytes, macrophages, neutrophils, and natural killer cells (NKs), that attack foreign agents or cells in the body and alter the rest of the immune system to the presence of the foreign agents. During an innate immune response, cytokines and chemokines are produced which in combination with the presentation of immunological antigens, work to activate adaptive immune cells and initiate a full blown immunologic response. “Adaptive immunity” or “acquired immunity” refers to antigen-specific immune response. The antigen must first be processed or presented by antigen presenting cells (APCs). An antigen-presenting cell or accessory cell is a cell that displays antigens directly or complexed with major histocompatibility complexes (MHCs) on their surfaces. Professional antigen-presenting cells, including macrophages, B cells, and dendritic cells, specialize in presenting foreign antigen to T helper cells in a MHC-II restricted manner, while other cell types can present antigen originating inside the cell to cytotoxic T cells in a MHC-I restricted manner. Once an antigen has been presented and recognized, the adaptive immune system activates an army of immune cells specifically designed to attack that antigen. Like the innate system, the adaptive system includes both humoral immunity components (B lymphocyte cells) and cell-mediated immunity (T lymphocyte cells) components. B cells are activated to secrete antibodies, which travel through the bloodstream and bind to the foreign antigen. Helper T cells (regulatory T cells, CD4+ cells) and cytotoxic T cells (CTL, CD8+ cells) are activated when their T cell receptor interacts with an antigen-bound MHC molecule. Cytokines and co-stimulatory molecules help the T cells mature, which mature cells, in turn, produce cytokines which allows the production of priming and expansion of additional T cells sustaining the response. Once activated, the helper T cells release cytokines which regulate and direct the activity of different immune cell types, including APCs, macrophages, neutrophils, and other lymphocytes, to kill and remove targeted cells. Helper T cells also secrete extra signals that assist in the activation of cytotoxic T cells which also help to sustain the immune response. Upon activation, CTL undergoes clonal selection, in which it gains functions, divides rapidly to produce an army of activated effector cells, and forms long-lived memory T cells ready to rapidly respond to future threats. Activated CTL then travels throughout the body searching for cells that bear that unique MHC Class I and antigen. The effector CTLs release cytotoxins that form pores in the target cell's plasma membrane, causing apoptosis. Adaptive immunity also includes a “memory” that makes future responses against a specific antigen more efficient. Upon resolution of the infection, T helper cells and cytotoxic T cells die and are cleared away by phagocytes, however, a few of these cells remain as memory cells. If the same antigen is encountered at a later time, these memory cells quickly differentiate into effector cells, shortening the time required to mount an effective response.

An “immune checkpoint inhibitor” or “immune checkpoint” refers to a molecule that completely or partially reduces, inhibits, interferes with, or modulates one or more immune checkpoint proteins. Immune checkpoint proteins regulate T-cell activation or function, and are known in the art. Non-limiting examples include CTLA-4 and its ligands CD 80 and CD86, and PD-1 and its ligands PD-L1 and PD-L2. Immune checkpoint proteins are responsible for co-stimulatory or inhibitory interactions of T-cell responses, and regulate and maintain self-tolerance and physiological immune responses.

A “co-stimulatory” molecule or “co-stimulator” is an immune modulator that increase or activates a signal that stimulates an immune response or inflammatory response.

Examples of bacteria suitable for the methods and compositions in the present invention include, but are not limited to, Bifidobacterium, Caulobacter, Clostridium, Escherichia coli, Listeria, Mycobacterium, Salmonella, Streptococcus, and Vibrio, e.g., Bifidobacterium adolescentis, Bifidobacterium bifidum, Bifidobacterium breve UCC2003, Bifidobacterium infantis, Bifidobacterium longum, Clostridium acetobutylicum, Clostridium butyricum, Clostridium butyricum M-55, Clostridium butyricum miyairi, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera (Cronin et al., 2012; Forbes, 2006; Jain and Forbes, 2001; Liu et al., 2014; Morrissey et al., 2010; Nuno et al., 2013; Patyar et al., 2010; Cronin, et al., Mol Ther 2010; 18:1397-407).

“Microorganism” refers to an organism or microbe of microscopic, submicroscopic, or ultramicroscopic size that typically consists of a single cell. Examples of microorganisms include bacteria, viruses, parasites, fungi, certain algae, protozoa, and yeast. In some aspects, the microorganism is modified (“modified microorganism”) from its native state to produce one or more effectors or immune modulators. In certain embodiments, the modified microorganism is a modified bacterium. In some embodiments, the modified microorganism is a genetically engineered bacterium. In certain embodiments, the modified microorganism is a modified yeast. In other embodiments, the modified microorganism is a genetically engineered yeast.

As used herein, the term “recombinant microorganism” refers to a microorganism, e.g., bacterial, yeast, or viral cell, or bacteria, yeast, or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed or engineered microorganism” refers to a microorganism, e.g., bacterial, yeast, or viral cell, or bacteria, yeast, or virus, that has been genetically modified from its native state to perform a specific function. Thus, a “programmed or engineered bacterial cell” or “programmed or engineered bacteria” refers to a bacterial cell or bacteria that has been genetically modified from its native state to perform a specific function. In certain embodiments, the programmed or engineered bacterial cell has been modified to express one or more proteins, for example, one or more proteins that have a therapeutic activity or serve a therapeutic purpose. The programmed or engineered bacterial cell may additionally have the ability to stop growing or to destroy itself once the protein(s) of interest have been expressed.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to certain strains belonging to the genus Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered or programmed to enhance or improve probiotic properties.

“Operably linked” refers a nucleic acid sequence, e.g., a gene encoding an enzyme for the production of a STING agonist, e.g., a diadenylate cyclase or a c-di-GAMP synthase, that is joined to a regulatory region sequence in a manner which allows expression of the nucleic acid sequence, e.g., acts in cis. A regulatory region is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

An “inducible promoter” refers to a regulatory region that is operably linked to one or more genes, wherein expression of the gene(s) is increased in the presence of an inducer of said regulatory region.

“Exogenous environmental condition(s)” refer to setting(s) or circumstance(s) under which the promoter described herein is induced. The phrase “exogenous environmental conditions” is meant to refer to the environmental conditions external to the intact (unlysed) engineered microorganism, but endogenous or native to environment or the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are low-oxygen, microaerobic, or anaerobic conditions, such as hypoxic and/or necrotic tissues. In some embodiments, the genetically engineered microorganism of the disclosure comprise an oxygen level-dependent promoter. In some aspects, bacteria have evolved transcription factors that are capable of sensing oxygen levels. Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase), ANR, and DNR. Corresponding FNR-responsive promoters, ANR (anaerobic nitrate respiration)-responsive promoters, and DNR (dissimilatory nitrate respiration regulator)-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 2.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrs, FNR, P-FNRS promoter and other such related designations to indicate the promoter PfnrS.

TABLE 2
Examples of transcription factors and responsive
genes and regulatory regions

| Transcription | Examples of responsive genes, promoters, |
|---|---|
| Factor | and/or regulatory regions: |
| FNR | nirB, ydfZ, pdhR, focA, ndH, hlyE, |
| | narK, narX, narG, yfiD, tdcD |
| ANR | arcDABC |
| DNR | norb, norC |

As used herein, a “non-native” nucleic acid sequence refers to a nucleic acid sequence not normally present in a microorganism, e.g., an extra copy of an endogenous sequence, or a heterologous sequence such as a sequence from a different species, strain, or substrain of bacteria or virus, or a sequence that is modified and/or mutated as compared to the unmodified sequence from bacteria or virus of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome. In some embodiments, the genetically engineered bacteria of the disclosure comprise a gene that is operably linked to a directly or indirectly inducible promoter that is not associated with said gene in nature, e.g., an FNR-responsive promoter (or other promoter described herein) operably linked to a gene encoding an immune modulator.

In one embodiment, the effector, or immune modulator, is a therapeutic molecule encoded by at least one non-native gene. In one embodiment, the effector, or immune modulator, is a therapeutic molecule produced by an enzyme encoded by at least one non-native gene. In one embodiment, the effector, or immune modulator, is at least one enzyme of a biosynthetic pathway encoded by at least one non-native gene. In another embodiment, the effector, or immune modulator, is at least one molecule produced by at least one enzyme of a biosynthetic pathway encoded by at least one non-native gene.

In one embodiment, the immune initiator is a therapeutic molecule encoded by at least one non-native gene. In one embodiment, the immune initiator is a therapeutic molecule produced by an enzyme encoded by at least one non-native gene. In one embodiment, the immune initiator is at least one enzyme of a biosynthetic pathway encoded by at least one non-native gene. In another embodiment, the immune initiator is at least one molecule produced by at least one enzyme of a biosynthetic pathway encoded by at least one non-native gene.

In one embodiment, the immune sustainer is a therapeutic molecule encoded by at least one non-native gene. In one embodiment, the immune sustainer is a therapeutic molecule produced by an enzyme encoded by at least one non-native gene. In one embodiment, the immune sustainer is at least one enzyme of a biosynthetic pathway encoded by at least one non-native gene. In another embodiment, the immune sustainer is at least one molecule produced by at least one enzyme of a biosynthetic pathway encoded by at least one non-native gene.

“Constitutive promoter” refers to a promoter that is capable of facilitating continuous transcription of a coding sequence or gene under its control and/or to which it is operably linked. Constitutive promoters and variants are well known in the art and non-limiting examples of constitutive promoters are described herein and in International Patent Application PCT/US2017/013072, filed Jan. 11, 2017 and published as WO2017/123675, the contents of which is herein incorporated by reference in its entirety. In some embodiments, such promoters are active in vitro, e.g., under culture, expansion and/or manufacture conditions. In some embodiments, such promoters are active in vivo, e.g., in conditions found in the in vivo environment, e.g., the gut and/or the microenvironment.

As used herein, “stably maintained” or “stable” bacterium or virus is used to refer to a bacterial or viral host cell carrying non-native genetic material, e.g., an immune modulator, such that the non-native genetic material is retained, expressed, and propagated. The stable bacterium or virus is capable of survival and/or growth in vitro, e.g., in medium, and/or in vivo, e.g., in hypoxic and/or necrotic tissues. For example, the stable bacterium or virus may be a genetically engineered bacterium comprising non-native genetic material encoding an immune modulator, in which the plasmid or chromosome carrying the non-native genetic material is stably maintained in the bacterium or virus, such that the immune modulator can be expressed in the bacterium or virus, and the bacterium or virus is capable of survival and/or growth in vitro and/or in vivo.

As used herein, the terms “modulate” and “treat” and their cognates refer to an amelioration of a microbial infection, e.g., the coronavirus disease 2019 (COVID-19), or at least one discernible symptom thereof. In another embodiment, “modulate” and “treat” refer to an amelioration of at least one measurable physical parameter, not necessarily discernible by the patient. For example, the symptoms for patients with COVID-19 vary depending on how serious the infection is. Patients with a mild to moderate upper-respiratory infection may develop symptoms such as runny nose, sneezing, headache, cough, sore throat, fever, or short of breath. In more severe cases, coronavirus infection can cause pneumonia, severe acute respiratory syndrome, kidney failure and even death. Further details regarding signs and symptoms of the various diseases or conditions are provided herein and are well known in the art. In another embodiment, “modulate” and “treat” refer to inhibiting the development of a microbial infection, e.g., COVID-19, either physically (e.g., stabilization of a discernible symptom), physiologically (e.g., stabilization of a physical parameter), or both. In another embodiment, “modulate” and “treat” refer to slowing the development or reversing the development of a microbial infection, e.g., COVID-19. As used herein, “prevent” and its cognates refer to delaying the onset or reducing the risk of acquiring a given disease.

Those in need of treatment may include individuals already having a particular microbial infection, as well as those at risk of having, or who may ultimately acquire the microbial infection. The need for treatment is assessed, for example, by the presence of one or more risk factors associated with the development of a microbial infection, the presence or progression of a microbial infection, or likely receptiveness to treatment of a subject having the microbial infection.

Those in need of treatment may include individuals already having a particular viral infection, as well as those at risk of having, or who may ultimately acquire the COVID-19. The need for treatment is assessed, for example, by the presence of one or more risk factors associated with the development of a viral infection, the presence or progression of a viral infection, or likely receptiveness to treatment of a subject having the viral infection.

As used herein, the term “conventional anti-viral treatment,” “conventional anti-viral therapy,” “conventional anti-microbial treatment,” or “conventional anti-microbial treatment” refers to treatment or therapy that is widely accepted and used by most healthcare professionals. It is different from alternative or complementary therapies, which are not as widely used.

As used herein a “pharmaceutical composition” refers to a preparation of genetically engineered microorganism of the disclosure with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial or viral compound. An adjuvant is included under these phrases.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition. A therapeutically effective amount may, for example, be sufficient to treat, prevent, reduce the severity, delay the onset, and/or reduce the risk of occurrence of one or more symptoms of a disorder. A therapeutically effective amount, as well as a therapeutically effective frequency of administration, can be determined by methods known in the art and discussed below.

In some embodiments, the term “therapeutic molecule” refers to a molecule or a compound that is results in prevention, delay of onset of symptoms, or amelioration of symptoms of a condition. In some embodiments, a therapeutic molecule may be, for example, a cytokine, a chemokine, a single chain antibody, a ligand, a metabolic converter, e.g., arginine, a kynurnenine consumer, or an adenosine consumer, a T cell co-stimulatory receptor, a T cell co-stimulatory receptor ligand, an engineered chemotherapy, or a lytic peptide, among others.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

### Bacteria

In one embodiment, the modified microorganism may be a bacterium, e.g., a genetically engineered bacterium. The modified microorganism, or genetically engineered microorganisms, such as the modified bacterium of the disclosure is capable of target-specific delivery of proteins (e.g., viral, bacterial, fungal, and cancer proteins) and/or an immune modulator, such as a STING agonist, to a cell or host. The engineered bacteria may be administered systemically, orally, locally and/or intratumorally. In some embodiments, the genetically engineered bacteria are capable of producing a displayed protein (e.g., viral, bacterial, fungal, and cancer protein), and producing an effector molecule, e.g., an immune modulator, e.g., immune stimulator or sustainer provided herein.

In certain embodiments, the modified microorganisms or genetically engineered bacteria are obligate anaerobic bacteria. In certain embodiments, the genetically engineered bacteria are facultative anaerobic bacteria. In certain embodiments, the genetically engineered bacteria are aerobic bacteria. In some embodiments, the genetically engineered bacteria are Gram-positive bacteria and lack LPS. In some embodiments, the genetically engineered bacteria are Gram-negative bacteria. In some embodiments, the genetically engineered bacteria are Gram-positive and obligate anaerobic bacteria. In some embodiments, the genetically engineered bacteria are Gram-positive and facultative anaerobic bacteria. In some embodiments, the genetically engineered bacteria are non-pathogenic bacteria. In some embodiments, the genetically engineered bacteria are commensal bacteria. In some embodiments, the genetically engineered bacteria are probiotic bacteria. In some embodiments, the genetically engineered bacteria are naturally pathogenic bacteria that are modified or mutated to reduce or eliminate pathogenicity. Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium butyricum miyairi, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, Vibrio cholera. In certain embodiments, the genetically engineered bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In certain embodiments, the genetically engineered bacteria are selected from the group consisting of Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli Nissle, Lactobacillus acidophilus, Lactobacillus plantarum, Lactobacillus reuteri, and Lactococcus lactis. In some embodiments, Lactobacillus is used for delivery of one or more immune modulators.

In some embodiments, the genetically engineered bacteria are obligate anaerobes. In some embodiments, the genetically engineered bacteria are Clostridia and capable of delivery of immune modulators. In some embodiments, the genetically engineered bacteria is selected from the group consisting of Clostridium novyi-NT, Clostridium histolyticium, Clostridium tetani, Clostridium oncolyticum, Clostridium sporogenes, and Clostridium beijerinckii (Liu et al., 2014). In some embodiments, the Clostridium is naturally non-pathogenic. In alternate embodiments, the Clostridium is naturally pathogenic but modified to reduce or eliminate pathogenicity. For example, Clostridium novyi are naturally pathogenic, and Clostridium novyi-NT are modified to remove lethal toxins. Clostridium novyi-NT and Clostridium sporogenes have been used to deliver single-chain HIF-1a antibodies to treat cancer (Groot et al., 2007).

In some embodiments, the genetically engineered bacteria facultative anaerobes. In some embodiments, the genetically engineered bacteria are Salmonella, e.g., Salmonella typhimurium, and are capable of tumor-specific delivery of immune modulators. Salmonella are non-spore-forming Gram-negative bacteria that are facultative anaerobes. In some embodiments, the Salmonella are naturally pathogenic but modified to reduce or eliminate pathogenicity. For example, Salmonella typhimurium is modified to remove pathogenic sites (attenuated). In some embodiments, the genetically engineered bacteria are Bifidobacterium and capable of immune modulators. Bifidobacterium are Gram-positive, branched anaerobic bacteria. In some embodiments, the Bifidobacterium is naturally non-pathogenic. In alternate embodiments, the Bifidobacterium is naturally pathogenic but modified to reduce or eliminate pathogenicity. Bifidobacterium and Salmonella have been shown to preferentially target and replicate in the hypoxic and necrotic regions of tumors (Yu et al., 2014).

In some embodiments, the genetically engineered bacteria are Gram-negative bacteria. In some embodiments, the genetically engineered bacteria are E. coli. In some embodiments, the genetically engineered bacteria are Escherichia coli strain Nissle 1917 (E. coli Nissle), a Gram-negative bacterium of the Enterobacteriaceae family that “has evolved into one of the best characterized probiotics” (Ukena et al., 2007). The strain is characterized by its complete harmlessness (Schultz, 2008), and has GRAS (generally recognized as safe) status (Reister et al., 2014, emphasis added).

The genetically engineered bacteria of the invention may be destroyed, e.g., by defense factors in tissues or blood serum (Sonnenborn et al., 2009). In some embodiments, the genetically engineered bacteria are administered repeatedly. In some embodiments, the genetically engineered bacteria are administered once.

In certain embodiments, the modified microorganism comprising the display protein is E. coli Nissle strain SYN1557 (delta PAL::CmR).

In certain embodiments, the effectors and/or immune modulator(s) described herein are expressed in one species, strain, or subtype of genetically engineered bacteria. In alternate embodiments, the effector and/or immune modulator is expressed in two or more species, strains, and/or subtypes of genetically engineered bacteria. One of ordinary skill in the art would appreciate that the genetic modifications disclosed herein may be modified and adapted for other species, strains, and subtypes of bacteria.

Further examples of bacteria which are suitable are described in International Patent Publication WO/2014/043593, the contents of which is herein incorporated by reference in its entirety. In some embodiments, such bacteria are mutated to attenuate one or more virulence factors.

In some embodiments, the genetically engineered bacteria of the disclosure proliferate and colonize a host. In some embodiments, colonization persists for several days, several weeks, several months, several years or indefinitely. In some embodiments, the genetically engineered bacteria do not proliferate in the host and bacterial counts drop off quickly post administration, e.g., less than a week post administration, until no longer detectable.

### Bacteriophages

In some embodiments, the genetically engineered bacteria of the disclosure comprise one or more lysogenic, dormant, temperate, intact, defective, cryptic, or satellite phage or bacteriocins/phage tail or gene transfer agents in their natural state. In some embodiments, the prophage or bacteriophage exists in all isolates of a particular bacterium of interest. In some embodiments, the bacteria are genetically engineered derivatives of a parental strain comprising one or more of such bacteriophage. In any of the embodiments described herein, the bacteria may comprise one or more modifications or mutations within a prophage or bacteriophage genome which alters the properties or behavior of the bacteriophage. In some embodiments, the modifications or mutations prevent the prophage from entering or completing the lytic process. In some embodiments, the modifications or mutations prevent the phage from infecting other bacteria of the same or a different type. In some embodiments, the modifications or mutations alter the fitness of the bacterial host. In some embodiments, the modifications or mutations no not alter the fitness of the bacterial host. In some embodiments, the modifications or mutations have an impact on the desired effector function, e.g., on levels of expression of the effector molecule, e.g., immune modulator, e.g., immune stimulator or sustainer, of the genetically engineered bacterium. In some embodiments, the modifications or mutations have no impact on the desired function e.g., on levels of expression of the effector molecule or on levels of activity of the effector molecule.

Phage genome size varies, ranging from the smallest Leuconostoc phage L5 (2,435 bp), ˜11.5 kbp (e.g. Mycoplasma phage P1), ˜21 kbp (e.g. Lactococcus phage c2), and ˜30 kbp (e.g. Pasteurella phage F108) to the almost 500 kbp genome of Bacillus megaterium phage G (Hatfull and Hendrix; Bacteriophages and their Genomes, Curr Opin Virol. 2011 Oct. 1; 1(4): 298-303, and references therein). Phage genomes may encode less than 10 genes up to several hundreds of genes. Temperate phages or prophages are typically integrated into the chromosome(s) of the bacterial host, although some examples of phages that are integrated into bacterial plasmids also exist (Little, Loysogeny, Prophage Induction, and Lysogenic Conversion. In: Waldor M K, Friedman D I, Adhya S, editors. Phages Their Role in Bacterial Pathogenesis and Biotechnology. Washington D.C.: ASM Press; 2005. pp. 37-54). In some cases, the phages are always located at the same position within the bacterial host chromosome(s), and this position is specific to each phage, i.e., different phages are located at different positions. Other phages can integrate at numerous different locations.

Accordingly, the bacteria of the disclosure comprise one or more phages genomes which may vary in length, from at least about 1 bp to 10 kb, from at least about 10 kb to 20 kb, from at least about 20 kb to 30 kb, from at least about 30 kb to 40 kb, from at least about 30 kb to 40 kb, from at least about 40 kb to 50 kb, from at least about 50 kb to 60 kb, from at least about 60 kb to 70 kb, from at least about 70 kb to 80 kb, from at least about 80 kb to 90 kb, from at least about 90 kb to 100 kb, from at least about 100 kb to 120 kb, from at least about 120 kb to 140 kb, from at least about 140 kb to 160 kb, from at least about 160 kb to 180 kb, from at least about 180 kb to 200 kb, from at least about 200 kb to 180 kb, from at least about 160 kb to 250 kb, from at least about 250 kb to 300 kb, from at least about 300 kb to 350 kb, from at least about 350 kb to 400 kb, from at least about 400 kb to 500 kb, from at least about 500 kb to 1000 kb. In one embodiment, the genetically engineered bacteria comprise a bacteriophage genome greater than 1000 kb in length.

In some embodiments, the bacteria of the disclosure comprise one or more phages genomes, which comprise one or more genes encoding one or more polypeptides. In one embodiment, the genetically engineered bacteria comprise a bacteriophage genome comprising at least about 1 to 5 genes, at least about 5 to 10 genes, at least about 10 to 15 genes, at least about 15 to 20 genes, at least about 20 to 25 genes, at least about 25 to 30 genes, at least about 30 to 35 genes, at least about 35 to 40 genes, at least about 40 to 45 genes, at least about 45 to 50 genes, at least about 50 to 55 genes, at least about 55 to 60 genes, at least about 60 to 65 genes, at least about 65 to 70 genes, at least about 70 to 75 genes, at least about 75 to 80 genes, at least about 80 to 85 genes, at least about 85 to 90 genes, at least about 90 to 95 genes, at least about 95 to 100 genes, at least about 100 to 115 genes, at least about 115 to 120 genes, at least about 120 to 125 genes, at least about 125 to 130 genes, at least about 130 to 135 genes, at least about 135 to 140 genes, at least about 140 to 145 genes, at least about 145 to 150 genes, at least about 150 to 160 genes, at least about 160 to 170 genes, at least about 170 to 180 genes, at least about 180 to 190 genes, at least about 190 to 200 genes, at least about 200 to 300 genes. In one embodiment, the genetically engineered bacteria comprise a bacteriophage genome comprising more than about 300 genes.

In some embodiments, the phage is always or almost always located at the same location or position within the bacterial host chromosome(s) in a particular species. In some embodiments, the phages are found integrated at different locations within the host chromosome in a particular species. In some embodiments, the phage is located on a plasmid.

In some embodiments, the prophage may be a defective or a cryptic prophage. Defective prophages can no longer undergo a lytic cycle. Cryptic prophages may not be able to undergo a lytic cycle or never have undergone a lytic cycle (Bobay et al., 2014). In some embodiments, the bacteria comprise one or more satellite phage genomes. Satellite phages are otherwise functional phages that do not carry their own structural protein genes, and have genomes that are configures for encapsulation by the structural proteins of other specific phages (Six and Klug Bacteriophage P4: a satellite virus depending on a helper such as prophage P2, Virology, Volume 51, Issue 2, February 1973, Pages 327-344).

In some embodiments, the bacteria comprise one or more tailiocins. Many bacteria, both gram positive and gram negative, produce a variety of particles resembling phage tails that are functional without an associated phage head (termed tailiocins), and many of which have been shown to have bacteriocin properties (reviewed in Ghequire and Mot, The Tailocin Tale: Peeling off Phage; Trends in Microbiology, October 2015, Vol. 23, No. 10). Phage tail-like bacteriocins are classified two different families: contractile phage tail-like (R-type) and noncontractile but flexible ones (F-type). In some embodiments, the bacteria comprise one or more gene transfer agents. Gene transfer agents (GTAs) are phage-like elements that are encoded by some bacterial genomes. Although GTAs resemble phages, they lack the hallmark capabilities that define typical phages, and they package random fragments of the host cell DNA and then transfer them horizontally to other bacteria of the same species (reviewed in Lang et al., Gene transfer agents: phage-like elements of genetic exchange, Nat Rev Microbiol. 2012 Jun. 11; 10(7): 472-482). There, the DNA can replace the resident cognate chromosomal region by homologous recombination. However, these particles cannot propagate as viruses, as the vast majority of the particles do not carry the genes that encode the GTA. In some embodiments, the bacteria comprise one or more filamentous virions. Filamentous virions integrate as dsDNA prophages (reviewed in Marvin D A, et al, Structure and assembly of filamentous bacteriophages, Prog Biophys Mol Biol. 2014 April; 114(2):80-122). In any of these embodiments, the bacteria described herein comprising defective or a cryptic prophage, satellite phage genomes, tailiocins, gene transfer agents, filamentous virions, which may comprise one or more modifications or mutations within their sequence.

Prophages can be either identified experimentally or computationally. The experimental approach involves inducing the host bacteria to release phage particles by exposing them to UV light or other DNA-damaging conditions. However, in some cases, the conditions under which a prophage is induced is unknown, and therefore the absence of plaques in a plaque assay does not necessarily prove the absence of a prophage. Additionally, this approach can show only the existence of viable phages, but will not reveal defective prophages. As such, computational identification of prophages from genomic sequence data has become the most preferred route.

Co-pending International Patent Application PCT/US18/38840, filed Jun. 21, 2018, herein incorporated by reference in their entireties, provide non-limiting examples of probiotic bacteria which contain number of potential bacteriophages contained in the bacterial genome as determined by Phaster scoring. Phaster scoring is described in detail at phaster.ca and in Zhou, et al. (“PHAST: A Fast Phage Search Tool” Nucl. Acids Res. (2011) 39(suppl 2): W347-W352) and Arndt et al. (Arndt, et al. (2016) PHASTER: a better, faster version of the PHAST phage search tool. Nucleic Acids Res., 2016 May 3). In brief, three methods are applied with different criteria to score for prophage regions (as intact, questionable, or incomplete) within a provided bacterial genome sequence.

In any of the embodiments described herein, the bacteria described herein may comprise one or more modifications or mutations within an existing prophage or bacteriophage genome. In some embodiments, these modifications alter the properties or behavior of the prophage. In some embodiments, the modifications or mutations prevent the prophage from entering or completing the lytic process. In some embodiments, the modifications or mutations prevent the phage from infecting other bacteria of the same or a different type. In some embodiments, the modifications or mutations alter the fitness of the bacterial host. In some embodiments, the modifications or mutations do not alter the fitness of the bacterial host. In some embodiments, the modifications or mutations have an impact on the desired effector function, e.g., of a genetically engineered bacterium. In some embodiments, the modifications or mutations do not have an impact on the desired effector function, e.g., of a genetically engineered bacterium.

In some embodiments, the modifications or mutations reduce entry or completion of prophage lytic process at least about 1- to 2-fold, at least about 2- to 3-fold, at least about 3- to 4-fold, at least about 4- to 5-fold, at least about 5- to 10-fold, at least about 10 to 100-fold, at least about 100- to 1000-fold. In some embodiments, the modifications or mutations completely prevent entry or completion of prophage lytic process.

In some embodiments, the modifications or mutations reduce entry or completion of prophage lytic process by at least about 1% to 10%, at least about 10% to 20%, at least about 20% to 30%, at least about 30% to 40%, at least about 40% to 50%, at least about 50% to 60%, at least about 60% to 70%, at least about 70% to 80%, at least about 80% to 90%, or at least about 90% to 100%.

In some embodiments, the mutations include one or more deletions within the phage genome sequence. In some embodiments, the mutations include one or more insertions into the phage genome sequence. In some embodiments, an antibiotic cassette can be inserted into one or more positions within the phage genome sequence. In some embodiments, the mutations include one or more substitutions within the phage genome sequence. In some embodiments, the mutations include one or more inversions within the phage genome sequence. In some embodiments, the modifications within the phage genome are combinations of two or more of insertions, deletions, substitutions, or inversions within one or more phage genome genes. In any of the embodiments described herein, the modifications may result in one or more frameshift mutations in one or more genes within the phage genome.

An any of these embodiments, the mutations can be located within or encompass one or more genes encoding proteins of various functions, e.g., lysis, e.g., proteases or lysins, toxins, antibiotic resistance, translation, structural (e.g., head, tail, collar, or coat proteins), bacteriophage assembly, recombination(e.g., integrases, invertases, or transposases), or replication (e.g., primases, tRNA related proteins), phage insertion, attachment, packaging, or terminases.

In some embodiments, described herein genetically engineered bacteria are engineered Escherichia coli strain Nissle 1917 (E. coli Nissle). As described in co-pending International Patent Application PCT/US18/38840, filed Jun. 21, 2018, herein incorporated by reference in their entireties, in more detail herein in the examples, routine testing procedures identified bacteriophage production from Escherichia coli Nissle 1917 (E. coli Nissle) and related engineered derivatives. To determine the source of the bacteriophage, a collaborative bioinformatics assessment of the genomes of E. coli Nissle, and engineered derivatives was conducted to analyze genomic sequences of the strains for evidence of prophages, to assess any identified prophage elements for the likelihood of producing functional phage, to compare any functional phage elements with other known phage identified among bacterial genomic sequences, and to evaluate the frequency with which prophage elements are found in other sequenced Escherichia coli (E. coli) genomes. The assessment tools included phage prediction software (PHAST and PHASTER), SPAdes genome assembler software, software for mapping low-divergent sequences against a large reference genome (BWA MEM), genome sequence alignment software (MUMmer), and the National Center for Biotechnology Information (NCBI) nonredundant database. The assessment results showed that E. coli Nissle and engineered derivatives analyzed contain three candidate prophage elements, with two of the three (Phage 2 and Phage 3) containing most genetic features characteristic of intact phage genomes. Two other possible phage elements were also identified. Of note, the engineered strains did not contain any additional phage elements that were not identified in parental E. coli Nissle, indicating that plaque-forming units produced by these strains originate from one of these endogenous phages (Phage 3). Interestingly, Phage 3 is unique to E. coli Nissle among a collection of almost 6000 sequenced E. coli genomes, although related sequences limited to short regions of homology with other putative prophage elements are found in a small number of genomes. Phage 3, but not any of the other Phage, was found to be inducible and result in bacterial lysis upon induction.

Prophages are very common among E. coli strains, with E. coli Nissle containing a relatively small number of prophage sequences compared to the average number found in a well-characterized set of sequenced E. coli genomes. As such, prophage presence in the engineered strains is part of the natural state of this species and the prophage features of the engineered strains analyzed were consistent with the progenitor strain, E. coli Nissle.

In some embodiments, the bacteria described herein may comprise one or more modifications or mutations within the E. coli Nissle Phage 3 genome which alters the properties or behavior of Phage 3. In some embodiments, the modifications or mutations prevent Phage 3 from entering or completing the lytic process. In some embodiments, the modifications or mutations prevent the E. coli Nissle Phage 3 from infecting other bacteria of the same or a different type. In some embodiments, the modifications or mutations improve the fitness of the bacterial host. In some embodiments, the no effect fitness of the bacterial host is observed. In some embodiments, the modifications or mutations have an impact on the desired effector function, e.g., expression of the immune modulator. In some embodiments, no impact on the desired effector function, e.g., expression of the immune modulator, is observed.

In some embodiments, the mutations introduced into the bacterial chassis include one or more deletions within the E. coli Nissle Phage 3 genome sequence. In some embodiments, the mutations include one or more insertions into the E. coli Nissle Phage 3 genome sequence. In some embodiments, an antibiotic cassette can be inserted into one or more positions within the E. coli Nissle Phage 3 genome sequence. Mutations within Phage 3 are described in more details in Co-pending U.S. provisional applications 62/523,202 and 62/552,829, herein incorporated by reference in their entireties.

In one specific embodiment, at least about 9000 to 10000 bp of the E. coli Nissle Phage 3 genome are mutated, e.g., in one example, 9687 bp of the E. coli Nissle Phage 3 genome are deleted.

In any of the embodiments described herein, the modifications encompass are located in one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345.

In one embodiment, the mutation is a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the mutation is a complete or partial deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and ECOLIN_10175. In one specific embodiment, the mutation is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a deletion mutation of ECOLIN_10175.

### Effector Molecules

### Oncolysis and Activation of an Innate Immune Response

In certain embodiments, the effector molecule(s), or immune modulators(s) of the disclosure generates an innate immune response. In certain embodiments, the immune modulators(s) of the disclosure generates a local immune response. In some aspects, the effector molecule, or immune modulator, is able to activate systemic immunity against displayed proteins (e.g., viral, bacterial, fungal, and cancer proteins). In certain embodiments, the immune modulators(s) generates a systemic or adaptive immune response. In some embodiments, the immune modulators(s) result in long-term immunological memory. Examples of suitable immune modulators(s), e.g., immune initiators and/or immune sustainers are described herein.

In some embodiments, one or more immune modulators may be produced by a modified microorganism described herein. In other embodiments, one or more immune modulators may be administered in combination with a modified microorganism capable of producing a second immune modulator(s). For example, one or more immune initiators may be administered in combination with a modified microorganism capable of producing one or more immune sustainers. In another embodiment, one or more immune sustainers may be administered in combination with a modified microorganism capable of producing one or more immune initiators. Alternatively, one or more first immune initiators may be administered in combination with a modified microorganism capable of producing one or more second immune initiators. Alternatively, one or more first immune sustainers may be administered in combination with a modified microorganism capable of producing one or more second immune sustainers.

### Displayed Proteins/Vaccines

By introducing a displayed protein, e.g., viral, bacterial, fungal, or cancer protein, to the local environment, an immune response can be raised against the particular microbe, cancer, or infected cell of interest known to be associated with that protein.

By introducing viral proteins, e.g., a spike viral protein, to the local environment, an immune response can be raised against the particular virus or infected cell of interest known to be associated with that protein. As used herein the term “viral protein” is meant to refer to virus-specific proteins, and/or virus-associated proteins, e.g., a spike protein of SARV-CoV-2, e.g., the receptor binding domain (RBD) of a spike protein of SARV-CoV-2. The engineered microorganisms can be engineered such that the peptides, e.g. viral proteins, e.g., the receptor binding domain (RBD) of a spike protein of SARV-CoV-2, can be anchored in the microbial cell wall (e.g., at the microbial cell surface). Thus, in some embodiments, the genetically engineered bacteria, are engineered to produce one or more viral proteins. Non-limiting examples of such viral proteins which may be produced by the bacteria of the disclosure described e.g., in Liu W J., et al. 2017, Antiviral Research 137:82-92; Huang J., et al. 2007, Vaccine 25: 6981-6991; Chen H., et al., 2005, J Immunol 175: 591-598; Ahmed S. F., et al., 2020, Viruses 12: 254; and Grifoni A., et al., Cell Host & Microbe 27: 1-10; the contents of each of which is herein incorporated by reference in its entirety or otherwise known in the art.

In any of these embodiments, the genetically engineered bacteria comprising gene sequence(s) encoding displayed proteins further comprise gene sequence(s) encoding one or more further effector molecule(s), i.e., therapeutic molecule(s) or a metabolic converter(s). In any of these embodiments, the circuit encoding antigens may be combined with a circuit encoding one or more immune initiators or immune sustainers as described herein, in the same or a different bacterial strain (combination circuit or mixture of strains). The circuit encoding the immune initiators or immune sustainers may be under the control of a constitutive or inducible promoter, e.g., low oxygen inducible promoter or any other constitutive or inducible promoter described herein. In any of these embodiments, the gene sequence(s) encoding proteins may be combined with gene sequence(s) encoding one or more STING agonist producing enzymes, as described herein, in the same or a different bacterial strain (combination circuit or mixture of strains). In some embodiments, the gene sequences which are combined with the gene sequence(s) encoding proteins encode DacA. DacA may be under the control of a constitutive or inducible promoter, e.g., low oxygen inducible promoter such as FNR or any other constitutive or inducible promoter described herein. In some embodiments, the dacA gene is integrated into the chromosome. In some embodiments, the gene sequences which are combined with the gene sequence(s) encoding proteins encode cGAS. cGAS may be under the control of a constitutive or inducible promoter, e.g., low oxygen inducible promoter such as FNR or any other constitutive or inducible promoter described herein. In some embodiments, the gene encoding cGAS is integrated into the chromosome. In any of these combination embodiments, the bacteria may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

### STING A Agonists

Stimulator of interferon genes (STING) protein was shown to be a critical mediator of the signaling triggered by cytosolic nucleic acid derived from DNA viruses, bacteria, and tumor-derived DNA. The ability of STING to induce type I interferon production lead to studies in the context of antitumor immune response, and as a result, STING has emerged to be a potentially potent target in many different immunotherapies. A large part of the effects caused by STING activation may depend upon production of IFN-β by APCs and improved antigen presentation by these cells, which promotes CD8+ T cell priming against viral proteins. However, STING protein is also expressed broadly in a variety of cell types including myeloid-derived suppressor cells (MDSCs) and cancer cells, in which the function of the pathway has not yet been well characterized (Sokolowska, O. & Nowis, D; STING Signaling in Cancer Cells: Important or Not?; Archivum Immunologiae et Therapiae Experimentalis; Arch. Immunol. Ther. Exp. (2018) 66: 125).

Stimulator of interferon genes (STING), also known as transmembrane protein 173 (TMEM173), mediator of interferon regulatory factor 3 activation (MITA), MPYS or endoplasmic reticulum interferon stimulator (ERIS), is a dimeric protein which is mainly expressed in macrophages, T cells, dendritic cells, endothelial cells, and certain fibroblasts and epithelial cells. STING plays an important role in the innate immune response—mice lacking STING are viable though prone to lethal infection following exposure to a variety of microbes. STING functions as a cytosolic receptor for the second messengers in the form of cytosolic cyclic dinucleotides (CDNs), such as cGAMP and the bacterial second messengers c-di-GMP and c-di-AMP. Upon stimulation by the CDN a conformational change in STING occurs. STING translocates from the ER to the Golgi apparatus and its carboxyterminus is liberated, This leads to the activation of TBK1 (TANK-binding kinase 1)/IRF3 (interferon regulatory factor 3), NF-κB, and STAT6 signal transduction pathways, and thereby promoting type I interferon and proinflammatory cytokine responses. CDNs include canonical cyclic di-GMP (c[G(30-50)pG(30-50)p] or cyclic di-AMP or cyclic GAMP (cGMP-AMP) (Barber, STING-dependent cytosolic DNA sensing pathways; Trends Immunol. 2014 February; 35(2):88-93).

CDNs can be exogenously (i.e., bacterially) and/or endogenously produced (i.e., within the host by a host enzyme upon exposure to dsDNA). STING is able to recognize various bacterial second messenger molecules cyclic diguanylate monophosphate (c-di-GMP) and cyclic diadenylate monophosphate (c-di-AMP), which triggers innate immune signaling response (Ma et al. The cGAS-STING Defense Pathway and Its Counteraction by Viruses; Cell Host & Microbe 19, Feb. 10, 2016). Additionally cyclic GMPAMP (cGAMP) can also bind to STING and result inactivation of IRF3 and β-interferon production. Both 3′5′-3′5′ cGAMP (3′3′ cGAMP) produced by Vibrio cholerae, and the metazoan secondary messenger cyclic [G(2′,5′)pA(3′5′)] (2′3′ cGAMP), could activate the innate immune response through STING pathway (Yi et al., Single Nucleotide Polymorphisms of Human STING Can Affect Innate Immune Response to Cyclic Dinucleotides; PLOS One (2013). 8(10)e77846, an references therein). Bacterial and metazoan (e.g., human) c-di-GAMP synthases (cGAS) utilizes GTP and ATP to generate cGAMP capable of STING activation. In contrast to prokaryotic CDNs, which have two canonical 30-50 phosphodiester linkages, the human cGAS product contains a unique 20-50 bond resulting in a mixed linkage cyclic GMP-AMP molecule, denoted as 2′,3′ cGAMP (as described in (Kranzusch et al., Ancient Origin of cGAS-STING Reveals Mechanism of Universal 2′,3′ cGAMP Signaling; Molecular Cell 59, 891-903, Sep. 17, 2015 and references therein). The bacterium Vibrio cholerae encodes an enzyme called DncV that is a structural homolog of cGAS and synthesizes a related second messenger with canonical 3′-5′ bonds (3′,3′ cGAMP).

Components of the stimulator of interferon genes (STING) pathway plays an important role in the detection of tumor cells by the immune system. In preclinical studies, cyclic dinucleotides (CDN), naturally occurring or rationally designed synthetic derivatives, are able to promote an aggressive antitumor response. For example, when co-formulated with an irradiated GM-CSF-secreting whole-cell vaccine in the form of STINGVAX, synthetic CDNs increased the antitumor efficacy and STINGVAX combined with PD-1 blockade induced regression of established tumors (Fu et al., STING agonist formulated cancer vaccines can cure established tumors resistant to PD-1 blockade; Sci Transl Med. 2015 Apr. 15; 7(283): 283ra52). In another example, Smith et al. conducted a study showing that STING agonists may augment CAR T therapy by stimulating the immune response to eliminate tumor cells that are not recognized by the adoptively transferred lymphocytes and thereby improve the effectiveness of CAR T cell therapy (Smith et al., Biopolymers co-delivering engineered T cells and STING agonists can eliminate heterogeneous tumors; J Clin Invest. 2017 Jun. 1; 127(6):2176-2191).

In some embodiments, the genetically engineered bacterium is capable of producing one or more STING agonists. Non limiting examples of STING agonists which can be produced by the genetically engineered bacteria of the disclosure include 3′3′ cGAMP, 2′3′cGAMP, 2′2′-cGAMP, 2′2′-cGAMP VacciGrade™ (Cyclic [G(2′,5′)pA(2′,5′)p]), 2′3′-cGAMP, 2′3′-cGAMP VacciGrade™ (Cyclic [G(2′,5′)pA(3′,5′)p]), 2′3′-cGAM(PS)2 (Rp/Sp), 3′3′-cGAMP, 3′3′-cGAMP VacciGrade™ (Cyclic [G(3′,5′)pA(3′,5′)p]), c-di-AMP, c-di-AMP VacciGrade™ (Cyclic diadenylate monophosphate Th1/Th2 response), 2′3′-c-di-AMP, 2′3′-c-di-AM(PS)2 (Rp,Rp) (Bisphosphorothioate analog of c-di-AMP, Rp isomers), 2′3′-c-di-AM(PS)2 (Rp,Rp) VacciGrade™ c-di-GMP, c-di-GMP VacciGrade™, 2′3′-c-di-GMP, and c-di-IMP. In some embodiments, the genetically engineered bacterium is that comprises a gene encoding one or more enzymes for the production of one or more STING agonists. Cyclic-di-GAMP synthase (cdi-GAMP synthase or cGAS) produces the cyclic-di-GAMP from one ATP and one GTP. In some embodiments, the enzymes are c-di-GAMP synthases (cGAS). In one embodiment, the genetically engineered bacteria comprise one or more gene sequences for the expression of an enzyme in class EC 2.7.7.86. In some embodiments, such enzymes are bacterial enzymes. In some embodiments, the enzyme is a bacterial c-di-GMP synthase. In some embodiments, the enzyme is a bacterial c-GAMP synthase (GMP-AMP synthase). In some embodiments, the bacteria are capable of producing 3′3′ c-dGAMP.

In some embodiments, the bacteria are capable of producing 3′3′-cGAMP. According to the instant disclosure several enzymes suitable for production of 3′3′-cGAM P from genetically engineered bacteria were identified. These enzymes include the Vibrio cholerae cGAS orthologs from Verminephrobacter eiseniae (EF01-2 Earthworm symbiont), Kingella denitrificans (ATCC 33394), and Neisseria bacilliformis (ATCC BAA-1200). Accordingly, in some embodiments, the genetically engineered bacteria comprise gene sequences encoding cGAS from Vibrio cholerae. Accordingly, in some embodiments, the genetically engineered bacteria comprise gene sequences encoding one or more Vibrio cholerae cGAS orthologs from species selected from Verminephrobacter eiseniae (EF01-2 Earthworm symbiont), Kingella denitrificans (ATCC 33394), and Neisseria bacilliformis (ATCC BAA-1200). In some embodiments, the bacteria comprise a gene sequence encoding DncV. In some embodiments, DncV is from Vibrio cholerae. In one embodiment, the DncV orthologue is from Verminephrobacter eiseniae. In one embodiment, the DncV orthrolog is from Kingella denitrificans. In one embodiment, the DncV orthrolog is from Neisseria bacilliformis. In some embodiments, the genetically engineered bacteria comprise a gene sequence encoding a DncV orthologue from a species selected from Enhydrobacter aerosaccus, Kingella denitrificans, Neisseria bacilliformis, Phaeobacter gallaeciensi, Citromicrobium sp., Roseobacter litoralis, Roseovarius sp., Methylobacterium populi, Erythrobacter sp., Erythrobacter litoralis, Methylophaga thiooxydans, Methylophaga thiooxydans, Herminiimonas arsenicoxydans, Verminephrobacter eiseniae, Methylobacter tundripaludum, Psychrobacter arcticus, Vibrio cholerae, Vibrio sp, Aeromonas salmonicida, Serratia odorifera, Verminephrobacter eiseniae, and Methylovorus glucosetrophus.

In some embodiments, the genetically engineered bacteria are capable of producing 2′3′-cGAMP. Human cGAS is known to produce 2′3′-cGAMP. In some embodiments, the genetically engineered bacteria comprise gene sequences encoding human cGAS.

In some embodiments, the genetically engineered bacteria are capable of increasing c-GAMP (2′3′ or 3′3′) levels in the microenvironment. In some embodiments, the genetically engineered bacteria are capable of increasing c-GAMP levels in the intracellular space In some embodiments, the genetically engineered bacteria are capable of increasing c-GAMP levels inside of a eukaryotic cell. In some embodiments, the genetically engineered bacteria are capable of increasing c-GAMP (2′3′ or 3′3′) levels inside of an immune cell. In some embodiments, the cell is a phagocyte. In some embodiments, the cell is a macrophage. In some embodiments, the cell is a dendritic cell. In some embodiments, the cell is a neutrophil. In some embodiments, the cell is a MDSC. In some embodiments, the genetically engineered bacteria are capable of increasing c-GAMP (2′3′ or 3′3′) inside of a cell. In some embodiments, the genetically engineered bacteria are capable of increasing c-GAMP levels in vitro in the bacterial cell and/or in the growth medium.

In one embodiment, the genetically engineered bacteria comprise gene sequence(s) encoding bacterial c-di-GAMP synthase from Vibrio cholerae. In some embodiments, the enzyme is DncV.

In one embodiment, the genetically engineered bacteria comprise gene sequence(s) encoding c-di-AMP synthase from Verminephrobacter eiseniae. In one embodiment, the bacterial c-di-GAMP synthase is DcnV orthologue from Verminephrobacter eiseniae (EF01-2 Earthworm symbiont). In some embodiments, the genetically engineered bacteria comprise c-di-GAMP synthase gene sequence(s) encoding one or more polypeptide(s) comprising SEQ ID NO: 1262 or functional fragments thereof. In some embodiments, genetically engineered bacteria comprise a gene sequence encoding a polypeptide that has at least about 80%, at least about 85%, at least about 90%, at least about 95%, or at least about 99% identity to SEQ ID NO: 1262 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1262. In some specific embodiments, the polypeptide comprises SEQ ID NO: 1262. In other specific embodiments, the polypeptide consists of SEQ ID NO: 1262. In certain embodiments, the bacterial c-di-GAMP synthase gene sequence has at least about 80% identity with SEQ ID NO: 1265. In certain embodiments, the gene sequence has at least about 90% identity with SEQ ID NO: 1265. In certain embodiments, the gene sequence has at least about 95% identity with SEQ ID NO: 1265. In some embodiments, the gene sequence has at least about 85%, 86%, 87%, 88%, 89%, 90%, 91%, 92%, 93%, 94%, 95%, 96%, 97%, 98%, or 99% identity with SEQ ID NO: 1265. In some specific embodiments, the gene sequence comprises SEQ ID NO: 1265. In other specific embodiments, the gene sequence consists of SEQ ID NO: 1265.

In one embodiment, the genetically engineered bacteria comprise gene sequence(s) encoding c-di-AMP synthase from Kingella denitrificans (ATCC 33394). In one embodiment, the bacterial c-di-GAMP synthase is DcnV orthologue from Kingella denitrificans. In some embodiments, the genetically engineered bacteria comprise c-di-GAMP synthase gene sequence(s) encoding one or more polypeptide(s) comprising SEQ ID NO: 1260 or functional fragments thereof. In some embodiments, genetically engineered bacteria comprise a gene sequence encoding a polypeptide that has at least about 80%, at least about 85%, at least about 90%, at least about 95%, or at least about 99% identity to SEQ ID NO: 1260 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1260. In some specific embodiments, the polypeptide comprises SEQ ID NO: 1260. In other specific embodiments, the polypeptide consists of SEQ ID NO: 1260. In certain embodiments, the bacterial c-di-GAMP synthase gene sequence has at least about 80% identity with SEQ ID NO: 1263. In certain embodiments, the gene sequence has at least about 90% identity with SEQ ID NO: 1263. In certain embodiments, the gene sequence has at least about 95% identity with SEQ ID NO: 1263. In some embodiments, the gene sequence has at least about 85%, 86%, 87%, 88%, 89%, 90%, 91%, 92%, 93%, 94%, 95%, 96%, 97%, 98%, or 99% identity with SEQ ID NO: 1263. In some specific embodiments, the gene sequence comprises SEQ ID NO: 1263. In other specific embodiments, the gene sequence consists of SEQ ID NO: 1263.

In one embodiment, the genetically engineered bacteria comprise gene sequence(s) encoding c-di-AMP synthase from Neisseria bacilliformis (ATCC BAA-1200). In one embodiment, the bacterial c-di-GAMP synthase is DcnV orthologue from Neisseria bacilliformis. In some embodiments, the genetically engineered bacteria comprise c-di-GAMP synthase gene sequence(s) encoding one or more polypeptide(s) comprising SEQ ID NO: 1261 or functional fragments thereof. In some embodiments, genetically engineered bacteria comprise a gene sequence encoding a polypeptide that is at least about 80%, at least about 85%, at least about 90%, at least about 95%, or at least about 99% identity to SEQ ID NO: 1261 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1261. In some specific embodiments, the polypeptide comprises SEQ ID NO: 1261. In other specific embodiments, the polypeptide consists of SEQ ID NO: 1261. In certain embodiments, the c-di-GAMP synthase sequence has at least about 80% identity with SEQ ID NO: 1264. In certain embodiments, the gene sequence has at least about 90% identity with SEQ ID NO: 1264. In certain embodiments, the gene sequence has at least about 95% identity with SEQ ID NO: 1264. In some embodiments, the gene sequence has at least about 85%, 86%, 87%, 88%, 89%, 90%, 91%, 92%, 93%, 94%, 95%, 96%, 97%, 98%, or 99% identity with SEQ ID NO: 1264. In some specific embodiments, the gene sequence comprises SEQ ID NO: 1264. In other specific embodiments, the gene sequence consists of SEQ ID NO: 1264.

In one embodiment, the genetically engineered bacteria comprise gene sequence(s) encoding mammalian c-di-GAMP enzymes. In some embodiments, the STING agonist producing enzymes are human enzymes. In some embodiments, the gene sequence(s) are codon-optimized for expression in a microorganism host cell. In one embodiment, the genetically engineered bacteria comprise gene sequence(s) encoding the human polypeptide cGAS. In some embodiments, the genetically engineered bacteria comprise human cGAS gene sequence(s) encoding one or more polypeptide(s) comprising SEQ ID NO: 1254 or functional fragments thereof. In some embodiments, genetically engineered bacteria comprise a gene sequence encoding a polypeptide that is at least about 80%, at least about 85%, at least about 90%, at least about 95%, or at least about 99% identity to SEQ ID NO: 1254 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1254. In some specific embodiments, the polypeptide comprises SEQ ID NO: 1254. In other specific embodiments, the polypeptide consists of SEQ ID NO: 1254. In certain embodiments, the human cGAS sequence has at least about 80% identity with SEQ ID NO: 1255. In certain embodiments, the gene sequence has at least about 90% identity with SEQ ID NO: 1255. In certain embodiments, the gene sequence has at least about 95% identity with SEQ ID NO: 1255. In some embodiments, the gene sequence has at least about 85%, 86%, 87%, 88%, 89%, 90%, 91%, 92%, 93%, 94%, 95%, 96%, 97%, 98%, or 99% identity with SEQ ID NO: 1255. In some specific embodiments, the gene sequence comprises SEQ ID NO: 1264. In other specific embodiments, the gene sequence consists of SEQ ID NO: 1255.

In some embodiments, the bacteria are capable of producing cyclic-di-GMP. Accordingly, in some embodiments, the genetically engineered bacteria comprise gene sequence(s) encoding one or more diguanylate cyclase(s).

In some embodiments, the genetically engineered bacteria are capable of increasing cyclic-di-GMP levels in the microenvironment. In some embodiments, the genetically engineered bacteria are capable of increasing cyclic-di-GMP levels in the intracellular space In some embodiments, the genetically engineered bacteria are capable of increasing cyclic-di-GMP levels inside of a eukaryotic cell. In some embodiments, the genetically engineered bacteria are capable of increasing cyclic-di-GMP levels inside of an immune cell. In some embodiments, the cell is a phagocyte. In some embodiments, the cell is a macrophage. In some embodiments, the cell is a dendritic cell. In some embodiments, the cell is a neutrophil. In some embodiments, the cell is a MDSC. In some embodiments, the genetically engineered bacteria are capable of increasing c cyclic-di-GMP levels inside of a cell. In some embodiments, the genetically engineered bacteria are capable of increasing c-GMP levels in vitro in the bacterial cell and/or in the growth medium.

In some embodiments, the genetically engineered bacteria are capable of producing c-diAMP. Diadenylate cyclase produces one molecule cyclic-di-AMP from two ATP molecules. In one embodiment, the genetically engineered bacteria comprise one or more gene sequences for the expression of a diadenylate cyclase. In one embodiment, the genetically engineered bacteria comprise one or more gene sequences for the expression of an enzyme in class EC 2.7.7.85. In one embodiment, the diadenylate cyclase is a bacterial diadenylate cyclase. In one embodiment, the diadenylate cyclase is DacA. In one embodiment, the DacA is from Listeria monocytogenes.

In some embodiments, the genetically engineered bacteria comprise DacA gene sequence(s) encoding one or more polypeptide(s) comprising SEQ ID NO: 1257 or functional fragments thereof. In some embodiments, genetically engineered bacteria comprise a gene sequence encoding a polypeptide that has at least about 80%, at least about 85%, at least about 90%, at least about 95%, or at least about 99% identity to SEQ ID NO: 1257 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1257. In some specific embodiments, the polypeptide comprises SEQ ID NO: 1257. In other specific embodiments, the polypeptide consists of SEQ ID NO: 1257. In certain embodiments, the Dac A sequence has at least about 80% identity with SEQ ID NO: 1258. In certain embodiments, the gene sequence has at least about 90% identity with SEQ ID NO: 1258. In certain embodiments, the gene sequence has at least about 95% identity with SEQ ID NO: 1258. In some embodiments, the gene sequence has at least about 85%, 86%, 87%, 88%, 89%, 90%, 91%, 92%, 93%, 94%, 95%, 96%, 97%, 98%, or 99% identity with SEQ ID NO: 1258. In some specific embodiments, the gene sequence comprises SEQ ID NO: 1258. In other specific embodiments, the gene sequence consists of SEQ ID NO: 1258.

In some embodiments, the genetically engineered bacteria comprise DacA gene sequence(s) operably linked to a promoter which is inducible under low oxygen conditions, e.g., an FNR inducible promoter as described herein. In certain embodiments, the sequence of the DacA gene operably linked to the FNR inducible promoter has at least about 80% identity with SEQ ID NO: 1284. In certain embodiments, the sequence of the DacA gene operably linked to the FNR inducible promoter has at least about 90% identity with SEQ ID NO: 1258. In certain embodiments, the sequence of the DacA gene operably linked to the FNR inducible promoter has at least about 95% identity with SEQ ID NO: 1258. In some embodiments, the sequence of the DacA gene operably linked to the FNR inducible promoter has at least about 85%, 86%, 87%, 88%, 89%, 90%, 91%, 92%, 93%, 94%, 95%, 96%, 97%, 98%, or 99% identity with SEQ ID NO: 1258. In some specific embodiments, the sequence of the DacA gene operably linked to the FNR inducible promoter comprises SEQ ID NO: 1258. In other specific embodiments the sequence of the DacA gene operably linked to the FNR inducible promoter consists of SEQ ID NO: 1258.

Other suitable diadenylate cyclases are known in the art and include those include in the EggNog database (http://eggnogdb.embl.de). Non-limiting examples of diadenylate cyclases which can be expressed by the bacteria include Megasphaera sp. UPII 135-E (HMPREF1040_0026), Streptococcus anginosus SK52=DSM 20563 (HMPREF9966_0555), Streptococcus mitis bv. 2 str. SK95 (HMPREF9965_1675), Streptococcus infantis SK1076 (HMPREF9967_1568), Acetonema longum DSM 6540 (ALO_03356), Sporosarcina newyorkensis 2681 (HMPREF9372_2277), Listeria monocytogenes str. Scott A (BN418_2551), Candidatus arthromitus sp. SFB-mouse-Japan (SFBM_1354), Haloplasma contractile SSD-17B 2 seqs HLPCO_01750, HLPCO_08849), Lactobacillus kefiranofaciens ZW3 (WANG_0941), Mycoplasma anatis 1340 (GIG_03148), Streptococcus constellatus subsp. pharyngis SK1060=CCUG 46377 (HMPREF1042_1168), Streptococcus infantis SK970 (HMPREF9954_1628), Paenibacillus mucilaginosus KNP414 (YBBP), Nostoc sp. PCC 7120 (ALL2996), Mycoplasma columbinum SF7 (MCSF7_01321), Lactobacillus ruminis SPM0211 (LRU_01199), Candidatus arthromitus sp. SFB-rat-Yit (RATSFB_1182), Clostridium sp. SY8519 (CXIVA_02190), Brevibacillus laterosporus LMG 15441 (BRLA_C02240), Weissella koreensis KACC 15510 (WKK_01955), Brachyspira intermedia PWS/A (BINT_2204), Bizionia argentinensis JUB59 (BZARG_2617), Streptococcus salivarius 57.1 (SSAL_01348), Alicyclobacillus acidocaldarius subsp. acidocaldarius Tc-4-1 (TC41_3001), Sulfobacillus acidophilus TPY (TPY_0875), Streptococcus pseudopneumoniae IS7493 (SPPN_07660), Megasphaera elsdenii DSM 20460 (MELS_0883), Streptococcus infantarius subsp. infantarius CJ18 (SINF_1263), Blattabacterium sp. (Mastotermes darwiniensis) str. MADAR (MADAR_511), Blattabacterium sp. (Cryptocercus punctulatus) str. Cpu (BLBCPU_093), Synechococcus sp. CC9605 (SYNCC9605_1630), Thermus sp. CCB_US3_UFI (AEV17224.1), Mycoplasma haemocanis str. Illinois (MHC_04355), Streptococcus macedonicus ACA-DC 198 (YBBP), Mycoplasma hyorhinis GDL-1 (MYM_0457), Synechococcus elongatus PCC 7942 (SYNPCC7942_0263), Synechocystis sp. PCC 6803 (SLL0505), Chlamydophila pneumoniae CWL029 (YBBP), Microcoleus chthonoplastes PCC 7420 (MC7420_6818), Persephonella marina EX-H1 (PERMA_1676), Desulfitobacterium hafniense Y51 (DSY4489), Prochlorococcus marinus str. AS9601 (A9601_11971), Flavobacteria bacterium BBFL7 (BBFL7_02553), Sphaerochaeta globus str. Buddy (SPIBUDDY_2293), Sphaerochaeta pleomorpha str. Grapes (SPIGRAPES_2501), Staphylococcus aureus subsp. aureus Mu50 (SAV2163), Streptococcus pyogenes M1 GAS (SPY_1036), Synechococcus sp. WH 8109 (SH8109_2193), Prochlorococcus marinus subsp. marinus str. CCMP1375 (PRO_1104), Prochlorococcus marinus str. MIT 9515 (P9515_11821), Prochlorococcus marinus str. MIT 9301 (P9301_11981), Prochlorococcus marinus str. NATL1A (NATL1_14891), Listeria monocytogenes EGD-e (LMO2120), Streptococcus pneumoniae TIGR4 2 seqs SPNET_02000368, SP_1561), Streptococcus pneumoniae R6 (SPR1419), Staphylococcus epidermidis RP62A (SERP1764), Staphylococcus epidermidis ATCC 12228 (SE_1754), Desulfobacterium autotrophicum HRM2 (HRM2_32880), Desulfotalea psychrophila LSv54 (DP1639), Cyanobium sp. PCC 7001 (CPCC7001_1029), Chlamydophila pneumoniae TW-183 (YBBP), Leptospira interrogans serovar Lai str. 56601 (LA_3304), Clostridium perfringens ATCC 13124 (CPF_2660), Thermosynechococcus elongatus BP-1 (TLR1762), Bacillus anthracis str. Ames (BA_0155), Clostridium thermocellum ATCC 27405 (CTHE_1166), Leuconostoc mesenteroides subsp. mesenteroides ATCC 8293 (LEUM_1568), Oenococcus oeni PSU-1 (OEOE_1656), Trichodesmium erythraeum IMS101 (TERY_2433), Tannerella forsythia ATCC 43037 (BFO_1347), Sulfurihydrogenibium azorense Az-Fu1 (SULAZ_1626), Candidatus koribacter versatilis Ellin345 (ACID345_0278), Desulfovibrio alaskensis G20 (DDE_1515), Carnobacterium sp. 17-4 (YBBP), Streptococcus mutans UA159 (SMU_1428C), Mycoplasma agalactiae (MAG3060), Streptococcus agalactiae NEM316 (GBS0902), Clostridium tetani E88 (CTC_02549), Ruminococcus champanellensis 18P13 (RUM_14470), Croceibacter atlanticus HTCC2559 (CA2559_13513), Streptococcus uberis 0140J (SUB1092), Chlamydophila abortus S26/3 (CAB642), Lactobacillus plantarum WCFS1 (LP_0818), Oceanobacillus iheyensis HTE831 (OB0230), Synechococcus sp. RS9916 (RS9916_31367), Synechococcus sp. RS9917 (RS9917_00967), Bacillus subtilis subsp. subtilis str. 168 (YBBP), Aquifex aeolicus VF5 (AQ_1467), Borrelia burgdorferi B31 (BB_0008), Enterococcus faecalis V583 (EF_2157), Bacteroides thetaiotaomicron VPI-5482 (BT_3647), Bacillus cereus ATCC 14579 (BC_0186), Chlamydophila caviae GPIC (CCA_00671), Synechococcus sp. CB0101 (SCB01_010100000902), Synechococcus sp. CB0205 (SCB02_010100012692), Candidatus Solibacter usitatus Ellin6076 (ACID_1909), Geobacillus kaustophilus HTA426 (GK0152), Verrucomicrobium spinosum DSM 4136 (VSPID_010100022530), Anabaena variabilis ATCC 29413 (AVA_0913), Porphyromonas gingivalis W83 (PG_1588), Chlamydia muridarum Nigg (TC_0280), Deinococcus radiodurans R1 (DR_0007), Geobacter sulfurreducens PCA 2 seqs GSU1807, GSU0868), Mycoplasma arthritidis 158L3-1 (MARTH_ORF527), Mycoplasma genitalium G37 (MG105), Treponema denticola ATCC 35405 (TDE_1909), Treponema pallidum subsp. pallidum str. Nichols (TP_0826), butyrate-producing bacterium SS3/4 (CK3_23050), Carboxydothermus hydrogenoformans Z-2901 (CHY_2015), Ruminococcus albus 8 (CUS_5386), Streptococcus mitis NCTC 12261 (SM12261_1151), Gloeobacter violaceus PCC 7421 (GLL0109), Lactobacillus johnsonii NCC 533 (U_0892), Exiguobacterium sibiricum 255-15 (EXIG_0138), Mycoplasma hyopneumoniae J (MHJ_0485), Mycoplasma synoviae 53 (MS53_0498), Thermus thermophilus HB27 (TT_C1660), Onion yellows phytoplasma OY-M (PAM_584), Streptococcus thermophilus LMG 18311 (OSSG), Candidatus Protochlamydia amoebophila UWE25 (PC1633), Chlamydophila felis Fe/C-56 (CF0340), Bdellovibrio bacteriovorus HD100 (BD1929), Prevotella ruminicola 23 (PRU_2261), Moorella thermoacetica ATCC 39073 (MOTH_2248), Leptospira interrogans serovar Copenhageni str. Fiocruz L1-130 (LIC_10844), Mycoplasma mobile 163K (MMOB4550), Synechococcus elongatus PCC 6301 (SYC1250_C), Cytophaga hutchinsonii ATCC 33406 (CHU_3222), Geobacter metallireducens GS-15 2 seqs GMET_1888, GMET_1168), Bacillus halodurans C-125 (BH0265), Bacteroides fragilis NCTC 9343 (BF0397), Chlamydia trachomatis D/UW-3/CX (YBBP), Clostridium acetobutylicum ATCC 824 (CA_C3079), Clostridium difficile 630 (CD0110), Lactobacillus acidophilus NCFM (LBA0714), Lactococcus lactis subsp. lactis 111403 (YEDA), Listeria innocua Clip11262 (LIN2225), Mycoplasma penetrans HF-2 (MYPE2120), Mycoplasma pulmonis UAB CTIP (MYPU_4070), Thermoanaerobacter tengcongensis MB4 (TTE2209), Pediococcus pentosaceus ATCC 25745 (PEPE_0475), Bacillus licheniformis DSM 13=ATCC 14580 2 seqs YBBP, BL02701), Staphylococcus haemolyticus JCSC1435 (SH0877), Desulfuromonas acetoxidans DSM 684 (DACE_0543), Thermodesulfovibrio yellowstonii DSM 11347 (THEYE_A0044), Mycoplasma bovis PG45 (MBOVPG45_0394), Anaeromyxobacter dehalogenans 2CP-C(ADEH_1497), Clostridium beijerinckii NCIMB 8052 (CBEI_0200), Borrelia garinii PBi (BG0008), Symbiobacterium thermophilum IAM 14863 (STH192), Alkaliphilus metalliredigens QYMF (AMET_4313), Thermus thermophilus HB8 (TTHA0323), Coprothermobacter proteolyticus DSM 5265 (COPRO5265_1086), Thermomicrobium roseum DSM 5159 (TRD_0688), Salinibacter ruber DSM 13855 (SRU_1946), Dokdonia donghaensis MED134 (MED134_03354), Polaribacter irgensii 23-P (PI23P_01632), Psychroflexus torquis ATCC 700755 (P700755_02202), Robiginitalea biformata HTCC2501 (RB2501_10597), Polaribacter sp. MED152 (MED152_11519), Maribacter sp. HTCC2170 (FB2170_01652), Microscilla marina ATCC 23134 (M23134_07024), Lyngbya sp. PCC 8106 (L8106_18951), Nodularia spumigena CCY9414 (N9414_23393), Synechococcus sp. BL107 (BL107_11781), Bacillus sp. NRRL B-14911 (B14911_19485), Lentisphaera araneosa HTCC2155 (LNTAR_18800), Lactobacillus sakei subsp. sakei 23K (LCA_1359), Mariprofundus ferrooxydans PV-1 (SPV1_13417), Borrelia hermsii DAH (BH0008), Borrelia turicatae 91E135 (BT0008), Bacillus weihenstephanensis KBAB4 (BCERKBAB4_0149), Bacillus cytotoxicus NVH 391-98 (BCER98_0148), Bacillus pumilus SAFR-032 (YBBP), Geobacter sp. FRC-32 2 seqs GEOB_2309, GEOB_3421), Herpetosiphon aurantiacus DSM 785 (HAUR_3416), Synechococcus sp. RCC307 (SYNRCC307_0791), Synechococcus sp. CC9902 (SYNCC9902_1392), Deinococcus geothermalis DSM 11300 (DGEO_0135), Synechococcus sp. PCC 7002 (SYNPCC7002_A0098), Synechococcus sp. WH 7803 (SYNWH7803_1532), Pedosphaera parvula Ellin514 (CFLAV_PD5552), Synechococcus sp. JA-3-3Ab (CYA_2894), Synechococcus sp. JA-2-3Ba(2-13) (CYB_1645), Aster yellows witches-broom phytoplasma AYWB (AYWB_243), Paenibacillus sp. JDR-2 (PJDR2_5631), Chloroflexus aurantiacus J-10-fl (CAUR_1577), Lactobacillus gasseri ATCC 33323 (LGAS_1288), Bacillus amyloliquefaciens FZB42 (YBBP), Chloroflexus aggregans DSM 9485 (CAGG_2337), Acaryochloris marina MBIC11017 (AM1_0413), Blattabacterium sp. (Blattella germanica) str. Bge (BLBBGE_101), Simkania negevensis Z (YBBP), Chlamydophila pecorum E58 (G5S_1046), Chlamydophila psittaci 6BC 2 seqs CPSIT_0714, G50_0707), Carnobacterium sp. AT7 (CAT7_06573), Finegoldia magna ATCC 29328 (FMG_1225), Syntrophomonas wolfei subsp. wolfei str. Goettingen (SWOL_2103), Syntrophobacter fumaroxidans MPOB (SFUM_3455), Pelobacter carbinolicus DSM 2380 (PCAR_0999), Pelobacter propionicus DSM 2379 2 seqs PPRO_2640, PPRO_2254), Thermoanaerobacter pseudethanolicus ATCC 33223 (TETH39_0457), Victivallis vadensis ATCC BAA-548 (VVAD_PD2437), Staphylococcus saprophyticus subsp. saprophyticus ATCC 15305 (SSP0722), Bacillus coagulans 36D1 (BCOA_1105), Mycoplasma hominis ATCC 23114 (MHO_0510), Lactobacillus reuteri 100-23 (LREU23DRAFT_3463), Desulfotomaculum reducens MI-1 (DRED_0292), Leuconostoc citreum KM20 (LCK_01297), Paenibacillus polymyxa E681 (PPE_04217), Akkermansia muciniphila ATCC BAA-835 (AMUC_0400), Alkaliphilus oremlandii OhILAs (CLOS_2417), Geobacter uraniireducens Rf4 2 seqs GURA_1367, GURA_2732), Caldicellulosiruptor saccharolyticus DSM 8903 (CSAC_1183), Pyramidobacter piscolens W5455 (HMPREF7215_0074), Leptospira borgpetersenii serovar Hardjo-bovis L550 (LBL_0913), Roseiflexus sp. RS-1 (ROSERS_1145), Clostridium phytofermentans ISDg (CPHY_3551), Brevibacillus brevis NBRC 100599 (BBR47_02670), Exiguobacterium sp. AT1b (EAT1B_1593), Lactobacillus salivarius UCC118 (LSL_1146), Lawsonia intracellularis PHE/MN1-00 (LI0190), Streptococcus mitis B6 (SMI_1552), Pelotomaculum thermopropionicum SI (PTH_0536), Streptococcus pneumoniae D39 (SPD_1392), Candidatus Phytoplasma mali (ATP_00312), Gemmatimonas aurantiaca T-27 (GAU_1394), Hydrogenobaculum sp. Y04AAS1 (HY04AAS1_0006), Roseiflexus castenholzii DSM 13941 (RCAS_3986), Listeria welshimeri serovar 6b str. SLCC5334 (LWE2139), Clostridium novyi NT (NTOICX_1162), Lactobacillus brevis ATCC 367 (LVIS_0684), Bacillus sp. B14905 (BB14905_08668), Algoriphagus sp. PR1 (ALPR1_16059), Streptococcus sanguinis SK36 (SSA_0802), Borrelia afzelii PKo 2 seqs BAPKO_0007, AEL69242.1), Lactobacillus delbrueckii subsp. bulgaricus ATCC 11842 (LDB0651), Streptococcus suis 05ZYH33 (SSU05_1470), Kordia algicida OT-1 (KAOT1_10521), Pedobacter sp. BAL39 (PBAL39_03944), Flavobacteriales bacterium ALC-1 (FBALC1_04077), Cyanothece sp. CCY0110 (CY0110_30633), Plesiocystis pacifica SIR-1 (PPSIR1_10140), Clostridium cellulolyticum H10 (CCEL_1201), Cyanothece sp. PCC 7425 (CYAN7425_4701), Staphylococcus carnosus subsp. carnosus TM300 (SCA_1665), Bacillus pseudofirmus OF4 (YBBP), Leeuwenhoekiella blandensis MED217 (MED217_04352), Geobacter lovleyi SZ 2 seqs GLOV_3055, GLOV_2524), Streptococcus equi subsp. zooepidemicus (SEZ_1213), Thermosinus carboxydivorans Nor (TCARDRAFT_1045), Geobacter bemidjiensis Bem (GBEM_0895), Anaeromyxobacter sp. Fw109-5 (ANAE109_2336), Lactobacillus helveticus DPC 4571 (LHV_0757), Bacillus sp. m3-13 (BM3-1_010100010851), Gramella forsetii KT0803 (GFO_0428), Ruminococcus obeum ATCC 29174 (RUMOBE_03597), Ruminococcus torques ATCC 27756 (RUMTOR_00870), Dorea formicigenerans ATCC 27755 (DORFOR_00204), Dorea longicatena DSM 13814 (DORLON_01744), Eubacterium ventriosum ATCC 27560 (EUBVEN_01080), Desulfovibrio piger ATCC 29098 (DESPIG_01592), Parvimonas micra ATCC 33270 (PEPMIC_01312), Pseudoflavonifractor capillosus ATCC 29799 (BACCAP_01950), Clostridium scindens ATCC 35704 (CLOSCI_02389), Eubacterium hallii DSM 3353 (EUBHAL_01228), Ruminococcus gnavus ATCC 29149 (RUMGNA_03537), Subdoligranulum variabile DSM 15176 (SUBVAR_05177), Coprococcus eutactus ATCC 27759 (COPEUT_01499), Bacteroides ovatus ATCC 8483 (BACOVA_03480), Parabacteroides merdae ATCC 43184 (PARMER_03434), Faecalibacterium prausnitzii A2-165 (FAEPRAA2165_01954), Clostridium sp. L2-50 (CLOL250_00341), Anaerostipes caccae DSM 14662 (ANACAC_00219), Bacteroides caccae ATCC 43185 (BACCAC_03225), Clostridium bolteae ATCC BAA-613 (CLOBOL_04759), Borrelia duttonii Ly (BDU_14), Cyanothece sp. PCC 8801 (PCC8801_0127), Lactococcus lactis subsp. cremoris MG1363 (LLMG_0448), Geobacillus thermodenitrificans NG80-2 (GTNG_0149), Epulopiscium sp. N.t. morphotype B (EPULO_010100003839), Lactococcus garvieae Lg2 (LCGL_0304), Clostridium leptum DSM 753 (CLOLEP_03097), Clostridium spiroforme DSM 1552 (CLOSPI_01608), Eubacterium dolichum DSM 3991 (EUBDOL_00188), Clostridium kluyveri DSM 555 (CKL_0313), Porphyromonas gingivalis ATCC 33277 (PGN_0523), Bacteroides vulgatus ATCC 8482 (BVU_0518), Parabacteroides distasonis ATCC 8503 (BDI_3368), Staphylococcus hominis subsp. hominis C80 (HMPREF0798_01968), Staphylococcus caprae C87 (HMPREF0786_02373), Streptococcus sp. C150 (HMPREF0848_00423), Sulfurihydrogenibium sp. YO3AOP1 (SYO3AOP1_0110), Desulfatibacillum alkenivorans AK-01 (DALK_0397), Bacillus selenitireducens MLS10 (BSEL_0372), Cyanothece sp. ATCC 51142 (CCE_1350), Lactobacillus jensenii 1153 (LBJG_01645), Acholeplasma laidlawii PG-8A (ACL_1368), Bacillus coahuilensis m4-4 (BCOAM_010100001120), Geobacter sp. M18 2 seqs GM18_0792, GM18_2516), Lysinibacillus sphaericus C3-41 (BSPH_4568), Clostridium botulinum NCTC 2916 (CBN_3506), Clostridium botulinum C str. Eklund (CBC_A1575), Alistipes putredinis DSM 17216 (ALIPUT_00190), Anaerofustis stercorihominis DSM 17244 (ANASTE_01539), Anaerotruncus colihominis DSM (ANACOL_02706), Clostridium bartlettii DSM 16795 (CLOBAR_00759), Clostridium ramosum DSM 1402 (CLORAM_01482), Borrelia valaisiana VS116 (BVAVS116_0007), Sorangium cellulosum So ce 56 (SCE7623), Microcystis aeruginosa NIES-843 (MAE_25390), Bacteroides stercoris ATCC 43183 (BACSTE_02634), Candidatus Amoebophilus asiaticus 5a2 (AASI_0652), Leptospira biflexa serovar Patoc strain Patoc 1 (Paris) (LEPBI_I0735), Clostridium sp. 7_2_43FAA (CSBG_00101), Desulfovibrio sp. 3_1_syn3 (HMPREF0326_02254), Ruminococcus sp. 5_1_39BFAA (RSAG_02135), Clostridiales bacterium 1_7_47FAA (CBFG_00347), Bacteroides fragilis 3_1_12 (BFAG_02578), Natranaerobius thermophilus JW/NM-WN-LF (NTHER_0240), Macrococcus caseolyticus JCSC5402 (MCCL_0321), Streptococcus gordonii str. Challis substr. CH1 (SGO_0887), Dethiosulfovibrio peptidovorans DSM 11002 (DPEP_2062), Coprobacillus sp. 29_1 (HMPREF9488_03448), Bacteroides coprocola DSM 17136 (BACCOP_03665), Coprococcus comes ATCC 27758 (COPCOM_02178), Geobacillus sp. WCH70 (GWCH70_0156), uncultured Termite group 1 bacterium phylotype Rs-D17 (TGRD_209), Dyadobacter fermentans DSM 18053 (DFER_0224), Bacteroides intestinalis DSM 17393 (BACINT_00700), Ruminococcus lactaris ATCC 29176 (RUMLAC_01257), Blautia hydrogenotrophica DSM 10507 (RUMHYD_01218), Candidatus Desulforudis audaxviator MP104C (DAUD_1932), Marvinbryantia formatexigens DSM 14469 (BRYFOR_07410), Sphaerobacter thermophilus DSM 20745 (STHE_1601), Veillonella parvula DSM 2008 (VPAR_0292), Methylacidiphilum infernorum V4 (MINF_1897), Paenibacillus sp. Y412MC10 (GYMC10_5701), Bacteroides finegoldii DSM 17565 (BACFIN_07732), Bacteroides eggerthii DSM 20697 (BACEGG_03561), Bacteroides pectinophilus ATCC 43243 (BACPEC_02936), Bacteroides plebeius DSM 17135 (BACPLE_00693), Desulfohalobium retbaense DSM 5692 (DRET_1725), Desulfotomaculum acetoxidans DSM 771 (DTOX_0604), Pedobacter heparinus DSM 2366 (PHEP_3664), Chitinophaga pinensis DSM 2588 (CPIN_5466), Flavobacteria bacterium MS024-2A (FLAV2ADRAFT_0090), Flavobacteria bacterium MS024-3C (FLAV3CDRAFT_0851), Moorea producta 3L (LYNGBM3L_14400), Anoxybacillus flavithermus WK1 (AFLV_0149), Mycoplasma fermentans PG18 (MBIO_0474), Chthoniobacter flavus Ellin428 (CFE428DRAFT_3031), Cyanothece sp. PCC 7822 (CYAN7822_1152), Borrelia spielmanii A14S (BSPA14S_0009), Heliobacterium modesticaldum Icel (HM1_1522), Thermus aquaticus Y51MC23 (TAQDRAFT_3938), Clostridium sticklandii DSM 519 (CLOST_0484), Tepidanaerobacter sp. Rel (TEPRE1_0323), Clostridium hiranonis DSM 13275 (CLOHIR_00003), Mitsuokella multacida DSM 20544 (MITSMUL_03479), Haliangium ochraceum DSM 14365 (HOCH_3550), Spirosoma linguale DSM 74 (SLIN_2673), unidentified eubacterium SCB49 (SCB49_03679), Acetivibrio cellulolyticus CD2 (ACELC_020100013845), Lactobacillus buchneri NRRL B-30929 (LBUC_1299), Butyrivibrio crossotus DSM 2876 (BUTYVIB_02056), Candidatus Azobacteroides pseudotrichonymphae genomovar. CFP2 (CFPG_066), Mycoplasma crocodyli MP145 (MCRO_0385), Arthrospira maxima CS-328 (AMAXDRAFT_4184), Eubacterium eligens ATCC 27750 (EUBELI_01626), Butyrivibrio proteoclasticus B316 (BPR_I2587), Chloroherpeton thalassium ATCC 35110 (CTHA_1340), Eubacterium biforme DSM 3989 (EUBIFOR_01794), Rhodothermus marinus DSM 4252 (RMAR_0146), Borrelia bissettii DN127 (BBIDN127_0008), Capnocytophaga ochracea DSM 7271 (COCH_2107), Alicyclobacillus acidocaldarius subsp. acidocaldarius DSM 446 (AACI_2672), Caldicellulosiruptor bescii DSM 6725 (ATHE_0361), Denitrovibrio acetiphilus DSM 12809 (DACET_1298), Desulfovibrio desulfuricans subsp. desulfuricans str. ATCC 27774 (DDES_1715), Anaerococcus lactolyticus ATCC 51172 (HMPREF0072_1645), Anaerococcus tetradius ATCC 35098 (HMPREF0077_0902), Finegoldia magna ATCC 53516 (HMPREF0391_10377), Lactobacillus antri DSM 16041 (YBBP), Lactobacillus buchneri ATCC 11577 (HMPREF0497_2752), Lactobacillus ultunensis DSM 16047 (HMPREF0548_0745), Lactobacillus vaginalis ATCC 49540 (HMPREF0549_0766), Listeria grayi DSM 20601 (HMPREF0556_11652), Sphingobacterium spiritivorum ATCC 33861 (HMPREF0766_11787), Staphylococcus epidermidis M23864:W1 (HMPREF0793_0092), Streptococcus equinus ATCC 9812 (HMPREF0819_0812), Desulfomicrobium baculatum DSM 4028 (DBAC_0255), Thermanaerovibrio acidaminovorans DSM 6589 (TACI_0837), Thermobaculum terrenum ATCC BAA-798 (TTER_1817), Anaerococcus prevotii DSM 20548 (APRE_0370), Desulfovibrio salexigens DSM 2638 (DESAL_1795), Brachyspira murdochii DSM 12563 (BMUR_2186), Meiothermus silvanus DSM 9946 (MESIL_0161), Bacillus cereus Rock4-18 (BCERE0024_1410), Cylindrospermopsis raciborskii CS-505 (CRC_01921), Raphidiopsis brookii D9 (CRD_01188), Clostridium carboxidivorans P7 2 seqs CLCAR_0016, CCARBDRAFT_4266), Clostridium botulinum E1 str. BoNT E Beluga (CLO_3490), Blautia hansenii DSM 20583 (BLAHAN_07155), Prevotella copri DSM 18205 (PREVCOP_04867), Clostridium methylpentosum DSM 5476 (CLOSTMETH_00084), Lactobacillus casei BL23 (LCABL_11800), Bacillus megaterium QM B1551 (BMQ_0195), Treponema primitia ZAS-2 (TREPR_1936), Treponema azotonutricium ZAS-9 (TREAZ_0147), Holdemania filiformis DSM 12042 (HOLDEFILI_03810), Filifactor alocis ATCC 35896 (HMPREF0389_00366), Gemella haemolysans ATCC 10379 (GEMHA0001_0912), Selenomonas sputigena ATCC 35185 (SELSP_1610), Veillonella dispar ATCC 17748 (VEIDISOL_01845), Deinococcus deserti VCD115 (DEIDE_19700), Bacteroides coprophilus DSM 18228 (BACCOPRO_00159), Nostoc azollae 0708 (AAZO_4735), Erysipelotrichaceae bacterium 5_2_54FAA (HMPREF0863_02273), Ruminococcaceae bacterium D16 (HMPREF0866_01061), Prevotella bivia JCVIHMP010 (HMPREF0648_0338), Prevotella melaninogenica ATCC 25845 (HMPREF0659_A6212), Porphyromonas endodontalis ATCC 35406 (POREN0001_0251), Capnocytophaga sputigena ATCC 33612 (CAPSP0001_0727), Capnocytophaga gingivalis ATCC 33624 (CAPGI0001_1936), Clostridium hylemonae DSM 15053 (CLOHYLEM_04631), Thermosediminibacter oceani DSM 16646 (TOCE_1970), Dethiobacter alkaliphilus AHT 1 (DEALDRAFT_0231), Desulfonatronospira thiodismutans AS03-1 (DTHIO_PD2806), Clostridium sp. D5 (HMPREF0240_03780), Anaerococcus hydrogenalis DSM 7454 (ANHYDRO_01144), Kyrpidia tusciae DSM 2912 (BTUS_0196), Gemella haemolysans M341 (HMPREF0428_01429), Gemella morbillorum M424 (HMPREF0432_01346), Gemella sanguinis M325 (HMPREF0433_01225), Prevotella oris C735 (HMPREF0665_01741), Streptococcus sp. M143 (HMPREF0850_00109), Streptococcus sp. M334 (HMPREF0851_01652), Bilophila wadsworthia 3_1_6 (HMPREF0179_00899), Brachyspira hyodysenteriae WA1 (BHWA1_01167), Enterococcus gallinarum EG2 (EGBG_00820), Enterococcus casseliflavus EC20 (ECBG_00827), Enterococcus faecium C68 (EFXG_01665), Syntrophus aciditrophicus SB (SYN_02762), Lactobacillus rhamnosus GG 2 seqs OSSG, LRHM_0937), Acidaminococcus intestini RyC-MR95 (ACIN_2069), Mycoplasma conjunctivae HRC/581 (MCJ_002940), Halanaerobium praevalens DSM 2228 (HPRAE_1647), Aminobacterium colombiense DSM 12261 (AMICO_0737), Clostridium cellulovorans 743B (CLOCEL_3678), Desulfovibrio magneticus RS-1 (DMR_25720), Spirochaeta smaragdinae DSM 11293 (SPIRS_1647), Bacteroidetes oral taxon 274 str. F0058 (HMPREF0156_01826), Lachnospiraceae oral taxon 107 str. F0167 (HMPREF0491_01238), Lactobacillus coleohominis 101-4-CHN (HMPREF0501_01094), Lactobacillus jensenii 27-2-CHN (HMPREF0525_00616), Prevotella buccae D17 (HMPREF0649_02043), Prevotella sp. oral taxon 299 str. F0039 (HMPREF0669_01041), Prevotella sp. oral taxon 317 str. F0108 (HMPREF0670_02550), Desulfobulbus propionicus DSM 2032 2 seqs DESPR_2503, DESPR_1053), Thermoanaerobacterium thermosaccharolyticum DSM 571 (TTHE_0484), Thermoanaerobacter italicus Ab9 (THIT_1921), Thermovirga lienii DSM 17291 (TLIE_0759), Aminomonas paucivorans DSM 12260 (APAU_1274), Streptococcus mitis SK321 (SMSK321_0127), Streptococcus mitis SK597 (SMSK597_0417), Roseburia hominis A2-183 (RHOM_12405), Oribacterium sinus F0268 (HMPREF6123_0887), Prevotella bergensis DSM 17361 (HMPREF0645_2701), Selenomonas noxia ATCC 43541 (YBBP), Weissella paramesenteroides ATCC 33313 (HMPREF0877_0011), Lactobacillus amylolyticus DSM 11664 (HMPREF0493_1017), Bacteroides sp. D20 (HMPREF0969_02087), Clostridium papyrosolvens DSM 2782 (CPAP_3968), Desulfurivibrio alkaliphilus AHT2 (DAAHT2_0445), Acidaminococcus fermentans DSM 20731 (ACFER_0601), Abiotrophia defectiva ATCC 49176 (GCWU000182_00063), Anaerobaculum hydrogeniformans ATCC BAA-1850 (HMPREF1705_01115), Catonella morbi ATCC 51271 (GCWU000282_00629), Clostridium botulinum D str. 1873 (CLG_B1859), Dialister invisus DSM 15470 (GCWU000321_01906), Fibrobacter succinogenes subsp. succinogenes S85 2 seqs FSU_0028, FISUC_2776), Desulfovibrio fructosovorans JJ (DESFRDRAFT_2879), Peptostreptococcus stomatis DSM 17678 (HMPREF0634_0727), Staphylococcus warneri L37603 (STAWA0001_0094), Treponema vincentii ATCC 35580 (TREVI00011289), Porphyromonas uenonis 60-3 (PORUE0001_0199), Peptostreptococcus anaerobius 653-L (HMPREF0631_1228), Peptoniphilus lacrimalis 315-B (HMPREF0628_0762), Candidatus Phytoplasma australiense (PA0090), Prochlorococcus marinus subsp. pastoris str. CCMP1986 (PMM1091), Synechococcus sp. WH 7805 (WH7805_04441), Blattabacterium sp. (Periplaneta americana) str. BPLAN (BPLAN_534), Caldicellulosiruptor obsidiansis OB47 (COB47_0325), Oribacterium sp. oral taxon 078 str. F0262 (GCWU000341_01365), Hydrogenobacter thermophilus TK-6 2 seqs AD046034.1, HTH_1665), Clostridium saccharolyticum WM1 (CLOSA_1248), Prevotella sp. oral taxon 472 str. F0295 (HMPREF6745_1617), Paenibacillus sp. oral taxon 786 str. D14 (POTG_03822), Roseburia inulinivorans DSM 16841 2 seqs ROSEINA2194_02614, ROSEINA2194_02613), Granulicatella elegans ATCC 700633 (HMPREF0446_01381), Prevotella tannerae ATCC 51259 (GCWU000325_02844), Shuttleworthia satelles DSM 14600 (GCWU000342_01722), Phascolarctobacterium succinatutens YIT 12067 (HMPREF9443_01522), Clostridium butyricum E4 str. BoNT E BL5262 (CLP_3980), Caldicellulosiruptor hydrothermalis 108 (CALHY_2287), Caldicellulosiruptor kristjanssonii 177R1B (CALKR_0314), Caldicellulosiruptor owensensis OL (CALOW_0228), Eubacterium cellulosolvens 6 (EUBCEDRAFT_1150), Geobacillus thermoglucosidasius C56-YS93 (GEOTH_0175), Thermincola potens JR (THERJR_0376), Nostoc punctiforme PCC 73102 (NPUN_F5990), Granulicatella adiacens ATCC 49175 (YBBP), Selenomonas flueggei ATCC 43531 (HMPREF0908_1366), Thermocrinis albus DSM 14484 (THAL_0234), Deferribacter desulfuricans SSM1 (DEFDS_1031), Ruminococcus flavefaciens FD-1 (RFLAF_010100012444), Desulfovibrio desulfuricans ND132 (DND132_0877), Clostridium lentocellum DSM 5427 (CLOLE_3370), Desulfovibrio aespoeensis Aspo-2 (DAES_1257), Syntrophothermus lipocalidus DSM 12680 (SLIP_2139), Marivirga tractuosa DSM 4126 (FTRAC_3720), Desulfarculus baarsii DSM 2075 (DEBA_0764), Synechococcus sp. CC9311 (SYNC_1030), Thermaerobacter marianensis DSM 12885 (TMAR_0236), Desulfovibrio sp. FW1012B (DFW101_0480), Jonquetella anthropi E3_33 E1 (GCWU000246_01523), Syntrophobotulus glycolicus DSM 8271 (SGLY_0483), Thermovibrio ammonificans HB-1 (THEAM_0892), Truepera radiovictrix DSM 17093 (TRAD_1704), Bacillus cellulosilyticus DSM 2522 (BCELL_0170), Prevotella veroralis F0319 (HMPREF0973_02947), Erysipelothrix rhusiopathiae str. Fujisawa (ERH_0115), Desulfurispirillum indicum S5 (SELIN_2326), Cyanothece sp. PCC 7424 (PCC7424_0843), Anaerococcus vaginalis ATCC 51170 (YBBP), Aerococcus viridans ATCC 11563 (YBBP), Streptococcus oralis ATCC 35037 2 seqs HMPREF8579_1682, SMSK23_1115), Zunongwangia profunda SM-A87 (ZPR_0978), Halanaerobium hydrogeniformans (HALSA_1882), Bacteroides xylanisolvens XB1A (BXY_29650), Ruminococcus torques L2-14 (RTO_16490), Ruminococcus obeum A2-162 (CK5_33600), Eubacterium rectale DSM 17629 (EUR_24910), Faecalibacterium prausnitzii SL3/3 (FPR_27630), Ruminococcus sp. SRI/5 (CK1_39330), Lachnospiraceae bacterium 3_1_57FAA_CT1 (HMPREF0994_01490), Lachnospiraceae bacterium 9_1_43BFAA (HMPREF0987_01591), Lachnospiraceae bacterium 1_4_56FAA (HMPREF0988_01806), Erysipelotrichaceae bacterium 3_1_53 (HMPREF0983_01328), Ethanoligenens harbinense YUAN-3 (ETHHA_1605), Streptococcus dysgalactiae subsp. dysgalactiae ATCC 27957 (SDD27957_06215), Spirochaeta thermophila DSM 6192 (STHERM_C18370), Bacillus sp. 2_A_57_CT2 (HMPREF1013_05449), Bacillus clausii KSM-K16 (ABC0241), Thermodesulfatator indicus DSM 15286 (THEIN_0076), Bacteroides salanitronis DSM 18170 (BACSA_1486), Oceanithermus profundus DSM 14977 (OCEPR_2178), Prevotella timonensis CRIS 5C-B1 (HMPREF9019_2028), Prevotella buccalis ATCC 35310 (HMPREF0650_0675), Prevotella amnii CRIS 21A-A (HMPREF9018_0365), Bulleidia extructa W1219 (HMPREF9013_0078), Bacteroides coprosuis DSM 18011 (BCOP_0558), Prevotella multisaccharivorax DSM 17128 (PREMU_0839), Cellulophaga algicola DSM 14237 (CELAL_0483), Synechococcus sp. WH 5701 (WH5701_10360), Desulfovibrio africanus str. Walvis Bay (DESAF_3283), Oscillibacter valericigenes Sjm18-20 (OBV_23340), Deinococcus proteolyticus MRP (DEIPR_0134), Bacteroides helcogenes P 36-108 (BACHE_0366), Paludibacter propionicigenes WB4 (PALPR_1923), Desulfotomaculum nigrificans DSM 574 (DESNIDRAFT_2093), Arthrospira platensis NIES-39 (BAI89442.1), Mahella australiensis 50-1 BON (MAHAU_1846), Thermoanaerobacter wiegelii Rt8.B1 (THEWI_2191), Ruminococcus albus 7 (RUMAL_2345), Staphylococcus lugdunensis HKU09-01 (SLGD_00862), Megasphaera genomosp. type_1 str. 28L (HMPREF0889_1099), Clostridiales genomosp. BVAB3 str. UPII9-5 (HMPREF0868_1453), Pediococcus claussenii ATCC BAA-344 (PECL_571), Prevotella oulorum F0390 (HMPREF9431_01673), Turicibacter sanguinis PC909 (CUW_0305), Listeria seeligeri FSL N1-067 (NT03LS_2473), Solobacterium moorei F0204 (HMPREF9430_01245), Megasphaera micronuciformis F0359 (HMPREF9429_00929), Capnocytophaga sp. oral taxon 329 str. F0087 2 seqs HMPREF9074_00867, HMPREF9074_01078), Streptococcus anginosus F0211 (HMPREF0813_00157), Mycoplasma suis KI3806 (MSUI04040), Mycoplasma gallisepticum str. F (MGF_2771), Deinococcus maricopensis DSM 21211 (DEIMA_0651), Odoribacter splanchnicus DSM 20712 (ODOSP_0239), Lactobacillus fermentum CECT 5716 (LC40_0265), Lactobacillus iners AB-1 (LINEA_010100006089), cyanobacterium UCYN-A (UCYN_03150), Lactobacillus sanfranciscensis TMW 1.1304 (YBBP), Mucilaginibacter paludis DSM 18603 (MUCPA_1296), Lysinibacillus fusiformis ZC1 (BFZC1_03142), Paenibacillus vortex V453 (PVOR_30878), Waddlia chondrophila WSU 86-1044 (YBBP), Flexistipes sinusarabici DSM 4947 (FLEXSI_0971), Paenibacillus curdlanolyticus YK9 (PAECUDRAFT_1888), Clostridium cf. saccharolyticum K10 (CLS_03290), Alistipes shahii WAL 8301 (AL1_02190), Eubacterium cylindroides T2-87 (EC1_00230), Coprococcus catus GD/7 (CC1_32460), Faecalibacterium prausnitzii L2-6 (FP2_09960), Clostridium clariflavum DSM 19732 (CLOCL_2983), Bacillus atrophaeus 1942 (BATR1942_19530), Mycoplasma pneumoniae FH (MPNE_0277), Lachnospiraceae bacterium 2_1_46FAA (HMPREF9477_00058), Clostridium symbiosum WAL-14163 (HMPREF9474_01267), Dysgonomonas gadei ATCC BAA-286 (HMPREF9455_02764), Dysgonomonas mossii DSM 22836 (HMPREF9456_00401), Thermus scotoductus SA-01 (TSC_C24350), Sphingobacterium sp. 21 (SPH21_1233), Spirochaeta caldaria DSM 7334 (SPICA_1201), Prochlorococcus marinus str. MIT 9312 (PMT9312_1102), Prochlorococcus marinus str. MIT 9313 (PMT_1058), Faecalibacterium cf. prausnitzii KLE1255 (HMPREF9436_00949), Lactobacillus crispatus ST1 (LCRIS_00721), Clostridium ljungdahlii DSM 13528 (CLJU_C40470), Prevotella bryantii B14 (PBR_2345), Treponema phagedenis F0421 (HMPREF9554_02012), Clostridium sp. BNL1100 (CLO1100_2851), Microcoleus vaginatus FGP-2 (MICVADRAFT_1377), Brachyspira pilosicoli 95/1000 (BP951000_0671), Spirochaeta coccoides DSM 17374 (SPICO_1456), Haliscomenobacter hydrossis DSM 1100 (HALHY_5703), Desulfotomaculum kuznetsovii DSM 6115 (DESKU_2883), Runella slithyformis DSM 19594 (RUNSL_2859), Leuconostoc kimchii IMSNU 11154 (LKI_08080), Leuconostoc gasicomitatum LMG 18811 (OSSG), Pedobacter saltans DSM 12145 (PEDSA_3681), Paraprevotella xylaniphila YIT 11841 (HMPREF9442_00863), Bacteroides clarus YIT 12056 (HMPREF9445_01691), Bacteroides fluxus YIT 12057 (HMPREF9446_03303), Streptococcus urinalis 2285-97 (STRUR_1376), Streptococcus macacae NCTC 11558 (STRMA_0866), Streptococcus ictaluri 707-05 (STRIC_0998), Oscillochloris trichoides DG-6 (OSCT_2821), Parachlamydia acanthamoebae UV-7 (YBBP), Prevotella denticola F0289 (HMPREF9137_0316), Parvimonas sp. oral taxon 110 str. F0139 (HMPREF9126_0534), Calditerrivibrio nitroreducens DSM 19672 (CALNI_1443), Desulfosporosinus orientis DSM 765 (DESOR_0366), Streptococcus mitis bv. 2 str. F0392 (HMPREF9178_0602), Thermodesulfobacterium sp. OPB45 (TOPB45_1366), Synechococcus sp. WH 8102 (SYNW0935), Thermoanaerobacterium xylanolyticum LX-11 (THEXY_0384), Mycoplasma haemofelis Ohio2 (MHF_1192), Capnocytophaga canimorsus Cc5 (CCAN_16670), Pediococcus acidilactici DSM 20284 (HMPREF0623_1647), Prevotella marshii DSM 16973 (HMPREF0658_1600), Peptoniphilus duerdenii ATCC BAA-1640 (HMPREF9225_1495), Bacteriovorax marinus SJ (BMS_2126), Selenomonas sp. oral taxon 149 str. 67H29BP (HMPREF9166_2117), Eubacterium yurii subsp. margaretiae ATCC 43715 (HMPREF0379_1170), Streptococcus mitis ATCC 6249 (HMPREF8571_1414), Streptococcus sp. oral taxon 071 str. 73H25AP (HMPREF9189_0416), Prevotella disiens FB035-09AN (HMPREF9296_1148), Aerococcus urinae ACS-120-V-Col10a (HMPREF9243_0061), Veillonella atypica ACS-049-V-Sch6 (HMPREF9321_0282), Cellulophaga lytica DSM 7489 (CELLY_2319), Thermaerobacter subterraneus DSM 13965 (THESUDRAFT_0411), Desulfurobacterium thermolithotrophum DSM 11699 (DESTER_0391), Treponema succinifaciens DSM 2489 (TRESU_1152), Marinithermus hydrothermalis DSM 14884 (MARKY_1861), Streptococcus infantis SK1302 (SIN_0824), Streptococcus parauberis NCFD 2020 (SPB_0808), Streptococcus porcinus str. Jelinkova 176 (STRPO_0164), Streptococcus criceti HS-6 (STRCR_1133), Capnocytophaga ochracea F0287 (HMPREF1977_0786), Prevotella oralis ATCC 33269 (HMPREF0663_10671), Porphyromonas asaccharolytica DSM 20707 (PORAS_0634), Anaerococcus prevotii ACS-065-V-Col13 (HMPREF9290_0962), Peptoniphilus sp. oral taxon 375 str. F0436 (HMPREF9130_1619), Veillonella sp. oral taxon 158 str. F0412 (HMPREF9199_0189), Selenomonas sp. oral taxon 137 str. F0430 (HMPREF9162_2458), Cyclobacterium marinum DSM 745 (CYCMA_2525), Desulfobacca acetoxidans DSM 11109 (DESAC_1475), Listeria ivanovii subsp. ivanovii PAM 55 (LIV_2111), Desulfovibrio vulgaris str. Hildenborough (DVU_1280), Desulfovibrio vulgaris str. ‘Miyazaki F’ (DVMF_0057), Muricauda ruestringensis DSM 13258 (MURRU_0474), Leuconostoc argentinum KCTC 3773 (LARGK3_010100008306), Paenibacillus polymyxa SC2 (PPSC2_C4728), Eubacterium saburreum DSM 3986 (HMPREF0381_2518), Pseudoramibacter alactolyticus ATCC 23263 (HMP0721_0313), Streptococcus parasanguinis ATCC 903 (HMPREF8577_0233), Streptococcus sanguinis ATCC 49296 (HMPREF8578_1820), Capnocytophaga sp. oral taxon 338 str. F0234 (HMPREF9071_1325), Centipeda periodontii DSM 2778 (HMPREF9081_2332), Prevotella multiformis DSM 16608 (HMPREF9141_0346), Streptococcus peroris ATCC 700780 (HMPREF9180_0434), Prevotella salivae DSM 15606 (HMPREF9420_1402), Streptococcus australis ATCC 700641 2 seqs HMPREF9961_0906, HMPREF9421_1720), Streptococcus cristatus ATCC 51100 2 seqs HMPREF9422_0776, HMPREF9960_0531), Lactobacillus acidophilus 30SC (LAC30SC_03585), Eubacterium limosum KIST612 (ELI_0726), Streptococcus downei F0415 (HMPREF9176_1204), Streptococcus sp. oral taxon 056 str. F0418 (HMPREF9182_0330), Oribacterium sp. oral taxon 108 str. F0425 (HMPREF9124_1289), Streptococcus vestibularis F0396 (HMPREF9192_1521), Treponema brennaborense DSM 12168 (TREBR_1165), Leuconostoc fallax KCTC 3537 (LFALK3_010100008689), Eremococcus coleocola ACS-139-V-Col8 (HMPREF9257_0233), Peptoniphilus harei ACS-146-V-Sch2b (HMPREF9286_0042), Clostridium sp. HGF2 (HMPREF9406_3692), Alistipes sp. HGB5 (HMPREF9720_2785), Prevotella dentalis DSM 3688 (PREDE_0132), Streptococcus pseudoporcinus SPIN 20026 (HMPREF9320_0643), Dialister microaerophilus UPII 345-E (HMPREF9220_0018), Weissella cibaria KACC 11862 (WCIBK1_010100001174), Lactobacillus coryniformis subsp. coryniformis KCTC 3167 (LCORCK3_010100001982), Synechococcus sp. PCC 7335 (S7335_3864), Owenweeksia hongkongensis DSM 17368 (OWEHO_3344), Anaerolinea thermophila UNI-1 (ANT_09470), Streptococcus oralis Uo5 (SOR_0619), Leuconostoc gelidum KCTC 3527 (LGELK3_010100006746), Clostridium botulinum BKT015925 (CBC4_0275), Prochlorococcus marinus str. MIT 9211 (P9211_10951), Prochlorococcus marinus str. MIT 9215 (P9215_12271), Staphylococcus aureus subsp. aureus NCTC 8325 (SAOUHSC_02407), Staphylococcus aureus subsp. aureus COL (SACOL2153), Lactobacillus animalis KCTC 3501 (LANIK3_010100000290), Fructobacillus fructosus KCTC 3544 (FFRUK3_010100006750), Acetobacterium woodii DSM 1030 (AWO_C28200), Planococcus donghaensis MPA1U2 (GPDM_12177), Lactobacillus farciminis KCTC 3681 (LFARK3_010100009915), Melissococcus plutonius ATCC 35311 (MPTP_0835), Lactobacillus fructivorans KCTC 3543 (LFRUK3_010100002657), Paenibacillus sp. HGF7 (HMPREF9413_5563), Lactobacillus oris F0423 (HMPREF9102_1081), Veillonella sp. oral taxon 780 str. F0422 (HMPREF9200_1112), Parvimonas sp. oral taxon 393 str. F0440 (HMPREF9127_1171), Tetragenococcus halophilus NBRC 12172 (TEH_13100), Candidatus Chloracidobacterium thermophilum B (CABTHER_A1277), Ornithinibacillus scapharcae TW25 (OTW25_010100020393), Lacinutrix sp. 5H-3-7-4 (LACAL_0337), Krokinobacter sp. 4H-3-7-5 (KRODI_0177), Staphylococcus pseudintermedius ED99 (SPSE_0659), Staphylococcus aureus subsp. aureus MSHR1132 (CCE59824.1), Paenibacillus terrae HPL-003 (HPL003_03660), Caldalkalibacillus thermarum TA2.A1 (CATHTA2_0882), Desmospora sp. 8437 (HMPREF9374_2897), Prevotella nigrescens ATCC 33563 (HMPREF9419_1415), Prevotella pallens ATCC 700821 (HMPREF9144_0175), Streptococcus infantis X (HMPREF1124.

In some embodiments, the genetically engineered bacteria are capable of increasing c-di-AMP levels. In some embodiments, the genetically engineered bacteria are capable of increasing c-diAMP levels in the intracellular space. In some embodiments, the genetically engineered bacteria are capable of increasing c-diAMP levels inside of a eukaryotic cell. In some embodiments, the genetically engineered bacteria are capable of increasing c-diAMP levels inside of an immune cell. In some embodiments, the cell is a phagocyte. In some embodiments, the cell is a macrophage. In some embodiments, the cell is a dendritic cell. In some embodiments, the cell is a neutrophil. In some embodiments, the cell is a MDSC. In some embodiments, the genetically engineered bacteria are capable of increasing c-GAMP (2′3′ or 3′3′) and/or cyclic-di-GMP levels inside of a cell. In some embodiments, the genetically engineered bacteria are capable of increasing c-di-AMP levels in vitro in the bacterial cell and/or in the growth medium.

In any of these embodiments, the bacteria genetically engineered to produce cyclic-di-AMP produce at least about 0% to 2% to 4%, 4% to 6%, 6% to 8%, 8% to 10%, 10% to 12%, 12% to 14%, 14% to 16%, 16% to 18%, 18% to 20%, 20% to 25%, 25% to 30%, 30% to 35%, 35% to 40%, 40% to 45% 45% to 50%, 50% to 55%, 55% to 60%, 60% to 65%, 65% to 70% to 80%, 80% to 90%, or 90% to 100% more cyclic-di-AMP than unmodified bacteria of the same bacterial subtype under the same conditions. In yet another embodiment, the genetically engineered bacteria produce at least about 0 to 1.0-fold, 1.0-1.2-fold, 1.2-1.4-fold, 1.4-1.6-fold, 1.6-1.8-fold, 1.8-2-fold, or two-fold more cyclic-di-AMP than unmodified bacteria of the same bacterial subtype under the same conditions. In yet another embodiment, the genetically engineered bacteria produce at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more cyclic-di-AMP than unmodified bacteria of the same bacterial subtype under the same conditions.

In any of these embodiments, the bacteria genetically engineered to produce cyclic-di-AMP consume at least about 0% to 2% to 4%, 4% to 6%, 6% to 8%, 8% to 10%, 10% to 12%, 12% to 14%, 14% to 16%, 16% to 18%, 18% to 20%, 20% to 25%, 25% to 30%, 30% to 35%, 35% to 40%, 40% to 45% 45% to 50%, 50% to 55%, 55% to 60%, 60% to 65%, 65% to 70% to 80%, 80% to 90%, or 90% to 100% more ATP than unmodified bacteria of the same bacterial subtype under the same conditions. In yet another embodiment, the genetically engineered bacteria consume at least about 0 to 1.0-fold, 1.0-1.2-fold, 1.2-1.4-fold, 1.4-1.6-fold, 1.6-1.8-fold, 1.8-2-fold, or two-fold more ATP than unmodified bacteria of the same bacterial subtype under the same conditions. In yet another embodiment, the genetically engineered bacteria produce at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more cyclic-di-AMP than unmodified bacteria of the same bacterial subtype under the same conditions.

In any of these embodiments, the bacteria genetically engineered to produce cyclic-di-GAMP produce at least about 0% to 2% to 4%, 4% to 6%, 6% to 8%, 8% to 10%, 10% to 12%, 12% to 14%, 14% to 16%, 16% to 18%, 18% to 20%, 20% to 25%, 25% to 30%, 30% to 35%, 35% to 40%, 40% to 45% 45% to 50%, 50% to 55%, 55% to 60%, 60% to 65%, 65% to 70% to 80%, 80% to 90%, or 90% to 100% more arginine than unmodified bacteria of the same bacterial subtype under the same conditions. In yet another embodiment, the genetically engineered bacteria produce at least about 0 to 1.0-fold, 1.0-1.2-fold, 1.2-1.4-fold, 1.4-1.6-fold, 1.6-1.8-fold, 1.8-2-fold, or two-fold more cyclic-di-GAMP than unmodified bacteria of the same bacterial subtype under the same conditions. In yet another embodiment, the genetically engineered bacteria produce at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more cyclic-di-GAMP than unmodified bacteria of the same bacterial subtype under the same conditions.

In any of these embodiments, the bacteria genetically engineered to produce cyclic-di-GAMP consume at least about 0% to 2% to 4%, 4% to 6%, 6% to 8%, 8% to 10%, 10% to 12%, 12% to 14%, 14% to 16%, 16% to 18%, 18% to 20%, 20% to 25%, 25% to 30%, 30% to 35%, 35% to 40%, 40% to 45% 45% to 50%, 50% to 55%, 55% to 60%, 60% to 65%, 65% to 70% to 80%, 80% to 90%, or 90% to 100% more ATP than unmodified bacteria of the same bacterial subtype under the same conditions. In yet another embodiment, the genetically engineered bacteria consume at least about 0 to 1.0-fold, 1.0-1.2-fold, 1.2-1.4-fold, 1.4-1.6-fold, 1.6-1.8-fold, 1.8-2-fold, or two-fold more ATP and/or GTP than unmodified bacteria of the same bacterial subtype under the same conditions. In yet another embodiment, the genetically engineered bacteria consume at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more ATP and/or GTP than unmodified bacteria of the same bacterial subtype under the same conditions.

In any of these embodiments, the genetically engineered bacteria increase STING agonist production rate by at least about 0% to 2% to 4%, 4% to 6%, 6% to 8%, 8% to 10%, 10% to 12%, 12% to 14%, 14% to 16%, 16% to 18%, 18% to 20%, 20% to 25%, 25% to 30%, 30% to 35%, 35% to 40%, 40% to 45% 45% to 50%, 50% to 55%, 55% to 60%, 60% to 65%, 65% to 70% to 80%, 80% to 90%, or 90% to 100% relative to unmodified bacteria of the same bacterial subtype under the same conditions. In yet another embodiment, the genetically engineered bacteria increase the STING agonist production rate by at least about 0 to 1.0-fold, 1.0-1.2-fold, 1.2-1.4-fold, 1.4-1.6-fold, 1.6-1.8-fold, 1.8-2-fold, or two-fold more relative to unmodified bacteria of the same bacterial subtype under the same conditions. In yet another embodiment, the genetically engineered bacteria increase STING agonist production rate by about three-fold, four-fold, five-fold, six-fold, seven-fold, eight-fold, nine-fold, ten-fold, fifteen-fold, twenty-fold, thirty-fold, forty-fold, or fifty-fold, hundred-fold, five hundred-fold, or one-thousand-fold relative to unmodified bacteria of the same bacterial subtype under the same conditions.

In one embodiment, the genetically engineered bacteria increase STING agonist production by at least about 80% to 100% relative to unmodified bacteria of the same bacterial subtype under the same conditions, after 4 hours. In one embodiment, the genetically engineered bacteria increase STING agonist production by at least about 90% to 100% relative to unmodified bacteria of the same bacterial subtype under the same conditions after 4 hours. In one specific embodiment, the genetically engineered bacteria increase STING agonist production by at least about 95% to 100% relative to unmodified bacteria of the same bacterial subtype under the same conditions, after 4 hours. In one specific embodiment, the genetically engineered bacteria increase the STING agonist production by at least about 99% to 100% relative to unmodified bacteria of the same bacterial subtype under the same conditions, after 4 hours. In yet another embodiment, the genetically engineered bacteria increase the STING agonist production by at least about 10-50 fold after 4 hours. In yet another embodiment, the genetically engineered bacteria increase STING agonist production by at least about 50-100 fold after 4 hours. In yet another embodiment, the genetically engineered bacteria increase STING agonist production by at least about 100-500 fold after 4 hours. In yet another embodiment, the genetically engineered bacteria increase STING agonist production by at least about 500-1000 fold after 4 hours. In yet another embodiment, the genetically engineered bacteria increase the STING agonist production by at least about 1000-5000 fold after 4 hours. In yet another embodiment, the genetically engineered bacteria increase the STING agonist production by at least about 5000-10000 fold after 4 hours. In yet another embodiment, the genetically engineered bacteria increase STING agonist production by at least about 10000-1000 fold after 4 hours.

In any of these STING agonist production embodiments, the genetically engineered bacteria are capable of reducing viral infection, e.g., viral infected cell growth and/or proliferation (in vitro during cell culture and/or in vivo) by at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to an unmodified bacteria of the same subtype under the same conditions.

In some embodiments, the genetically engineered bacteria comprising gene sequences encoding dacA (and/or another enzyme for the production of a STING agonists, e.g., cGAS) are able to increase IFN-$1 mRNA or protein levels in macrophages and/or dendritic cells, e.g., in cell culture. In some embodiments, the IFN-β1 mRNA or protein increase dependent on the dose of bacteria administered. In some embodiments, the genetically engineered bacteria comprising gene sequences encoding dacA (and/or another enzyme for the production of a STING agonists, e.g., cGAS) are able to increase IFN-β1 mRNA or protein levels in macrophages and/or dendritic cells. In some embodiments, the IFN-beta1 mRNA or protein increase is dependent on the dosage of bacteria administered.

In one embodiment, IFN-beta1 mRNA or protein production in target cells is about two-fold, about 3-fold, about 4-fold as compared to levels of IFN-beta1 production observed upon administration of an unmodified bacteria of the same subtype under the same conditions, e.g., at day 2 after first injection of the bacteria. In some embodiments, the genetically engineered bacteria induce the production of at least about 6,000 to 25,000, 15,000 to 25,000, 6,000 to 8,000, 20,000 to 25,000 pg/ml IFN b1 mRNA in bone marrow-derived dendritic cells, e.g., at 4 hours post-stimulation.

In some embodiments, the genetically engineered bacteria comprising gene sequences encoding dacA (or another enzyme for the production of a STING agonists) can dose-dependently increase IFN-b1 production in bone marrow-derived dendritic cells, e.g., at 2 or 4 hours post stimulation.

In some embodiments, the genetically engineered bacteria comprising gene sequences encoding dacA (or another enzyme for the production of a STING agonists) are able to reduce viral infection, e.g., at 4 or 9 days after a regimen of 3 bacterial treatments, relative to an unmodified bacteria of the same subtype under the same conditions.

Strain activity of the STING agonist producing strain can be defined by conducting in vitro measurements c-di-AMP production (in the cell or in the medium). C-di-AMP production can be measured over a time period of 1, 2, 3, 4, 5, 6 hours or greater. In one example, c-di-AMP levels can be measured at 0, 2, or 4 hours. Unmodified Nissle can be used as a baseline in such measurements. If STING agonist producing enzyme is under the control of a promoter which is induced by a chemical inducer, the inducer needs to be added. If STING agonist producing enzyme is under the control of a promoter which is induced by exogenous environmental conditions, such as low-oxygen conditions, the bacterial cells are induced under these conditions, e.g., low oxygen conditions. As an additional baseline measurement, STING agonist producing strains which are inducible can be left uninduced. After the incubation time, levels of c-diAMP can be measured by LC-MS as described herein. In some embodiments, the induced STING agonist producing strain is capable of producing c-di-AMP at a concentration of at least about 0.01 mM to 1.4 mM per 10\hat{ }9. In some embodiments, the induced STING agonist producing strain is capable of producing c-di-AMP at a concentration of at least about 0.01 mM to 0.02 mM, 0.02 mM to 0.03 mM, 0.03 mM to 0.04 mM, 0.04 mM to 0.05 mM, 0.05 mM to 0.06 mM, 0.06 mM to 0.07 mM, 0.07 mM to 0.08 mM, 0.08 mM to 0.09 mM, 0.09 mM to 0.10 mM, 0.10 mM to 0.12 mM per 10\hat{ }9 e.g., after 2 or 4 hours. In some embodiments, the induced STING agonist producing strain is capable of producing c-di-AMP at a concentration of at least about 0.1 mM to 0.2 mM, 0.2 mM to 0.3 mM, 0.3 mM to 0.4 mM, 0.4 mM to 0.5 mM, 0.5 mM to 0.6 mM, 0.6 mM to 0.7 mM, 0.7 mM to 0.8 mM, 0.8 mM to 0.9 mM, 0.9 mM to 1 mM, 1 mM to 1.2 mM, 1.2 mM to 1.3 mM, 1.3 mM to 1.4 mM per 10\hat{ }9 e.g., after 2 or 4 hours.

Strain activity of the STING agonist producing strain may also be measured using in vitro measurements of activity. In a non-limiting example of an in vitro strain activity measurement, IFN-beta1 induction in RAW 264.7 cells (or other macrophage or dendritic cell) in culture may be measured. Activity of the strain can be measured at various multiplicities of infection (MOI) at various time points. For example, activity can be measured at 1, 2, 3, 4, 5, 6 hours or greater. In one example activity can be measured at 45 minutes or 4 hours. Unmodified Nissle can be used as a baseline in such measurements. If STING agonist producing enzyme is under the control of a promoter which is induced by a chemical inducer, the inducer needs to be added. If STING agonist producing enzyme is under the control of a promoter which is induced by exogenous environmental conditions, such as low-oxygen conditions, the bacterial cells are induced under these conditions, e.g., low oxygen conditions. As an additional baseline measurement, STING agonist producing strains which are inducible can be left uninduced. After the incubation time, IFN-beta levels can be measured from protein extracts or RNA levels can be analyzed, e.g., via PCT based methods. In some embodiments, the induced STING agonist producing strain can elicit a dose-dependent induction of IFN-b levels. In some embodiments, 10\hat{ }1 to 10\hat{ }2 (multiplicities of infection (MOI) can induce at least about 20 to 25 times, 25 to 30 times, 30 to 35 times, 35 to 40 times or more greater IFN-beta levels as the unmodified Nissle baseline strain of the same subtype under the same conditions, eg., after 4 hours. In some embodiments, 10\hat{ }1 to 10\hat{ }2 (multiplicities of infection (MOI) can induce at least about 10,000 to 12,000, 12,000 to 15,000, 15,000 to 20,000 or 20,000 to 25,000 pg/ml media IFN-beta e.g., after 4 hours.

In some embodiments, 10\hat{ }1 to 10\hat{ }2 (multiplicities of infection (MOI) can induce at least about 10 to 12 times, 12 to 15 times, 15 to 20 times, 20 to 25 times or more greater IFN-beta levels as the wild type Nissle baseline strain of the same subtype under the same conditions, e.g., after 45 minutes. In some embodiments, 10\hat{ }1 to 10\hat{ }2 (multiplicities of infection (MOI) can induce at least about 4,000 to 6,000, 6,000 to 8,000, 8,000 to 10,000 or 10,000 to 12,000 pg/ml media IFN-beta e.g., after 45 minutes.

In some embodiments, the bacteria genetically engineered to produce STING agonists are capable of increasing the response rate by at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more as compared to an unmodified bacteria of the same subtype under the same conditions. In some embodiments, the genetically engineered bacteria comprising gene sequences encoding dacA, achieve a 100% response rate.

In some embodiments, the response rate is at least about 0 to 1.0-fold, 1.0-1.2-fold, 1.2-1.4-fold, 1.4-1.6-fold, 1.6-1.8-fold, 1.8-2-fold, or two-fold than observed with than unmodified bacteria of the same bacterial subtype under the same conditions. In yet another embodiment, the response rate is about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more than observed with unmodified bacteria of the same bacterial subtype under the same conditions.

In some embodiments, the genetically engineered bacteria comprising gene sequences encoding diadenylate cyclases, e.g., DacA, di-GAMP synthases, and/or other STING agonist producing polypeptides increase total T cell numbers in the lymph nodes. In some embodiments, the increase in total T cell numbers in the lymph nodes is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more as compared to an unmodified bacteria of the same subtype under the same conditions. In some embodiments, the increase in total T cell numbers is at least about 0 to 1.0-fold, 1.0-1.2-fold, 1.2-1.4-fold, 1.4-1.6-fold, 1.6-1.8-fold, 1.8-2-fold, or two-fold than observed with than unmodified bacteria of the same bacterial subtype under the same conditions. In yet another embodiment, the increase in total T cell numbers is about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold more than observed with unmodified bacteria of the same bacterial subtype under the same conditions.

In some embodiments, the genetically engineered bacteria comprising gene sequences encoding diadenylate cyclases, e.g., DacA, di-GAMP synthases, and/or other STING agonist producing polypeptides increase the percentage of activated effector CD4 and CD8 T cells in lymph nodes.

In some embodiments, the percentage of activated effector CD4 and CD8 T cells in the lymph nodes is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more as compared to an unmodified bacteria of the same subtype under the same conditions. In some embodiments, the percentage of activated effector CD4 and CD8 T cells is at least about 0 to 1.0-fold, 1.0-1.2-fold, 1.2-1.4-fold, 1.4-1.6-fold, 1.6-1.8-fold, 1.8-2-fold, or two-fold than observed with than unmodified bacteria of the same bacterial subtype under the same conditions. In yet another embodiment, the percentage of activated effector CD4 and CD8 T cells is about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold more than observed with unmodified bacteria of the same bacterial subtype under the same conditions. In one embodiment, the gene encoded by the bacteria is DacA and the percentage of activated effector CD4 and CD8 T cells is two to four fold more than observed with unmodified bacteria of the same bacterial subtype under the same conditions.

In some embodiments, the genetically engineered bacteria comprising gene sequences encoding diadenylate cyclases, e.g., DacA, di-GAMP synthases, and/or other STING agonist producing polypeptides achieve early rise of innate cytokines and a later rise of an effector-T-cell response.

In some embodiments, the genetically engineered bacteria comprising gene sequences encoding dacA (or other enzymes for production of STING agonists) in the target cells are able to overcome immunological suppression and generating robust innate and adaptive immune responses. In some embodiments, the genetically engineered bacteria comprising gene sequences encoding dacA inhibit proliferation or accumulation of regulatory T cells.

In some embodiments, the genetically engineered bacteria comprising gene sequences encoding dacA, cGAS, and/or other enzymes for production of STING agonists, achieve early rise of innate cytokines, including but not limited to IL-6, IL-1beta, and MCP-1.

In some embodiments IL-6 is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more induced as compared to an unmodified bacteria of the same subtype under the same conditions. In some embodiments, IL-6 is at least about 0 to 1.0-fold, 1.0-1.2-fold, 1.2-1.4-fold, 1.4-1.6-fold, 1.6-1.8-fold, 1.8-2-fold, or two-fold more induced than observed with than unmodified bacteria of the same bacterial subtype under the same conditions. In yet another embodiment, the IL-6 is about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more induced than observed with unmodified bacteria of the same bacterial subtype under the same conditions. In one embodiment, the gene encoded by the bacteria is dacA and the levels of induced IL-6 is about two to three-fold greater than observed with unmodified bacteria of the same bacterial subtype under the same conditions.

In some embodiments, the levels of IL-1beta in the target cells is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of IL-1beta are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of IL-1beta are about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, a di-GAMP synthase, and/or other STING agonist producing polypeptide and levels of IL-1beta are about 2 fold, 3 fold, or 4 fold more than observed with unmodified bacteria of the same bacterial subtype under the same conditions.

In some embodiments, the levels of MCP1 in the target cells is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of MCP1 are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of MCP1 are about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, a di-GAMP synthase, and/or other STING agonist producing polypeptide and levels of MCP1 are about 2-fold, 3-fold, or 4-fold more than observed with unmodified bacteria of the same bacterial subtype under the same conditions.

In some embodiments, the genetically engineered bacteria comprising gene sequences encoding diadenylate cyclases, e.g., DacA, di-GAMP synthases, and/or other STING agonist producing polypeptides achieve activation of molecules relevant towards an effector-T-cell response, including but not limited to, Granzyme B, IL-2, and IL-15.

In some embodiments, the levels of granzyme B in the target cells is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of granzyme B are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of granzyme B are about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, a di-GAMP synthase, and/or other STING agonist producing polypeptide and levels of granzyme B are about 2 fold, 3 fold, or 4 fold more than observed with unmodified bacteria of the same bacterial subtype under the same conditions.

In some embodiments, the levels of IL-2 in the target cells is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of IL-2 are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of IL-2 are about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is DacA and the levels of IL-2 are about 3 fold, 4 fold, or 5 fold more than observed with unmodified bacteria of the same bacterial subtype under the same conditions.

In some embodiments, the levels of IL-15 in the target cells is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of IL-15 are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of IL-15 are at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, gene encoded by the bacteria is DacA and the levels of IL-15 are about 2-fold, 3-fold, -fold, or 5-fold more than observed with unmodified bacteria of the same bacterial subtype under the same conditions.

In some embodiments, the levels of IFNg in the target cells is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of IFNg are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of IFNg are at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, di-GAMP synthase, and/or other STING agonist producing polypeptide and levels of IFNg are about 2 fold, 3 fold, or 4 fold more than observed with unmodified bacteria of the same bacterial subtype under the same conditions.

In some embodiments, the levels of IL-12 in the target cells is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of IL-12 are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of IL-12 are at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, a di-GAMP synthase, and/or other STING agonist producing polypeptide and levels of IL-12 are about 2 fold, 3 fold, or 4 fold more than observed with unmodified bacteria of the same bacterial subtype under the same conditions.

In some embodiments, the levels of TNF-a in the target cells is at least about 0% to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of TNF-a are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of TNF-a are at least about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, a di-GAMP synthase, and/or other STING agonist producing polypeptide and levels of TNF-a are at least about 2 fold, 3 fold, or 4 fold more than observed with unmodified bacteria of the same bacterial subtype under the same conditions.

In some embodiments, the levels of GM-CSF in the target cells is at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more elevated as compared to an unmodified bacteria of the same subtype under the same conditions. In some embodiments, the levels of GM-CSF are at least about 0 to 1.0-fold, 1.0-1.2-fold, 1.2-1.4-fold, 1.4-1.6-fold, 1.6-1.8-fold, 1.8-2-fold, or two-fold or more elevated than observed with than unmodified bacteria of the same bacterial subtype under the same conditions. In yet another embodiment, levels of GM-CSF are about 2 to 3-fold, 3 to 4-fold, 4 to 5-fold, 5 to 6-fold, 6 to 7-fold, 7 to 8-fold, 8 to 9-fold, 9 to 10-fold, 10 to 15-fold, 15 to 20-fold, 20 to 30-fold, 30 to 40-fold, or 40 to 50-fold, 50 to 100-fold, 100 to 500-fold, or 500 to 1000-fold or more elevated than observed with unmodified bacteria of the same bacterial subtype under the same conditions. In one embodiment, the gene encoded by the bacteria is a diadenylate cyclase, e.g., DacA, a di-GAMP synthase, and/or other STING agonist producing polypeptide and levels of GM-CSF are at least about 2 fold, 3 fold, or 4 fold more than observed with unmodified bacteria of the same bacterial subtype under the same conditions.

In some embodiments, administration of the genetically engineered bacteria comprising gene sequences encoding one or more of a diadenylate cyclase, e.g., DacA, a di-GAMP synthase, and/or other STING agonist producing polypeptide results in long-term immunological memory. In some embodiments, long term immunological memory is established, exemplified by at least about 0 to 10%, 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, 98% or more protection from secondary viral infection challenge compared to naïve age-matched controls.

In some embodiments, the c-di-GAMP synthases, diadenylate cyclases, or other STING agonist producing polypeptides are modified and/or mutated, e.g., to enhance stability, or to increase STING agonism. In some embodiments, c-di-GAMP synthases from Vibrio cholerae or the orthologs thereof (e.g., from Verminephrobacter eiseniae, Kingella denitrificans, and/or Neisseria bacilliformis) or human cGAS is modified and/or mutated, e.g., to enhance stability, or to increase STING agonism. In some embodiments, the diadenylate cyclase from Listeria monocytogenes is modified and/or mutated, e.g., to enhance stability, or to increase STING agonism.

In some embodiments, the genetically engineered bacteria and/or other microorganisms are capable of producing one or more diadenylate cyclases, c-di-GAMP synthases and/or other STING agonist producing polypeptides under inducing conditions, e.g., under a condition(s) associated with immune suppression. In some embodiments, the genetically engineered bacteria and/or other microorganisms are capable of producing the diadenylate cyclases, c-di-GAMP synthases and/or other STING agonist producing polypeptides in low-oxygen conditions or hypoxic conditions, in the presence of certain molecules or metabolites, in the presence of molecules or metabolites associated with viral infection, or certain tissues, immune suppression, or inflammation, or in the presence of a metabolite that may or may not be present in the gut, circulation, or the target site, and which may be present in vitro during strain culture, expansion, production and/or manufacture such as arabinose, cumate, and salicylate. In some embodiments, the one or more genetically engineered bacteria comprise gene sequence(s) encoding the diadenylate cyclases, c-di-GAMP synthases and/or other STING agonist producing polypeptides, wherein the diadenylate cyclases, c-di-GAMP synthases and/or other STING agonist producing polypeptides are operably linked to a promoter inducible by exogenous environmental conditions of the target cells. In some embodiments, the one or more genetically engineered bacteria comprise gene sequence(s) encoding the diadenylate cyclases, c-di-GAMP synthases and/or other STING agonist producing polypeptides, wherein the diadenylate cyclases, c-di-GAMP synthases and/or other STING agonist producing polypeptides is operably linked to a promoter inducible by cumate or salicylate as described herein. In some embodiments, the gene sequences encoding diadenylate cyclases, c-di-GAMP synthases and/or other STING agonist producing polypeptides are operably linked to a constitutive promoter. In some embodiments, the gene sequences encoding diadenylate cyclases, c-di-GAMP synthases and/or other STING agonist producing polypeptides are present on one or more plasmids (e.g., high copy or low copy) or are integrated into one or more sites in the bacteria and/or other microorganism chromosome(s).

In any of these embodiments, any of the STING agonist producing strains described herein may comprise an auxotrophic modification. In any of these embodiments, the STING agonist producing strains may comprise an auxotrophic modification in DapA, e.g., a deletion or mutation in DapA. In any of these embodiments, the STING agonist producing strains may further comprise an auxotrophic modification in ThyA e.g., a deletion or mutation in ThyA. In any of these embodiments, the STING agonist producing strains may comprise a DapA and a ThyA auxotrophy. In any of these embodiments, the bacteria may further comprise an endogenous phage modification, e.g., a mutation or deletion, in an endogenous phage. In a non-limiting example the bacterial host is E. coli Nissle and the phage modification comprises a modification in Nissle Phage 3, described herein. In one example, the phage modification is a deletion of one or more genes, e.g., a 10 kb deletion.

In any of these embodiments describing genetically engineered bacteria comprising gene sequences encoding one or more diadenylate cyclases, c-di-GAMP synthases or other STING agonist producing polypeptides, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively the genetically engineered bacteria comprising gene sequences encoding one or more diadenylate cyclases, c-di-GAMP synthases or other STING agonist producing polypeptides may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In certain embodiments, one or more genetically engineered bacteria comprise gene sequence(s) encoding diadenylate cyclase e.g., DacA, e.g., from Listeria monocytogenes, wherein diadenylate cyclase gene is operably linked to a promoter inducible under exogenous environmental conditions. In one embodiment, the diadenylate cyclase gene is operably linked to a promoter inducible under low oxygen conditions, e.g., a FNR promoter. In certain embodiments, one or more genetically engineered bacteria comprise gene sequence(s) encoding diadenylate cyclase, e.g., dacA, e.g., from Listeria monocytogenes, wherein diadenylate cyclase is operably linked to a promoter inducible by cumate or salicylate as described herein. In certain embodiments, the diadenylate cyclase gene sequences are integrated into the bacterial chromosome. Suitable integration sites are described herein. In a non-limiting example the diadenylate cyclase gene is integrated at HA910. In certain embodiments, the bacteria comprising gene sequences encoding the diadenylate cyclase further comprise an auxotrophic modification. In some embodiments, the modification, e.g., a mutation or deletion is in the dapA gene. In some embodiments, the modification, e.g., a mutation or deletion is in the thyA gene. In some embodiments, the modification, e.g., a mutation or deletion is in both dapA and thyA genes. In any of these embodiments, the bacteria may further comprise a phage modification, e.g., a mutation or deletion in an endogenous prophage. In one example, the prophage modification is a deletion of one or more genes, e.g., a 10 kb deletion. In a non-limiting example, the genetically engineered bacteria comprising gene sequences encoding diadenylate cyclase are derived from E. coli Nissle and the prophage modification comprises a deletion or mutation in Nissle Prophage 3, described herein.

In certain embodiments genetically engineered bacteria comprising gene sequences encoding one or more diadenylate cyclases, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively the genetically engineered bacteria comprising gene sequences encoding one or more diadenylate cyclases may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In one specific embodiment, one or more genetically engineered bacteria comprise gene sequence(s) encoding diadenylate cyclase e.g., DacA, e.g., from Listeria monocytogenes, wherein the diadenylate cyclase gene is operably linked to a promoter inducible under low oxygen conditions, e.g., a FNR promoter. The dacA gene sequences are integrated into the bacterial chromosome, e.g., at integration site HA910. The bacteria further comprise a auxotrophic modification, e.g., a mutation or deletion in dapA or thyA or both genes. The bacteria may further comprise an endogenous phage modification, e.g., a mutation or deletion, in an endogenous phage, e.g., a 10 kb deletion. In one specific embodiment, the genetically engineered bacteria are derived from E. coli Nissle and the phage modification comprises a deletion or mutation in Nissle Phage 3, e.g., as described herein.

In another specific embodiment, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively the genetically engineered bacteria may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In certain embodiments, one or more genetically engineered bacteria comprise gene sequence(s) encoding cGAMP synthase e.g., human cGAS, wherein the cGAS gene is operably linked to a promoter inducible under exogenous environmental conditions. In one embodiment, the cGAS gene is operably linked to a promoter inducible under low oxygen conditions, e.g., a FNR promoter. In certain embodiments, one or more genetically engineered bacteria comprise gene sequence(s) encoding cGAS, e.g., human cGAS, wherein the cGAS gene is operably linked to a promoter inducible by cumate or salicylate as described herein. In certain embodiments, the cGAS gene sequences are integrated into the bacterial chromosome. Suitable integration sites are described herein and known in the art. In certain embodiments, the bacteria comprising gene sequences encoding cGAS further comprise an auxotrophic modification, e.g., a mutation or deletion in dapA or thyA or both genes. In some embodiments, the modification, e.g., a mutation or deletion is in the dapA gene. In some embodiments, the modification, e.g., a mutation or deletion is in thyA gene. In some embodiments, the modification, e.g., a mutation or deletion is in both dapA and thyA genes. In any of these embodiments, the bacteria may further comprise a prophage modification, e.g., a mutation or deletion, in an endogenous prophage. In one example, the prophage modification is a deletion of one or more genes, e.g., a 10 kb deletion. In a non-limiting example, the genetically engineered bacteria comprising gene sequences encoding cGAS are derived from E. coli Nissle and the prophage modification comprises a deletion or mutation in Nissle Phage 3, described herein.

In any of these embodiments describing genetically engineered bacteria comprising gene sequences encoding one or more cGAS, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively the genetically engineered bacteria comprising gene sequences encoding one or more cGAS may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In one embodiment, one or more genetically engineered bacteria comprise gene sequence(s) encoding cGAS e.g., human cGAS, wherein the cGAS gene is operably linked to a promoter inducible under low oxygen conditions, e.g., an FNR promoter. The cGAS gene sequences are integrated into the bacterial chromosome. The bacteria further comprise an auxotrophic modification, e.g., a mutation or deletion in dapA or thyA or both genes. The bacteria may further comprise an endogenous phage modification, e.g., a mutation or deletion, in an endogenous phage, e.g., a 10 kb deletion. In one specific embodiment, the genetically engineered bacteria are derived from E. coli Nissle and the phage modification comprises a deletion or mutation in Nissle Phage 3, e.g., as described herein.

In another specific embodiment, the genetically engineered bacteria comprising gene sequences encoding one or more cGAS, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively the genetically engineered bacteria comprising gene sequences encoding one or more cGAS may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In certain embodiments, one or more genetically engineered bacteria comprise gene sequence(s) encoding diadenylate cyclase e.g., DacA, e.g., from Listeria monocytogenes, and cGAMP synthase e.g., human cGAS. In certain embodiments, the diadenylate cyclase gene and/or the cGAS gene are operably linked to a promoter inducible under exogenous environmental conditions. In certain embodiments, the diadenylate cyclase gene and/or cGAS gene are operably linked to a promoter inducible by cumate or salicylate, or another chemical inducer. In certain embodiments, the diadenylate cyclase gene and/or cGAS gene are operably linked to a constitutive promoter. In one embodiment, the diadenylate cyclase gene and/or cGAS gene is operably linked to a promoter inducible under low oxygen conditions, e.g., an FNR promoter. In certain embodiments, one or more genetically engineered bacteria comprise gene sequence(s) encoding diadenylate cyclase gene, e.g., dacA, e.g., from Listeria monocytogenes, and cGAS, e.g., human cGAS, wherein the diadenylate cyclase gene and/or cGAS gene is operably linked to a promoter inducible by cumate or salicylate as described herein. In certain embodiments, the diadenylate cyclase and cGAS gene sequences are integrated into the bacterial chromosome. Suitable integration sites are described herein and known in the art. In certain embodiments, the bacteria comprising gene sequences encoding diadenylate cyclase and cGAS further comprise a mutation or deletion in dapA or thyA or both genes. In any of these embodiments, the bacteria may further comprise a prophage modification, e.g., a mutation or deletion, in an endogenous prophage. In one example, the prophage modification is a deletion of one or more genes, e.g., a 10 kb deletion. In a non-limiting example, the genetically engineered bacteria comprising gene sequences encoding diadenylate cyclase and cGAS are derived from E. coli Nissle and the prophage modification comprises a deletion or mutation in Nissle Phage 3, described herein.

In any of these embodiments describing genetically engineered bacteria comprising gene sequences encoding one or more diadenylate cyclases and cGAS producing polypeptides, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively the genetically engineered bacteria comprising gene sequences encoding one or more diadenylate cyclases and cGAS polypeptides may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In one specific embodiment, one or more genetically engineered bacteria comprise gene sequence(s) encoding diadenylate cyclase e.g., DacA, e.g., from Listeria monocytogenes, and cGAS e.g., human cGAS, wherein the diadenylate cyclase gene and/or cGAS gene is operably linked to a promoter inducible under low oxygen conditions, e.g., an FNR promoter. The diadenylate cyclase gene and cGAS gene sequences are integrated into the bacterial chromosome. The bacteria further comprise an auxotrophic modification, e.g., a mutation or deletion in dapA or thyA or both genes. The bacteria may further comprise an endogenous phage modification, e.g., a mutation or deletion, in an endogenous phage, e.g., a 10 kb deletion. In one specific embodiment, the genetically engineered bacteria are derived from E. coli Nissle and the phage modification comprises a deletion or mutation in Nissle Phage 3, e.g., as described herein.

In another specific embodiment, the genetically engineered bacteria comprising gene sequences encoding one or more diadenylate cyclases and cGAS polypeptides, the genetically engineered bacteria may further comprise gene sequence(s) encoding kynureninase, e.g., kynureninase from Pseudomonas fluorescens and (optionally) having a modification, e.g., mutation or deletion in the TrpE gene. Alternatively, the genetically engineered bacteria comprising gene sequences encoding one or more diadenylate cyclases and cGAS polypeptides may be combined or administered with genetically engineered bacteria comprising gene sequence(s) encoding kynureninase, e.g., kynureninase from Pseudomonas fluorescens and (optionally) having a modification, e.g., mutation or deletion in the TrpE gene.

In any of these embodiments, the one or more bacteria genetically engineered to produce one or more STING agonists may be administered alone or in combination with one or more immune checkpoint inhibitors described herein, including but not limited to anti-CTLA4, anti-PD1, or anti-PD-L1 antibodies. In some embodiments, the one or more genetically engineered bacteria which produce STING agonists evoke immunological memory when administered in combination with checkpoint inhibitor therapy.

In any of these embodiments, the one or more bacteria genetically engineered to produce STING agonists may be genetically engineered to produce and secrete or display on their surface one or more immune checkpoint inhibitors described herein, including but not limited to anti-CTLA4, anti-PD1, or anti-PD-L1 antibodies. In some embodiments, the one or more genetically engineered bacteria which comprise gene sequences encoding one or more enzymes for STING agonist production and gene sequences encoding one or more immune checkpoint inhibitor antibodies, e.g., scFv antibodies, promote immunological memory upon rechallenge/reoccurrence of a viral infection.

In any of these embodiments, the one or more bacteria genetically engineered to produce one or more STING agonists may be administered alone or in combination with one or more immune stimulatory agonists described herein, e.g., agonistic antibodies, including but not limited to anti-OX40, anti-41BB, or anti-GITR antibodies. In some embodiments, the one or more genetically engineered bacteria which produce STING agonists evoke immunological memory when administered in combination with anti-OX40, anti-41BB, or anti-GITR antibodies.

In any of these embodiments, the one or more bacteria genetically engineered to produce STING agonists may be genetically engineered to produce and secrete or display on their surface one or more immune stimulatory agonists described herein, e.g., agonistic antibodies, including but not limited to anti-OX40, anti-41BB, or anti-GITR antibodies. In some embodiments, the one or more genetically engineered bacteria comprising gene sequences encoding one or more STING agonist producing enzymes and gene sequences encoding one or more costimulatory antibodies, e.g., selected from anti-OX40, anti-41BB, or anti-GITR antibodies evoke immunological memory.

Also, in some embodiments, the genetically engineered bacteria and/or other microorganisms are further capable of expressing any one or more of the described circuits and further comprise one or more of the following: (1) one or more auxotrophies, such as any auxotrophies known in the art and provided herein, e.g., dapA and thyA auxotrophy, (2) one or more kill switch circuits, such as any of the kill-switches described herein or otherwise known in the art, (3) one or more antibiotic resistance circuits, (4) one or more transporters for importing biological molecules or substrates, such any of the transporters described herein or otherwise known in the art, (5) one or more secretion circuits, such as any of the secretion circuits described herein and otherwise known in the art, (6) one or more surface display circuits, such as any of the surface display circuits described herein and otherwise known in the art (7) one or more circuits for the production or degradation of one or more metabolites (e.g., kynurenine, tryptophan, adenosine, arginine) described herein, (8) one or more immune initiators (e.g. STING agonist, CD40L, SIRPα) described herein, (9) one or more immune sustainers (e.g. IL-15, IL-12, CXCL10) described herein, and (10) combinations of one or more of such additional circuits.

### Regulating Expression

In some embodiments, the bacterial cell comprises a stably maintained plasmid or chromosome carrying the gene(s) encoding payload (s), such that the payload(s) can be expressed in the host cell, and the host cell is capable of survival and/or growth in vitro, e.g., in medium, and/or in vivo. In some embodiments, bacterial cell comprises two or more distinct payloads or operons, e.g., two or more payload genes. In some embodiments, bacterial cell comprises three or more distinct transporters or operons, e.g., three or more payload genes. In some embodiments, bacterial cell comprises 4, 5, 6, 7, 8, 9, 10, or more distinct payloads or operons, e.g., 4, 5, 6, 7, 8, 9, 10, or more payload genes.

Herein the terms “payload” “polypeptide of interest” or “polypeptides of interest”, “protein of interest”, “proteins of interest”, “payloads” “effector molecule”, “effector” refers to one or more effector molecules described herein and/or one or more enzyme(s) or polypeptide(s) function as enzymes needed for the production of such effector molecules. Non-limiting examples of payloads include a viral COVID19 protein, a STING agonist, etc.

As used herein, the term “polypeptide of interest” or “polypeptides of interest”, “protein of interest”, “proteins of interest”, “payload”, “payloads” further includes any or a plurality of any of the viral proteins, STING agonists, tryptophan synthesis enzymes, kynurenine degrading enzymes, adenosine degrading enzymes, arginine producing enzymes, and other metabolic pathway enzymes described herein. As used herein, the term “gene of interest” or “gene sequence of interest” includes any or a plurality of any of the gene(s) an/or gene sequence(s) and or gene cassette(s) encoding one or more immune modulator(s) described herein.

In some embodiments, the genetically engineered bacteria comprise multiple copies of the same payload gene(s). In some embodiments, the gene encoding the payload is present on a plasmid and operably linked to a directly or indirectly inducible promoter. In some embodiments, the gene encoding the payload is present on a plasmid and operably linked to a constitutive promoter. In some embodiments, the gene encoding the payload is present on a plasmid and operably linked to a promoter that is induced under low-oxygen or anaerobic conditions. In some embodiments, the gene encoding the payload is present on plasmid and operably linked to a promoter that is induced by exposure to tetracycline or arabinose, cumate, and salicylate, or another chemical or nutritional inducer described herein.

In some embodiments, the gene encoding the payload is present on a chromosome and operably linked to a directly or indirectly inducible promoter. In some embodiments, the gene encoding the payload is present on a chromosome and operably linked to a constitutive promoter. In some embodiments, the gene encoding the payload is present in the chromosome and operably linked to a promoter that is induced under low-oxygen or anaerobic conditions. In some embodiments, the gene encoding the payload is present on chromosome and operably linked to a promoter that is induced by exposure to tetracycline or arabinose, cumate, and salicylate, or another chemical or nutritional inducer described herein.

In some embodiments, the genetically engineered bacteria comprise two or more payloads, all of which are present on the chromosome. In some embodiments, the genetically engineered bacteria comprise two or more payloads, all of which are present on one or more same or different plasmids. In some embodiments, the genetically engineered bacteria comprise two or more payloads, some of which are present on the chromosome and some of which are present on one or more same or different plasmids.

In any of the embodiments described above, the one or more payload(s) for producing the effector or immune modulator combinations are operably linked to one or more directly or indirectly inducible promoter(s). In some embodiments, the one or more payload(s) are operably linked to a directly or indirectly inducible promoter that is induced under exogeneous environmental conditions, e.g., conditions found in tissue specific conditions. In some embodiments, the one or more payload(s) are operably linked to a directly or indirectly inducible promoter that is induced by metabolites found in the tissue specific conditions. In some embodiments, the one or more payload(s) are operably linked to a directly or indirectly inducible promoter that is induced under low-oxygen or anaerobic conditions. In some embodiments, the one or more payload(s) are operably linked to a directly or indirectly inducible promoter that is induced under inflammatory conditions (e.g., RNS, ROS), as described herein. In some embodiments, the one or more payload(s) are operably linked to a directly or indirectly inducible promoter that is induced under immunosuppressive conditions, e.g., as found in the target site, as described herein. In some embodiments, the two or more gene sequence(s) are linked to a directly or indirectly inducible promoter that is induced by exposure a chemical or nutritional inducer, which may or may not be present under in vivo conditions and which may be present during in vitro conditions (such as strain culture, expansion, manufacture), such as tetracycline or arabinose, cumate, and salicylate, or others described herein. In some embodiments, the two or more payloads are all linked to a constitutive promoter.

In some embodiments, the promoter is induced under in vivo conditions, e.g., the gut, as described herein. In some embodiments, the promoters is induced under in vitro conditions, e.g., various cell culture and/or cell manufacturing conditions, as described herein. In some embodiments, the promoter is induced under in vivo conditions, e.g., the gut, as described herein, and under in vitro conditions, e.g., various cell culture and/or cell production and/or manufacturing conditions, as described herein.

In some embodiments, the promoter that is operably linked to the gene encoding the payload is directly induced by exogenous environmental conditions (e.g., in vivo and/or in vitro and/or production/manufacturing conditions). In some embodiments, the promoter that is operably linked to the gene encoding the payload is indirectly induced by exogenous environmental conditions (e.g., in vivo and/or in vitro and/or production/manufacturing conditions).

### FNR Dependent Regulation

The genetically engineered bacteria of the invention comprise a gene or gene cassette for producing an immune modulator, wherein the gene or gene cassette is operably linked to a directly or indirectly inducible promoter that is controlled by exogenous environmental condition(s). In some embodiments, the inducible promoter is an oxygen level-dependent promoter and an immune modulator is expressed in low-oxygen, microaerobic, or anaerobic conditions. For example, in low oxygen conditions, the oxygen level-dependent promoter is activated by a corresponding oxygen level-sensing transcription factor, thereby driving production of an immune modulator.

Bacteria have evolved transcription factors that are capable of sensing oxygen levels. Different signaling pathways may be triggered by different oxygen levels and occur with different kinetics. An oxygen level-dependent promoter is a nucleic acid sequence to which one or more oxygen level-sensing transcription factors is capable of binding, wherein the binding and/or activation of the corresponding transcription factor activates downstream gene expression. In one embodiment, the genetically engineered bacteria comprise a gene or gene cassette for producing a payload under the control of an oxygen level-dependent promoter. In a more specific aspect, the genetically engineered bacteria comprise a gene or gene cassette for producing a payload under the control of an oxygen level-dependent promoter that is activated under low-oxygen or anaerobic environments.

In certain embodiments, the bacterial cell comprises a gene encoding a payload which is operably linked to a fumarate and nitrate reductase regulator (FNR) responsive promoter. In certain embodiments, the bacterial cell comprises a gene encoding a payload expressed under the control of a fumarate and nitrate reductase regulator (FNR) responsive promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive. FNR responsive promoters include, but are not limited to, the FNR responsive promoters of SEQ ID NO: 563-579. Underlined sequences are predicted ribosome binding sites, and bolded sequences are restriction sites used for cloning.

FNR promoter sequences are known in the art, and any suitable FNR promoter sequence(s) may be used in the genetically engineered bacteria of the invention. Any suitable FNR promoter(s) may be combined with any suitable payload.

As used herein the term “payload” refers to one or more effector molecules, e.g. immune modulator(s), including but not limited to immune initiators and immune sustainers described herein.

Non-limiting FNR promoter sequences are provided in SEQ ID NO: 563-579. In some embodiments, the genetically engineered bacteria of the disclosure comprise a payload, e.g., an effector or an immune modulator, which is operably linked to a low oxygen inducible, e.g., FNR regulated promoter comprising: SEQ ID NO: 563, SEQ ID NO: 564, SEQ ID NO: 565, SEQ ID NO: 566, SEQ ID NO: 567, SEQ ID NO: 568, SEQ ID NO: 569, nirB1 promoter (SEQ ID NO: 570), nirB2 promoter (SEQ ID NO: 571), nirB3 promoter (SEQ ID NO: 572), ydfZ promoter (SEQ ID NO: 573), nirB promoter fused to a strong ribosome binding site (SEQ ID NO: 574), ydfZ promoter fused to a strong ribosome binding site (SEQ ID NO: 575), fnrS, an anaerobically induced small RNA gene (fnrS1 promoter SEQ ID NO: 576 or fnrS2 promoter SEQ ID NO: 577), nirB promoter fused to a crp binding site (SEQ ID NO: 578), and fnrS fused to a crp binding site (SEQ ID NO: 579). In some embodiments, the FNR-responsive promoter is at least about 80%, at least about 85%, at least about 90%, at least about 95%, or at least about 99% homologous to a sequence selected from SEQ ID NOs: 563-579. In another embodiment, the genetically engineered bacteria comprise a gene sequence comprising an FNR-responsive promoter comprising a sequence selected from SEQ ID NOs: 563-579. In yet another embodiment, the FNR-responsive promoter consists of a sequence selected from SEQ ID NOs: 563-579.

In some embodiments, the genetically engineered bacteria of the disclosure comprise a gene encoding an effector molecule, e.g., an immune initiator or immune stimulator, which is operably linked to an FNR-responsive promoter which is at least about 80%, at least about 85%, at least about 90%, at least about 95%, or at least about 99% homologous to a sequence selected from SEQ ID NOs: 1281 or SEQ ID NO: 1282. In another embodiment, the genetically engineered bacteria comprise encode an effector molecule operably linked to an FNR-responsive promoter comprising a sequence selected from SEQ ID NOs: 1281 or SEQ ID NO: 1282. In yet another embodiment, the FNR-responsive promoter consists of a sequence selected from SEQ ID NOs: 1281 or SEQ ID NO: 1282.

In some embodiments, multiple distinct FNR nucleic acid sequences are inserted in the genetically engineered bacteria. In alternate embodiments, the genetically engineered bacteria comprise a gene encoding a payload expressed under the control of an alternate oxygen level-dependent promoter, e.g., DNR (Trunk et al., 2010) or ANR (Ray et al., 1997). In these embodiments, expression of the payload gene is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

In another embodiment, the genetically engineered bacteria comprise the gene or gene cassette for producing an immune modulator expressed under the control of anaerobic regulation of arginine deiminase and nitrate reduction transcriptional regulator (ANR). In P. aeruginosa, ANR is “required for the expression of physiological functions which are inducible under oxygen-limiting or anaerobic conditions” (Winteler et al., 1996; Sawers 1991). P. aeruginosa ANR is homologous with E. coli FNR, and “the consensus FNR site (TTGAT----ATCAA) was recognized efficiently by ANR and FNR” (Winteler et al., 1996). Like FNR, in the anaerobic state, ANR activates numerous genes responsible for adapting to anaerobic growth. In the aerobic state, ANR is inactive. Pseudomonas fluorescens, Pseudomonas putida, Pseudomonas syringae, and Pseudomonas mendocina all have functional analogs of ANR (Zimmermann et al., 1991). Promoters that are regulated by ANR are known in the art, e.g., the promoter of the arcDABC operon (see, e.g., Hasegawa et al., 1998).

In other embodiments, the one or more gene sequence(s) for producing a payload are expressed under the control of an oxygen level-dependent promoter fused to a binding site for a transcriptional activator, e.g., CRP. CRP (cyclic AMP receptor protein or catabolite activator protein or CAP) plays a major regulatory role in bacteria by repressing genes responsible for the uptake, metabolism, and assimilation of less favorable carbon sources when rapidly metabolizable carbohydrates, such as glucose, are present (Wu et al., 2015). This preference for glucose has been termed glucose repression, as well as carbon catabolite repression (Deutscher, 2008; Görke and Stülke, 2008). In some embodiments, the gene or gene cassette for producing an immune modulator is controlled by an oxygen level-dependent promoter fused to a CRP binding site. In some embodiments, the one or more gene sequence(s) for a payload are controlled by a FNR promoter fused to a CRP binding site. In these embodiments, cyclic AMP binds to CRP when no glucose is present in the environment. This binding causes a conformational change in CRP, and allows CRP to bind tightly to its binding site. CRP binding then activates transcription of the gene or gene cassette by recruiting RNA polymerase to the FNR promoter via direct protein-protein interactions. In the presence of glucose, cyclic AMP does not bind to CRP and transcription of the gene or gene cassette for producing a payload is repressed. In some embodiments, an oxygen level-dependent promoter (e.g., an FNR promoter) fused to a binding site for a transcriptional activator is used to ensure that the gene or gene cassette for producing a payload is not expressed under anaerobic conditions when sufficient amounts of glucose are present, e.g., by adding glucose to growth media in vitro.

In some embodiments, the genetically engineered bacteria comprise an oxygen level-dependent promoter from a different species, strain, or substrain of bacteria. In some embodiments, the genetically engineered bacteria comprise an oxygen level-sensing transcription factor, e.g., FNR, ANR or DNR, from a different species, strain, or substrain of bacteria. In some embodiments, the genetically engineered bacteria comprise an oxygen level-sensing transcription factor and corresponding promoter from a different species, strain, or substrain of bacteria. The heterologous oxygen-level dependent transcriptional regulator and/or promoter increases the transcription of genes operably linked to said promoter, e.g., one or more gene sequence(s) for producing the payload(s) in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the payload, in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the payload, in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., (2006). In some embodiments, both the oxygen level-sensing transcriptional regulator and corresponding promoter are mutated relative to the wild-type sequences from bacteria of the same subtype in order to increase expression of the payload in low-oxygen conditions.

In some embodiments, the bacterial cells comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the payload are present on different plasmids. In some embodiments, the gene encoding the oxygen level-sensing transcriptional regulator and the gene encoding the payload are present on the same plasmid.

In some embodiments, the gene encoding the oxygen level-sensing transcriptional regulator is present on a chromosome. In some embodiments, the gene encoding the oxygen level-sensing transcriptional regulator and the gene encoding the payload are present on different chromosomes. In some embodiments, the gene encoding the oxygen level-sensing transcriptional regulator and the gene encoding the payload are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the payload. In some embodiments, expression of the transcriptional regulator is controlled by the same promoter that controls expression of the payload. In some embodiments, the transcriptional regulator and the payload are divergently transcribed from a promoter region.

### RNS-Dependent Regulation

In some embodiments, the genetically engineered bacterium that expresses a payload under the control of a promoter that is activated by inflammatory conditions. In one embodiment, the gene for producing the payload is expressed under the control of an inflammatory-dependent promoter that is activated in inflammatory environments, e.g., a reactive nitrogen species or RNS promoter. In some embodiments, the genetically engineered bacteria of the invention comprise a tunable regulatory region that is directly or indirectly controlled by a transcription factor that is capable of sensing at least one reactive nitrogen species. Suitable RNS inducible promoters, e.g., inducible by reactive nitrogen species are described in International Patent Application PCT/US2017/013072, filed Jan. 11, 2017, published as WO2017/123675, the contents of which is herein incorporated by reference in its entirety.

Examples of transcription factors that sense RNS and their corresponding RNS-responsive genes, promoters, and/or regulatory regions include, but are not limited to, those shown in Table 3.

TABLE 3
Examples of RNS-sensing transcription
factors and RNS-responsive genes

| RNS-sensing | Primarily | Examples of responsive |
|---|---|---|
| transcription | capable | genes, promoters, |
| factor: | of sensing: | and/or regulatory regions: |
| NsrR | NO | norB, aniA, nsrR, hmpA, ytfE, |
| | | ygbA, hcp, hcr, nrfA, aox |
| NorR | NO | norVW, nor |
| DNR | NO | norCB, nir, nor, nos |

### ROS-Dependent Regulation

In some embodiments, the genetically engineered bacterium that expresses a payload under the control of a promoter that is activated by conditions of cellular damage. In one embodiment, the gene for producing the payload is expressed under the control of a cellular damaged-dependent promoter that is activated in environments in which there is cellular or tissue damage, e.g., a reactive oxygen species or ROS promoter. In some embodiments, the genetically engineered bacteria of the invention comprise a tunable regulatory region that is directly or indirectly controlled by a transcription factor that is capable of sensing at least one reactive oxygen species. Suitable ROS inducible promoters, e.g., inducible by reactive oxygen species are described in International Patent Application PCT/US2017/013072, filed Jan. 11, 2017, published as WO2017/123675, the contents of which is herein incorporated by reference in its entirety.

Examples of transcription factors that sense ROS and their corresponding ROS-responsive genes, promoters, and/or regulatory regions include, but are not limited to, those shown in Table 4.

TABLE 4
Examples of ROS-sensing transcription factors and ROS-responsive genes

| ROS-sensing | Primarily capable of | Examples of responsive genes, |
|---|---|---|
| transcription factor: | sensing: | promoters, and/or regulatory regions: |
| OxyR | H₂O₂ | ahpC; ahpF; dps; dsbG; fhuF; flu; fur; |
| | | gor; grxA; hemH; katG; oxyS; sufA; suf73; |
| | | sufC; sufD; sufE; sufS; trxC; uxuA; yaaA; |
| | | yaeH; yaiA; ybjM; ydcH; ydeN; ygaQ; |
| | | yljA; ytfK |
| PerR | H₂O₂ | katA; ahpCF; mrgA; zoaA; fur; |
| | | hemAXCDBL; srfA |
| OhrR | Organic peroxides NaOCl | ohrA |
| SoxR | •O₂⁻ | soxS |
| | NO• | |
| | (also capable of sensing H₂O₂) | |
| RosR | H₂O₂ | rbtT; tnp16a; rluC1; tnp5a; mscL; tnp2d; |
| | | phoD; tnp15b; pstA; tnp5b; xylC; gabD1; |
| | | rluC2; cgtS9; azlC; narKGHJI; rosR |

### Other Promoters

In some embodiments, the genetically engineered bacteria comprise the gene or gene cassette for producing an immune modulator expressed under the control of an inducible promoter that is responsive to specific molecules or metabolites in the environment, e.g., a specific tissue, or the mammalian gut. Any molecule or metabolite found in the mammalian gut, in a healthy and/or disease state, may be used to induce payload expression.

In alternate embodiments, the gene or gene cassette for producing an immune modulator is operably linked to a nutritional or chemical inducer which is not present in the environment, e.g., a specific tissue, or the mammalian gut. In some embodiments, the nutritional or chemical inducer is administered prior, concurrently or sequentially with the genetically engineered bacteria.

### Other Inducible Promoters

In some embodiments, one or more gene sequence(s) encoding polypeptides of interest described herein is present on a plasmid and operably linked to promoter a directly or indirectly inducible by one or more nutritional and/or chemical inducer(s) and/or metabolite(s). In some embodiments, the bacterial cell comprises a stably maintained plasmid or chromosome carrying the gene encoding the immune modulator, which is induced by one or more nutritional and/or chemical inducer(s) and/or metabolite(s), such that the immune modulator can be expressed in the host cell, and the host cell is capable of survival and/or growth in vitro, e.g., under culture conditions, and/or in vivo, e.g., in the gut.

In some embodiments, expression of one or more displayed proteins (e.g., viral, bacterial, fungal, and cancer protein) and or one or more immune modulator(s) and/or other polypeptide(s) of interest is driven directly or indirectly by one or more arabinose, cumate, and salicylate inducible promoter(s) in vivo. In some embodiments, the promoter is directly or indirectly induced by a chemical and/or nutritional inducer and/or metabolite which is co-administered with the genetically engineered bacteria of the invention. In some embodiments, inducers are administered intranasally at a defined time before bacterial injection into the target site. In some embodiments, inducers are administered intranasally at a defined time after bacterial injection into the target site. In some embodiments, inducers are administered intranasally concurrently with bacterial injection into the target site. In some embodiments, inducers are administered intravenously at a defined time before bacterial injection into the target site. In some embodiments, inducers are administered intravenously at a defined time after bacterial injection into the target site. In some embodiments, inducers are administered intravenously concurrently with bacterial injection into the target site. In some embodiments, inducers are administered subcutaneously at a defined time before bacterial injection into the target site. In some embodiments, inducers are administered subcutaneously at a defined time after bacterial injection into the target site. In some embodiments, inducers are administered subcutaneously concurrently with bacterial injection into the target site.

In some embodiments, inducers are administered intranasally at a defined time before bacterial injection into the target site. In some embodiments, inducers are administered intranasally at a defined time after bacterial injection into the target site. In some embodiments, inducers are administered intranasally concurrently with bacterial injection into the target site. In some embodiments, inducers are administered intravenously at a defined time before bacterial injection into the target site. In some embodiments, inducers are administered intravenously at a defined time after bacterial injection into the target site. In some embodiments, inducers are administered intravenously concurrently with intravenous bacterial administration. In some embodiments, inducers are administered subcutaneously at a defined time before bacterial injection into the target site. In some embodiments, inducers are administered subcutaneously at a defined time after bacterial injection into the target site. In some embodiments, inducers are administered subcutaneously concurrently with intravenous bacterial administration.

In some embodiments, expression of one or more display proteins comprising a displayed protein (e.g., viral, bacterial, fungal, and cancer protein) and/or one or more immune modulator(s) and/or other polypeptide(s) of interest, is driven directly or indirectly by one or more promoter(s) induced by a chemical and/or nutritional inducer and/or metabolite during in vitro growth, preparation, or manufacturing of the strain prior to in vivo administration. In some embodiments, the promoter(s) induced by a chemical and/or nutritional inducer and/or metabolite are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with one or more displayed proteins (e.g., viral, bacterial, fungal, and cancer protein), and/or immune modulator(s) and/or other polypeptide(s) of interest prior to administration. In some embodiments, the cultures, which are induced by a chemical and/or nutritional inducer and/or metabolite, are grown aerobically. In some embodiments, the cultures, which are induced by a chemical and/or nutritional inducer and/or metabolite, are grown anaerobically.

In one embodiment, the gene encoding the effector or the immune modulator is operably linked to a promoter that is induced by salicylate or a derivative thereof. After over 100 years of clinical use, salicylate remains one of the world's most extensively used ‘over-the-counter’ drugs, and it is still recognized as the standard analgesic/antipyretic/anti-inflammatory agent by which newer drugs are assessed (Clissold; Salicylate and related derivatives of salicylic acid; Drugs. 1986; 32 Suppl 4:8-26). In an non-limiting example, the immune modulator is operably linked to a promoter PSal, as part of the salicylate PSal/NahR biosensor circuit (Part:BBa_J61051), originally adapted from Pseudomonas putida. The nahR gene was mined from the 83 kb naphthalene degradation plasmid NAH7 of Pseudomonas putida, encoding a 34 kDa protein which binds to nah and sal promoters to activate transcription in response to the inducer salicylate (Dunn, N. W., and I. C. Gunsalus (1973) Transmissible plasmid encoding early enzymes of naphthalene oxidation in Pseudomonas putida. J. Bacteriol. 114:974-979). In this system NahR is constitutively expressed by a constitutive promoter (Pc), and the expression of the protein of interest, e.g., the immune modulator is positively regulated by NahR in the presence of inducers (e.g., salicylate). Thus, in some embodiments, the genetically engineered bacteria comprise a gene sequence encoding an immune modulator which is operably linked to salicylate inducible promoter (e.g., PSal). In some embodiments, the genetically engineered bacteria further comprise gene sequence(s) encoding NahR, which are operably linked to a promoter. In some embodiments, NahR is under control of a constitutive promoter described herein or known in the art. In some embodiments, NahR is under control of an inducible promoter described herein or known in the art. In some embodiments described herein, the Biobrick BBa_J61051 (containing the gene encoding NahR driven by a constitutive promoter and the PSal promoter was cloned preceding dacA.

In one embodiment, expression of one or more immune modulator protein(s) of interest, e.g., one or more therapeutic polypeptide(s), is driven directly or indirectly by one or more salicylate inducible promoter(s).

In some embodiments, the salicylate inducible promoter is useful for or induced during in vivo expression of the one or more protein(s) of interest. In some embodiments, expression of one or more immune modulator protein(s) of interest is driven directly or indirectly by one or more salicylate inducible promoter(s) in vivo. In some embodiments, the promoter is directly or indirectly induced by a molecule that is co-administered with the genetically engineered bacteria of the invention, e.g., salicylate.

In some embodiments, salicylate is administered intranasally at a defined time before bacterial injection into the target site. In some embodiments, salicylate is administered intranasally at a defined time after bacterial injection into the target site. In some embodiments, salicylate is administered intranasally concurrently with bacterial injection into the target site. In some embodiments, salicylate is administered intravenously at a defined time before bacterial injection into the target site. In some embodiments, salicylate is administered intravenously at a defined time after bacterial injection into the target site. In some embodiments, salicylate is administered intravenously concurrently with bacterial injection into the target site. In some embodiments, salicylate is administered subcutaneously at a defined time before bacterial injection into the target site. In some embodiments, salicylate is administered subcutaneously at a defined time after bacterial injection into the target site. In some embodiments, salicylate is administered subcutaneously concurrently with bacterial injection into the target site.

In some embodiments, salicylate is administered intranasally at a defined time before bacterial injection into the target site. In some embodiments, salicylate is administered intranasally at a defined time after bacterial injection into the target site. In some embodiments, salicylate is administered intranasally concurrently with bacterial injection into the target site. In some embodiments, salicylate is administered intravenously at a defined time before bacterial injection into the target site. In some embodiments, salicylate is administered intravenously at a defined time after bacterial injection into the target site. In some embodiments, salicylate is administered intravenously concurrently with intravenous bacterial administration. In some embodiments, salicylate is administered subcutaneously at a defined time before bacterial injection into the target site. In some embodiments, salicylate is administered subcutaneously at a defined time after bacterial injection into the target site. In some embodiments, salicylate is administered subcutaneously concurrently with intravenous bacterial administration.

In some embodiments, expression of one or more protein(s) of interest, is driven directly or indirectly by one or more salicylate inducible promoter(s) during in vitro growth, preparation, or manufacturing of the strain prior to in vivo administration. In some embodiments, the salicylate inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with the payload prior to administration, e.g., salicylate. In some embodiments, the cultures, which are induced by salicylate, are grown aerobically. In some embodiments, the cultures, which are induced by salicylate, are grown anaerobically.

In some embodiments, the salicylate inducible promoter drives the expression of one or more protein(s) of interest from a low-copy plasmid or a high copy plasmid or a biosafety system plasmid described herein. In some embodiments, the salicylate inducible promoter drives the expression of one or more protein(s) of interest from a construct which is integrated into the bacterial chromosome. Exemplary insertion sites are described herein.

In some embodiments, one or more protein(s) of interest are linked to and are driven by the native salicylate inducible promoter In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 1273 or SEQ ID NO: 1274.

In one embodiment, the genetically engineered bacteria comprise a gene sequence comprising SEQ ID NO: 1273 or SEQ ID NO: 1274. In another embodiment, the genetically engineered bacteria comprise a gene sequence which consists of SEQ ID NO: 1273 or SEQ ID NO: 1274.

In some embodiments, the salicylate inducible construct further comprises a gene encoding NahR, which in some embodiments is divergently transcribed from a constitutive or inducible promoter. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 1278. In another embodiment, the genetically engineered bacteria comprise a gene sequence comprising SEQ ID NO: 1278. In another embodiment, the genetically engineered bacteria comprise a gene sequence which consists of SEQ ID NO: 1278.

In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by SEQ ID NO: 1280. In another embodiment, the genetically engineered bacteria comprise a gene sequence encoding a polypeptide comprising SEQ ID NO: 1280. In yet another embodiment, the polypeptide expressed by the genetically engineered bacteria consists of SEQ ID NO: 1280.

In one embodiment, the gene encoding the immune modulator is operably linked to a promoter that is induced by cumate or a derivative thereof. Suitable derivatives are known in the art and are for example described in U.S. Pat. No. 7,745,592. Benefits of cumate induction include that Cumate is non-toxic, water-soluble and inexpensive. The basic mechanism by which the cumate-regulated expression functions in the native P. putida F1 and how it is applied to other bacterial chassis, including but not limited to, E. coli has been previously described (see e.g., Choi et al., Novel, Versatile, and Tightly Regulated Expression System for Escherichia coli Strains; Appl. Environ. Microbiol. August 2010 vol. 76 no. 15 5058-5066). Essentially, the cumate circuit or switch includes four components: a strong promoter, a repressor-binding DNA sequence or operator, expression of cymR, a repressor, and cumate as the inducer. The addition of the inducer changes causes the formation of a complex between cumate and CymR and results in the removal of the repressor from its DNA binding site, allowing expression of the gene of interest. A construct comprising the cymR gene driven by a constitutive promoter and a cymR responsive promoter was cloned in front of the DacA gene to allow cumate inducible expression of DacA is described elsewhere herein.

In one embodiment, expression of one or more immune modulator protein(s) of interest, e.g., one or more therapeutic polypeptide(s), is driven directly or indirectly by one or more promoter(s) inducible by cumate or a derivative thereof.

In some embodiments, the cumate inducible promoter is useful for or induced during in vivo expression of the one or more protein(s) of interest. In some embodiments, expression of one or more immune modulator protein(s) of interest is driven directly or indirectly by one or more cumate inducible promoter(s) in vivo. In some embodiments, the promoter is directly or indirectly induced by a molecule that is co-administered with the genetically engineered bacteria of the invention, e.g., cumate.

In some embodiments, cumate is administered intranasally at a defined time before bacterial injection into the target site. In some embodiments, cumate is administered intranasally at a defined time after bacterial injection into the target site. In some embodiments, cumate is administered intranasally concurrently with bacterial injection into the target site. In some embodiments, cumate is administered intravenously at a defined time before bacterial injection into the target site. In some embodiments, cumate is administered intravenously at a defined time after bacterial injection into the target site. In some embodiments, cumate is administered intravenously concurrently with bacterial injection into the target site. In some embodiments, cumate is administered subcutaneously at a defined time before bacterial injection into the target site. In some embodiments, cumate is administered subcutaneously at a defined time after bacterial injection into the target site. In some embodiments, cumate is administered subcutaneously concurrently with bacterial injection into the target site.

In some embodiments, cumate is administered intranasally at a defined time before bacterial injection into the target site. In some embodiments, cumate is administered intranasally at a defined time after bacterial injection into the target site. In some embodiments, cumate is administered intranasally concurrently with bacterial injection into the target site. In some embodiments, cumate is administered intravenously at a defined time before bacterial injection into the target site. In some embodiments, cumate is administered intravenously at a defined time after bacterial injection into the target site. In some embodiments, cumate is administered intravenously concurrently with intravenous bacterial administration. In some embodiments, cumate is administered subcutaneously at a defined time before bacterial injection into the target site. In some embodiments, cumate is administered subcutaneously at a defined time after bacterial injection into the target site. In some embodiments, cumate is administered subcutaneously concurrently with intravenous bacterial administration

In some embodiments, expression of one or more protein(s) of interest, is driven directly or indirectly by one or more cumate inducible promoter(s) during in vitro growth, preparation, or manufacturing of the strain prior to in vivo administration. In some embodiments, the cumate inducible promoter(s) are induced in culture, e.g., grown in a flask, fermenter or other appropriate culture vessel, e.g., used during cell growth, cell expansion, fermentation, recovery, purification, formulation, and/or manufacture. In some embodiments, the promoter is directly or indirectly induced by a molecule that is added to in the bacterial culture to induce expression and pre-load the bacterium with the payload prior to administration, e.g., cumate. In some embodiments, the cultures, which are induced by cumate, are grown aerobically. In some embodiments, the cultures, which are induced by cumate, are grown anaerobically.

In some embodiments, the cumate inducible promoter drives the expression of one or more protein(s) of interest from a low-copy plasmid or a high copy plasmid or a biosafety system plasmid described herein. In some embodiments, the cumate inducible promoter drives the expression of one or more protein(s) of interest from a construct which is integrated into the bacterial chromosome. Exemplary insertion sites are described herein.

In some embodiments, one or more protein(s) of interest are operably linked to by the native cumate inducible promoter. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 1270 or SEQ ID NO: 1271.

In one embodiment, the genetically engineered bacteria comprise a gene sequence comprising SEQ ID NO: 1270 or SEQ ID NO: 1271. In another embodiment, the genetically engineered bacteria comprise a gene sequence which consists of SEQ ID NO: 1270 or SEQ ID NO: 1271

In some embodiments, the cumate inducible construct further comprises a gene encoding CymR, which in some embodiments is divergently transcribed from a constitutive or inducible promoter. In some embodiments, the genetically engineered bacteria comprise one or more gene sequence(s) having at least 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 1268. In another embodiment, the genetically engineered bacteria comprise a gene sequence comprising SEQ ID NO: 1268. In another embodiment, the genetically engineered bacteria comprise a gene sequence which consists of SEQ ID NO: 1268.

In some embodiments, the genetically engineered bacteria comprise one or more gene sequence(s) encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% identity with the polypeptide encoded by SEQ ID NO: 1269. In another embodiment, the genetically engineered bacteria comprise a gene sequence encoding a polypeptide comprising SEQ ID NO: 1269. In yet another embodiment, the polypeptide expressed by the genetically engineered bacteria consists of SEQ ID NO: 1269.

Other inducible promoters contemplated in the disclosure are described in are described in International Patent Application PCT/US2017/013072, filed Jan. 11, 2017, published as WO2017/123675, the contents of which is herein incorporated by reference in its entirety. Such promoters include arabinose inducible, rhamnose inducible, and IPTG inducible promoters, tetracycline inducible promoters, temperature inducible promoters, and PSSB promoter. These promoters can be used in combination with each other or with other inducible promoters, such as low oxygen inducible promoters, or constitutive promoters to fine tune expression of different effectors, e.g., in one bacterium or in a composition of more than one strain of bacteria.

### Constitutive Promoters

In some embodiments, the gene encoding the payload is present on a plasmid and operably linked to a constitutive promoter. In some embodiments, the gene encoding the payload is present on a chromosome and operably linked to a constitutive promoter.

In some embodiments, the constitutive promoter is active under in vivo conditions, as described herein. In some embodiments, the promoters is active under in vitro conditions, e.g., various cell culture and/or cell manufacturing conditions, as described herein. In some embodiments, the constitutive promoter is active under in vivo conditions, as described herein, and under in vitro conditions, e.g., various cell culture and/or cell production and/or manufacturing conditions, as described herein.

In some embodiments, the constitutive promoter that is operably linked to the gene encoding the payload is active in various exogenous environmental conditions (e.g., in vivo and/or in vitro and/or production/manufacturing conditions).

In some embodiments, the constitutive promoter is active in exogenous environmental conditions specific to the target sites. In some embodiments, the constitutive promoter is active in exogenous environmental conditions specific to the pulmonary system of a mammal. In some embodiments, the constitutive promoter is active in the presence of molecules or metabolites that are specific to the pulmonary system of a mammal. In some embodiments, the constitutive promoter is directly or indirectly induced by a molecule that is co-administered with the bacterial cell. In some embodiments, the constitutive promoter is active in the presence of molecules or metabolites or other conditions, that are present during in vitro culture, cell production and/or manufacturing conditions. Bacterial constitutive promoters are known in the art and are described in are described in International Patent Application PCT/US2017/013072, filed Jan. 11, 2017, published as WO2017/123675, the contents of which is herein incorporated by reference in its entirety. Examples are included herein in SEQ ID NO: 598-739 and a subset is shown in Table 5.

TABLE 5
Promoters

| | | SEQ |
|---|---|---|
| | | ID |
| Name | Description | NO |
| Plpp | The Plpp promoter is a natural | 740 |
| | promoter taken from the Nissle | |
| | genome. In situ it is used to | |
| | drive production of lpp, which | |
| | is known to be the most | |
| | abundant protein in the cell. | |
| | Also, in some previous RNAseq | |
| | experiments I was able to | |
| | confirm that the lpp mRNA is | |
| | one of the most abundant mRNA | |
| | in Nissle during exponential | |
| | growth. | |
| PapFAB46 | See, e.g., Kosuri, S., | 741 |
| | Goodman, D. B. & Cambray, G. | |
| | Composability of regulatory | |
| | sequences controlling | |
| | transcription and translation | |
| | in Escherichia coli. in | |
| | 1-20 (2013). | |
| | doi:10.1073/pnas. | |
| PJ23101 | UP clement helps recruit | 742 |
| UP | RNA polymerase | |
| element | (gaaaaatttttttaaaaaaaaaac | |
| | (SEQ ID NO: 1250)) | |
| PJ23107 | UP clement helps recruit | 743 |
| UP | RNA polymerase | |
| element | (gaaaaatttttttaaaaaaaaaac | |
| | (SEQ ID NO: 1250)) | |
| PSYN23119 | UP element at 5′ end; | 744 |
| | consensus −10 region is | |
| | TATAAT; the | |
| | consensus −35 is TTGACA; | |
| | the extended −10 region | |
| | is generally TGNTATAAT | |
| | (TGGTATAAT in this sequence) | |

In some embodiments, the promoter is Plpp or a derivative thereof. In some embodiments, the promoter comprises a sequence from SEQ ID NO:740. In some embodiments, the constitutive promoter is at least about 80%, at least about 85%, at least about 90%, at least about 95%, or at least about 99% homologous to the sequence of SEQ ID NO: 740. In some embodiments, the promoter is PapFAB46 or a derivative thereof. In some embodiments, the promoter comprises a sequence from SEQ ID NO:741. In some embodiments, the constitutive promoter is at least about 80%, at least about 85%, at least about 90%, at least about 95%, or at least about 99% homologous to the sequence of SEQ ID NO: 741. In some embodiments, the promoter is PJ23101+UP element or a derivative thereof. In some embodiments, the promoter comprises a sequence from SEQ ID NO:742. In some embodiments, the constitutive promoter is at least about 80%, at least about 85%, at least about 90%, at least about 95%, or at least about 99% homologous to the sequence of SEQ ID NO: 742. In some embodiments, the promoter is PJ23107+UP element or a derivative thereof. In some embodiments, the promoter comprises a sequence from SEQ ID NO:743. In some embodiments, the constitutive promoter is at least about 80%, at least about 85%, at least about 90%, at least about 95%, or at least about 99% homologous to the sequence of SEQ ID NO: 743. In some embodiments, the promoter is PSYN23119 or a derivative thereof. In some embodiments, the promoter comprises a sequence from SEQ ID NO:744. In some embodiments, the constitutive promoter is at least about 80%, at least about 85%, at least about 90%, at least about 95%, or at least about 99% homologous to the sequence of SEQ ID NO: 744.

Additional promoters which may be linked to the payload include apFAB124 (tcgacatttatcccttgcggcgaatacttacagccatagcaa (SEQ ID NO: 1443)); apfab338(GGCGCGCC TTGACAATTAATCATCCGGCTCCTAGGATGTGTGGAGGGAC (SEQ ID NO: 1444)), apFAB66 (GGCGCGCC TTGACATCAGGAAAATTTTTCTGTATAATAGATTCATCTCAA (SEQ ID NO: 1445)), and apFAB54 (GGCGCGCC TTGACATAAAGTCTAACCTATAGGATACTTACAGCCATACAAG (SEQ ID NO: 1446)). In some embodiments, the promoter is apFAB124 or a derivative thereof. In some embodiments, the promoter comprises a sequence of apFAB124. In some embodiments, the constitutive promoter is at least about 80%, at least about 85%, at least about 90%, at least about 95%, or at least about 99% homologous to the sequence of apFAB124. In some embodiments, the promoter is apFAB338 or a derivative thereof. In some embodiments, the promoter comprises a sequence of apFAB338. In some embodiments, the constitutive promoter is at least about 80%, at least about 85%, at least about 90%, at least about 95%, or at least about 99% homologous to the sequence of apFAB338. In some embodiments, the promoter is apFAB66 or a derivative thereof. In some embodiments, the promoter comprises a sequence of apFAB66. In some embodiments, the constitutive promoter is at least about 80%, at least about 85%, at least about 90%, at least about 95%, or at least about 99% homologous to the sequence of apFAB66. In some embodiments, the promoter is apFAB54 or a derivative thereof. In some embodiments, the promoter comprises a sequence of apFAB54. In some embodiments, the constitutive promoter is at least about 80%, at least about 85%, at least about 90%, at least about 95%, or at least about 99% homologous to the sequence of apFAB54.

### Ribosome Binding Sites

In some embodiments, ribosome binding sites are added, switched out or replaced. By testing a few ribosome binding sites, expression levels can be fine-tuned to the desired level. In some embodiments, RBS which are suitable for prokaryotic expression and can be used to achieve the desired expression levels are selected. Non-limiting examples of RBS are listed at Registry of standard biological parts and are described in are described in International Patent Application PCT/US2017/013072, filed Jan. 11, 2017, published as WO2017/123675, the contents of which is herein incorporated by reference in its entirety. Suitable examples are shown in SEQ ID NO: 1018-1050 and 869-871, 873-877, 880-887.

### Induction of Payloads During Strain Culture

Induction of payloads during culture is described in International Patent Application PCT/US2017/013072, filed Jan. 11, 2017, published as WO2017/123675, the contents of which is herein incorporated by reference in its entirety.

In some embodiments, it is desirable to pre-induce payload or protein of interest expression and/or payload activity prior to administration. Such payload or protein of interest may be an effector intended for secretion or may be an enzyme which catalyzes a metabolic reaction to produce an effector. In other embodiments, the protein of interest is an enzyme which catabolizes a harmful metabolite. In such situations, the strains are pre-loaded with active payload or protein of interest. In such instances, the genetically engineered bacteria of the invention express one or more protein(s) of interest, under conditions provided in bacterial culture during cell growth, expansion, purification, fermentation, and/or manufacture prior to administration in vivo. Such culture conditions can be provided in a flask, fermenter or other appropriate culture vessel, e.g., used during cell growth, cell expansion, fermentation, recovery, purification, formulation, and/or manufacture. As used herein, the term “bacterial culture” or bacterial cell culture” or “culture” refers to bacterial cells or microorganisms, which are maintained or grown in vitro during several production processes, including cell growth, cell expansion, recovery, purification, fermentation, and/or manufacture. As used herein, the term “fermentation” refers to the growth, expansion, and maintenance of bacteria under defined conditions. Fermentation may occur under a number of cell culture conditions, including anaerobic or low oxygen or oxygenated conditions, in the presence of inducers, nutrients, at defined temperatures, and the like.

Culture conditions are selected to achieve optimal activity and viability of the cells, while maintaining a high cell density (high biomass) yield. A number of cell culture conditions and operating parameters are monitored and adjusted to achieve optimal activity, high yield and high viability, including oxygen levels (e.g., low oxygen, microaerobic, aerobic), temperature of the medium, and nutrients and/or different growth media, chemical and/or nutritional inducers and other components provided in the medium.

In some embodiments, the one or more protein(s) of interest and are directly or indirectly induced, while the strains is grown up for in vivo administration. Without wishing to be bound by theory, pre-induction may boost in vivo activity. If the bacterial residence time in a particular pulmonary compartment is relatively short, the bacteria may pass through without reaching full in vivo induction capacity. In contrast, if a strain is pre-induced and preloaded, the strains are already fully active, allowing for greater activity more quickly as the bacteria reach the pulmonary system. Ergo, no transit time is “wasted”, in which the strain is not optimally active. As the bacteria continue to move through the pulmonary system, in vivo induction occurs under environmental conditions of the pulmonary system. Similarly, systemic administration or intranasal delivery, as described herein, of other bacterium may allow for greater activity more quickly as the bacteria reach the target site.

In one embodiment, expression of one or more payload(s), is induced during cell growth, cell expansion, fermentation, recovery, purification, formulation, and/or manufacture. In one embodiment, expression of several different proteins of interest is induced during cell growth, cell expansion, fermentation, recovery, purification, formulation, and/or manufacture.

In some embodiments, the strains are administered without any pre-induction protocols during strain growth prior to in vivo administration.

### Anaerobic Induction

In some embodiments, cells are induced under anaerobic or low oxygen conditions in culture. In such instances, cells are grown (e.g., for 1.5 to 3 hours) until they have reached a certain OD, e.g., ODs within the range of 0.1 to 10, indicating a certain density e.g., ranging from 1×10\hat{ }8 to 1λ10\hat{ }11, and exponential growth and are then switched to anaerobic or low oxygen conditions for approximately 3 to 5 hours. In some embodiments, strains are induced under anaerobic or low oxygen conditions, e.g. to induce FNR promoter activity and drive expression of one or more payload(s) and/or transporters under the control of one or more FNR promoters.

In one embodiment, expression of one or more payload(s), is under the control of one or more FNR promoter(s) and is induced during cell growth, cell expansion, fermentation, recovery, purification, formulation, and/or manufacture under anaerobic or low oxygen conditions. In one embodiment, expression of several different proteins of interest is under the control of one or more FNR promoter(s) and is induced during cell growth, cell expansion, fermentation, recovery, purification, formulation, and/or manufacture under anaerobic or low oxygen conditions.

Without wishing to be bound by theory, strains that comprise one or more payload(s) under the control of an FNR promoter, may allow expression of payload(s) from these promoters in vitro, under anaerobic or low oxygen culture conditions, and in vivo.

In some embodiments, promoters linked to the payload of interest may be inducible by arabinose, cumate, and salicylate, IPTG, rhamnose, tetracycline, and/or other chemical and/or nutritional inducers can be induced under anaerobic or low oxygen conditions in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) and/or under the control of one or more FNR promoter(s), and one or more payload gene sequence(s) under the control of a one or more constitutive promoter(s) described herein.

### Aerobic Induction

In some embodiments, it is desirable to prepare, pre-load and pre-induce the strains under aerobic conditions. This allows more efficient growth and viability, and, in some cases, reduces the build-up of toxic metabolites. In such instances, cells are grown (e.g., for 1.5 to 3 hours) until they have reached a certain OD, e.g., ODs within the range of 0.1 to 10, indicating a certain density e.g., ranging from 1×10\hat{ }8 to 1×10 \hat{ }11, and exponential growth and are then induced through the addition of the inducer or through other means, such as shift to a permissive temperature, for approximately 3 to 5 hours.

In some embodiments, promoters inducible by arabinose, cumate, and salicylate, IPTG, rhamnose, tetracycline, and/or other chemical and/or nutritional inducers described herein or known in the art can be induced under aerobic conditions in the presence of the chemical and/or nutritional inducer during cell growth, cell expansion, fermentation, recovery, purification, formulation, and/or manufacture. In one embodiment, expression of one or more payload(s) is under the control of one or more promoter(s) regulated by chemical and/or nutritional inducers and is induced during cell growth, cell expansion, fermentation, recovery, purification, formulation, and/or manufacture under aerobic conditions.

In some embodiments, genetically engineered strains comprise gene sequence(s) which are induced under aerobic culture conditions. In some embodiments, these strains further comprise FNR inducible gene sequence(s) for in vivo activation. In some embodiments, these strains do not further comprise FNR inducible gene sequence(s) for in vivo activation.

### Microaerobic Induction

In some embodiments, viability, growth, and activity are optimized by pre-inducing the bacterial strain under microaerobic conditions. In some embodiments, microaerobic conditions are best suited to “strike a balance” between optimal growth, activity and viability conditions and optimal conditions for induction; in particular, if the expression of the one or more payload(s) are driven by an anaerobic and/or low oxygen promoter, e.g., a FNR promoter. In such instances, cells are for example grown (e.g., for 1.5 to 3 hours) until they have reached a certain OD, e.g., ODs within the range of 0.1 to 10, indicating a certain density e.g., ranging from 1×10\hat{ }8 to 1×10\hat{ }11, and exponential growth and are then induced through the addition of the inducer or through other means, such as shift to at a permissive temperature, for approximately 3 to 5 hours.

In one embodiment, expression of one or more payload(s) is under the control of one or more FNR promoter(s) and is induced during cell growth, cell expansion, fermentation, recovery, purification, formulation, and/or manufacture under microaerobic conditions.

Without wishing to be bound by theory, strains that comprise one or more payload(s) under the control of an FNR promoter, may allow expression of payload(s) from these promoters in vitro, under microaerobic culture conditions, and in vivo, under the low oxygen conditions.

In some embodiments, promoters inducible by arabinose, cumate, and salicylate, IPTG, rhamnose, tetracycline, and/or other chemical and/or nutritional inducers can be induced under microaerobic conditions in the presence of the chemical and/or nutritional inducer. In particular, strains may comprise a combination of gene sequence(s), some of which are under control of FNR promoters and others which are under control of promoters induced by chemical and/or nutritional inducers. In some embodiments, strains may comprise one or more payload gene sequence(s) under the control of one or more FNR promoter(s), and one or more payload gene sequence(s) under the control of a one or more constitutive promoter(s) described herein.

In one embodiment, expression of one or more payload(s) is under the control of one or more promoter(s) regulated by chemical and/or nutritional inducers and is induced during cell growth, cell expansion, fermentation, recovery, purification, formulation, and/or manufacture under microaerobic conditions.

Induction of Strains Using Phasing, Pulsing and/or Cycling

In some embodiments, cycling, phasing, or pulsing techniques are employed during cell growth, expansion, recovery, purification, fermentation, and/or manufacture to efficiently induce and grow the strains prior to in vivo administration. This method is used to “strike a balance” between optimal growth, activity, cell health, and viability conditions and optimal conditions for induction; in particular, if growth, cell health or viability are negatively affected under inducing conditions. In such instances, cells are grown (e.g., for 1.5 to 3 hours) in a first phase or cycle until they have reached a certain OD, e.g., ODs within the range of 0.1 to 10, indicating a certain density e.g., ranging from 1×10\hat{ }8 to 1×10\hat{ }11, and are then induced through the addition of the inducer or through other means, such as shift to a permissive temperature (if a promoter is thermoregulated), or change in oxygen levels (e.g., reduction of oxygen level in the case of induction of an FNR promoter driven construct) for approximately 3 to 5 hours. In a second phase or cycle, conditions are brought back to the original conditions which support optimal growth, cell health and viability. Alternatively, if a chemical and/or nutritional inducer is used, then the culture can be spiked with a second dose of the inducer in the second phase or cycle.

In some embodiments, two cycles of optimal conditions and inducing conditions are employed (i.e., growth, induction, recovery and growth, induction). In some embodiments, three cycles of optimal conditions and inducing conditions are employed. In some embodiments, four or more cycles of optimal conditions and inducing conditions are employed. In a non-liming example, such cycling and/or phasing is used for induction under anaerobic and/or low oxygen conditions (e.g., induction of FNR promoters). In one embodiment, cells are grown to the optimal density and then induced under anaerobic and/or low oxygen conditions. Before growth and/or viability are negatively impacted due to stressful induction conditions, cells are returned to oxygenated conditions to recover, after which they are then returned to inducing anaerobic and/or low oxygen conditions for a second time. In some embodiments, these cycles are repeated as needed.

In some embodiments, growing cultures are spiked once with the chemical and/or nutritional inducer. In some embodiments, growing cultures are spiked twice with the chemical and/or nutritional inducer. In some embodiments, growing cultures are spiked three or more times with the chemical and/or nutritional inducer. In a non-limiting example, cells are first grown under optimal growth conditions up to a certain density, e.g., for 1.5 to 3 hour) to reach an of 0.1 to 10, until the cells are at a density ranging from 1×10\hat{ }8 to 1×10\hat{ }11. Then the chemical inducer, e.g., arabinose, cumate, and salicylate or IPTG, is added to the culture. After 3 to 5 hours, an additional dose of the inducer is added to re-initiate the induction. Spiking can be repeated as needed.

In some embodiments, payload(s) induced during cell growth, cell expansion, fermentation, recovery, purification, formulation, and/or manufacture by using phasing or cycling or pulsing or spiking techniques are under the control of different inducible promoters, for example two different chemical inducers. In other embodiments, the payload is induced under low oxygen conditions or microaerobic conditions and a second payload is induced by a chemical inducer.

### Secretion

In any of the embodiments described herein, in which the genetically engineered microorganism produces a protein, polypeptide, peptide, or other immune modulatory, DNA, RNA, small molecule or other molecule intended to be secreted from the microorganism, such as a protein (e.g., viral, bacterial, fungal, and cancer protein) and/or STING agonist, the engineered microorganism may comprise a secretion mechanism and corresponding gene sequence(s) encoding the secretion system.

In some embodiments, the genetically engineered bacteria further comprise a native secretion mechanism or non-native secretion mechanism that is capable of secreting the immune modulator from the bacterial cytoplasm in the extracellular environment. Many bacteria have evolved sophisticated secretion systems to transport substrates across the bacterial cell envelope. Substrates, such as small molecules, proteins, and DNA, may be released into the extracellular space or periplasm (such as the gut lumen or other space), injected into a target cell, or associated with the bacterial membrane.

In Gram-negative bacteria, secretion machineries may span one or both of the inner and outer membranes. In order to translocate a protein, e.g., therapeutic polypeptide, to the extracellular space, the polypeptide must first be translated intracellularly, mobilized across the inner membrane and finally mobilized across the outer membrane. Many effector proteins (e.g., therapeutic polypeptides)—particularly those of eukaryotic origin—contain disulphide bonds to stabilize the tertiary and quaternary structures. While these bonds are capable of correctly forming in the oxidizing periplasmic compartment with the help of periplasmic chaperones, in order to translocate the polypeptide across the outer membrane the disulphide bonds must be reduced and the protein unfolded again.

Suitable secretion systems for secretion of heterologous polypeptides, e.g., effector molecules, from gram negative and gram positive bacteria are described in International Patent Application PCT/US2017/013072, filed Jan. 11, 2017, published as WO2017/123675, the contents of which is herein incorporated by reference in its entirety. Such secretion systems include Double membrane-spanning secretion systems include, but are not limited to, the type I secretion system (T1SS), the type II secretion system (T2SS), the type III secretion system (T3SS), the type IV secretion system (T4SS), the type VI secretion system (T6SS), and the resistance-nodulation-division (RND) family of multi-drug efflux pumps, and type VII secretion system (T7SS). Alternatively, hemolysin-based secretion systems, Type V autotransporter secretion systems, traditional or modified type III or a type III-like secretion systems (T3SS), a flagellar type III secretion pathway may be used. In some embodiments, non-native single membrane-spanning secretion systems, e.g. Tat or Tat-like systems or Sec or Sec like systems may be used. Any of the secretion systems described herein and in PCT/US2017/013072 may according to the disclosure be employed to secrete the polypeptides of interest.

One way to secrete properly folded proteins in gram-negative bacteria—particularly those requiring disulphide bonds—is to target the reducing-environment periplasm in conjunction with a destabilizing outer membrane. In this manner the protein is mobilized into the oxidizing environment and allowed to fold properly. In contrast to orchestrated extracellular secretion systems, the protein is then able to escape the periplasmic space in a correctly folded form by membrane leakage. These “leaky” gram-negative mutants are therefore capable of secreting bioactive, properly disulphide-bonded polypeptides. In some embodiments, the genetically engineered bacteria have a “leaky” or de-stabilized outer membrane (DOM). Destabilizing the bacterial outer membrane to induce leakiness can be accomplished by deleting or mutagenizing genes responsible for tethering the outer membrane to the rigid peptidoglycan skeleton, including for example, lpp, ompC, ompA, ompF, tolA, tolB, and pal. Lpp is the most abundant polypeptide in the bacterial cell existing at ˜500,000 copies per cell and functions as the primary ‘staple’ of the bacterial cell wall to the peptidoglycan. TolA-PAL and OmpA complexes function similarly to Lpp and are other deletion targets to generate a leaky phenotype. Additionally, leaky phenotypes have been observed when periplasmic proteases are inactivated. The periplasm is very densely packed with protein and therefore encode several periplasmic proteins to facilitate protein turnover. Removal of periplasmic proteases such as degS, degP or nlpI can induce leaky phenotypes by promoting an excessive build-up of periplasmic protein. Mutation of the proteases can also preserve the effector polypeptide by preventing targeted degradation by these proteases.

Moreover, a combination of these mutations may synergistically enhance the leaky phenotype of the cell without major sacrifices in cell viability. Thus, in some embodiments, the engineered bacteria have one or more deleted or mutated membrane genes. In some embodiments, the engineered bacteria have a deleted or mutated lpp gene. In some embodiments, the engineered bacteria have one or more deleted or mutated gene(s), selected from ompA, ompA, and ompF genes. In some embodiments, the engineered bacteria have one or more deleted or mutated gene(s), selected from tolA, tolB, and pal genes. in some embodiments, the engineered bacteria have one or more deleted or mutated periplasmic protease genes. In some embodiments, the engineered bacteria have one or more deleted or mutated periplasmic protease genes selected from degS, degP, and nlpl. In some embodiments, the engineered bacteria have one or more deleted or mutated gene(s), selected from lpp, ompA, ompF, tolA, tolB, pal, degS, degP, and nlpl genes.

To minimize disturbances to cell viability, the leaky phenotype can be made inducible by placing one or more membrane or periplasmic protease genes, e.g., selected from lpp, ompA, ompF, tolA, tolB, pal, degS, degP, and nlpl, under the control of an inducible promoter. For example, expression of lpp or other cell wall stability protein or periplasmic protease can be repressed in conditions where the therapeutic polypeptide needs to be delivered (secreted). For instance, under inducing conditions a transcriptional repressor protein or a designed antisense RNA can be expressed which reduces transcription or translation of a target membrane or periplasmic protease gene. Conversely, overexpression of certain peptides can result in a destabilized phenotype, e.g., overexpression of colicins or the third topological domain of TolA, wherein peptide overexpression can be induced in conditions in which the therapeutic polypeptide needs to be delivered (secreted). These sorts of strategies would decouple the fragile, leaky phenotypes from biomass production. Thus, in some embodiments, the engineered bacteria have one or more membrane and/or periplasmic protease genes under the control of an inducible promoter.

In some embodiments in which the one or more proteins of interest or therapeutic proteins are secreted or exported from the microorganism, the engineered microorganism comprises gene sequence(s) that includes a secretion tag. In some embodiments, the one or more proteins of interest or therapeutic proteins include a “secretion tag” of either RNA or peptide origin to direct the one or more proteins of interest or therapeutic proteins to specific secretion systems. The secretion tag can be from the sec or the tat system.

In some embodiments, the genetically engineered bacterial comprise a native or non-native secretion system described herein for the secretion of an immune modulator, e.g., a cytokine, antibody (e.g., scFv), metabolic enzyme (e.g., kynureninase), and others described herein.

In some embodiments, the secretion tag is selected from PhoA, OmpF, cvaC, TorA, fdnG, dmsA, PelB, HlyA secretion signal, and HlyA secretion signal. In some embodiments, the secretion tag is the PhoA secretion signal. In some embodiments, the secretion tag comprises a sequence selected from SEQ ID NO: 745 or SEQ ID NO: 746. In some embodiments, the secretion tag is the OmpF secretion signal. In some embodiments, the secretion tag is the OmpF secretion signal. In some embodiments, the secretion tag comprises SEQ ID NO: 747. In some embodiments, the secretion tag is the cvaC secretion signal. In some embodiments, the secretion tag comprises SEQ ID NO: 748. In some embodiments, the secretion tag is the torA secretion signal. In some embodiments, the secretion tag comprises SEQ ID NO: 749. In some embodiments, the secretion tag is the fdnG secretion signal. In some embodiments, the secretion tag comprises SEQ ID NO: 750. In some embodiments, the secretion tag is the dmsA secretion signal. In some embodiments, the secretion tag comprises SEQ ID NO: 751. In some embodiments, the secretion tag is the PelB secretion signal. In some embodiments, the secretion tag comprises SEQ ID NO: 752. In some embodiments, the secretion tag is the HlyA secretion signal. In some embodiments, the secretion tag comprises a sequence selected from SEQ ID NO: 753 and SEQ ID NO: 754.

In some embodiments, the genetically engineered bacteria encode a polypeptide comprising a secretion tag selected from Adhesin (ECOLIN_19880), DsbA (ECOLIN_21525), GltI (ECOLIN_03430), GspD (ECOLIN_16495), HdeB (ECOLIN_19410), MalE (ECOLIN_22540), OppA (ECOLIN_07295), PelB, PhoA (ECOLIN_02255), PpiA (ECOLIN_18620), TolB, tort, OmpA, PelB, DsbA mglB, and lamB secretion tags. Exemplary sequences of secretion tags are shown in SEQ ID NO: 1222, SEQ ID NO: 1223, SEQ ID NO: 1224, SEQ ID NO: 1225, SEQ ID NO: 1226, SEQ ID NO: 1227, SEQ ID NO: 1228, SEQ ID NO: 1229, SEQ ID NO: 1230, SEQ ID NO: 1141, SEQ ID NO: 1142, SEQ ID NO: 1143, SEQ ID NO: 1144, SEQ ID NO: 1145, SEQ ID NO: 1253, SEQ ID NO: 1157, SEQ ID NO: 1158, SEQ ID NO: 1159, SEQ ID NO: 1160, SEQ ID NO: 1161, SEQ ID NO: 1162, SEQ ID NO: 1163, SEQ ID NO: 1164, SEQ ID NO: 1165, SEQ ID NO: 1166, and SEQ ID NO: 1167.

In some embodiments, a secretion tag polypeptide sequence may be selected from SEQ ID NO: 1218, SEQ ID NO: 1219, SEQ ID NO: 1181, SEQ ID NO: 1220, SEQ ID NO: 1221, SEQ ID NO: 1180, SEQ ID NO: 1184, SEQ ID NO: 1186, SEQ ID NO: 1190, SEQ ID NO: 1182, SEQ ID NO: 1135, SEQ ID NO: 1183, SEQ ID NO: 1188, SEQ ID NO: 1187, SEQ ID NO: 747, SEQ ID NO: 1185, and SEQ ID NO: 1189.

Any secretion tag or secretion system can be combined with any immune modulator described herein intended for secretion. In some embodiments, the secretion system is used in combination with one or more genomic mutations, which leads to the leaky or diffusible outer membrane phenotype (DOM), including but not limited to, lpp, nlP, tolA, PAL. In some embodiments, the therapeutic proteins secreted by the genetically engineered bacteria are modified to increase resistance to proteases, e.g. intestinal proteases.

In some embodiments, the therapeutic polypeptides of interest, e.g., the immune modulators, e.g., immune initiators and/or immune sustainers described herein, are secreted via a diffusible outer membrane (DOM) system. In some embodiments, the therapeutic polypeptide of interest is fused to a N-terminal Sec-dependent secretion signal. Non-limiting examples of such N-terminal Sec-dependent secretion signals include PhoA, OmpF, OmpA, and cvaC. In alternate embodiments, the therapeutic polypeptide of interest is fused to a Tat-dependent secretion signal. Exemplary Tat-dependent tags include TorA, FdnG, and DmsA.

In certain embodiments, the genetically engineered bacteria comprise deletions or mutations in one or more of the outer membrane and/or periplasmic proteins. Non-limiting examples of such proteins, one or more of which may be deleted or mutated, include lpp, pal, tolA, and/or nlpI. In some embodiments, lpp is deleted or mutated. In some embodiments, pal is deleted or mutated. In some embodiments, tolA is deleted or mutated. In other embodiments, nlpI is deleted or mutated. In yet other embodiments, certain periplasmic proteases are deleted or mutated, e.g., to increase stability of the polypeptide in the periplasm. Non-limiting examples of such proteases include degP and ompT. In some embodiments, degP is deleted or mutated. In some embodiments, ompT is deleted or mutated. In some embodiments, degP and ompT are deleted or mutated.

### Surface Display

In some embodiments, the genetically engineered bacteria and/or microorganisms encode one or more gene(s) and/or gene cassette(s) encoding a display protein comprising an anchor domain, a linker, and a displayed protein (e.g., viral, bacterial, fungal, and cancer protein), and/or an immune modulator which is anchored or displayed on the surface of the bacteria and/or microorganisms.

In some embodiments, a viral spike protein is displayed as a viral protein on the surface of the bacteria and/or microorganisms. In other embodiments, the receptor binding domain (RBD) of a spike protein, e.g., a RBD of S protein from SARS-CoV-2, is displayed on the surface of the bacteria and/or microorganisms. Additional non-limiting examples of such viral proteins which may be produced by the bacteria of the disclosure include those peptides and/or epitopes described e.g., in Liu W J., et al. 2017, Antiviral Research 137:82-92; Huang J., et al. 2007, Vaccine 25: 6981-6991; Chen H., et al., 2005, J Immunol 175: 591-598; Ahmed S. F., et al., 2020, Viruses 12: 254; and Grifoni A., et al., Cell Host & Microbe 27: 1-10; the contents of each of which is herein incorporated by reference in its entirety or otherwise known in the art.

Examples of the immune modulators which are displayed or anchored to the bacteria and/or microorganism, are any of the immune modulators described herein, and include but are not limited to antibodies, e.g., scFv fragments, and tissue-specific antigens or neoantigens. In a non-limiting example, the antibodies or scFv fragments which are anchored or displayed on the bacterial cell surface are directed against checkpoint inhibitors described herein, including, but not limited to, CLTLA4, PD-1, PD-L1.

Suitable systems for surface display of heterologous polypeptides, e.g., effector molecules, on the surface of gram negative and gram positive bacteria are described in International Patent Application PCT/US2017/013072, filed Jan. 11, 2017, published as WO2017/123675, the contents of which is herein incorporated by reference in its entirety

In some embodiments, the genetically engineered bacteria comprise a gene sequence encoding a therapeutic polypeptide comprising an invasin display tag. In one embodiment, the genetically engineered bacteria comprise a gene sequence encoding a polypeptide comprising SEQ ID NO: 990.

In some embodiments, the genetically engineered bacteria comprise a gene sequence encoding a therapeutic polypeptide comprising an LppOmpA display tag. In one embodiment, the genetically engineered bacteria comprise a gene sequence encoding a polypeptide comprising SEQ ID NO: 991.

In some embodiments, the genetically engineered bacteria comprise a gene sequence encoding a therapeutic polypeptide comprising an intimin N display tag. In one embodiment, the genetically engineered bacteria comprise a gene sequence encoding a polypeptide comprising SEQ ID NO: 992.

In some embodiments, the genetically engineered bacteria comprise a display anchor which is at least about 80%, at least about 85%, at least about 90%, at least about 95%, or at least about 99% homologous to a sequence selected from SEQ ID NO: 990, SEQ ID NO: 991, and SEQ ID NO: 992. In another embodiment, the genetically engineered bacteria comprise a gene sequence encoding display anchor comprising a sequence selected from SEQ ID NO: 990, SEQ ID NO: 991, and SEQ ID NO: 992. In yet another embodiment, the display anchor expressed by the genetically engineered bacteria consists of a sequence selected from SEQ ID NO: 990, SEQ ID NO: 991, and SEQ ID NO: 992.

In some embodiments, one or more ScFvs are displayed on the bacterial cell surface, alone or in combination with other therapeutic polypeptides of interest.

In some embodiments, a cell surface display strategy or circuit is combined with a secretion strategy or circuit in one bacterium. In some embodiments, the same polypeptide is both displayed and secreted. In some embodiments, a first polypeptide is displayed and a second is secreted. In some embodiments, a display strategy or circuit strategy is combined with a circuit for the intracellular production of an enzyme and consequentially intracellular catabolism of its substrate. In some embodiments, a display strategy or display circuit is combined with a circuit for the intracellular production of a gut barrier enhancer molecule and/or an anti-inflammatory effector molecule.

In some embodiments, the expression of the surface displayed polypeptide or fusion protein is driven by an inducible promoter. In alternate embodiments, expression of the surface displayed polypeptides or polypeptide fusion proteins is driven by a constitutive promoter.

In some embodiments, the expression of the surface displayed polypeptide or fusion protein is plasmid based. In some embodiments, the gene sequence(s) encoding the antibodies or scFv fragments for surface display is chromosomally inserted.

### Essential Genes, Auxotrophs, Kill Switches, and Host-Plasmid Dependency

As used herein, the term “essential gene” refers to a gene that is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37:D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in a gene required for cell survival and/or growth. In one embodiment, the essential gene is a DNA synthesis gene, for example, thyA. In another embodiment, the essential gene is a bacterial cell wall synthesis gene, for example, dapA. In yet another embodiment, the essential gene is an amino acid gene, for example, serA or metA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thil, as long as the corresponding wild-type gene product is not produced in the bacteria. Exemplary bacterial genes which may be disrupted or deleted to produce an auxotrophic strain as described in International Patent Application PCT/US2017/013072, filed Jan. 11, 2017, published as WO2017/123675, the contents of which is herein incorporated by reference in its entirety. These include, but are not limited to, genes required for oligonucleotide synthesis, amino acid synthesis, and cell wall synthesis. Table 6 lists exemplary bacterial genes which may be disrupted or deleted to produce an auxotrophic strain. These include, but are not limited to, genes required for oligonucleotide synthesis, amino acid synthesis, and cell wall synthesis.

TABLE 6
Non-limiting Examples of Bacterial Genes
Useful for Generation of an Auxotroph

| Amino Acid | Oligonucleotide | Cell Wall |
|---|---|---|
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Auxotrophic mutations are useful in some instances in which biocontainment strategies may be required to prevent unintended proliferation of the genetically engineered bacterium in a natural ecosystem. Any auxotrophic mutation in an essential gene described above or known in the art can be useful for this purpose, e.g. DNA synthesis genes, amino acid synthesis genes, or genes for the synthesis of cell wall. Accordingly, in some embodiments, the genetically engineered bacteria comprise modifications, e.g., mutation(s) or deletion(s) in one or more auxotrophic genes, e.g., to prevent growth and proliferation of the bacterium in the natural environment. In some embodiments, the modification may be located in a non-coding region. In some embodiments, the modifications result in attenuation of transcription or translation. In some embodiments, the modifications, e.g., mutations or deletions, result in reduced or no transcription or reduced or no translation of the essential gene. In some embodiments, the modifications, e.g., mutations or deletions, result in transcription and/or translation of a non-functional version of the essential gene. In some embodiments, the modifications, e.g., mutations or deletions result in in truncated transcription or translation of the essential gene, resulting in a truncated polypeptide. In some embodiments, the modification, e.g., mutation is located within the coding region of the gene.

While unable to grow in the natural ecosystem, certain auxotrophic mutations may allow growth and proliferation in the mammalian host administered the bacteria. For example, an essential pathway that is rendered non-functional by the auxotrophic mutation may be complemented by production of the metabolite by the host. As a result, the bacterium administered to the host can take up the metabolite from the environment and can proliferate and colonize the target site. Thus, in some embodiments, the auxotrophic gene is an essential gene for the production of a metabolite, which is also produced by the mammalian host in vivo. In some embodiments, metabolite production by the host may allow uptake of the metabolite by the bacterium and permit survival and/or proliferation of the bacterium within the target site. In some embodiments, bacteria comprising such auxotrophic mutations are capable of proliferating and colonizing the target site to the same extent as a bacterium of the same subtype which does not carry the auxotrophic mutation.

In some embodiments, the bacteria are capable of colonizing and proliferating in the target microenvironment. In some embodiments, the target colonizing bacteria comprise one or more auxotrophic mutations. In some embodiments, the target colonizing bacteria do not comprise one or more auxotrophic modifications or mutations. In a non-limiting example, greater numbers of bacteria are detected after 24 hours and 72 hours than were originally injected into the subject. In some embodiments, CFUs detected 24 hours post injection are at least about 1 to 2 logs greater than administered. In some embodiments, CFUs detected 24 hours post injection are at least about 2 to 3 logs greater than administered. In some embodiments, CFUs detected 24 hours post injection are at least about 3 to 4 logs greater than administered. In some embodiments, CFUs detected 24 hours post injection are at least about 4 to 5 logs greater than administered. In some embodiments, CFUs detected 24 hours post injection are at least about 5 to 6 logs greater than administered. In some embodiments, CFUs detected 72 hours post injection are at least about 1 to 2 logs greater than administered. In some embodiments, CFUs detected 72 hours post injection are at least about 2 to 3 logs greater than administered. In some embodiments, CFUs detected 72 hours post injection are at least about 3 to 4 logs greater than administered. In some embodiments, CFUs detected 72 hours post injection are at least about 4 to 5 logs greater than administered. In some embodiments, CFUs detected 72 hours post injection are at least about 5 to 6 logs greater than administered. In some embodiments, CFUs can be measured at later time points, such as after at least one week, after at least 2 or more weeks, after at least one month, after at least two or more months post injection.

Non-limiting examples of such auxotrophic genes, which allow proliferation and colonization of the target, are thyA and uraA, as shown herein. Accordingly, in some embodiments, the genetically engineered bacteria of the disclosure may comprise an auxotrophic modification, e.g., mutation or deletion, in the thyA gene. In some embodiments, the genetically engineered bacteria of the disclosure may comprise an auxotrophic modification, e.g., mutation or deletion, in the uraA gene. In some embodiments, the genetically engineered bacteria of the disclosure may comprise auxotrophic modification, e.g., mutation or deletion, in the thyA gene and the uraA gene.

Alternatively, the auxotrophic gene is an essential gene for the production of a metabolite which cannot be produced by the host within the target, i.e., the auxotrophic mutation is not complemented by production of the metabolite by the host within the target microenvironment. As a result, the this mutation may affect the ability of the bacteria to grow and colonize the target and bacterial counts decrease over time. This type of auxotrophic mutation can be useful for the modulation of in vivo activity of the immune modulator or duration of activity of the immune modulator, e.g., within a target. An example of this method of fine-tuning levels and timing of immune modulator release is described herein using a auxotrophic modification, e.g., mutation, in dapA. Diaminopimelic acid (Dap) is a characteristic component of certain bacterial cell walls, e.g., of gram negative bacteria. Without diaminopimelic acid, bacteria are unable to form proteoglycan, and as such are unable to grow. DapA is not produced by mammalian cells, and therefore no alternate source of DapA is provided in the target. As such, a dapA auxotrophy may present a particularly useful strategy to modulate and fine tune timing and extent of bacterial presence in the target and/or levels and timing of immune modulator expression and production. Accordingly, in some embodiments, the genetically engineered bacteria of the disclosure comprise an mutation in an essential gene for the production of a metabolite which cannot be produced by the host within the target. In some embodiments, the auxotrophic mutation is in a gene which is essential for the production and maintenance of the bacterial cell wall known in the art or described herein, or a mutation in a gene that is essential to another structure that is unique to bacteria and not present in mammalian cells. In some embodiments, bacteria comprising such auxotrophic mutations are capable of proliferating and colonizing the target to a substantially lesser extent than a bacterium of the same subtype which does not carry the auxotrophic mutation. Control of bacterial growth (and by extent effector levels) may be further combined with other regulatory strategies, including but not limited to, metabolite or chemically inducible promoters described herein.

In a non-limiting example, lower numbers of bacteria are detected after 24 hours and 72 hours than were originally injected into the subject. In some embodiments, CFUs detected 24 hours post injection are at least about 1 to 2 logs lower than administered. In some embodiments, CFUs detected 24 hours post injection are at least about 2 to 3 logs lower than administered. In some embodiments, CFUs detected 24 hours post injection are at least about 3 to 4 logs lower than administered. In some embodiments, CFUs detected 24 hours post injection are at least about 4 to 5 logs lower than administered. In some embodiments, CFUs detected 24 hours post injection are at least about 5 to 6 logs lower than administered. In some embodiments, CFUs detected 72 hours post injection are at least about 1 to 2 logs lower than administered. In some embodiments, CFUs detected 72 hours post injection are at least about 2 to 3 logs lower than administered. In some embodiments, CFUs detected 72 hours post injection are at least about 3 to 4 logs lower than administered. In some embodiments, CFUs detected 72 hours post injection are at least about 4 to 5 logs lower than administered. In some embodiments, CFUs detected 72 hours post injection are at least about 5 to 6 logs lower than administered. In some embodiments, CFUs can be measured at later time points, such as after at least one week, after at least 2 or more weeks, after at least one month, after at least two or more months post injection.

In some embodiments, the genetically engineered bacteria of the disclosure comprise a auxotrophic modification, e.g., mutation, in dapA. A non-limiting example described herein is a genetically engineered bacterium comprising gene sequences encoding dacA for c-di-AMP production. Production of the STING agonist can be temporally regulated or restricted through the introduction of a dapA auxotrophy. In some embodiments, the dapA auxotrophy provides a means for tunable STING agonist production.

Auxotrophic modifications may also be used to screen for mutant bacteria that produce the effector molecule for various applications. In one example, the auxotrophy is useful to monitor purity or “sterility” of batches in small and large scale production of a bacterial strain. In this case, the auxotrophy presents a means to distinguish the engineered bacterium from a potential contaminant. In a non-limiting example, during the manufacturing process of the live biotherapeutic (i.e., large scale), an auxotrophy can be a useful tool to demonstrate purity or “sterility” of the drug substance. This method to determine purity of the culture is particularly useful in the absence of an antibiotic resistance gene, which is often used for this purpose in experimental strains, but which may be removed during the development of the live therapeutic drug product.

trpE is another auxtrophic mutation described herein. Bacteria carrying this mutation cannot produce tryptophan. Genetically engineered bacteria described herein with a trpE mutation further comprise kynureninase. Kynureninase allows the bacterium to convert kynurenine into the tryptophan precursor anthranilate and therefore the bacterium can grow in the absence of tryptophan if kynurenine is present.

In some embodiments, the genetically engineered bacteria comprise auxotrophic mutation(s) in one essential gene. In some embodiments, the genetically engineered bacteria comprise auxotrophic mutation(s) in two essential genes (double auxotrophy). In some embodiments, the genetically engineered bacteria comprise auxotrophic mutation(s) in three or more essential gene(s).

In some embodiments, the genetically engineered bacteria comprise auxotrophic mutation(s) in dapA and thyA. In some embodiments, the genetically engineered bacteria comprise auxotrophic mutation(s) in dapA and uraA. In some embodiments, the genetically engineered bacteria comprise auxotrophic mutation(s) in thyA and uraA. In some embodiments, the genetically engineered bacteria comprise auxotrophic mutation(s) in dapA, thyA and uraA.

In some embodiments, the genetically engineered bacteria comprise auxotrophic mutation(s) in trpE. In some embodiments, the genetically engineered bacteria comprise auxotrophic mutation(s) in trpE and thyA. In some embodiments, the genetically engineered bacteria comprise auxotrophic mutation(s) in trpE and dapA. In some embodiments, the genetically engineered bacteria comprise auxotrophic mutation(s) in trpE and uraA. In some embodiments, the genetically engineered bacteria comprise auxotrophic mutation(s) in trpE, dapA and thyA. In some embodiments, the genetically engineered bacteria comprise auxotrophic mutation(s) in trpE, dapA and uraA. In some embodiments, the genetically engineered bacteria comprise auxotrophic mutation(s) in trpE, thyA and uraA. In some embodiments, the genetically engineered bacteria comprise auxotrophic mutation(s) in trpE, dapA, thyA and uraA.

In another non-limiting example, a conditional auxotroph can be generated. The chromosomal copy of dapA or thyA is knocked out. Another copy of thyA or dapA is introduced, e.g., under control of a low oxygen promoter. Under anaerobic conditions, dapA or thyA—as the case may be—are expressed, and the strain can grow in the absence of dap or thymidine. Under aerobic conditions, dapA or thyA expression is shut off, and the strain cannot grow in the absence of dap or thymidine. Such a strategy can also be employed to allow survival of bacteria under anaerobic conditions, e.g., the gut or conditions of the target microenvironment, but prevent survival under aerobic conditions.

In some embodiments, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain, “ACS Synthetic Biology (2015) DOI: 10.1021/acssynbio.5b00085, the entire contents of which are expressly incorporated herein by reference). SLiDE bacterial cells are described in International Patent Application PCT/US2017/013072, filed Jan. 11, 2017, published as WO2017/123675, the contents of which is herein incorporated by reference in its entirety.

In some embodiments, the genetically engineered bacteria of the invention also comprise a kill switch. Suitable kill switches are described in International Patent Application PCT/US2016/39427, filed Jun. 24, 2016, published as WO2016/210373, the contents of which are herein incorporated by reference in their entirety. The kill switch is intended to actively kill engineered microbes in response to external stimuli. As opposed to an auxotrophic mutation where bacteria die because they lack an essential nutrient for survival, the kill switch is triggered by a particular factor in the environment that induces the production of toxic molecules within the microbe that cause cell death.

In some embodiments, the genetically engineered bacteria of the invention also comprise a plasmid that has been modified to create a host-plasmid mutual dependency. In certain embodiments, the mutually dependent host-plasmid platform is as described in Wright et al., 2015. These and other systems and platforms are described in International Patent Application PCT/US2017/013072, filed Jan. 11, 2017, published as WO2017/123675, the contents of which is herein incorporated by reference in its entirety.

### Genetic Regulatory Circuits

In some embodiments, the genetically engineered bacteria comprise multi-layered genetic regulatory circuits for expressing the constructs described herein. Suitable multi-layered genetic regulatory circuits are described in International Patent Application PCT/US2016/39434, filed on Jun. 24, 2016, published as WO2016/210378, the contents of which is herein incorporated by reference in its entirety. The genetic regulatory circuits are useful to screen for mutant bacteria that produce an immune modulator or rescue an auxotroph. In certain embodiments, the invention provides methods for selecting genetically engineered bacteria that produce one or more genes of interest.

### Pharmaceutical Compositions and Formulations

Pharmaceutical compositions comprising the genetically engineered microorganisms of the invention may be used to treat, manage, ameliorate, and/or prevent viral infection, e.g., the coronavirus disease 2019 (COVID-19). Pharmaceutical compositions of the invention comprising one or more genetically engineered bacteria, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise the genetic modifications described herein, e.g., one or more genes encoding one or more viral protein, e.g., a spike protein of SARV-CoV-2, and one or more effectors, e.g., immune modulators. In alternate embodiments, the pharmaceutical composition comprises two or more species, strains, and/or subtypes of bacteria that are each engineered to comprise the genetic modifications described herein, e.g., one or more genes encoding one or more effectors, e.g., immune modulators.

In some embodiments, the genetically engineered bacteria are administered systemically. In some embodiments, the genetically engineered bacteria are administered intranasally. The pharmaceutical compositions of the invention may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tableting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, intranasal, intratumoral, peritumor, immediate-release, pulsatile-release, delayed-release, or sustained release). Suitable dosage amounts for the genetically engineered bacteria may range from about 10⁴ to 10¹² bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal.

The genetically engineered bacteria may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection. In other embodiments, the genetically engineered microorganisms may be administered intra-arterially, intramuscularly, or intraperitoneally. In some embodiments, the genetically engineered bacteria colonize about 20%, 30%, 40%, 50%, 60%, 70%, 80%, 90% or more of the target.

The genetically engineered microorganisms of the disclosure may be administered via intranasal delivery, resulting in bacteria or virus that is directly deposited within the target site. Intranasal delivery of the engineered bacteria or virus may elicit a potent localized inflammatory response as well as an adaptive immune response against the target cells. Bacteria or virus are suspended in solution before being withdrawn into a 1-ml syringe.

Single insertion points or multiple insertion points can be used in percutaneous injection protocols. Using a single insertion point, the solution may be injected percutaneously along multiple tracks, as far as the radial reach of the needle allows. In other embodiments, multiple injection points may be used if the target is larger than the radial reach of the needle. The needle can be pulled back without exiting, and redirected as often as necessary until the full dose is injected and dispersed. To maintain sterility, a separate needle is used for each injection. Needle size and length varies depending on the tissue type.

In some embodiments, the target site is injected percutaneously with an 18-gauge multipronged needle (Quadra-Fuse, Rex Medical). The device consists of an 18 gauge puncture needle 20 cm in length. The needle has three retractable prongs, each with four terminal side holes and a connector with extension tubing clamp. The prongs are deployed from the lateral wall of the needle. The needle can be introduced percutaneously into the center of the target and can be positioned at the deepest margin of the target. The prongs are deployed to the margins of the target. The prongs are deployed at maximum length and then are retracted at defined intervals. Optionally, one or more rotation-injection-rotation maneuvers can be performed, in which the prongs are retracted, the needle is rotated by a 60 degrees, which is followed by repeat deployment of the prongs and additional injection.

In some embodiments, bacteria, e.g., E. coli Nissle, or spores, e.g., Clostridium novyi NT, are dissolved in sterile phosphate buffered saline (PBS) for systemic injection.

In some embodiments, the treatment regimen will include one or more intranasal administrations. In some embodiments, a treatment regimen will include an initial dose, which followed by at least one subsequent dose. One or more doses can be administered sequentially in two or more cycles.

For example, a first dose may be administered at day 1, and a second dose may be administered after 1, 2, 3, 4, 5, 6, days or 1, 2, 3, or 4 weeks or after a longer interval. Additional doses may be administered after 1, 2, 3, 4, 5, 6, days or after 1, 2, 3, or 4 weeks or longer intervals. In some embodiments, the first and subsequent administrations have the same dosage. In other embodiments, different doses are administered. In some embodiments, more than one dose is administered per day, for example, two, three or more doses can be administered per day.

The routes of administration and dosages described are intended only as a guide. The optimum route of administration and dosage can be readily determined by a skilled practitioner. The dosage may be determined according to various parameters, especially according to the location of the target, the size of the target, the age, weight and condition of the patient to be treated and the route and method of administration.

In one embodiment, Clostridium spores are delivered systemically. In another embodiment, Clostridium spores are delivered via intranasal delivery. In one embodiment, E. coli Nissle are delivered via intranasal delivery. In other embodiments, E. coli Nissle is administered via intravenous injection or orally, as described in a mouse model in for example in Danino et al. 2015, or Stritzker et al., 2007, the contents of which is herein incorporated by reference in its entirety. E. coli Nissle mutations to reduce toxicity include but are not limited to msbB mutants resulting in non-myristoylated LPS and reduced endotoxin activity, as described in Stritzker et al., 2010 (Stritzker et al, Bioengineered Bugs 1:2, 139-145; Myristylation negative msbB-mutants of probiotic E. coli Nissle 1917 retain tissue specific colonization properties but show less side effects in immunocompetent mice.

For intravenous injection a preferred dose of bacteria is the dose in which the greatest number of bacteria is found in the target tissue and the lowest amount found in other tissues. In mice, Stritzker et al (International Journal of Medical Microbiology 297 (2007) 151-162; Tissue specific colonization, tissue distribution, and gene induction by Escherichia coli Nissle 1917 in live mice) found that the lowest number of bacteria needed for successful target colonization was 2e4 CFU, in which half of the mice showed target colonization. Injection of 2e5 and 2e6 CFU resulted in colonization of all targets, and numbers of bacteria in the targets increased. However, at higher concentrations, bacterial counts became detectable in the liver and the spleen.

In some embodiments, the microorganisms of the disclosure may be administered orally. In one embodiment, the genetically engineered microorganism is delivered intranasally. In one embodiment, the genetically engineered microorganisms is delivered intrapleurally. In one embodiment, the genetically engineered microorganism is delivered subcutaneously. In one embodiment, the genetically engineered microorganism is delivered intravenously. In one embodiment, the genetically engineered microorganism is delivered intrapleurally.

In some embodiments, the genetically engineered microorganisms of the invention may be administered intranasally according to a regimen which requires multiple injections. In some embodiments, the same bacterial strains are administered in each injection. In some embodiments, a first strain is injected first and a second strain is injected at a later timepoint. For example, a strain capable of producing an immune initiator, e.g., STING agonist, may be administered concurrently or sequentially with a strain capable of producing another immune initiator, e.g., a co-stimulatory molecule, e.g., agonistic anti-OX40, 41BB, or GITR. Additional injections of the two immune initiators, either concurrently or sequentially, can follow. In another example, a strain capable of producing an immune initiator, e.g., STING agonist, may be administered first, and a strain capable of producing an immune sustainer, e.g., kynurenine consumption, or anti-PD-1/anti-PD-L1 secretion or anti-PD-1/anti-PD-L1 surface display, may be administered second. Additional injections of STING agonist producing strains and/or anti-PD-1/anti-PD-L1 producing strains can follow. Optionally, antibiotics can be used to clear a first strain from the target before injection of a second strain. Alternatively, an auxotrophic modification, e.g., mutation in the dapA gene, which limits colonization, can be incorporated into the first strain, which may eliminate the bacteria of the first strain prior to injection of a second strain.

The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, Pa. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria of the invention may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinized maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered bacteria are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

In some embodiments, enteric coating materials may be used, in one or more coating layers (e.g., outer, inner and/o intermediate coating layers). Enteric coated polymers remain unionized at low pH, and therefore remain insoluble. But as the pH increases in the gastrointestinal tract, the acidic functional groups are capable of ionization, and the polymer swells or becomes soluble in the intestinal fluid.

Materials used for enteric coatings include Cellulose acetate phthalate (CAP), Poly(methacrylic acid-co-methyl methacrylate), Cellulose acetate trimellitate (CAT), Poly(vinyl acetate phthalate) (PVAP) and Hydroxypropyl methylcellulose phthalate (HPMCP), fatty acids, waxes, Shellac (esters of aleurtic acid), plastics and plant fibers. Additionally, Zein, Aqua-Zein (an aqueous zein formulation containing no alcohol), amylose starch and starch derivatives, and dextrins (e.g., maltodextrin) are also used. Other known enteric coatings include ethylcellulose, methylcellulose, hydroxypropyl methylcellulose, amylose acetate phthalate, cellulose acetate phthalate, hydroxyl propyl methyl cellulose phthalate, an ethylacrylate, and a methylmethacrylate.

Coating polymers also may comprise one or more of, phthalate derivatives, CAT, HPMCAS, polyacrylic acid derivatives, copolymers comprising acrylic acid and at least one acrylic acid ester, Eudragit™ S (poly(methacrylic acid, methyl methacrylate)1:2); Eudragit L100™ S (poly(methacrylic acid, methyl methacrylate)1:1); Eudragit L30D™, (poly(methacrylic acid, ethyl acrylate)1:1); and (Eudragit L100-55) (poly(methacrylic acid, ethyl acrylate)1:1) (Eudragit™ L is an anionic polymer synthesized from methacrylic acid and methacrylic acid methyl ester), polymethyl methacrylate blended with acrylic acid and acrylic ester copolymers, alginic acid, ammonia alginate, sodium, potassium, magnesium or calcium alginate, vinyl acetate copolymers, polyvinyl acetate 30D (30% dispersion in water), a neutral methacrylic ester comprising poly(dimethylaminoethylacrylate) (“Eudragit E™), a copolymer of methylmethacrylate and ethylacrylate with trimethylammonioethyl methacrylate chloride, a copolymer of methylmethacrylate and ethylacrylate, Zein, shellac, gums, or polysaccharides, or a combination thereof.

Coating layers may also include polymers which contain Hydroxypropylmethylcellulose (HPMC), Hydroxypropylethylcellulose (HPEC), Hydroxypropylcellulose (HPC), hydroxypropylethylcellulose (HPEC), hydroxymethylpropylcellulose (HMPC), ethylhydroxyethylcellulose (EHEC) (Ethulose), hydroxyethylmethylcellulose (HEMC), hydroxymethylethylcellulose (HMEC), propylhydroxyethylcellulose (PHEC), methylhydroxyethylcellulose (M H EC), hydrophobically modified hydroxyethylcellulose (NEXTON), carboxymethyl hydroxyethylcellulose (CMHEC), Methylcellulose, Ethylcellulose, water soluble vinyl acetate copolymers, gums, polysaccharides such as alginic acid and alginates such as ammonia alginate, sodium alginate, potassium alginate, acid phthalate of carbohydrates, amylose acetate phthalate, cellulose acetate phthalate (CAP), cellulose ester phthalates, cellulose ether phthalates, hydroxypropylcellulose phthalate (HPCP), hydroxypropylethylcellulose phthalate (HPECP), hydroxyproplymethylcellulose phthalate (HPMCP), hydroxyproplymethylcellulose acetate succinate (HPMCAS).

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. For example, see U.S. 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD₅₀, ED₅₀, EC₅₀, and IC₅₀ may be determined, and the dose ratio between toxic and therapeutic effects (LD₅₀/ED₅₀) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the genetically engineered microorganisms and composition thereof is formulated for intravenous administration, intratumor administration, or peritumor administration. The genetically engineered microorganisms may be formulated as depot preparations. Such long acting formulations may be administered by implantation or by injection. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, the genetically engineered OVs are prepared for delivery, taking into consideration the need for efficient delivery and for overcoming the host antiviral immune response. Approaches to evade antiviral response include the administration of different viral serotypes as part of the treatment regimen (serotype switching), formulation, such as polymer coating to mask the virus from antibody recognition and the use of cells as delivery vehicles.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The genetically engineered bacteria of the invention may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### Methods of Treatment

Another aspect provides methods of treating microbial infections, e.g., COVID-19. In some embodiments, the invention provides methods for reducing, ameliorating, or eliminating one or more symptom(s) associated with COVID-19. In some embodiments, the symptom(s) associated thereof include, but are not limited to, runny nose, sneezing, headache, cough, sore throat, fever, or short of breath. In more severe cases, coronavirus infection can cause pneumonia, severe acute respiratory syndrome, kidney failure and even death.

The method may comprise preparing a pharmaceutical composition with at least one genetically engineered species, strain, or subtype of bacteria described herein, and administering the pharmaceutical composition to a subject in a therapeutically effective amount. The genetically engineered microorganisms may be administered intravenously, intranasally, intra-arterially, intramuscularly, intraperitoneally, orally, or topically. In some embodiments, the genetically engineered microorganisms are administered intravenously, i.e., systemically.

In certain embodiments, administering the pharmaceutical composition to the subject reduces viral infection in a subject. In some embodiments, the methods of the present disclosure may reduce viral infection by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to levels in an untreated or control subject.

For genetically engineered microorganisms expressing immune-based immune modulators, responses patterns may be different than for traditional cytotoxic therapies. Thus, the pharmaceutical composition comprising the gene or gene cassette for producing the immune modulator may be re-administered at a therapeutically effective dose and frequency. In alternate embodiments, the genetically engineered bacteria are not destroyed within hours or days after administration and may propagate in the target site.

The pharmaceutical composition may be administered alone or in combination with one or more additional therapeutic agents, e.g., as described herein and known in the art. An important consideration in selecting the one or more additional therapeutic agents is that the agent(s) should be compatible with the genetically engineered bacteria of the invention, e.g., the agent(s) must not kill the bacteria.

In certain embodiments, the pharmaceutical composition may be administered to a subject by administering a first genetically engineered bacterium to the subject, wherein the first genetically engineered bacterium comprises at least one gene encoding a first immune initiator; and administering a second genetically engineered bacterium to the subject, wherein the second genetically engineered bacterium comprising at least one gene encoding a second immune initiator. In some embodiments, the administering steps are performed at the same time. In some embodiments, administering the first genetically engineered bacterium to the subject occurs before the administering of the second genetically engineered bacterium to the subject. In some embodiments, administering of the second genetically engineered bacterium to the subject occurs before the administering of the first genetically engineered bacterium to the subject. In some embodiments, the ratio of the first genetically engineered bacterium to the second genetically engineered bacterium is 5:1, 4:1, 3:1, 2:1, 1:1, 1:2, 1:3, 1:4, or 1:5. In some embodiments, the ratio of the second genetically engineered bacterium to the first genetically engineered bacterium is 5:1, 4:1, 3:1, 2:1, 1:1, 1:2, 1:3, 1:4, or 1:5.

### Treatment In Vivo

The modified microorganisms may be evaluated in vivo, e.g., in an animal model. Any suitable animal model of a disease or condition associated with COVID-19 may be used. The genetically engineered bacteria may be administered to the animal systemically or locally, e.g., via oral administration (gavage), intravenous, or subcutaneous injection or via intranasal injection, and treatment efficacy determined.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The Examples do not in any way limit the disclosure.

The disclosure provides herein a sequence having at least about 80%, at least about 85%, at least about 90%, at least about 95%, or at least about 99% homologous to the sequence any of the SEQ ID NOs described in the Examples, below.

### Example 1. Development of a Recombinant Bacteria for Surface Display of Proteins

Microbial infection can be caused by bacteria, fungi, and viruses. A vaccine for the prevention and/or treatment of a bacterial, viral, or fungal infection is developed by utilizing synthetic biology techniques to engineer probiotic bacteria that express one or more proteins (e.g., viral, bacterial, fungal, and cancer) and immune activators/adjuvants. This vaccine is based on an engineered E. coli Nissle (EcN) bacterial strain that expresses one or more proteins (e.g., viral, bacterial, fungal, and cancer) and can be administered, e.g., intranasally, to induce protective immunity systemically and/or at mucosal surfaces.

Engineered E. coli Nissle cells are designed to display one or more proteins (e.g., viral, bacterial, fungal, and cancer) and immune activators/adjuvants on the Nissle membrane. The proteins and immune activators/adjuvants are expressed as fusion proteins referred to herein as “display proteins” comprising an anchor domain(s), a linker, and a displayed protein(s). In some embodiments, the displayed protein is a reporter protein, e.g., GFP (FIG. 1).

To begin with, to create an E. coli Nissle derivative capable of secreting bioactive proteins, a engineered a diffusible outer membrane (DOM) phenotype was generated by deleting the gene encoding the periplasmic protein peptidoglycan associated lipoprotein (PAL, MQLNKVLKGLMIALPVMAIAACSSNKNASNDGSEGMLGAGTGMDANGGNGNMSSEEQAR LQMQQLQQNNIVYFDLDKYDIRSDFAQMLDAHANFLRSNPSYKVTVEGHADERGTPEYNIS LGERRANAVKMYLQGKGVSADQISIVSYGKEKPAVLGHDEAAYAKNRRAVLVY (SEQ ID NO: 1483). This alteration results in an increased rate of diffusion of periplasmic proteins to the external environment without compromising cell growth properties. The resulting ‘leaky’ chassis strain is designated SYN1557 (Nissle delta PAL::CmR).

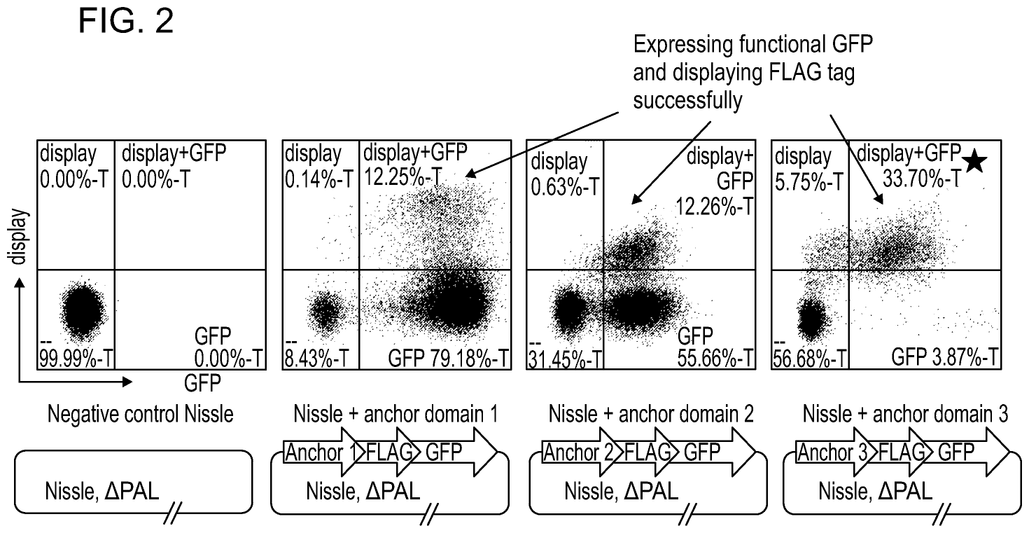

E. coli Nissle cells were further designed to express fusion proteins including an anchor domain, an AB epitope (e.g., FLAG tag), and a displayed reporter, e.g., GFP. Constructs were transformed into SYN1557 (deltaPAL, diffusible outer membrane (DOM) phenotype). As shown in FIG. 2, GFP and FLAG tag were displayed when combined with three different anchor domains and analyzed by flow cytometry. Briefly, a bacterial culture expressing either a negative control construct or surface display construct was centrifuged and the pellet was washed in PBS. The bacterial pellet was resuspended in PBS and antibody was added (e.g., anti-GFP and anti-FLAG). The mixture was incubated at room temperature for 30 minutes. After incubation, the cell and antibody mixture was centrifuged and the resulting cell pellet was resuspended in PBS. Centrifugation and resuspension of the cell pellet was repeated. The resulting cell pellet was resuspended in PBS and the cell suspension was analyzed by flow cytometry. Macquant VYB flow cytometer was used to analyze the samples and collected the data. The data were analyzed using Macsquantify software provided by Miltenyi. FSC=580V, SSC=340V, Y2 (red)=820V, B1 (green)=660V. The FSC and SSC were set up specifically for the visualization of E. coli Nissle. The gating strategy was set up facilitated by negative control and E. coli Nissle strain.

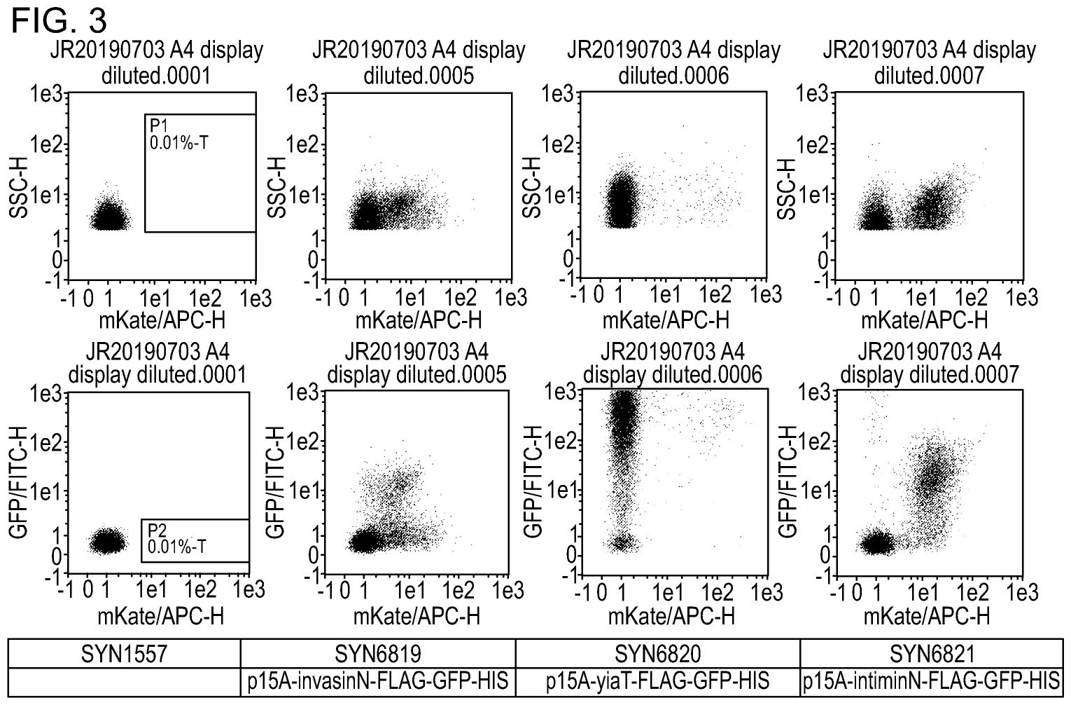

GFP was displayed when using constructs containing InvasionN, YiaT, or IntiminT as the anchor domain (FIG. 3). The constructs are shown in Table 7 below.

TABLE 7
Strains for GFP display (FIG. 3)

| Strain Number | Construct |
|---|---|
| SYN1557 | E. coli Nissle delta PAL:CmR |
| SYN6819 | p15A-invasinN-FLAG-GFP-HIS |
| SYN6820 | p15A-yiaT-FLAG-GFP-HIS |
| SYN6821 | p15A-intiminN-FLAG-GFP-HIS |

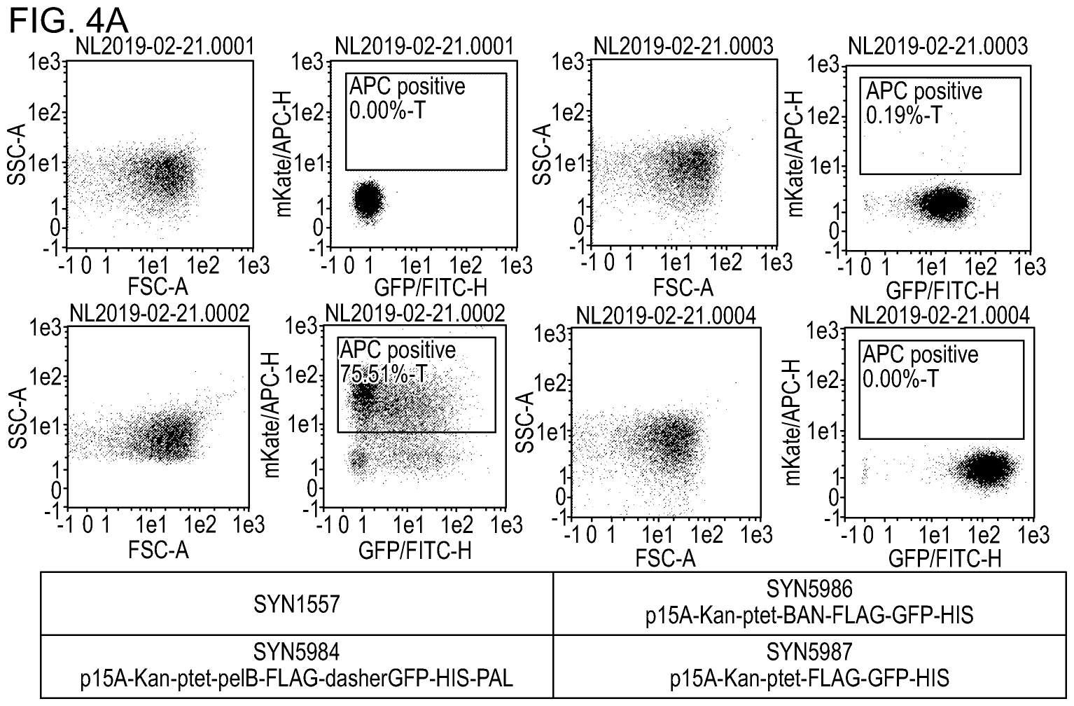

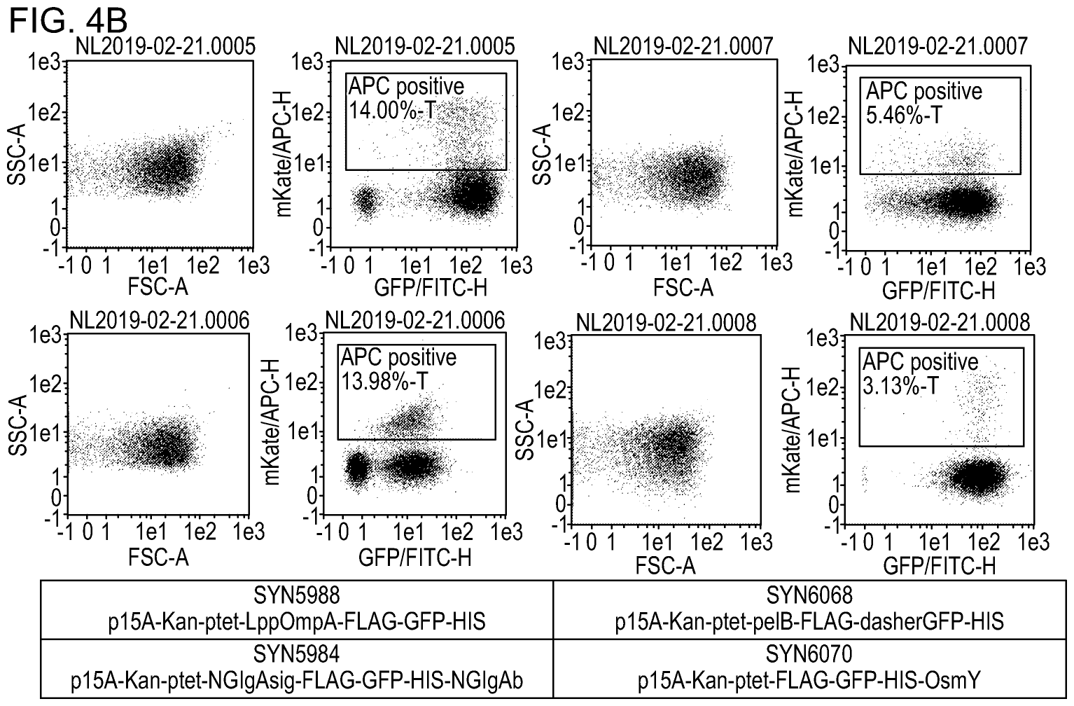

GFP was displayed when using constructs containing pelB-PAL, BAN, LppOmpA, NGIgAsig, or OsmY as the anchor domain (FIGS. 4A and 4B). The constructs are shown in Table 8 below. Construct sequences are shown in Tables 10 and 11 below. Amino acid sequences are shown in Table 12.

TABLE 8
Strains for GFP display (FIGs. 4A and 4B)

| Strain Number | Construct |
|---|---|
| SYN1557 | E. coli Nissle delta PAL:CmR |
| SYN5984 | p15A-Kan-ptet-pelB-FLAG-dasherGFP-HIS-PAL |
| SYN5986 | p15A-Kan-ptet-BAN-FLAG-GFP-HIS |
| SYN5987 | p15A-Kan-ptet-FLAG-GFP-HIS |
| SYN5988 | p15A-Kan-ptet-LppOmpA-FLAG-GFP-HIS |
| SYN5989 | p15A-Kan-ptet-NGIgAsig-FLAG-GFP-HIS-NGIgAb |
| SYN6068 | p15A-Kan-ptet-pelB-FLAG-dasherGFP-HIS |
| SYN6070 | p15A-Kan-ptet-FLAG-GFP-HIS-OsmY |

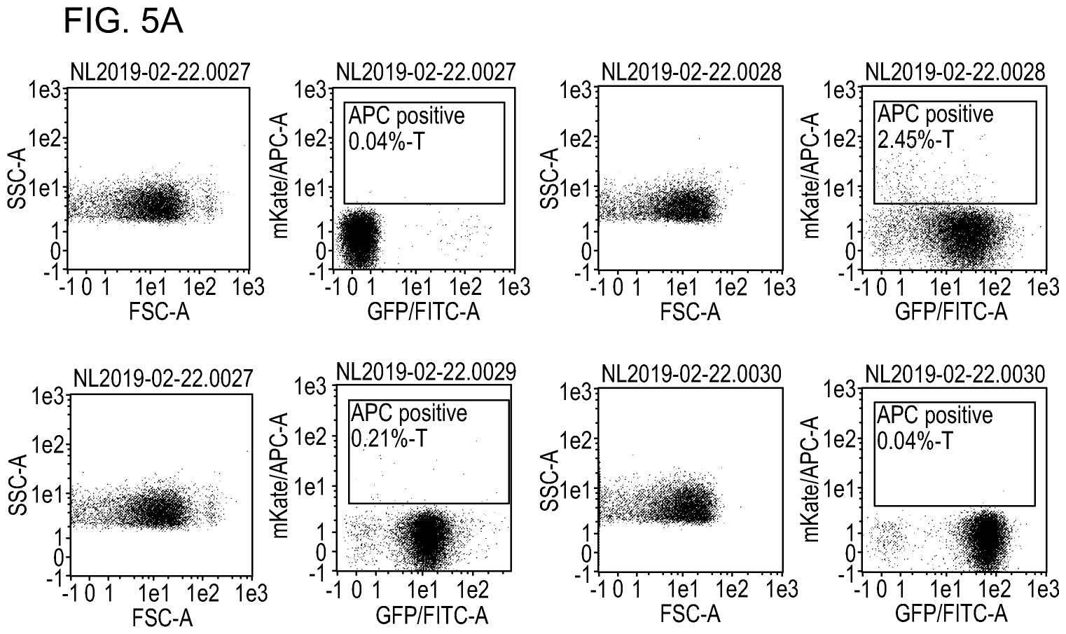

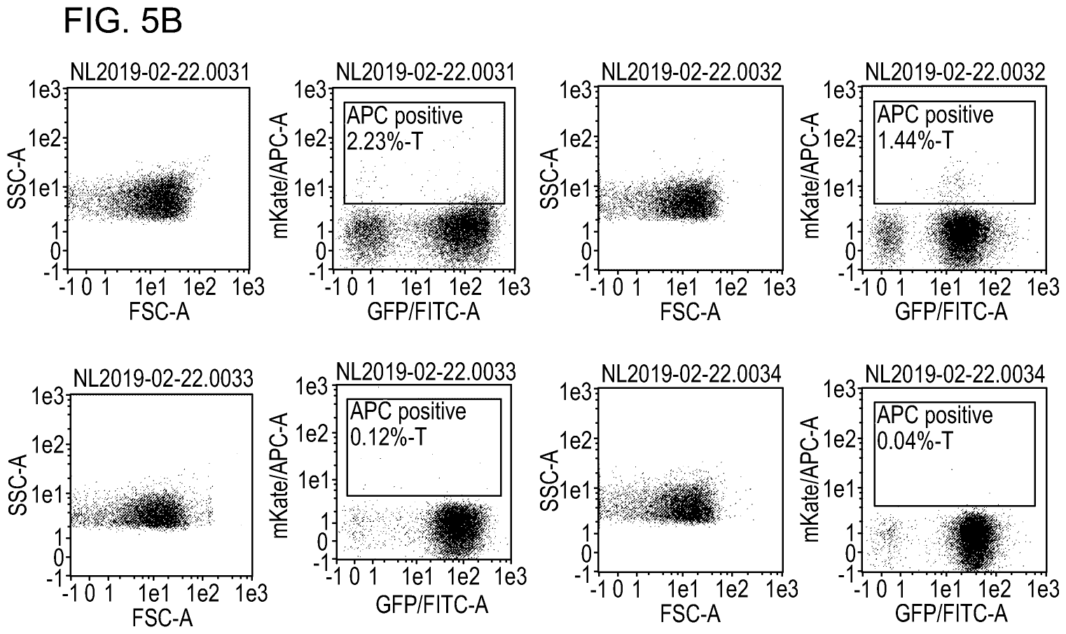

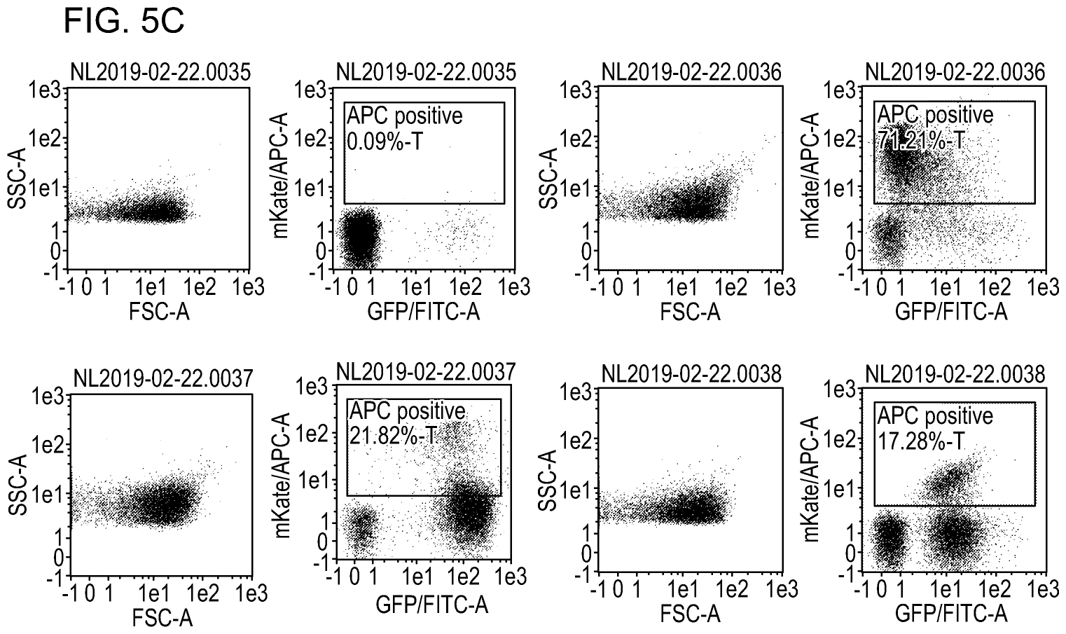

GFP was displayed when using constructs containing pelB-PAL, BAN, LppOmpA, NGIgAsig, or OsmY as the anchor domain (FIGS. 5A-5C). The constructs are shown in Table 9 below. Construct DNA sequences are shown in Tables 10 and 11 below. Amino acid sequences are shown in Table 12.

TABLE 9
Strains for GFP display (FIGs. 5A-5C)

| Strain Number | Construct |
|---|---|
| SYN094 | |
| SYN2612 | p15A-Kan-ptet-pelB-FLAG-dasherGFP-HIS-PAL |
| SYN5985 | p15A-Kan-ptet-BAN-FLAG-GFP-HIS |
| SYN2609 | p15A-Kan-ptet-FLAG-GFP-HIS |
| SYN2610 | p15A-Kan-ptet-LppOmpA-FLAG-GFP-HIS |
| SYN2611 | p15A-Kan-ptet-NGIgAsig-FLAG-GFP-HIS-NGIgAb |
| SYN6067 | p15A-Kan-ptet-pelB-FLAG-dasherGFP-HIS |
| SYN6069 | p15A-Kan-ptet-FLAG-GFP-HIS-OsmY |

TABLE 10
Construct DNA Sequences

| Construct | DNA sequence |
|---|---|
| p15A-Kan- | tagcggagtgtatactggcttactatgttggca |
| ptet-pelB- | ctgatgagggtgtcagtgaagtgcttcatgtgg |
| FLAG- | caggagaaaaaaggctgcaccggtgcgtcagca |
| dasherGFP- | gaatatgtgatacaggatatattccgcttcctc |
| HIS- | gctcactgactcgctacgctcggtcgttcgact |
| PAL | gcggcgagcggaaatggcttacgaacggggcgg |
| | agatttcctggaagatgccaggaagatacttaa |
| | cagggaagtgagagggccgcggcaaagccgttt |
| | ttccataggctccgcccccctgacaagcatcac |
| | gaaatctgacgctcaaatcagtggtggcgaaac |
| | ccgacaggactataaagataccaggcgtttccc |
| | ctggcggctccctcgtgcgctctcctgttcctg |
| | cctttcggtttaccggtgtcattccgctgttat |
| | ggccgcgtttgtctcattccacgcctgacactc |
| | agttccgggtaggcagttcgctccaagctggac |
| | tgtatgcacgaaccccccgttcagtccgaccgc |
| | tgcgccttatccggtaactatcgtcttgagtcc |
| | aacccggaaagacatgcaaaagcaccactggca |
| | gcagccactggtaattgatttagaggagttagt |
| | cttgaagtcatgcgccggttaaggctaaactga |
| | aaggacaagttttggtgactgcgctcctccaag |
| | ccagttacctcggttcaaagagttggtagctca |
| | gagaaccttcgaaaaaccgccctgcaaggcggt |
| | tttttcgttttcagagcaagagattacgcgcag |
| | accaaaacgatctcaagaagatcatcttattaa |
| | ggggtctgacgctcagtggaacggtgcaccctg |
| | cagggctagctgataaagcgttcgcgctgcatt |
| | cggcagtttaagacccactttcacatttaagtt |
| | gtttttctaatccgcatatgatcaattcaaggc |
| | cgaataagaaggctggctctgcaccttggtgat |
| | caaataattcgatagcttgtcgtaataatggcg |
| | gcatactatcagtagtaggtgtttccctttctt |
| | ctttagcgacttgatgctcttgatcttccaata |
| | cgcaacctaaagtaaaatgccccacagcgctga |
| | gtgcatataatgcattctctagtgaaaaacctt |
| | gttggcataaaaaggctaattgattttcgagag |
| | tttcatactgtttttctgtaggccgtgtaccta |
| | aatgtacttttgctccatcgcgatgacttagta |
| | aagcacatctaaaacttttagcgttattacgta |
| | aaaaatcttgccagctttccccttctaaagggc |
| | aaaagtgagtatggtgcctatctaacatctcaa |
| | tggctaaggcgtcgagcaaagcccgcttatttt |
| | ttacatgccaatacaatgtaggctgctctacac |
| | ctagcttctgggcgagtttacgggttgttaaac |
| | cttcgattccgacctcattaagcagctctaatg |
| | cgctgttaatcactttacttttatctaatctag |
| | acatcattaattcctaatttttgttgacactct |
| | atcattgatagagttattttaccactccctatc |
| | agtgatagagaaaagtgaaaggaggtaaattAT |
| | GAAATATCTTCTTCCAACGGCTGCTGCTGGTTT |
| | ATTGCTTCTTGCCGCCCAGCCTGCGATGGCTAC |
| | TAGTGAGTATAAGGATGACGACGACAAGagatc |
| | tacggcattgacggaaggtgcaaaactgtttga |
| | gaaagagatcccgtatatcaccgaactggaagg |
| | cgacgtcgaaggtatgaaatttatcattaaagg |
| | cgagggtaccggtgacgcgaccacgggtaccat |
| | taaagcgaaatacatctgcactacgggcgacct |
| | gccggtcccgtgggcaaccctggtgagcaccct |
| | gagctacggtgttcagtgtttcgccaagtaccc |
| | gagccacatcaaggatttctttaagagcgccat |
| | gccggaaggttatacccaagagcgtaccatcag |
| | cttcgaaggcgacggcgtgtacaagacgcgtgc |
| | tatggttacctacgaacgcggttctatctacaa |
| | tcgtgtcacgctgactggtgagaactttaagaa |
| | agacggtcacattctgcgtaagaacgttgcatt |
| | ccaatgcccgccaagcattctgtatattctgcc |
| | tgacaccgttaacaatggcatccgcgttgagtt |
| | caaccaggcgtacgatattgaaggtgtgaccga |
| | aaaactggttaccaaatgcagccaaatgaatcg |
| | tccgttggcgggctccgcggcagtgcatatccc |
| | gcgttatcatcacattacctaccacaccaaact |
| | gagcaaagaccgcgacgagcgccgtgatcacat |
| | gtgtctggtagaggtcgtgaaagcggttgatct |
| | ggacacgtatcagGGATCCGGCCACCACCATCA |
| | TCACCACTGCAGTAGCAATAAAAATGCTTCGAA |
| | TGACGGATCGGAAGGAATGTTAGGTGCTGGTAC |
| | TGGCATGGACGCCAACGGTGGCAACGGAAATAT |
| | GTCTAGCGAAGAACAAGCTCGTTTAGAAATGCA |
| | GCAATTACAGCAAAACAACATAGTCTACTTCGA |
| | CTTAGACAAGTACGACATCCGTAGCGATTTTGC |
| | ACAAATGTTGGATGCCCATGCCAACTTCCTTCG |
| | TTCTAATCCAAGCTACAAAGTCACTGTTGAGGG |
| | CCACGCGGATGAGAGAGGCACTCCTGAATATAA |
| | CATATCCCTGGGGGAGCGCAGAGCAAACGCCGT |
| | AAAAATGTACCTGCAAGGCAAGGGCGTCTCGGC |
| | CGATCAAATCTCGATCGTGAGCTATGGCAAAGA |
| | GAAACCGGCTGTATTGGGACATGACGAAGCTGC |
| | TTACAGCAAGAATCGGCGGGCCGTCTTGGTCTA |
| | CtaataaGCATGCTAATCAGCCGTGGAATTCGC |
| | AACGTAAAAAAACCCGCCCCGGCGGGTTTTTTT |
| | ATAGCGGTCTCaGGAGgAACGATTGGTAAAGCC |
| | GGTGaacgcatgagAAAGCGCGCGGAAGATCAC |
| | GTTCCGGGGGCTTTtttattgcgcGGACCAAAA |
| | CGAAAAAAGACGCTCGAAAGCGTCTCTTTTCTG |
| | GAATTTaaatgaaactgcaatttattcatatca |
| | ggattatcaataccatatttttgaaaaagccgt |
| | ttctgtaatgaaggagaaaactcaccgaggcag |
| | ttccataggatggcaagatcctggtatcggtct |
| | gcgattccgactcgtccaacatcaatacaacct |
| | attaatttcccctcgtcaaaaataaggttatca |
| | agtgagaaatcaccatgagtgacgactgaatcc |
| | ggtgagaatggcaaaagcttatgcatttctttc |
| | cagacttgttcaacaggccagccattacgctcg |
| | tcatcaaaatcactcgcatcaaccaaaccgtta |
| | ttcattcgtgattgcgcctgagcgagacgaaat |
| | acgcgatcgctgttaaaaggacaattacaaaca |
| | ggaatcgaatgcaaccggcgcaggaacactgcc |
| | agcgcatcaacaatattttcacctgaatcagga |
| | tattcttctaatacctggaatgctgttttcccg |
| | gggatcgcagtggtgagtaaccatgcatcatca |
| | ggagtacggataaaatgcttgatggtcggaaga |
| | ggcataaattccgtcagccagtttagtctgacc |
| | atctcatctgtaacatcattggcaacgctacct |
| | ttgccatgtttcagaaacaactctggcgcatcg |
| | ggcttcccatacaatcgatagattgtcgcacct |
| | gattgcccgacattatcgcgagcccatttatac |
| | ccatataaatcagcatccatgttggaatttaat |
| | cgcggcctcgagcaagacgtttcccgttgaata |
| | tggctcataacaccccttgtattactgtttatg |
| | taagcagacagttttattgttcatgatgatata |
| | tttttatcttgtgcaatgtaacatcagagattt |
| | tgagacacaacgtggctttgttgaataaatcga |
| | acttttgctgagttgaaggatcagatcacgcat |
| | cttcccgacaacgcagaccgttccgtggcaaag |
| | caaaagttcaaaatcaccaactggtccacctac |
| | aacaaagctctcatcaaccgtggctccctcact |
| | ttctggctggatgatggggcgattcaggcctgg |
| | tatgagtcagcaacaccttcttcacgaggcaga |
| | cctcagcgc (SEQ ID NO: 1484) |
| p15A-Kan- | tagcggagtgtatactggcttactatgttggca |
| ptet- | ctgatgagggtgtcagtgaagtgcttcatgtgg |
| BAN-FLAG- | caggagaaaaaaggctgcaccggtgcgtcagca |
| GFP-HIS | gaatatgtgatacaggatatatKcgcttcctcg |
| | ctcactgactcgctacgctcggtcgttcgactg |
| | cggcgagcggaaatggcttacgaacggggcgga |
| | gatttcctggaagatgccaggaagatacttaac |
| | agggaagtgagagggccgcggcaaagccgtttt |
| | tccataggctccgcccccctgacaagcatcacg |
| | aaatctgacgctcaaatcagtggtggcgaaacc |
| | cgacaggactataaagataccaggcgtttcccc |
| | tggcggctccctcgtgcgctctcctgttcctgc |
| | ctttcggtttaccggtgtcattccgctgttatg |
| | gccgcgtttgtctcattccacgcctgacactca |
| | gttccgggtaggcagttcgctccaagctggact |
| | gtatgcacgaaccccccgttcagtccgaccgct |
| | gcgccttatccggtaactatcgtcttgagtcca |
| | acccggaaagacatgcaaaagcaccactggcag |
| | cagccactggtaattgatttagaggagttagtc |
| | ttgaagtcatgcgccggttaaggctaaactgaa |
| | aggacaagttttggtgactgcgctcctccaagc |
| | cagttacctcggttcaaagagttggtagctcag |
| | agaaccttcgaaaaaccgccctgcaaggcggtt |
| | ttttcgttttcagagcaagagattacgcgcaga |
| | ccaaaacgatctcaagaagatcatcttattaag |
| | gggtctgacgctcagtggaacggtgcaccctgc |
| | agggctagctgataaagcgttcgcgctgcattc |
| | ggcagtttaagacccactttcacatttaagttg |
| | ttmctaatccgcatatgatcaattcaaggccga |
| | ataagaaggctggctctgcaccttggtgatcaa |
| | ataattcgatagcttgtcgtaataatggcggca |
| | tactatcagtagtaggtgtttccctttcttctt |
| | tagcgacttgatgctcttgatcttccaatacgc |
| | aacctaaagtaaaatgccccacagcgctgagtg |
| | catataatgcattctctagtgaaaaaccttgtt |
| | ggcataaaaaggctaattgattttcgagagttt |
| | catactgtttttctgtaggccgtgtacctaaat |
| | gtacttttgctccatcgcgatgacttagtaaag |
| | cacatctaaaacttttagcgttattacgtaaaa |
| | aatcttgccagctttccccttctaaagggcaaa |
| | agtgagtatggtgcctatctaacatctcaatgg |
| | ctaaggcgtcgagcaaagcccgcttatttttta |
| | catgccaatacaatgtaggctgctctacaccta |
| | gcttctgggcgagtttacgggttgttaaacctt |
| | cgattccgacctcattaagcagctctaatgcgc |
| | tgttaatcactttacttttatctaatctagaca |
| | tcattaattcctaatttttgttgacactctatc |
| | attgatagagttattttaccactccctatagtg |
| | atagagaaaagtgaaaggaggtaaattATGACT |
| | AGTGCTTTTGACCCCAATCTGGTTGGGCCTACG |
| | TTACCTCCAATTCCGCCTTTCACTCTGCCTACG |
| | GACTATAAGGATGACGACGACAAGagatctacg |
| | gcattgacggaaggtgcaaaactgtttgagaaa |
| | gagatcccgtatatcaccgaactggaaggcgac |
| | gtcgaaggtatgaaatttatcattaaaggcgag |
| | ggtaccggtgacgcgaccacgggtaccattaaa |
| | gcgaaatacatctgcactacgggcgacctgccg |
| | gtcccgtgggcaaccctggtgagcaccctgagc |
| | tacggtgttcagtgtttcgccaagtacccgagc |
| | cacatcaaggatttctttaagagcgccatgccg |
| | gaaggttatacccaagagcgtaccatcagcttc |
| | gaaggcgacggcgtgtacaagacgcgtgctatg |
| | gttacctacgaacgcggttctatctacaatcgt |
| | gtcacgctgactggtgagaactttaagaaagac |
| | ggtcacattctgcgtaagaacgttgcattccaa |
| | tgcccgccaagcattctgtatattctgcctgac |
| | accgttaacaatggcatccgcgttgagttcaac |
| | caggcgtacgatattgaaggtgtgaccgaaaaa |
| | ctggttaccaaatgcagccaaatgaatcgtccg |
| | ttggcgggctccgcggcagtgcatatcccgcgt |
| | tatcatcacattacctaccacaccaaactgagc |
| | aaagaccgcgacgagcgccgtgatcacatgtgt |
| | ctggtagaggtcgtgaaagcggttgatctggac |
| | acgtatcagGGATCCGGCCACCACCATCATCAC |
| | CACtaataaGCATGCTAATCAGCCGTGGAATTC |
| | GGTCTCaGGAGgAACGATTGGTAAACCCGGTGa |
| | acgcatgagAAAGCCCCCGGAAGATCACCTTCC |
| | GGGGGCTTTtttattgcgcGGACCAAAACGAAA |
| | AAAGACGCTCGAAAGCGTCTCTTTTCTGGAATT |
| | TGGTACCGAGGcgtaatgctctgccagtgttac |
| | aaccaattaaccaattctgattagaaaaactca |
| | tcgagcatcaaatgaaactgcaatttattcata |
| | tcaggattatcaataccatatttttgaaaaagc |
| | cgtttctgtaatgaaggagaaaactcaccgagg |
| | cagttccataggatggcaagatcctggtatcgg |
| | tctgcgattccgactcgtccaacatcaatacaa |
| | cctattaatttcccctcgtcaaaaataaggtta |
| | tcaagtgagaaatcaccatgagtgacgactgaa |
| | tccggtgagaatggcaaaagcttatgcatttct |
| | ttccagacttgttcaacaggccagccattacgc |
| | tcgtcatcaaaatcactcgcatcaaccaaaccg |
| | ttattcattcgtgattgcgcctgagcgagacga |
| | aatacgcgatcgctgttaaaaggacaattacaa |
| | acaggaatcgaatgcaaccggcgcaggaacact |
| | gccagcgcatcaacaatattttcacctgaatca |
| | ggatattcttctaatacctggaatgctgttttc |
| | ccggggatcgcagtggtgagtaaccatgcatca |
| | tcaggagtacggataaaatgcttgatggtcgga |
| | agaggcataaattccgtcagccagtttactctg |
| | accatctcatctgtaacatcattggcaacgcta |
| | cctttgccatgtttcagaaacaactctggcgca |
| | tcgggcttcccatacaatcgatagattgtcgca |
| | cctgattgcccgacattatcgcgagcccattta |
| | tacccatataaatcagcatccatgttggaattt |
| | aatcgcggcctcgagcaagacgtttcccgttga |
| | atatggctcataacaccccttgtattactgttt |
| | atgtaagcagacagttttattgttcatgatgat |
| | atatttttatcttgtgcaatgtaacatcagaga |
| | ttttgagacacaacgtggctttgttgaataaat |
| | cgaacttttgctgagttgaaggatcagatcacg |
| | catcttcccgacaacgcagaccgttccgtggca |
| | aagcaaaagttcaaaatcaccaactggtccacc |
| | tacaacaaagctctcatcaaccgtggctccctc |
| | actttctggctggatgatggggcgattcaggcc |
| | tggtatgagtcagcaacaccttcttcacgaggc |
| | agacctcagcgc (SEQ ID NO: 1485) |
| p15A-Kan- | tggcataaaaaggctaattgattttcgagagtt |
| ptet- | tcatactgtttttctgtaggccgtgtacctaaa |
| LppOmpA- | tgtacttttgctccatcgcgatgacttagtaaa |
| FLAG-GFP- | gcacatctaaaacttttagcgttattacgtaaa |
| HIS | aaatcttgccagctttccccttctaaagggcaa |
| | aagtgagtatggtgcctatctaacatctcaatg |
| | gctaaggcgtcgagcaaagcccgcttatttttt |
| | acatgccaatacaatgtaggctgctctacacct |
| | agcttctgggcgagtttacgggttgttaaacct |
| | tcgattccgacctcattaagcagctctaatgcg |
| | ctgttaatcactttacttttatctaatctagac |
| | atcattaattcctaatttttgttgacactctat |
| | cattgatagagttattttaccactccctatcag |
| | tgatagagaaaagtgaaaggaggtaaattATGA |
| | CTAGTAAAGCAACAAAACTTGTGTTAGGCGCGG |
| | TTATACTTGGCTCCACCCTGCTTGCAGGTTGCT |
| | CGTCTAACGCGAAGATCGACCAGGGTATCAATC |
| | CTTACGTCGGGTTTGAAATGGGATACGATTGGT |
| | TGGGACGTATGCCTTATAAGGGAAGTGTTGAAA |
| | ACGGCGCTTATAAGGCGCAGGGAGTACAGTTAA |
| | CGGCCAAGCTTGGGTACCCCATAACAGACGATT |
| | TAGATATTTATACCCGTTTAGGAGGAATGGTTT |
| | GGAGAGCCGACACGAAGTCTAATGTATATGGTA |
| | AGAACCACGACACGGGAGTATCCCCCGTCTTTG |
| | CAGGGGGAGTGGAATATGCTATCACACCAGAGA |
| | TCGCTACCCGTITGGAATATCAATGGACGAATA |
| | ATATAGGCGACGCCCATACGATAGGAACGCGGC |
| | CCGACAACGGCATCCCTGGGGTcGACTATAAGG |
| | ATGACGACGACAAGcaattgacggcattgacgg |
| | aaggtgcaaaactgtttgagaaagagatcccgt |
| | atatcaccgaactggaaggcgacgtcgaaggta |
| | tgaaatttatcattaaaggcgagggtaccggtg |
| | acgcgaccacgggtaccattaaagcgaaataca |
| | tctgcactacgggcgacctgccggtcccgtggg |
| | caaccctggtgagcaccctgagctacggtgttc |
| | agtgtttcgccaagtacccgagccacatcaagg |
| | atttctttaagagcgccatgccggaaggttata |
| | cccaagagcgtaccatcagcttcgaaggcgacg |
| | gcgtgtacaagacgcgtgctatggttacctacg |
| | aacgcggttctatctacaatcgtgtcacgctga |
| | ctggtgagaactttaagaaagacggtcacattc |
| | tgcgtaagaacgttgcattccaatgcccgccaa |
| | gcatttctgtatattctgcctgacaccgttaac |
| | aattggcatccgcgttgagttcaaccaggcgta |
| | cgatattgaaggtgtgaccgaaaaactggttac |
| | caaatgcagccaaatgaatcgtccgttggcggg |
| | ctccgcggcagtgcatatcccgcgttatcatca |
| | cattacctaccacaccaaactgagcaaagaccg |
| | cgacgagcgccgtgatcacatgtgtctggtaga |
| | ggtcgtgaaagcggttgatctggacacgtatca |
| | gGGATCCGGCCACCACCATCATCACCACtaata |
| | aGCATGCTAATCAGCCGTGGAATTCGGTCTCaG |
| | GAGgAACGATTGGTAAACCCGGTGaacgcatga |
| | gAAAGCCCCCGGAAGATCACCTTCCGGGGGCTT |
| | TtttattgcgcGGACCAAAACGAAAAAAGACGC |
| | TCGAAAGCGTCTCTTaaaactcatcgagcatca |
| | aatgaaactgcaatttattcatatcaggattat |
| | caataccatatttttgaaaaagccgtttctgta |
| | atgaaggagaaaactcaccgaggcagttccata |
| | ggatggcaagatcctggtatcggtctgcgattc |
| | cgactcgtccaacatcaatacaacctattaatt |
| | tcccctcgtcaaaaataaggttatcaagtgaga |
| | aatcaccatgagtgacgactgaatccggtgaga |
| | atggcaaaagcttatgcatttctttccagactt |
| | gttcaacaggccagccattacgctcgtcatcaa |
| | aatcactcgcatcaaccaaaccgttattcattc |
| | gtgattgcgcctgagcgagacgaaatacgcgat |
| | cgctgttaaaaggacaattacaaacaggaatcg |
| | aatgcaaccggcgcaggaacactgccagcgcat |
| | caacaatattttcacctgaatcaggatattctt |
| | ctaatacctggaatgctgttttcccggggatcg |
| | cagtggtgagtaaccatgcatcatcaggagtac |
| | ggataaaatgcttgatggtcggaagaggcataa |
| | attccgtcagccagtttagtctgaccatctcat |
| | ctgtaacatcattggcaacgctacctttgccat |
| | gtttcagaaacaactctggcgcatcgggcttcc |
| | catacaatcgatagattgtcgcacctgattgcc |
| | cgacattatcgcgagcccatttatacccatata |
| | aatcagcatccatgttggaatttaatcgcggcc |
| | tcgagcaagacgtttcccgttgaatatggctca |
| | taacaccccttgtattactgtttatgtaagcag |
| | acagttttattgttcatgatgatatatttttat |
| | cttgtgcaatgtaacatcagagattttgagaca |
| | caacgtggctttgttgaataaatcgaacttttg |
| | ctgagttgaaggatcagatcacgcatcttcccg |
| | acaacgcagaccgttccgtggcaaagcaaaagt |
| | tcaaaatcaccaactggtccacctacaacaaag |
| | ctctcatcaaccgtggctccctcactttctggc |
| | tggatgatggggcgattcaggcctggtatgagt |
| | cagcaacaccttcttcacgaggcagacctcagc |
| | gctagcggagtgtatactggcttactatgttgg |
| | cactgatgagggtgtcagtgaagtgcttcatgt |
| | ggcaggagaaaaaaggctgcaccggtgcgtcag |
| | cagaatatgtgatacaggatatattccgcttcc |
| | tcgctcactgactcgctacgctcggtcgttcga |
| | ctgcggcgagcggaaatggcttacgaacggggc |
| | ggagatttcctggaagatgccaggaagatactt |
| | aacttgggaagtgagagggccgcggcaaagccg |
| | tttttccataggctccgcccccctgacaagcat |
| | cacgaaatctgacgctcaaatcagtggtggcga |
| | aacccgacaggactataaagataccaggcgttt |
| | cccctggcggctccctcgtgcgctctcctgttc |
| | ctgcctttcggtttaccggtgtcattccgctgt |
| | tatggccgcgtttgtctcattccacgcctgaca |
| | ctcagttccgggtaggcagttcgctccaagctg |
| | gactgtatgcacgaaccccccgttcagtccgac |
| | cgctgcgccttatccggtaactatcgtcttgag |
| | tccaacccggaaagacatgcaaaagcaccactg |
| | gcagcagccactggtaattgatttagaggagtt |
| | agtcttgaagtcatgcgccggttaaggctaaac |
| | tgaaaggacaagttttggtgactgcgctcctcc |
| | aagccagttacctcggttcaaagagttggtagc |
| | tcagagaaccttcgaaaaaccgccctgcaaggc |
| | ggttttttcgttttcagagcaagagattacgcg |
| | cagaccaaaacgatctcaagaagatcatcttat |
| | taaggggtctgacgctcagtggaacggtgcacc |
| | ctgcagggctagctgataaagcgttcgcgctgc |
| | attcggcagtttaagacccactttcacatttaa |
| | gttgtttttctaatccgcatatgatcaattcaa |
| | ggccgaataagaaggctggctctgcaccttggt |
| | gatcaaataattcgatagcttgtcgtaataatg |
| | gcggcatactatcagtagtaggtgtttcccttt |
| | cttctttagcgacttgatgctcttgatcttcca |
| | atacgcaacctaaagtaaaatgccccacagcgc |
| | tgagtgcatataatgcattctctagtgaaaaac |
| | cttgt (SEQ ID NO: 1486) |
| p15A-Kan- | AACGAAAAAAGACGCTCGAAAGCGTCTCTTTTC |
| ptet- | TGGAATTTGGTACCGAGGcgtaatgctctgcca |
| NGIgAsig- | gtgttacaaccaattaaccaattctgattagaa |
| FLAG-GFP- | aaactcatcgagcatcaaatgaaactgcaattt |
| HIS- | attcatatcaggattatcaataccatatttttg |
| NGIgAb | aaaaagccgtttctgtaatgaaggagaaaactc |
| | accgaggcagttccataggatggcaagatcctg |
| | gtatcggtctgcgattccgactcgtccaacatc |
| | aatacaacctattaatttcccctcgtcaaaaat |
| | aaggttatcaagtgagaaatcaccatgagtgac |
| | gactgaatccggtgagaatggcaaaagcttatg |
| | catttctttccagacttgttcaacaggccagcc |
| | attacgctcgtcacaaaatcactcgcatcaacc |
| | aaaccgttattcattcggatgcgcctgagcgag |
| | acgaaatacgcgtttcgctgttaaaaggacaat |
| | tttcaaacaggaatcgaatgcaaccggcgcagg |
| | aacactgccagcgcatcaacaatattttcacct |
| | gaatcaggatattcttctaatacctggaatgct |
| | gttttcccggggatcgcagtggtgagtaaccat |
| | gcatcatcaggagtacggataaaatgcttgatg |
| | gtcggaagaggcataaattccgtcagccagttt |
| | agtctgaccatctcatctgtaacatcattggca |
| | acgctacctttgccatgtttcagaaacaactct |
| | ggcgcatcgggcttcccatacaatcgatagatt |
| | gtcgcacctgattgcccgacattatcgcgagcc |
| | catttatacccatataaatcagcatccatgttg |
| | gaatttaatcgcggcctcgagcaagacgtttcc |
| | cgttgaatatggctcataacaccccttgtatta |
| | ctgtttatgtaagcagacagttttattgttcat |
| | gatgatatatttttatcttgtgcaatgtaacat |
| | cagagattttgagacacaacgtggctttgtgaa |
| | taaatcgaacttgctgagttgaaggatcagatc |
| | acgcatcttcccgacaacgcagaccgttccgtg |
| | gcaaagcaaaagttcaaaatcaccaactggtcc |
| | acctacaacaaagctctcatcaaccgtggctcc |
| | ctcactttctggctggatgatggggcgattcag |
| | gcctggtatgagtcagcaacaccttcttcacga |
| | ggcagacctcagcgctagcggagtgtatactgg |
| | cttactatgttggcactgatgagggtgtcagtg |
| | aagtgcttcatgtggcaggagaaaaaaggctgc |
| | accggtgcgtcagcagaatatgtgatacaggat |
| | atattccgcttcctcgctcactgactcgctacg |
| | ctcggtcgttcgactgcggcgagcggaaatggc |
| | ttacgaacggggcggagatttcctggaagatgc |
| | caggaagatacttttacagggaagtgagagggc |
| | cgcggcaaagccgtttttccataggctccgccc |
| | ccctgacaagcatcacgaaatctgacgctcaaa |
| | tcagtggtggcgaaacccgacaggactataaag |
| | ataccaggcgtttcccctggcggctccctcgtg |
| | cgctctcctgttcctgcctttcggtttaccggt |
| | gtcattccgctgttatggccgcgtttgtctcat |
| | tccacgcctgacactcagttccgggtaggcagt |
| | tcgctccaagctggactgtatgcacgaaccccc |
| | cgttcagtccgaccgctgcgccttatccggtaa |
| | ctatcgtcttgagtccaacccggaaagacatgc |
| | aaaagcaccactggcagcagccactggtaattg |
| | atttagaggagttagtcttgaagtcatgcgccg |
| | gttaaggctaaactgaaaggacaagttttggtg |
| | actgcgctcctccaagccagttacctcggttca |
| | aagagttggtagctcagagaaccttcgaaaaac |
| | cgccctgcaaggcggttttttcgttttcagagc |
| | aagagattacgcgcagaccaaaacgatctcaag |
| | aagatcatcttattaaggggtctgacgctcagt |
| | ggaacggtgcaccctgcagggctagctgataaa |
| | gcgttcgcgctgcattcggcagtttaagaccca |
| | ctttcacatttaagttgtttttctaatccgcat |
| | atgatcaattcaaggccgaataagaaggctggc |
| | tctgcaccttggtgatcaaataattcgatagct |
| | tgtcgtaataatggcggcatactatcagtagta |
| | ggtgtttccctttcttctttagcgacttgatgc |
| | tcttgatcttccaatacgcaacctaaagtaaaa |
| | tgccccacagcgctgagtgcatataatgcattc |
| | tctagtgaaaaaccttgttggcataaaaaggct |
| | aattgattttcgagagtttcatactgttttctg |
| | taggccgtgtacctaaatgtacttttgctccat |
| | cgcgatgacttagtaaagcacatctttaaactt |
| | ttagcgttattacgtaaaaaatcttgccagctt |
| | tccccttctaaagggcaaaagtgagtatggtgc |
| | ctatctaacatctcaatggctaaggcgtcgagc |
| | aaagcccgcttattttttacatgccaatacaat |
| | gtaggctgctctacacctagcttctgggcgagt |
| | ttacgggttgttaaaccttcgattccgacctca |
| | ttaagcagctctaatgcgctgttaatcacttta |
| | cttttatctaatctagacatcattaattcctaa |
| | tttttgttgacactctatcattgatagagttat |
| | tttaccactccctatcagtgatagagaaaagtg |
| | aaaggaggtaaattATGACTAGTAAGGCAAAAC |
| | GCTTCAAGATTAACGCTATCAGTTTATCCATCT |
| | TTTTAGCGTATGCGTTGACTCCGTACTCAGAGG |
| | CAGTcGACTATAAGGATGACGACGACAAGcaat |
| | tgacggcattgacggaaggtgcaaaactgtttg |
| | agaaagagatcccgtatatcaccgaactggaag |
| | gcgacgtcgaaggtattgaaattatcataaagg |
| | cgagggtaccggtgacgcgaccacgggtaccat |
| | taaagcgaaatacatctgcactacgggcgacct |
| | gccggtcccgtgggcaaccctggtgagcaccct |
| | gagctacggtgttcagtgtttcgccaagtaccc |
| | gagccacatcaaggatttctttaagagcgccat |
| | gccggaaggttatacccaagagcgtaccatcag |
| | cttcgaaggcgacggcgtgtacaagacgcgtgc |
| | tatggttacctacgaacgcggttctatctacaa |
| | tcgtgtcacgctgactggtgagaactttaagaa |
| | agacggtcacattctgcgtaagaacgttgcatt |
| | ccaatgcccgccaagcattctgtatattttgcc |
| | tgacaccgttaacaatggcatccgcgttgagtt |
| | caaccaggcgtacgatattgaaggtgtgaccga |
| | aaaactggttaccaaatgcagccaaatgaatcg |
| | tccgttggcgggctccgcggcagtgcatatccc |
| | gcgttatcatcacattacctaccacaccaaact |
| | gagcaaagaccgcgacgagcgccgtgatcacat |
| | gtgtctggtagaggtcgtgaaagcggttgatct |
| | ggacacgtatcagGGATCCGGCCACCACCATCA |
| | TCACCACCCGCGTGCTGCACAACCGGGTACACA |
| | AGCTGCAGCCCAGGCCGACGCAGTATCCACGAA |
| | CACAAATTCAGCGTTGAGTGATGCTATGGCTTC |
| | AACTCAATCAATTCTTTTGGACACCGGCGCATA |
| | TTTAACTCGCCATATCGCCCAAAAGTCTCGTGC |
| | GGATGCAGAAAAAAATAGCGTGTGGATGTCGAA |
| | TACTGGGTATGGGCGCGACTACGCCTCTGCGCA |
| | GTACCGCCGTTTCAGCAGCAAACGCACCCAGAC |
| | TCAAATCGGGATCGATCGTAGTCTTAGTGAGAA |
| | TATGCAGATTGGCGGAGTTCTGACTTATTCCGA |
| | CTTTTCAACATACGTTCGACCAGGCAGGGGGCA |
| | AGAATACGTTTGTACAGGCCAATCTTTACGGAA |
| | AGTACTACTTGAATGATGCCTGGTATGTCGCTG |
| | GGGACATTGGCGCAGGGAGCTTGCGTTCCCGCC |
| | TTCAAACGCAACAAAAGGCGAATTTCAATCGCA |
| | CCAGCATTCAGACTGGCCTTACACTTGGGAATA |
| | CACTTAAGATTAATCAATTTGAGATTGTGCCCA |
| | GTGCTGGgATTCGTTATAGTCGTTTATCCAGCG |
| | CAGACTATAAGTTAGGCGACGACTCTGTGAAAG |
| | TTAGCTCAATGGCCGTGAAGACGTTGACGGCTG |
| | GTTTGGATTTCGCCTATCGCTTTAAAGTTGGAA |
| | ATCTGACCGTGAAGCCGCTGCTTTCGGCCGCTT |
| | ATTTTGCAAACTACGGCAAGGGCGGCGTTAATG |
| | TCGGTGGTAAGAGTTTCGCCTATAAAGCTGACA |
| | ATCAGCAGCAATATTCGGCCGGCGTCGCGTTGT |
| | TGTATCGCAACGTGACTTTAAATGTAAACGGCT |
| | CCATCACAAAGGGCAAGCAGTTGGAGAAGCAAA |
| | AGTCAGGCCAAATCAAGATTCAAATCCGTTTCt |
| | aataaGCATGCTAATCAGCCGTGGAATTCGGTC |
| | TCaGGAGgAACGATTGGTAAACCCGGTGaacgc |
| | atgagAAAGCCCCCGGAAGATCACCTTCCGGGG |
| | GCTTTtttcattgcgcGGACCAA |
| | (SEQ ID NO: 1487) |

TABLE 11
DNA Sequences

| Construct | Promoter | Anchor-5′ | Displayed protein (DNA) |
|---|---|---|---|
| p15A- | Gttgacactctat | TGCAGTAGCAATAAAAATGCTTCG | Tcggcattgacggaaggtgcaaaactg |
| Kan-ptet- | cattgatagagtta | AATGACGGATCGGAAGGAATGTTA | tttgagaaagagatcccgtatatcacc |
| pelB- | ttttaccactccct | GGTGCTGGTACTGGCATGGACGCC | gaactggaaggcgacgtcgaaggtatg |
| FLAG- | atcagtgatagag | AACGGTGGCAACGGAAATATGTCT | aaatttatcattaaaggcgagggtacc |
| dasherGF | aa (SEQ ID | AGCGAAGAACAAGCTCGTTTACAA | ggtgacgcgaccacgggtaccattaa |
| P-HIS- | NO: 1488) | ATGCAGCAATTACAGCAAAACAAC | agcgaaatacatctgcactacgggcg |
| PAL | | ATAGTCTACTTCGACTTAGACAAGT | acctgccggtcccgtgggcaaccctg |
| | | ACGACATCCGTAGCGATTTTGCACA | gtgagcaccctgagctacggtgttcag |
| | | AATGTTGGATGCCCATGCCAACTTC | tgtttcgccaagtacccgagccacatc |
| | | CTTCGTTCTAATCCAAGCTACAAAG | aaggatttctttaagagcgccatgccg |
| | | TCACTGTTGAGGGCCACGCGGATG | gaaggttatacccaagagcgtaccatc |
| | | AGAGAGGCACTCCTGAATATAACA | agcttcgaaggcgacggcgtgtacaa |
| | | TATCCCTGGGGGAGCGCAGAGCAA | gacgcgtgctatggttacctacgaacg |
| | | ACGCCGTAAAAATGTACCTGCAAG | cggttctatctacaatcgtgtcacgctg |
| | | GCAAGGGCGTCTCGGCCGATCAAA | actggtgagaactttaagaaagacggt |
| | | TCTCGATCGTGAGCTATGGCAAAG | cacattctgcgtaagaacgttgcattcc |
| | | AGAAACCGGCTGTATTGGGACATG | aatgcccgccaagcattctgtatattct |
| | | ACGAAGCTGCTTACAGCAAGAATC | gcctgacaccgttaacaatggcatccgc |
| | | GGCGGGCCGTCTTGGTCTAC(SEQ | gttgagttcaaccaggcgtacgatattg |
| | | ID NO: 1489) | aaggtgtgaccgaaaaactggttacca |
| | | | aatgcagccaaatgaatcgtccgttgg |
| | | | cgggctccgcggcagtgcatatcccg |
| | | | cgttatcatcacattacctaccacacca |
| | | | aactgagcaaagaccgcgacgagcg |
| | | | ccgtgatcacatgtgtctggtagaggtc |
| | | | gtgaaagcggttgatctggacacgtat |
| | | | cag (SEQ ID NO: 1495) |
| p15A- | Gttgacactctat | GCTTTTGACCCCAATCTGGTTGGGC | Acggcattgacggaaggtgcaaaact |
| Kan-ptet- | cattgatagagtta | CTACGTTACCTCCAATTCCGCCTTT | gtttgagaaagagatcccgtatatcacc |
| BAN- | ttttaccactccct | CACTCTGCCTACG (SEQ ID NO: | gaactggaaggcgacgtcgaaggtat |
| FLAG- | atcagtgatagag | 1490) | gaaatttatcattaaaggcgagggtacc |
| GFP-HIS | aa (SEQ ID | | ggtgacgcgaccacgggtaccattaa |
| | NO: 1488) | | agcgaaatacatctgcactacgggcg |
| | | | acctgccggtcccgtgggcaaccctg |
| | | | gtgagcaccctgagctacggtgttcag |
| | | | tgtttcgccaagtacccgagccacatc |
| | | | aaggatttctttaagagcgccatgccg |
| | | | gaaggttatacccaagagcgtaccatc |
| | | | agcttcgaaggcgacggcgtgtacaa |
| | | | gacgcgtgctatggttacctacgaacg |
| | | | cggttctatctacaatcgtgtcacgctg |
| | | | actggtgagaactttaagaaagacggt |
| | | | cacattctgcgtaagaacgttgcattcc |
| | | | aatgcccgccaagcattctgtatattctg |
| | | | cctgacaccgttaacaatggcatccgc |
| | | | gttgagttcaaccaggcgtacgatattg |
| | | | aaggtgtgaccgaaaaactggttacca |
| | | | aatgcagccaaatgaatcgtccgttgg |
| | | | cgggctccgcggcagtgcatatcccg |
| | | | cgttatcatcacattacctaccacacca |
| | | | aactgagcaaagaccgcgacgagcg |
| | | | ccgtgatcacatgtgtctggtagaggtc |
| | | | gtgaaagcggttgatctggacacgtat |
| | | | cag (SEQ ID NO: 1496) |
| p15A- | Gttgacactctat | ATGACTAGTTAAAGCAACAAAACT | Tcggcattgacggaaggtgcaaaact |
| Kan-ptet- | cattgatagagtta | TGTGTTAGGCGCGGTTATACTTGGC | gtttgagaaagagatcccgtatatcacc |
| LppOmpA- | ttttaccactccct | TCCACCCTGCTTGCAGGTTGCTCGT | gaactggaaggcgacgtcgaaggtat |
| FLAG- | atcagtgatagag | CTAACGCGAAGATCGACCAGGGTA | gaaatttatcattaaaggcgagggtacc |
| GFP-HIS | aa (SEQ ID | TCAATCCTTACGTCGGGTTTGAAAT | ggtgacgcgaccacgggtaccattaa |
| | NO: 1488) | GGGATACGATTGGTTGGGACGTAT | agcgaaatacatctgcactacgggcg |
| | | GCCTTATAAGGGAAGTGTTGAAAA | acctgccggtcccgtgggcaaccctg |
| | | CGGCGCTTATAAGGCGCAGGGAGT | gtgagcaccctgagctacggtgttcag |
| | | ACAGTTAACGGCCAAGCTTGGGTA | tgtttcgccaagtacccgagccacatc |
| | | CCCCATAACAGACGATTTAGATATT | aaggatttctttaagagcgccatgccg |
| | | TATACCCGTTTAGGAGGAATGGTTT | gaaggttatacccaagagcgtaccatc |
| | | GGAGAGCCGACACGAAGTCTAATG | agcttcgaaggcgacggcgtgtacaa |
| | | TATATGGTAAGAACCACGACACGG | gacgcgtgctatggttacctacgaacg |
| | | GAGTATCCCCCGTCTTTGCAGGGGG | cggttctatctacaatcgtgtcacgctg |
| | | AGTGGAATATGCTATCACACCAGA | actggtgagaactttaagaaagacggt |
| | | GATCGCTACCCGTTTGGAATATCAA | cacattctgcgtaagaacgttgcattcc |
| | | TGGACGAATAATATAGGCGACGCC | aatgcccgccaagcattctgtatattctg |
| | | CATACGATAGGAACGCGGCCCGAC | cctgacaccgttaacaatggcatccgc |
| | | AAC(SEQ ID NO: 1491) | gttgagttcaaccaggcgtacgatattg |
| | | | aaggtgtgaccgaaaaactggttacca |
| | | | aatgcagccaaatgaatcgtccgttgg |
| | | | cgggctccgcggcagtgcatatcccg |
| | | | cgttatcatcacattacctaccacacca |
| | | | aactgagcaaagaccgcgacgagcg |
| | | | ccgtgatcacatgtgtctggtagaggtc |
| | | | gtgaaagcggttgatctggacacgtat |
| | | | cag (SEQ ID NO: 1495) |
| p15A- | Gttgacactctat | CCGCGTGCTGCACAACCGCGTACA | Tcggcattgacggaaggtgcaaaact |
| Kan-ptet- | cattgatagagtta | CAAGCTGCAGCCCAGGCCGACGCA | gtttgagaaagagatcccgtatatcacc |
| NGIgAsig- | ttttaccactccct | GTATCCACGAACACAAATTCAGCG | gaactggaaggcgacgtcgaaggtat |
| FLAG- | atcagtgatagag | TTGAGTGATGCTATGGCTTCAACTC | gaaatttatcattaaaggcgagggtacc |
| GFP-HIS- | aa (SEQ ID | AATCAATTCTTTTGGACACCGGCGC | ggtgacgcgaccacgggtaccattaa |
| NGIgAb | NO: 1488) | ATATTTAACTCGCCATATCGCCCAA | agcgaaatacatctgcactacgggcg |
| | | AAGTCTCGTGCGGATGCAGAAAAA | acctgccggtcccgtgggcaaccctg |
| | | AATAGCGTGTGGATGTCGAATACT | gtgagcaccctgagctacggtgttcag |
| | | GGGTATGGGCGCGACTACGCCTCT | tgtttcgccaagtacccgagccacatc |
| | | GCGCAGTACCGCCGTTTCAGCAGC | aaggatttctttaagagcgccatgccg |
| | | AAACGCACCCAGACTCAAATCGGG | gaaggttatacccaagagcgtaccatc |
| | | ATCGATCGTAGTCTTAGTGAGAATA | agcttcgaaggcgacggcgtgtacaa |
| | | TGCAGATTGGCGGAGTTCTGACTTA | gacgcgtgctatggttacctacgaacg |
| | | TTCCGACTCTCAACATACGTTCGAC | cggttctatctacaatcgtgtcacgctg |
| | | CAGGCAGGGGGCAAGAATACGTTT | actggtgagaactttaagaaagacggt |
| | | GTACAGGCCAATCTTTACGGAAAG | cacattctgcgtaagaacgttgcattcc |
| | | TACTACTTGAATGATGCCTGGTATG | aatgcccgccaagcattctgtatattctg |
| | | TCGCTGGGGACATTGGCGCAGGGA | cctgacaccgttaacaatggcatccgc |
| | | GCTTGCGTTCCCGCCTTCAAACGCA | gttgagttcaaccaggcgtacgatattg |
| | | ACAAAAGGCGAATTTCAATCGCAC | aaggtgtgaccgaaaaactggttacca |
| | | CAGCATTCAGACTGGCCTTACACTT | aatgcagccaaatgaatcgtccgttgg |
| | | GGGAATACACTTAAGATTAATCAA | cgggctccgcggcagtgcatatcccg |
| | | TTTGAGATTGTGCCCAGTGCTGGgA | cgttatcatcacattacctaccacacca |
| | | TTCGTTATAGTCGTTTATCCAGCGC | aactgagcaaagaccgcgacgagcg |
| | | AGACTATAAGTTAGGCGACGACTC | ccgtgatcacatgtgtctggtagaggtc |
| | | TGTGAAAGTTAGCTCAATGGCCGT | gtgaaagcggttgatctggacacgtat |
| | | GAAGACGTTGACGGCTGGTTTGGA | cag (SEQ ID NO: 1495) |
| | | TTTCGCCTATCGCTTTAAAGTTGGA | |
| | | AATCTGACCGTGAAGCCGCTGCTTT | |
| | | CGGCCGCTTATTTTGCAAACTACGG | |
| | | CAAGGGCGGCGTTAATGTCGGTGG | |
| | | TAAGAGTTTCGCCTATAAAGCTGAC | |
| | | AATCAGCAGCAATATTCGGCCGGC | |
| | | GTCGCGTTGTTGTATCGCAACGTGA | |
| | | CTTTAAATGTAAACGGCTCCATCAC | |
| | | AAAGGGCAAGCAGTTGGAGAAGCA | |
| | | AAAGTCAGGCCAAATCAAGATTCA | |
| | | AATCCGTTTC (SEQ ID NO: 1449) | |
| p15A- | Gttgacactctat | ATGACAATGACTCGTTTGAAAATTT | Tcggcattgacggaaggtgcaaaact |
| Kan-ptet- | cattgatagagtta | CCAAAACCCTGCTGGCCGTAATGCT | gtttgagaaagagatcccgtatatcacc |
| FLAG- | ttttaccactccct | TACGTCCGCAGTCGCCACTGGTTCC | gaactggaaggcgacgtcgaaggtat |
| GFP-HIS- | atcagtgatagag | GCATACGCAGAGAATAATGCGCAG | gaaatttatcattaaaggcgagggtacc |
| OsmY | aa (SEQ ID | ACAACTAACGAGTCAGCAGGGCAA | ggtgacgcgaccacgggtaccattaa |
| | NO: 1488) | AAAGTAGACTCCAGTATGAATAAG | agcgaaatacatctgcactacgggcg |
| | | GTGGGCAATTTTATGGACGACTCA | acctgccggtcccgtgggcaaccctg |
| | | GCTATCACCGCTAAGGTGAAAGCG | gtgagcaccctgagctacggtgttcag |
| | | GCGCTGGTGGACCACGACAACATC | tgtttcgccaagtacccgagccacatc |
| | | AAGTCCACGGACATCTCAGTTAAA | aaggatttctttaagagcgccatgccg |
| | | ACGGACCAAAAGGTAGTAACCTTA | gaaggttatacccaagagcgtaccatc |
| | | AGCGGGTTCGTAGAAAGCCAGGCG | agcttcgaaggcgacggcgtgtacaa |
| | | CAAGCAGAGGAAGCGGTAAAAGTC | gacgcgtgctatggttacctacgaacg |
| | | GCTAAAGGCGTAGAAGGGGTCACT | cggttctatctacaatcgtgtcacgctg |
| | | TCGGTgTCAGACAAGTTGCATGTGC | actggtgagaactttaagaaagacggt |
| | | GTGACGCAAAGGAAGGATCAGTAA | cacattctgcgtaagaacgttgcattcc |
| | | AGGGTTATGCCGGAGATACGGCAA | aatgcccgccaagcattctgtatattctg |
| | | CGACCTCTGAGATCAAAGCGAAAT | cctgacaccgttaacaatggcatccgc |
| | | TACTGGCAGACGACATTGTTCCCTC | gttgagttcaaccaggcgtacgatattg |
| | | GCGTCATGTTAAAGTCGAAACGAC | aaggtgtgaccgaaaaactggttacca |
| | | TGATGGCGTAGTCCAGCTTTCGGGT | aatgcagccaaatgaatcgtccgttgg |
| | | ACAGTTGACTCCCAAGCACAAAGT | cgggctccgcggcagtgcatatcccg |
| | | GATCGCGCGGAATCTATTGCAAAG | cgttatcatcacattacctaccacacca |
| | | GCGGTGGACGGAGTGAAATCCGTC | aactgagcaaagaccgcgacgagcg |
| | | AAAAACGATCTTAAAACGAAA | ccgtgatcacatgtgtctggtagaggtc |
| | | (SEQ ID NO: 1492) | gtgaaagcggttgatctggacacgtat |
| | | | cag (SEQ ID NO: 1495) |
| p15A- | Gttgacactctat | ATGGTTTTCCAACCCATCAGCGAAT | Tcggcattgacggaaggtgcaaaact |
| invasinN- | cattgatagagtta | TTTTGCTGATTCGTAACGCTGGGAT | gtttgagaaagagatcccgtatatcacc |
| FLAG- | ttttaccactccct | GTCCATGTATTTTAACAAGATCATT | gaactggaaggcgacgtcgaaggtat |
| GFP-HIS | atcagtgatagag | TCTTTTAACATCATTTCACGTATCG | gaaatttatcattaaaggcgagggtacc |
| | aa (SEQ ID | TTATTTGCATTTTTCTTATCTGTGGT | ggtgacgcgaccacgggtaccattaa |
| | NO: 1488) | ATGTTCATGGCCGGTGCATCTGAAA | agcgaaatacatctgcactacgggcg |
| | | AGTATGATGCAAACGCACCCCAAC | acctgccggtcccgtgggcaaccctg |
| | | AGGTGCAGCCATACTCGGTTTCATC | gtgagcaccctgagctacggtgttcag |
| | | ATCAGCGTTCGAGAATCTGCACCCC | tgtttcgccaagtacccgagccacatc |
| | | AATAACGAGATGGAGTCGAGTATC | aaggatttctttaagagcgccatgccg |
| | | AACCCTTTTAGTGCTTCGGACACCG | gaaggttatacccaagagcgtaccatc |
| | | AGCGTAATGCAGCTATCATCGATC | agcttcgaaggcgacggcgtgtacaa |
| | | GTGCTAACAAGGAACAAGAAACGG | gacgcgtgctatggttacctacgaacg |
| | | AAGCAGTCAACAAAATGATCTCCA | cggttctatctacaatcgtgtcacgctg |
| | | CTGGCGCTCGTTTAGCTGCCAGCGG | actggtgagaactttaagaaagacggt |
| | | TCGCGCGTCCGATGTGGCGCACAG | cacattctgcgtaagaacgttgcattcc |
| | | TATGGTAGGGGATGCGGTCAACCA | aatgcccgccaagcattctgtatattctg |
| | | GGAGATTAAACAATGGCTGAATCG | cctgacaccgttaacaatggcatccgc |
| | | CTTCGGCACTGCTCAAGTGAATTTA | gttgagttcaaccaggcgtacgatattg |
| | | AATTTTGACAAGAACTTCTCGTTAA | aaggtgtgaccgaaaaactggttacca |
| | | AGGAGTCTTCGCTTGACTGGTTGGC | aatgcagccaaatgaatcgtccgttgg |
| | | CCCATGGTACGATTCGGCGTCATTC | cgggctccgcggcagtgcatatcccg |
| | | CTTTTCTTTTCTCAGTTGGGCATCC | cgttatcatcacattacctaccacacca |
| | | GTAACAAGGACAGTCGTAATACAC | aactgagcaaagaccgcgacgagcg |
| | | TTAACCTTGGTGTTGGCATTCGCAC | ccgtgatcacatgtgtctggtagaggtc |
| | | ATTAGAAAATGGTTGGTTGTATGGC | gtgaaagcggttgatctggacacgtat |
| | | CTGAACACCTTTTACGACAATGACT | cag (SEQ ID NO: 1495) |
| | | TAACGGGACACAATCACCGTATCG | |
| | | GGCTGGGCGCCGAGGCGTGGACTG | |
| | | ACTACTTGCAGTTAGCCGCGAATG | |
| | | GGTACTTCCGTCTTA | |
| | | ATGGTTGGCACTCTTCCCGTGACTT | |
| | | CAGCGACTACAAAGAACGCCCTGC | |
| | | TACCGGGGGAGATTTGCGTGCGAA | |
| | | TGCGTACCTGCCCGCTCTTCCGCAA | |
| | | CTTGGCGGGAAGTTAATGTATGAG | |
| | | CAGTATACTGGGGAACGCGTGGCT | |
| | | CTGTTCGGAAAGGACAACCTGCAG | |
| | | CGCAACCCATACGCTGTCACTGCG | |
| | | GGTATCAACTATACGCCAGTTCCGT | |
| | | TGCTGACGGTCGGCGTGGATCAAC | |
| | | GTATGGGGAAGTCGAGTAAACATG | |
| | | AAACGCAATGGAATTTACAAATGA | |
| | | ACTATCGCTTAGGGGAGAGTTTCCA | |
| | | AAGTCAGCTTAGCCCTTCGGCGGTC | |
| | | GCAGGGACTCGTTTGCTTGCTGAGT | |
| | | CCCGCTACAACCTGGTTGATCGCAA | |
| | | TAACAATATCGTACTGGAATACCA | |
| | | GAAACAACAAGTGGTTAAGCTGAC | |
| | | GTTGAGCCCTGCGACCATCAGTGG | |
| | | ATTGCCCGGACAAGTTTACCAGGT | |
| | | AAATGCCCAGGTCCAGGGGGCCTC | |
| | | TGCGGTTCGCGAAATTGTCTGGTCA | |
| | | GACGCAGAATTAATCGCTGCAGGA | |
| | | GGCACCTTAACGCCACTTTCCACTA | |
| | | CACAATTCAATTTAGTCCTTCCCCC | |
| | | ATACAAACGTACCGCCCAGGTATC | |
| | | GCGCGTAACTGATGACTTAACTGCT | |
| | | AATTTTTATTCACTGTCGGCGTTAG | |
| | | CAGTTGACCATCAAGGCAACCGTA | |
| | | GTAATTCCTTCACATTATCTGTAAC | |
| | | GGTGCAGCAGCCGCAACTGACGCT | |
| | | TACCGCAGCGGTCATTGGTGATGG | |
| | | GGCCCCAGCTAATGGGAAAACCGC | |
| | | AATCACTGTCGAgTTCACAGTTGCA | |
| | | GATTTTGAAGGCAAGCCGCTGGCG | |
| | | GGTCAGGAGGTTGTGATTACGACT | |
| | | AATAACGGTGCTCTTCCTAATAAGA | |
| | | TTACTGAAAAGACTGACGCTAACG | |
| | | GCGTTGCCCGCATTGCCCTTACGAA | |
| | | CACAACCGATGGGGTCACGGTAGT | |
| | | TACCGCAGAGGTCGAGGGGCAACG | |
| | | CCAATCCGTTGACACGCACTTCGTT | |
| | | AAGGGTACTATCGCGGCCGATAAA | |
| | | AGCACGCTGGCCGCGGT(SEQ ID | |
| | | NO: 1493) | |
| p15A- | Gttgacactctat | ATGTTAATCAATAGAAACATTGTCG | Tcggc attgacggaaggtgcaaaact |
| yiaT- | cattgatagagtta | CCCTTTTTGCCCTGCCATTCATGGC | gtttgagaaagagatcccgtatatcac |
| FLAG- | ttttaccactccct | GAGTGCGACCGCTTCCGAGTTAAG | cgaactggaaggcgacgtcgaaggtat |
| GFP-HIS | atcagtgatagag | CATAGGGGCCGGCGCTGCGTATAA | gaaatttatcattaaaggcgagggtac |
| | aa (SEQ ID | TGAGAGTCCTTACCGTGGCTATAAT | cggtgacgcgaccacgggtaccattaa |
| | NO: 1488) | GAAAACACAAAGGCAATCCCCCTT | agcgaaatacatctgcactacgggcg |
| | | ATCTCGTATGAAGGCGACACCTTCT | acctgccggtcccgtgggcaaccctg |
| | | ATGTACGTCAGACTACATTAGGTTT | gtgagcaccctgagctacggtgttcag |
| | | CATCTTAAGCCAGTCCGAGAAAAA | tgtttcgccaagtacccgagccacatc |
| | | CGAGTTATCGCTGACAGCTTCATGG | aaggatttctttaagagcgccatgccg |
| | | ATGCCATTGGAATTCGACCCGACA | gaaggttatacccaagagcgtaccatc |
| | | GATAATGATGATTATGCGATGCAG | agcttcgaaggcgacggcgtgtacaa |
| | | CAGCTGGACAAACGGGATTCGACA | gacgcgtgctatggttacctacgaacg |
| | | GCTATGGCCGGCGTGGCTTGGTATC | cggttctatctacaatcgtgtcacgctg |
| | | ACCACGAGCGGTGGGGAACTGTCA | actggtgagaactttaagaaagacggt |
| | | AAGCTTCCGCCGCCGCTGACGTGTT | cacattctgcgtaagaacgttgcattcc |
| | | AGACAACAGCAATGGCTGGGTGGG | aatgcccgccaagcattctgtatattct |
| | | GGAATTAAGCGTGTTCCATAAGAT | gcctgacaccgttaacaatggcatccgc |
| | | GCAGATAGGTAGATTATCCCTTACA | gttgagttcaaccaggcgtacgatattg |
| | | CCGGCTTTGGGAGTCCTGTATTATG | aaggtgtgaccgaaaaactggttacca |
| | | ATGAGAACTTTTCCGACTATTATTA | aatgcagccaaatgaatcgtccgttgg |
| | | TGGGATTTCTGAGTCTGAAAGTCGT | cgggctccgcggcagtgcatatcccgc |
| | | CGGAGCGGTCTTGCGTCGTACTCGG | gttatcatcacattacctaccacacca |
| | | CGCAAGACGCGTGGGTGCCTTATG | aactgagcaaagaccgcgacgagcgc |
| | | TATCTTTGACAGCGAAGTATCCAAT | cgtgatcacatgtgtctggtagaggtc |
| | | AGGCGAGCACGTGGTATTGATGGC | gtgaaagcggttgatctggacacgtat |
| | | AAGTGCTGGTTATTCCGAGTTGCCC | cag (SEQ ID NO: 1495) |
| | | GAAGAAATTACTGACAGTCCTATG | |
| | | ATAGACCGT (SEQ ID NO: 1450) | |
| p15A- | Gttgacactctat | ATGATTACGCATGGCTGTTATACCC | Acggcattgacggaaggtgcaaaact |
| intiminN- | cattgatagagtta | GTACGCGTCATAAACACAAGTTGA | gtttgagaaagagatcccgtatatcac |
| FLAG- | ttttaccactccct | AGAAAACTCTGATCATGTTATCCGC | cgaactggaaggcgacgtcgaaggtat |
| GFP-HIS | atcagtgatagag | TGGACTTGGACTTTmTTTACGTG | gaaatttatcattaaaggcgagggtac |
| | aa (SEQ ID | AATCAGAACTCTTTCGCTAATGGGG | cggtgacgcgaccacgggtaccattaa |
| | NO: 1488) | AAAATTATTTTAAACTGGGATCAG | agcgaaatacatctgcactacgggcg |
| | | ACAGCAAATTACTTACGCATGACTC | acctgccggtcccgtgggcaaccctg |
| | | ATACCAGAATCGTCTGTTTTATACG | gtgagcaccctgagctacggtgttcag |
| | | CTGAAAACTGGTGAaACCGTTGCAG | tgtttcgccaagtacccgagccacatc |
| | | ATTTAAGTAAAAGTCAGGACATTA | aaggatttctttaagagcgccatgccg |
| | | ACCTGTCAACTATTTGGTCACTTAA | gaaggttatacccaagagcgtaccatc |
| | | TAAACACTTATATTCGAGCGAATCG | agcttcgaaggcgacggcgtgtacaa |
| | | GAAATGATGAAAGCTGCACCGGGG | gacgcgtgctatggttacctacgaacg |
| | | CAACAAATCATCTTGCCCCTGAAG | cggttctatctacaatcgtgtcacgctg |
| | | AAATTGCCCTTTGAATACTCCGCTT | actggtgagaactttaagaaagacggt |
| | | TGCCCTTGCTGGGCTCGGCTCCTCT | cacattctgcgtaagaacgttgcattcc |
| | | GGTAGCCGCCGGAGGCGTTGCCGG | aatgcccgccaagcattctgtatattct |
| | | TCACACTAATAAGCTGACAAAAAT | gcctgacaccgttaacaatggcatccgc |
| | | GTCACCCGACGTGACGAAGAGCAA | gttgagttcaaccaggcgtacgatattg |
| | | CATGACGGATGATAAGGCTTTAAA | aaggtgtgaccgaaaaactggttacca |
| | | TTACGCAGCTCAGCAAGCGGCCTC | aatgcagccaaatgaatcgtccgttgg |
| | | GTTGGGAAGTCAGTTACAGAGTCG | cgggctccgcggcagtgcatatcccg |
| | | TTCGTTAAATGGTGATTATGCTAAG | cgttatcatcacattacctaccacacca |
| | | GATACCGCATTGGGTATTGCCGGC | aactgagcaaagaccgcgacgagcg |
| | | AACCAAGCGTCGAGCCAACTTCAG | ccgtgatcacatgtgtctggtagaggtc |
| | | GCATGGTTGCAACATTACGGCACT | gtgaaagcggttgatctggacacgtat |
| | | GCTGAAGTAAATCTGCAATCAGGT | cag (SEQ ID NO: 1496) |
| | | AATAATTTTGACGGTAGTTCCCTGG | |
| | | ATTTCCTTTTACCTTTTTACGATTCA | |
| | | GAAAAGATGTTGGCTTTCGGACAG | |
| | | GTGGGGGCGCGTTACATCGATTCA | |
| | | CGTTTTACCGCTAACTTGGGGGCCG | |
| | | GTCAACGCTTCTTCTTACCTGCCAA | |
| | | TATGTTGGGCTATAATGTATTTATC | |
| | | GACCAGGACTTCAGTGGTGACAAT | |
| | | ACACGTCTGGGAATTGGTGGAGAG | |
| | | TAtTGGCGCGATTACTTTAAGTCAT | |
| | | CTGTAAATGGCTATTTTCGCATGAG | |
| | | CGGTTGGCATGAAAGTTACAACAA | |
| | | GAAAGACTACGATGAGCGCCCCGC | |
| | | GAACGGGTTTGACATCCGTTTTAAT | |
| | | GGTTATTTGCCATCTTATCCCGCCT | |
| | | TGGGAGCTAAATTAATCTACGAGC | |
| | | AATACTATGGAGATAACGTAGCTTT | |
| | | GTTTAATAGCGACAAGTTACAGTCT | |
| | | AATCCAGGAGCGGCTACAGTGGGA | |
| | | GTTAATTATACCCCAATCCCACTGG | |
| | | TCACAATGGGAATCGATTATCGCC | |
| | | ACGGGACTGGTAATGAAAACGATT | |
| | | TATTATACTCCATGCAGTTTCGTTA | |
| | | TCAGTTCGATAAGAGTTGGTCGCA | |
| | | GCAGATTGAGCCTCAATATGTTAAC | |
| | | GAATTACGTACCTTGTCCGGCAGTC | |
| | | GCTACGATCTGGTACAACGCAATA | |
| | | ACAATATCATCCTTGAGTATAAGA | |
| | | AACAGGACATTCTGTCTTTGAACAT | |
| | | TCCACATGATATTAATGGTACCGAG | |
| | | CACTCAACACAAAAAATTCAGCTG | |
| | | ATTGTGAAATCAAAGTATGGACTG | |
| | | GACCGTATCGTGTGGGATGATAGC | |
| | | GCTCTGCGCAGTCAGGGTGGACAG | |
| | | ATCCAGCACTCGGGTAGCCAGTCT | |
| | | GCCCAAGACTACCAGGCTATCCTG | |
| | | CCAGCGTATGTCCAAGGGGGAAGT | |
| | | AACATCTACAAAGTTACAGCTCGC | |
| | | GCCTATGACCGCAACGGTAATTCTA | |
| | | GTAATAATGTGCAGTTGACAATTAC | |
| | | GGTGCTGTCCAATGGGCAGGTCGT | |
| | | CGATCAGGTAGGTGTGACGGATTTT | |
| | | ACAGCCGATAAAACCTCTGCGAAG | |
| | | GCACiATAACGCGGATACCATCACA | |
| | | TACACTGCCACTGTAAAAAAAAAC | |
| | | GGTGTCGCGCAGGCAAACGTTCCT | |
| | | GTTAGCTTCAACATCGTGTCGGGTA | |
| | | CAGCCACCCTTGGGGCCAACTCGG | |
| | | CAAAGACTGACGCGAATGGCAAGG | |
| | | CTACAGTCACGTTGAAATCCTCGAC | |
| | | ACCAGGACAGGTCGTTGTGTCTGCC | |
| | | AAGACAGCAGAGATGACCTCCGCC | |
| | | CTTAATGCATCTGCTGTTATCTTCTT | |
| | | CGATCAAACGAAGGCATCT (SEQ ID | |
| | | NO: 1494) | |

TABLE 12
Amino Acid Sequences

| Construct | Anchor | Displayed Protein (aa) |
|---|---|---|
| p15A-Kan- | CSSNKNASNDGSEGMLGAGT | TALTEGAKLFEKEIPYITELEGDVEGMKFIIKG |
| ptet-pelB- | GMDANGGNGNMSSEEQARL | EGTGDATTGTIKAKYICTTGDLPVPWATLVST |
| FLAG- | QMQQLQQNNIVYFDLDKYDI | LSYGVQCFAKYPSHIKDFFKSAMPEGYTQERT |
| dasherGFP- | RSDFAQMLDAHANFLRSNPSY | ISFEGDGVYKTRAMVTYERGSIYNRVTLTGEN |
| HIS-PAL | KVTVEGHADERGTPEYNISLG | FKKDGHILRKNVAFQCPPSILYILPDTVNNGI |
| | ERRANAVKMYLQGKGVSAD | RVEFNQAYDIEGVTEKLVTKCSQMNRPLAGSA |
| | QISIVSYGKEKPAVLGHDEAA | AVHIPRYHHITYHTKLSKDRDERRDHMCLVE |
| | YSKNRRAVLVY | VVKAVDLDTYQ |
| | (SEQ ID NO: 1497) | (SEQ ID NO: 1502) |
| p15A-Kan- | MTSAFDPNLVGPTLPPIPPFTL | TALTEGAKLFEKEIPYITELEGDVEGMKFIIKG |
| ptet-BAN- | PT (SEQ ID NO: 1498) | EGTGDATTGTIKAKYICTTGDLPVPWATLVST |
| FLAG- | | LSYGVQCFAKYPSHIKDFFKSAMPEGYTQERT |
| GFP-HIS | | ISFEGDGVYKTRAMVTYERGSIYNRVTLTGEN |
| | | FKKDGHILRKNVAFQCPPSILYILPDTVNNGI |
| | | RVEFNQAYDIEGVTEKLVTKCSQMNRPLAGSA |
| | | AVHIPRYHHITYHTKLSKDRDERRDHMCLVE |
| | | VVKAVDLDTYQ |
| | | (SEQ ID NO: 1502) |
| p15A-Kan- | MTSKATKLVLGAVILGSTLLA | TALTEGAKLFEKEIPYITELEGDVEGMKFIIKG |
| ptet- | GCSSNAKIDQGINPYVGFEMG | EGTGDATTGTIKAKYICTTGDLPVPWATLVST |
| LppOmpA- | YDWLGRMPYKGSVENGAYK | LSYGVQCFAKYPSHIKDFFKSAMPEGYTQERT |
| FLAG- | AQGVQLTAKLGYPITDDLDIY | ISFEGDGVYKTRAMVTYERGSIYNRVTLTGEN |
| GFP-HIS | TRLGGMVWRADTKSNVYGK | FKKDGHILRKNVAFQCPPSILYILPDTVNNGI |
| | NHDTGVSPVFAGGVEYAITPEI | RVEFNQAYDIEGVTEKLVTKCSQMNRPLAGSA |
| | ATRLEYQWTNNIGDAHTIGTR | AVHIPRYHHITYHTKLSKDRDERRDHMCLVE |
| | PDN (SEQ ID NO: 1499) | VVKAVDLDTYQ |
| | | (SEQ ID NO: 1502) |
| p15A-Kan- | PRAAQPRTQAAAQADAVSTN | TALTEGAKLFEKEIPYITELEGDVEGMKFIIKG |
| ptet- | TNSALSDAMASTQSILLDTGA | EGTGDATTGTIKAKYICTTGDLPVPWATLVST |
| NGIgAsig- | YLTRHIAQKSRADAEKNSVW | LSYGVQCFAKYPSHIKDFFKSAMPEGYTQERT |
| FLAG- | MSNTGYGRDYASAQYRRFSS | ISFEGDGVYKTRAMVTYERGSIYNRVTLTGEN |
| GFP-HIS- | KRTQTQIGIDRSLSENMQIGG | FKKDGHILRKNVAFQCPPSILYILPDTVNNGI |
| NGIgAb | VLTYSDSQHTFDQAGGKNTFV | RVEFNQAYDIEGVTEKLVTKCSQMNRPLAGSA |
| | QANLYGKYYLNDAWYVAGD | AVHIPRYHHITYHTKLSKDRDERRDHMCLVE |
| | IGAGSLRSRLQTQQKANFNRT | VVKAVDLDTYQ |
| | SIQTGLTLGNTLKINQFEIVP | (SEQ ID NO: 1502) |
| | SAGIRYSRLSSADYKLGDDSV | |
| | KVSSMAVKTLTAGLDFAYRFK | |
| | VGNLTVKPLLSAAYFANYGKG | |
| | GVNVGGKSFAYKADNQQQYS | |
| | AGVALLYRNVTLNVNGSITKG | |
| | KQLEKQKSGQIKIQIRF | |
| | (SEQ ID NO: 1464) | |
| p15A-Kan- | MTMTRLKISKTLLAVMLTSAV | TALTEGAKLFEKEIPYITELEGDVEGMKFIIKG |
| ptct-FLAG- | ATGSAYAENNAQTTNESAGQ | EGTGDATTGTIKAKYICTTGDLPVPWATLVST |
| GFP-HIS- | KVDSSMNKVGNFMDDSAITA | LSYGVQCFAKYPSHIKDFFKSAMPEGYTQERT |
| OsmY | KVKAALVDHDNIKSTDISVKT | ISFEGDGVYKTRAMVTYERGSIYNRVTLTGEN |
| | DQKVVTLSGFVESQAQAEEA | FKKDGHILRKNVAFQCPPSILYILPDTVNNGI |
| | VKVAKGVEGVTSVSDKLHVR | RVEFNQAYDIEGVTEKLVTKCSQMNRPLAGSA |
| | DAKEGSVKGYAGDTATTSEIK | AVHIPRYHHITYHTKLSKDRDERRDHMCLVE |
| | AKLLADDIVPSRHVKVETTDG | VVKAVDLDTYQTALTEGAKLFEKEIPYITELE |
| | VVQLSGTVDSQAQSDRAESIA | GDVEGMKFIIKGEGTGDATTGTIKAKYICTTG |
| | KAVDGVKSVKNDLKTK | DLPVPWATLVSTLSYGVQCFAKYPSHIKDFFK |
| | (SEQ ID NO: 1500) | SAMPEGYTQERTISFEGDGVYKTRAMVTYER |
| | | GSIYNRVTLTGENFKKDGHILRKNVAFQCPPSI |
| | | LYILPDTVNNGIRVEFNQAYDIEGVTEKLVTK |
| | | CSQMNRPLAGSAAVHIPRYHHITYHTKLSKDR |
| | | DERRDHMCLVEVVKAVDLDTYQ |
| | | (SEQ ID NO: 1503) |
| p15A- | MVFQPISEFLLIRNAGMSMYF | TALTEGAKLFEKEIPYITELEGDVEGMKFIIKG |
| invasinN- | NKIISFNIISRIVICIFLICGM | EGTGDATTGTIKAKYICTTGDLPVPWATLVST |
| FLAG- | FMAGASEKYDANAPQQVQPYSV | LSYGVQCFAKYPSHIKDFFKSAMPEGYTQERT |
| GFP-HIS | SSSAFENLHPNNEMESSINPFS | ISFEGDGVYKTRAMVTYERGSIYNRVTLTGEN |
| | ASDTERNAAIIDRANKEQETE | FKKDGHILRKNVAFQCPPSILYILPDTVNNGI |
| | AVNKMISTGARLAASGRASD | RVEFNQAYDIEGVTEKLVTKCSQMNRPLAGSA |
| | VAHSMVGDAVNQEIKQWLNR | AVHIPRYHHITYHTKLSKDRDERRDHMCLVE |
| | FGTAQVNLNFDKNFSLKESSL | VVKAVDLDTYQ |
| | DWLAPWYDSASFLFFSQLGIR | (SEQ ID NO: 1502) |
| | NKDSRNTLNLGVGIRTLENG | |
| | WLYGLNTFYDNDLTGHNHRI | |
| | GLGAEAWTDYLQLAANGYFR | |
| | LNGWHSSRDFSDYKERPATG | |
| | GDLRANAYLPALPQLGGKLM | |
| | YEQYTGERVALFGKDNLQRN | |
| | PYAVTAGINYTPVPLLTVGVD | |
| | QRMGKSSKHETQWNLQMNY | |
| | RLGESFQSQLSPSAVAGTRLL | |
| | AESRYNLVDRNNNIVLEYQK | |
| | QQVVKLTLSPATISGLPGQVY | |
| | QVNAQVQGASAVREIVWSDA | |
| | ELIAAGGTLTPLSTTQFNLVLP | |
| | PYKRTAQVSRVTDDLTANFYS | |
| | LSALAVDHQGNRSNSFTLSVT | |
| | VQQPQLTLTAAVIGDGAPANG | |
| | KTAITVEFTVADFEGKPLAGQ | |
| | EVVnTNNGALPNKITEKTDA | |
| | NGVARIALTNTTDGVTVVTAE | |
| | VEGQRQSVDTHFVKGTIAAD | |
| | KSTLAAV | |
| | (SEQ ID NO: 990) | |
| p15A-yiaT- | MLINRNIVALFALPFMASATA | TALTEGAKLFEKEIPYITELEGDVEGMKFTIKG |
| FLAG- | SELSIGAGAAYNESPYRGYNE | EGTGDATTGTIKAKYICTTGDLPVPWATLVST |
| GFP-HIS | NTKAIPLISYEGDTFYVRQTTL | LSYGVQCFAKYPSHIKDFFKSAMPEGYTQERT |
| | GFILSQSEKNELSLTASWMPLE | ISFEGDGVYKTRAMVTYERGSIYNRVTLTGEN |
| | FDPTDNDDYAMQQLDKRDST | FKKDGHILRKNVAFQCPPSILYILPDTVNNGI |
| | AMAGVAWYHHERWGTVKAS | RVEFNQAYDIEGVTEKLVTKCSQMNRPLAGSA |
| | AAADVLDNSNGWVGELSVFH | AVHIPRYHHITYHTKLSKDRDERRDHMCLVE |
| | KMQIGRLSLTPALGVLYYDEN | VVKAVDLDTYQ |
| | | (SEQ ID NO: 1502) |
| | FSDYYYGISESESRRSGLASYS | |
| | AQDAWVPYVSLTAKYPIGEH | |
| | VVLMASAGYSELPEEITDSPMI | |
| | DR (SEQ ID NO: 1465) | |
| p15A- | MITHGCYTRTRHKHKLKKTLI | TALTEGAKLFEKEIPYITELEGDVEGMKFIIKG |
| intiminN- | MLSAGLGLFFYVNQNSFANG | EGTGDATTGTIKAKYICTTGDLPVPWATLVST |
| FLAG- | ENYFKLGSDSKLLTHDSYQNR | LSYGVQCFAKYPSHIKDFFKSAMPEGYTQERT |
| GFP-HIS | LFYTLKTGETVADLSKSQDIN | ISFEGDGVYKTRAMVTYERGSIYNRVTLTGEN |
| | LSTIWSLNKHLYSSESEMMKA | FKKDGHILRKNVAFQCPPSILYILPDTVNNGI |
| | APGQQIILPLKKLPFEYSALP | RVEFNQAYDIEGVTEKLVTKCSQMNRPLAGSA |
| | LLGSAPLVAAGGVAGHTNKLT | AVHIPRYHHITYHTKLSKDRDERRDHMCLVE |
| | KMSPDVTKSNMTDDKALNYA | VVKAVDLDTYQ (SEQ ID NO: 1502) |
| | AQQAASLGSQLQSRSLNGDY | |
| | AKDTALGIAGNQASSQLQAW | |
| | LQHYGTAEVNLQSGNNFDGS | |
| | SLDFLLPFYDSEKMLAFGQVG | |
| | ARYIDSRFTANLGAGQRFFLP | |
| | ANMLGYNVFIDQDFSGDNTR | |
| | LGIGGEYWRDYFKSSVNGYFR | |
| | MSGWHESYNKKDYDERPANG | |
| | FDIRFNGYLPSYPALGAKLIY | |
| | EQYYGDNVALFNSDKLQSNP | |
| | GAATVGVNYTPIPLVTMGIDY | |
| | RHGTGNENDLLYSMQFRYQF | |
| | DKSWSQQIEPQYVNELRTLSG | |
| | SRYDLVQRNNNIILEYKKQDIL | |
| | SLNIPHDINGTEHSTQKIQLIV | |
| | KSKYGLDRIVWDDSALRSQGG | |
| | QIQHSGSQSAQDYQAILPAYV | |
| | QGGSNIYKVTARAYDRNGNS | |
| | SNNVQLTITVLSNGQVVDQVG | |
| | VTDFTADKTSAKADNADTITY | |
| | TATVKKNGVAQANVPVSFNI | |
| | VSGTATLGANSAKTDANGKA | |
| | TVTLKSSTPGQVVVSAKTAEM | |
| | TSALNASAVIFFDQTKAS | |
| | (SEQ ID NO: 1501) | |

### Example 2. Surface Display of Nanobody A4 and EGFR Using E. coli Nissle

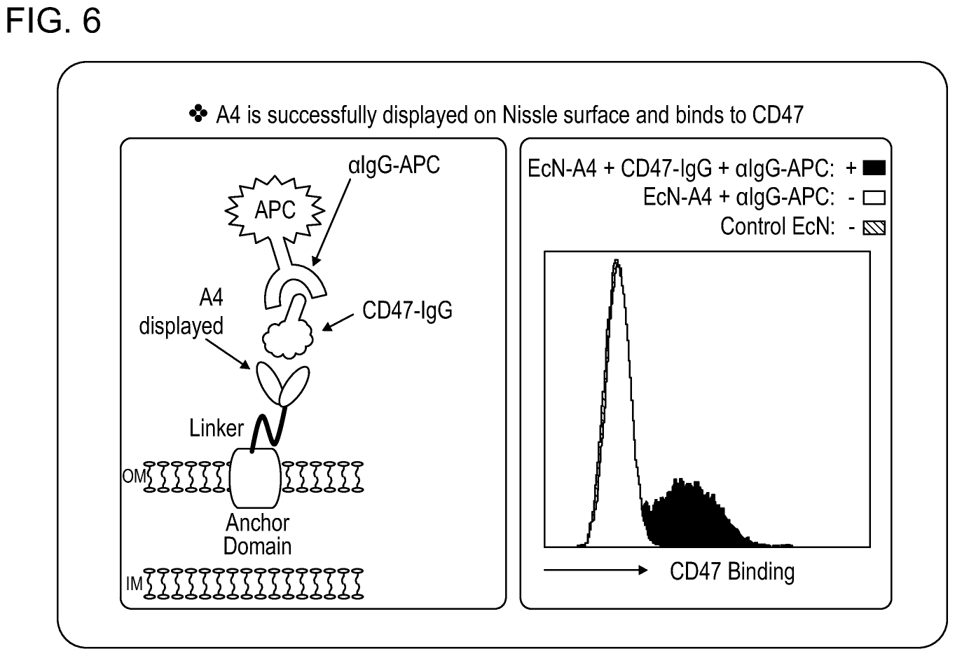

E. coli Nissle cells expressing and displaying nanobody A4 were designed and tested using flow cytometry. Generally, nanobody A4 was fused to an anchor domain by a linker (FIG. 6). In vitro staining and analysis by flow cytometry showed that displayed nanobody A4 bound to CD47-IgG.

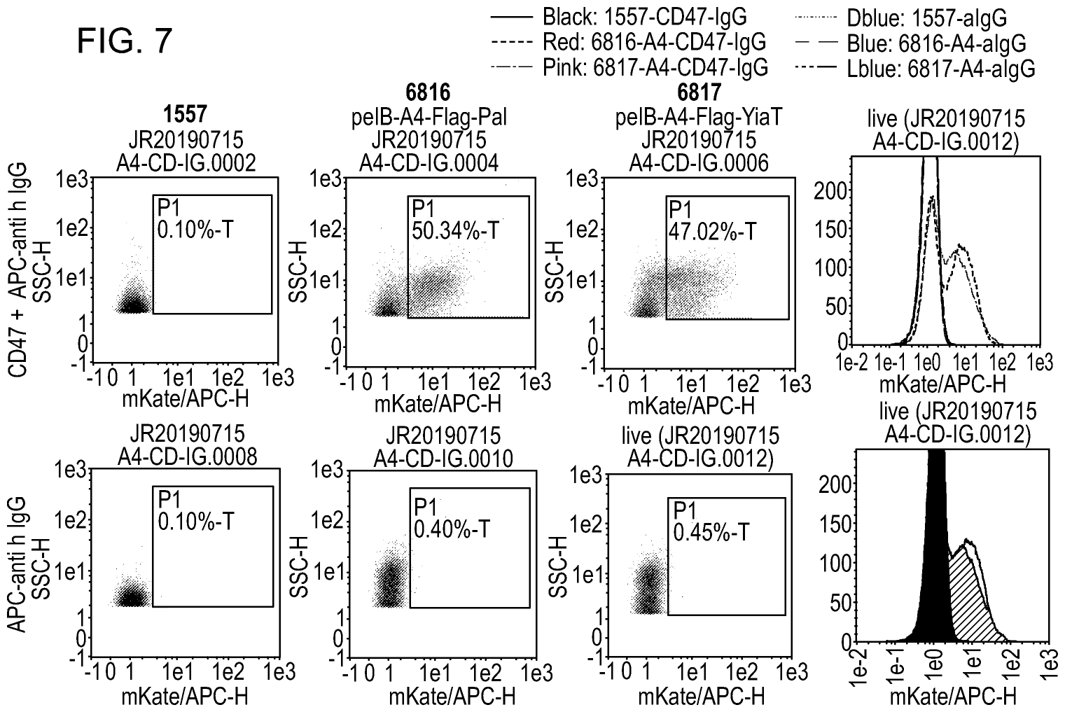

Nanbody A4 was shown to bind CD47 using either pelB-PAL or yiaT as an anchor domain (FIG. 7). The constructs are shown in Table 13 below.

TABLE 13
Strains for Nanobody A4 surface display (FIG. 7)

| Strain | | | | Displayed |
|---|---|---|---|---|
| Number | Construct | Promoter | Anchor | Protein |
| SYN1557 | E. coli | | | |
| | Nissle | | | |
| | delta | | | |
| | PAL: | | | |
| | CmR | | | |
| SYN6816 | pelB-A4- | GTTGACACTC | CSSNKNASN | MKYLLPT |
| | Flag-PAL | TATCATTGAT | DGSEGMLGA | AAAGLLL |
| | | AGAGTTATTT | GTGMDANGG | LAAQPAM |
| | | TACCACTCCC | NGNMSSEEQ | AQVQLVE |
| | | TATCAGTGAT | ARLQMQQLQ | SGGGLVE |
| | | AGAGAA | QNNIVYFDL | PGGSLRL |
| | | (SEQ ID NO: | DKYDIRSDF | SCAASGI |
| | | 1488) | AQMLDAHAN | IFKINDM |
| | | | FLRSNPSYK | GWYRQAP |
| | | | VTVEGHADE | GKRREWV |
| | | | RGTPEYNTS | AASTGGD |
| | | | LGERRANAV | EATYRDS |
| | | | KMYLQGKGV | VKDRFTI |
| | | | SADQISIVS | SRDAKNS |
| | | | YGKEKPAVL | VFLQMNS |
| | | | GHDEAAYSK | LKPEDTA |
| | | | NRRAVLVY | VYYCTAV |
| | | | (SEQ ID | ISTDRDG |
| | | | NO: | TEWRRYW |
| | | | 1497) | GQGTQVT |
| | | | | VSSGGLP |
| | | | | ET |
| | | | | (SEQ ID |
| | | | | NO: |
| | | | | 1505) |
| SYN6817 | pelB-A4- | GTTGACACTC | MLINRNIVA | MKYLLPT |
| | Flag- | TATCATTGAT | LFALPFMAS | AAAGLLL |
| | YiaT | AGAGTTATTT | ATASELSIG | LAAQPAM |
| | | TACCACTCCC | AGAAYNESP | AQVQLVE |
| | | TATCAGTGAT | YRGYNENTK | SGGGLVE |
| | | AGAGAA | AIPLISYEG | PGGSLRL |
| | | (SEQ ID NO: | DTFYVRQTT | SCAASGI |
| | | 1488) | LGFILSQSE | IFKINDM |
| | | | KNELSLTAS | GWYRQAP |
| | | | WMPLEFDPT | GKRREWV |
| | | | DNDDYAMQQ | AASTGGD |
| | | | LDKRDSTAM | EAIYRDS |
| | | | AGVAWYHHE | VKDRFTI |
| | | | RWGTVKASA | SRDAKNS |
| | | | AADVLDNSN | VFLQMNS |
| | | | GWVGELSVF | LKPEDTA |
| | | | HKMQIGRLS | VYYCTAV |
| | | | LTPALGVLY | ISTDRDG |
| | | | YDENFSDYY | TEWRRYW |
| | | | YGISESESR | GQGTQVT |
| | | | RSGLASYSA | VSSGGLP |
| | | | QDAWVPYVS | ET |
| | | | LTAKYPIGE | (SEQ ID |
| | | | HVVLMASAG | NO: |
| | | | YSELPEEIT | 1505) |
| | | | DSPMIDR | |
| | | | (SEQ ID | |
| | | | NO: | |
| | | | 1465) | |

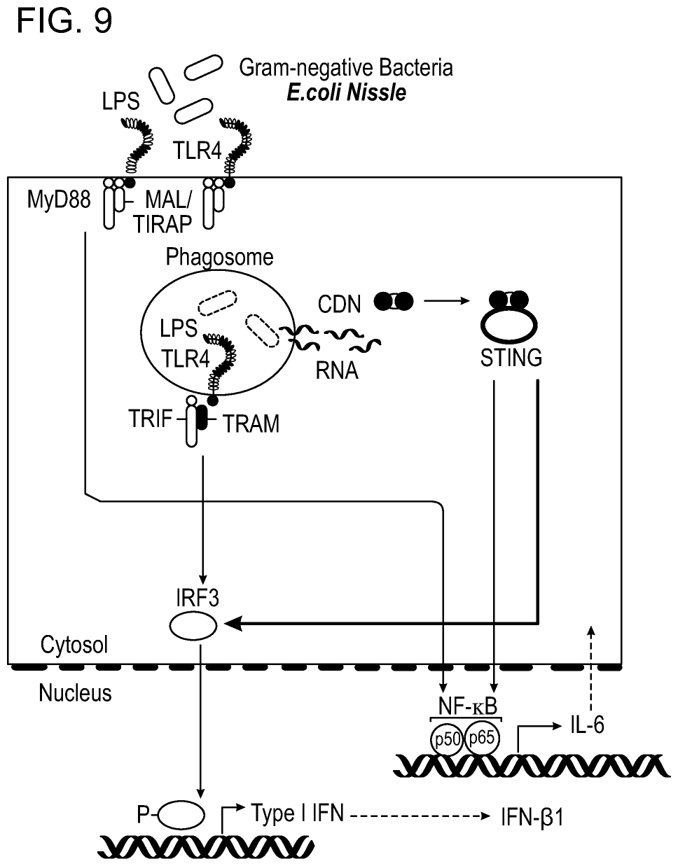

E. coli Nissle cells expressing and displaying aEGFR were designed and tested using flow cytometry. Integrated aEGFR-myc showed similar results as the negative control. Strains SYN7082, SYN7083, SYN7189, and SYN7192 showed EGFR expressing on the surface of the Nissle cells (FIG. 9). The constructs are shown in Table 14 below.

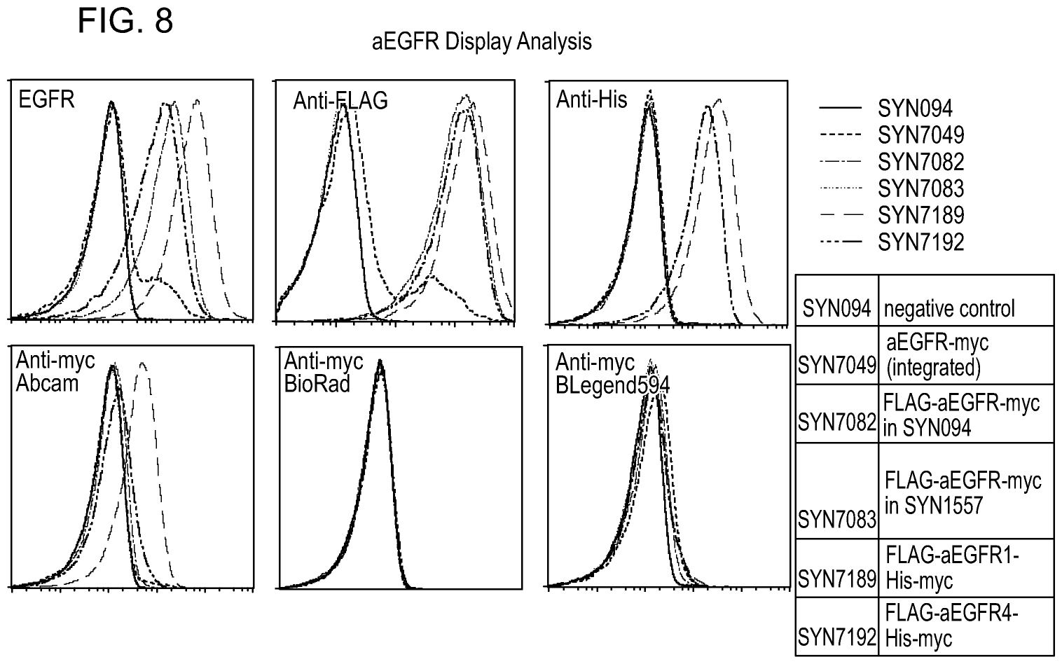

TABLE 14
Strains for aEGFR surface display (FIG. 8)

| Strain | | | | Displayed |
|---|---|---|---|---|
| Number | Construct | Promoter | Anchor | Protein |
| SYN094 | negative | TCATAAAAAA | MITHGCYTRTRHKHKLKK | MAQVQLQESGGG |
| | control | TTTATTTGCTT | TLIMLSAGLGLFFYVNQNS | LVQAGGSLLLSCA |
| | | TCAGGAAAAT | FANGENYFKLGSDSKLLTH | ASGRTFSSYAMG |
| | | TTTTCTGTATA | DSYQNRLFYTLKTGETVAD | WFRQAPGKEREFV |
| | | ATAGATTCAT | LSKSQDINLSTIWSLNKHLY | AAINWSGGSTSYA |
| | | AAATTTGAGA | SSESEMMKAAPGQQIILPL | DSVKGRFTISRDN |
| | | GAGGAGTT | KKLPFEYSALPLLGSAPLV | TKNTVYLQMNSL |
| | | (SEQ ID NO: | AAGGVAGHTNKLTKMSPD | KPEDTAAFYCAAT |
| | | 1506) | VTKSNMTDDKALNYAAQQ | YNPYSRDHYFPRMT |
| | | | AASLGSQLQSRSLNGDYAK | TEYDYWGQGTQVTV |
| | | | DTALGIAGNQASSQLQAW | SS |
| | | | LQHYGTAEVNLQSGNNFD | (SEQ ID |
| | | | GSSLDFLLPFYDSEKMLAF | NO: 1509) |
| | | | GQVGARYIDSRFTANLGAG | |
| | | | QRFFLPANMLGYNVFIDQD | |
| | | | FSGDNTRLGIGGEYWRDYF | |
| SYN7049 | aEGFR- | | KSSVNGYFRMSGWHESYN | |
| | myc | | KKDYDERPANGFDIRFNGY | |
| | (integrated ) | | LPSYPALGAKLIYEQYYGD | |
| | | | NVALFNSDKLQSNPGAATV | |
| | | | GVNYTPIPLVTMGIDYRHG | |
| | | | TGNENDLLYSMQFRYQFDK | |
| | | | SWSQQIEPQYVNELRTLSG | |
| | | | SRYDLVQRNNNIILEYKKQ | |
| | | | DILSLNIPHDINGTEHSTQ | |
| | | | KIQLIVKSKYGLDRIVWDD | |
| | | | SALRSQGGQIQHSGSQSAQ | |
| | | | DYQAILPAYVQGGSNIYKV | |
| | | | TARAYDRNGNSSNNVQLTI | |
| | | | TVLSNGQVVDQVGVTDFTA | |
| | | | DKTSAKADNADTITYTATV | |
| | | | KKNGVAQANVPVSFNIVSG | |
| | | | TATLGANSAKTDANGKATV | |
| | | | TLKSSTPGQVVVSAKTAEM | |
| | | | TSALNASAVIFFDGA | |
| | | | (SEQ ID NO: 1508) | |
| SYN7082 | FLAG- | GTTGACACTCT | MITHGCYTRTRHKHKLKK | AQVQLQESGGGL |
| | aEGFR- | ATCATTGATA | TLIMLSAGLGLFFYVNQNS | VQAGGSLLLSCAA |
| | myc in | GAGTTATTTTA | FANGENYFKLGSDSKLLTH | SGRTFSSYAMGWF |
| | SYN094 | CCACTCCCTAT | DSYQNRLFYTLKTGETVAD | RQAPGKEREFVAA |
| | | CAGTGATAGA | LSKSQDINLSTIWSLNKHLY | INWSGGSTSYADS |
| | | GAA (SEQ ID | SSESEMMKAAPGQQIILPL | VKGRFTISRDNTK |
| | | NO: 1488) | KKLPFEYSALPLLGSAPLV | NTVYLQMNSLKPE |
| | | | AAGGVAGHTNKLTKMSPD | DTAAFYCAATYNP |
| | | | VTKSNMTDDKALNYAAQQ | YSRDHYFPRMTTE |
| | | | AASLGSQLQSRSLNGDYAK | YDYWGQGTQVTV |
| | | | DTALGIAGNQASSQLQAW | SSEPKTPKPQP |
| | | | LQHYGTAEVNLQSGNNFDG | (SEQ ID NO: |
| | | | SSLDFLLPFYDSEKMLAFG | 1510) |
| | | | QVGARYIDSRFTANLGAGQ | |
| | | | RFFLPANMLGYNVFIDQDF | |
| | | | SGDNTRLGIGGEYWRDYFK | |
| | | | SSVNGYFRMSGWHESYNKK | |
| | | | DYDERPANGFDIRFNGYLP | |
| | | | SYPALGAKLIYEQYYGD | |
| | | | NVALFNSDKLQSNPGAAT | |
| | | | VGVNYTPIPLVTMGIDYRH | |
| | | | GTGNENDLLYSMQFRYQF | |
| | | | DKSWSQQIEPQYVNELRTL | |
| | | | SGSRYDLVQRNNNIILEYK | |
| | | | KQDILSLNIPHDINGTEHST | |
| | | | QKIQLIVKSKYGLDRIVWD | |
| | | | DSALRSQGGQIQHSGSQSA | |
| | | | QDYQAILPAYVQGGSNIYK | |
| | | | VTARAYDRNGNSSNNVQL | |
| | | | TITVLSNGQVVDQVGVTDF | |
| | | | TADKTSAKADNADTITYTA | |
| | | | TVKKNGVAQANVPVSFNIV | |
| | | | SGTATLGANSAKTDANGK | |
| | | | ATVTLKSSTPGQVVVSAKT | |
| | | | AEMTSALNASAVIFFDQTK | |
| | | | AS (SEQ ID NO: 1501) | |
| SYN7083 | FLAG- | GTTGACACTCT | MITHGCYTRTRHKHKLKK | AQVQLQESGGGL |
| | aEGFR- | ATCATTGATA | TLIMLSAGLGLFFYVNQNS | VQAGGSLLLSCAA |
| | myc in | GAGTTATTTTA | FANGENYFKLGSDSKLLTH | SGRTFSSYAMGWF |
| | SYN1557 | CCACTCCCTAT | DSYQNRLFYTLKTGETVAD | RQAPGKEREFVAA |
| | | CAGTGATAGA | LSKSQDINLSTIWSLNKHLY | INWSGGSTSYADS |
| | | GAA (SEQ ID | SSESEMMKAAPGQQIILPL | VKGRFTISRDNTK |
| | | NO: 1488) | KKLPFEYSALPLLGSAPLV | NTVYLQMNSLKPE |
| | | | AAGGVAGHTNKLTKMSPD | DTAAFYCAATYNP |
| | | | VTKSNMTDDKALNYAAQQ | YSRDHYFPRMTTE |
| | | | AASLGSQLQSRSLNGDYAK | YDYWGQGTQVTV |
| | | | DTALGIAGNQASSQLQAW | SSEPKTPKPQP |
| | | | LQHYGTAEVNLQSGNNFD | (SEQ ID |
| | | | GSSLDFLLPFYDSEKMLAF | NO: 1510) |
| | | | GQVGARYIDSRFTANLGAG | |
| | | | QRFFLPANMLGYNVFIDQD | |
| | | | FSGDNTRLGIGGEYWRDYF | |
| | | | KSSVNGYFRMSGWHESYN | |
| | | | KKDYDERPANGFDIRFNGY | |
| | | | LPSYPALGAKLIYEQYYGD | |
| | | | NVALFNSDKLQSNPGAAT | |
| | | | VGVNYTPIPLVTMGIDYRH | |
| | | | GTGNENDLLYSMQFRYQF | |
| | | | DKSWSQQIEPQYVNELRTL | |
| | | | SGSRYDLVQRNNNIILEYK | |
| | | | KQDILSLNIPHDINGTEHST | |
| | | | QKIQLIVKSKYGLDRIVWD | |
| | | | DSALRSQGGQIQHSGSQSA | |
| | | | QDYQAILPAYVQGGSNIYK | |
| | | | VTARAYDRNGNSSNNVQL | |
| | | | TITVLSNGQVVDQVGVTDF | |
| | | | TADKTSAKADNADTITYTA | |
| | | | TVKKNGVAQANVPVSFNIV | |
| | | | SGTATLGANSAKTDANGK | |
| | | | ATVTLKSSTPGQVVVSAKT | |
| | | | AEMTSALNASAVIFFDQTK | |
| | | | AS(SEQ ID NO: 1501) | |
| SYN7189 | FLAG- | GTTGACACTCT | MITHGCYTRTRHKHKLKK | QVKLEESGGGSVQ |
| | aEGFR1- | ATCATTGATA | TLIMLSAGLGLFFYVNQNS | TGGSLRLTCAASG |
| | His-myc | GAGTTATTTTA | FANGENYFKLGSDSKLLTH | RTSRSYGMGWFR |
| | | CCACTCCCTAT | DSYQNRLFYTLKTGETVAD | QAPGKEREFVSGIS |
| | | CAGTGATAGA | LSKSQDINLSTIWSLNKHLY | WRGDSTGYADSV |
| | | GAA (SEQ ID | SSESEMMKAAPGQQIILPL | KGRFTISRDNAKN |
| | | NO: 1488) | KKLPFEYSALPLLGSAPLV | TVDLQMNSLKPED |
| | | | AAGGVAGHTNKLTKMSPD | TAIYYCAAAAGSA |
| | | | VTKSNMTDDKALNYAAQQ | WYGTLYEYDYWG |
| | | | AASLGSQLQSRSLNGDYAK | QGTQVTVSS |
| | | | DTALGIAGNQASSQLQAW | (SEQ ID NO: |
| | | | LQHYGTAEVNLQSGNNFD | 1511) |
| | | | GSSLDFLLPFYDSEKMLAF | |
| | | | GQVGARYIDSRFTANLGAG | |
| | | | QRFFLPANMLGYNVFIDQD | |
| | | | FSGDNTRLGIGGEYWRDYF | |
| | | | KSSVNGYFRMSGWHESYN | |
| | | | KKDYDERPANGFDIRFNGY | |
| | | | LPSYPALGAKLIYEQYYGD | |
| | | | NVALFNSDKLQSNPGAAT | |
| | | | VGVNYTPIPLVTMGIDYRH | |
| | | | GTGNENDLLYSMQFRYQF | |
| | | | DKSWSQQIEPQYVNELRTL | |
| | | | SGSRYDLVQRNNNIILEYK | |
| | | | KQDILSLNIPHDINGTEHST | |
| | | | QKIQLIVKSKYGLDRIVWD | |
| | | | DSALRSQGGQIQHSGSQSA | |
| | | | QDYQAILPAYVQGGSNIYK | |
| | | | VTARAYDRNGNSSNNVQL | |
| | | | TITVLSNGQVVDQVGVTDF | |
| | | | TADKTSAKADNADTITYTA | |
| | | | TVKKNGVAQANVPVSFNIV | |
| | | | SGTATLGANSAKTDANGK | |
| | | | ATVTLKSSTPGQVVVSAKT | |
| | | | AEMTSALNASAVIFFDQTK | |
| | | | AS | |
| | | | (SEQ ID NO: 1501) | |
| SYN7192 | FLAG- | GTTGACACTCT | MITHGCYTRTRHKHKLKK | AQVKLEESGGGSV |
| | aEGFR4- | ATCATTGATA | TLIMLSAGLGLFFYVNQNS | QTGGSLRLTCAAS |
| | His-myc | GAGTTATTTTA | FANGENYFKLGSDSKLLTH | GRTSRSYGMGWF |
| | | CCACTCCCTAT | DSYQNRLFYTLKTGETVAD | RQAPGKEREFVSG |
| | | CAGTGATAGA | LSKSQDINLSTIWSLNKHLY | ISWRGDSTGYADS |
| | | GAA | SSESEMMKAAPGQQIILPL | VKGRFTISRDNAK |
| | | (SEQ ID | KKLPFEYSALPLLGSAPLV | NTVDLQMNSLKPE |
| | | NO: 1488) | AAGGVAGHTNKLTKMSPD | DTAIYYCAAAAGS |
| | | | VTKSNMTDDKALNYAAQQ | AWYGTLYEYDYW |
| | | | AASLGSQLQSRSLNGDYAK | GQGTQVTVSSSPS |
| | | | DTALGIAGNQASSQLQAW | TPPTPSPSTPPGLN |
| | | | LQHYGTAEVNLQSGNNFD | DIFEAQKIEWHGS |
| | | | GSSLDFLLPFYDSEKMLAF | S |
| | | | GQVGARYIDSRFTANLGAG | (SEQ ID |
| | | | QRFFLPANMLGYNVFIDQD | NO: |
| | | | FSGDNTRLGIGGEYWRDYF | 1504) |
| | | | KSSVNGYFRMSGWHESYN | |
| | | | KKDYDERPANGFDIRFNGY | |
| | | | LPSYPALGAKLIYEQYYGD | |
| | | | NVALFNSDKLQSNPGAAT | |
| | | | VGVNYTPIPLVTMGIDYRH | |
| | | | GTGNENDLLYSMQFRYQF | |
| | | | DKSWSQQIEPQYVNELRTL | |
| | | | SGSRYDLVQRNNNIILEYK | |
| | | | KQDILSLNIPHDINGTEHST | |
| | | | QKIQLIVKSKYGLDRIVWD | |
| | | | DSALRSQGGQIQHSGSQSA | |
| | | | QDYQAILPAYVQGGSNIYK | |
| | | | VTARAYDRNGNSSNNVQL | |
| | | | TITVLSNGQVVDQVGVTDF | |
| | | | TADKTSAKADNADTITYTA | |
| | | | TVKKNGVAQANVPVSFNIV | |
| | | | SGTATLGANSAKTDANGK | |
| | | | ATVTLKSSTPGQVVVSAKT | |
| | | | AEMTSALNASAVIFFDQTK | |
| | | | AS | |
| | | | (SEQ ID NO: 1501) | |

### Example 3. Development of Vaccine for Prevention of COVID19

Coronaviruses (CoV) are a large family of viruses that cause diseases in mammals and birds. Coronaviruses constitute the subfamily Orthocoronavirinae, in the family Coronaviridae. They are enveloped viruses with a positive-sense single-stranded RNA genome and a nucleocapsid of helical symmetry. The genome size of coronaviruses ranges from approximately 27 to 34 kilobases. The name coronavirus is derived from the Latin corona, meaning “crown” or “halo”, which refers to the characteristic appearance reminiscent of a crown or a solar corona around the virions (virus particles) when viewed under two-dimensional transmission electron microscopy, due to the surface covering in club-shaped protein spikes.

Coronaviruses can cause illness ranging from the common cold to more severe diseases. For example, infections with the human coronavirus strains CoV-229E, CoV-OC43, CoV-NL63 and CoV-HKU1 usually result in mild, self-limiting upper respiratory tract infections, such as a common cold, e.g., runny nose, sneezing, headache, cough, sore throat or fever (Zumla A. et al., Nature Reviews Drug Discovery 15(5): 327-47, 2016; (Cheng V. C., et al., Clin. Microbial. Rev. 20: 660-694, 2007; Chan J. F. et al., Clin. Microbial. Rev. 28: 465-522, 2015). Other infections may result in more severe diseases such as Middle East Respiratory Syndrome (MERS-CoV) and Severe Acute Respiratory Syndrome (SARS-CoV), diseases associated with pneumonia, severe acute respiratory syndrome, kidney failure and death.

MERS-CoV and SARS-CoV have received global attention over the past decades owing to their ability to cause community and health-care-associated outbreaks of severe infections in human populations. MERS-CoV is a viral respiratory disease that was first reported in Saudi Arabia in 2012 and has since spread to more than 27 other countries, according to the World Health Organization (de Groot, R. J. et al., J. Virol. 87: 7790-7792, 2013). SARS was first reported in Asia in 2003, and quickly spread to about two dozen countries before being contained after about four months (Lee N. et al., N. Engl. J. Med. 348: 1986-1994, 2003; Peiris J. S. et al., Lancet 36: 1319-1325, 2003). Detailed investigations found that SARS-CoV was transmitted from civet cats to humans and MERS-CoV from dromedary camels to humans (Cheng V. C., et al., Clin. Microbial. Rev. 20: 660-694, 2007; Chan J. F. et al., Clin. Microbial. Rev. 28: 465-522, 2015).

A recent outbreak of respiratory disease caused by a novel coronavirus, severe acute respiratory syndrome coronavirus 2 (SARS-CoV-2), was first identified in Wuhan City, China. This disease, named by the World Health Organization as coronavirus disease 2019 (“COVID-19”), presents a major threat to public health worldwide.

Coronaviruses viruses pose major challenges to clinical management because many questions regarding transmission and control remain unanswered. Moreover, there is currently no vaccine to prevent infections by coronavirus, and there are no specific antiviral treatments available or proven to be effective to treat or prevent coronavirus infection in subjects.

The capsid spike (S) protein of SARS-CoV2 virus initiates attachment to the angiotensin converting enzyme 2 (ACE2) receptor expressed on the surface of human epithelial cells, facilitating viral entry. The receptor binding domain (RBD) region of the spike protein interacts directly with the ACE2 receptor. Neutralizing antibodies directed against S protein have been observed in patients that have recovered from COVID-19, making S protein and its RBD region an attractive target for vaccine development.

A vaccine for the prevention of COVID19 (or SARS-CoV2) is developed by utilizing synthetic biology techniques to engineer probiotic bacteria that express viral proteins (S-protein RBD) and immune activators/adjuvants. This vaccine is based on an engineered E. coli Nissle (EcN) bacterial strain that expresses viral spike protein receptor bindind domain (RBD) from SARS-CoV2, the causative agent for COVID-19 on its cell surface, and can be administered intranasally to induce protective immunity systemically and at mucosal surfaces.

Specifically, SYNB1891, a clinical candidate for anti-tumor immunity currently in phase I clinical trials, is used as a starting point for engineering. This strain is designed to stimulate the immune system by producing immune activators/adjuvants.

To successfully generate neutralizing antibodies against the RBD region of SARS-CoV2 spike protein, it is essential that the RBD region expressed and displayed on the surface of EcN be conformationally similar to the native RBD region found on SARS-CoV2 spike protein. Even a slight change in the folding and conformation of RBD region on the surface of EcN can lead to the generation of antibodies that are not efficacious against neutralizing the virus. In addition, since spike protein is a glycosylated protein and EcN expresses proteins that are un-glycosylated, this has the potential to impact the conformation and therefore of efficacy of the generated antibody response against SARS CoV-2. Therefore, to minimize the risk of conformational dissimilarity, a bioinformatics approach is employed to perform a structural analysis of the RBD region and a library of RBD expression constructs is designed with construct having varying sizes of flanking sequences on either side to maximize the probability of correct folding.

A genomic library of RBD constructs containing linker regions of various lengths, fused to an appropriate outer membrane-anchoring domain is generated. Several potential anchoring domains were identified to facilitate the delivery of protein to the cell service. The RBD construct libraries are fused to the top 3 anchoring domains. To assess proper display on the cell surface, a high throughput assay is developed. Briefly, a structurally-specific α-RBD antibody (with conjugated fluorophore) is used to stain cells expressing members of the RBD library. Whole cells that acquire fluorescence indicate that the antibody has successfully bound to the cell surface, which also indicates that the RBD library member is likely to be expressed in a conformationally relevant manner. Additionally, RBD constructs may adopt the native trimeric structure on the cell surface, so a secondary assay using recombinant ACE2 protein followed by staining with fluorophore-conjugated α-ACE2 antibody can also be attempted as a secondary screen.

Viral sensing by innate immune cells triggers various signaling cascades including Stimulator of Interferon Genes (STING), leading to the production of interferons and proinflammatory cytokines critical for induction of effective innate and adaptive anti-viral immunity (Lee, H., et al., 2019. Exp Mol Med 51, 1-13). The engineered bacterial strain SYNB1891 produces the STING agonist that triggers STING activation and Type I interferon production in antigen-presenting cells leading to the induction of tumor antigen-specific cytotoxic T-cell responses, and in preclinical models, efficacious antitumor immunity with the formation of immunological memory. SYNB1891 could be further engineered to induce antigen-specific mucosal and systemic immunity to SARS-CoV2.

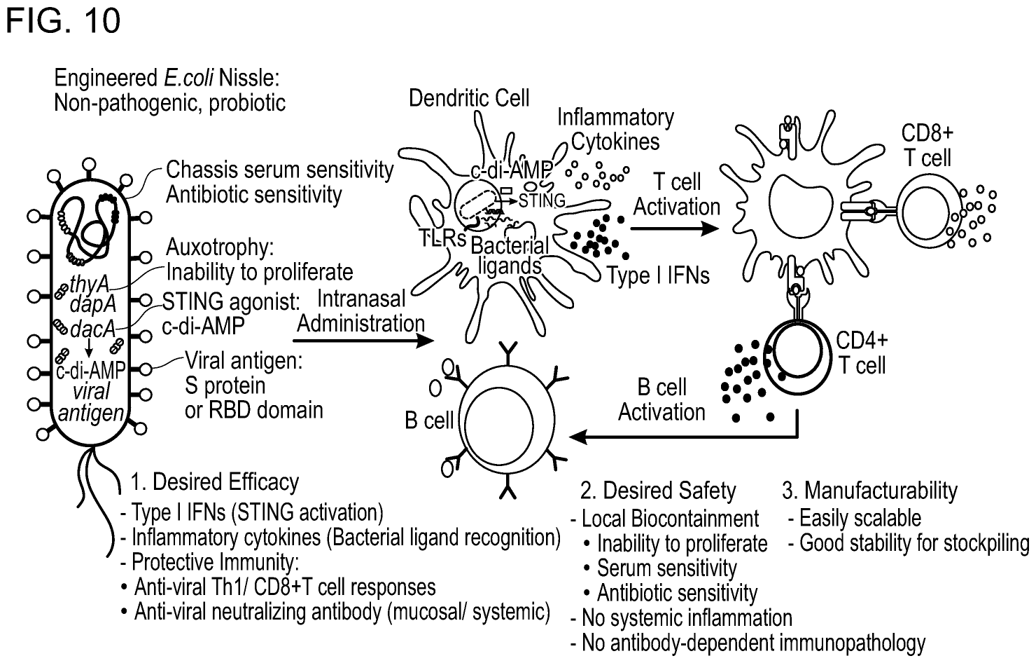

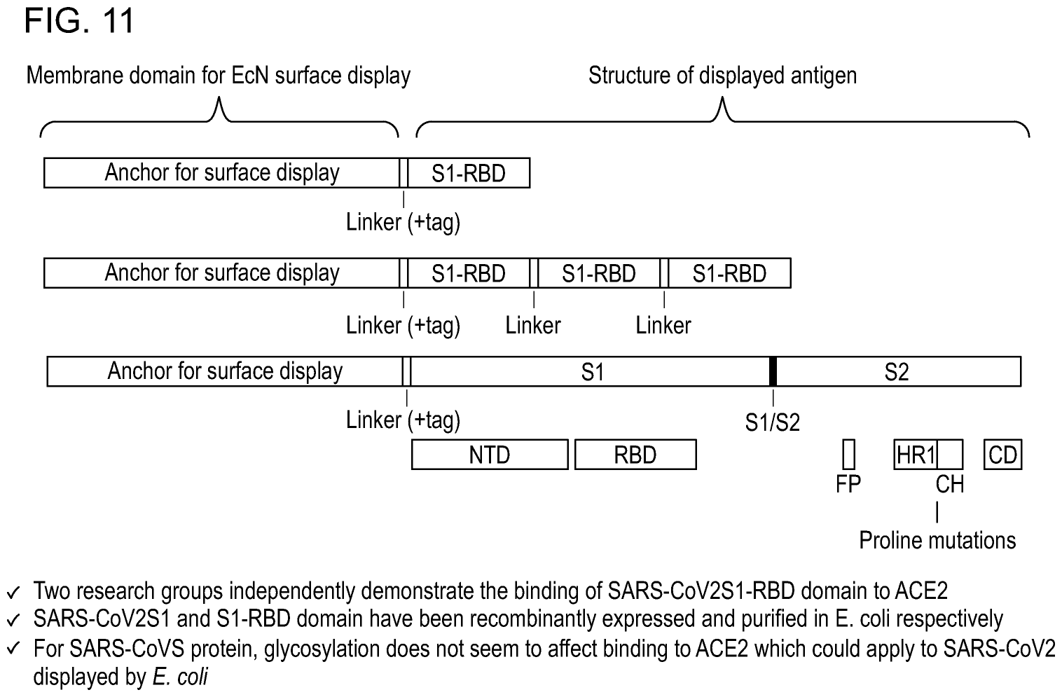

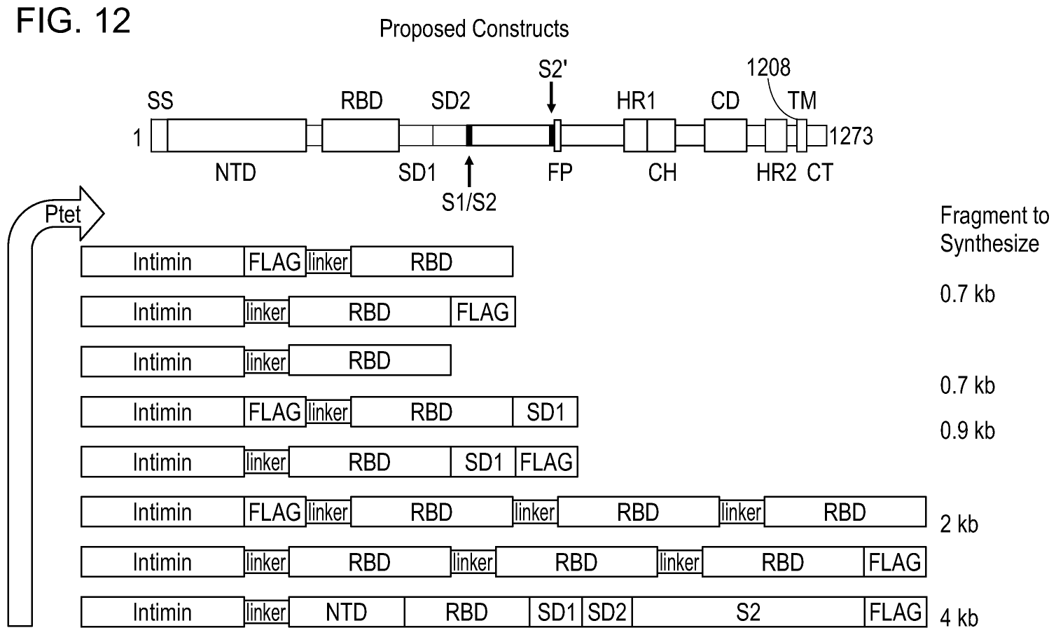

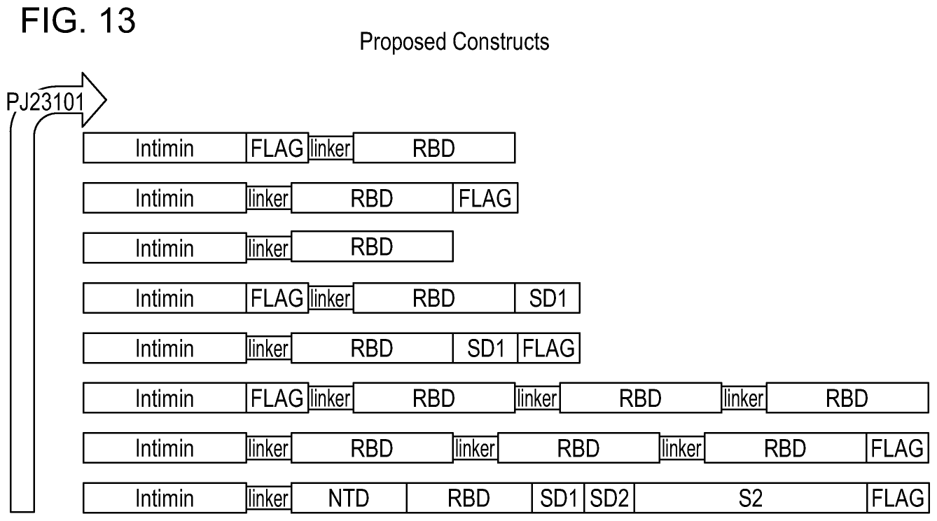

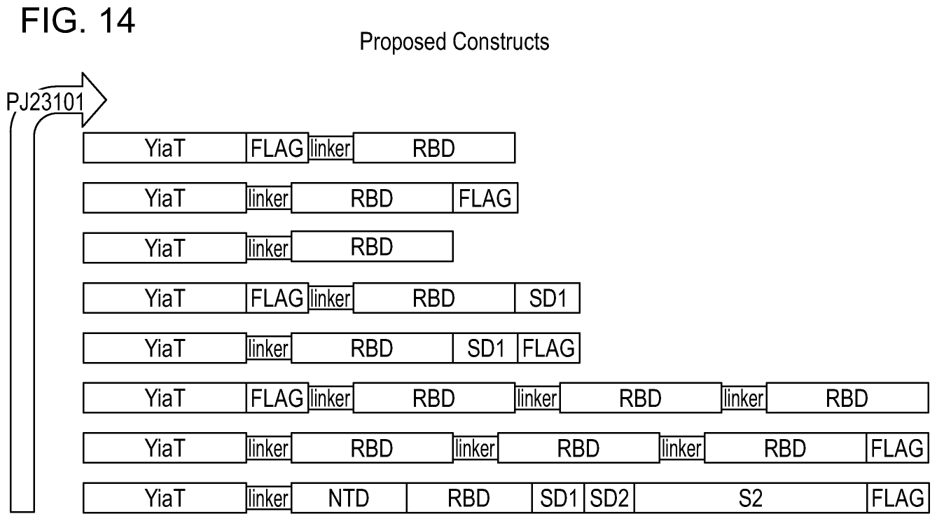

SYNB medicines are well suited to advance an engineered bacterial product as a vaccine candidate for COVID-19. In particular, the EcN based vaccine confers several advantages when compared to the current anti-viral vaccine approaches as described below and shown in FIG. 10. The EcN based vaccine displays a protein to induce an immune response, contains STING agonist, and is unable to proliferate.

Efficacy: Rationally designed, specific viral proteins and immune activators as well as additional functionalities can be engineered into a single cell to induce a protein specific mucosal and systemic immune response. The bacterial chassis itself provides adjuvant effects and allows direct uptake of concentrated protein and activator by antigen presenting cells.

Safety: EcN has been used orally in human populations for over 100 years with a very good safety profile. EcN exhibits serum sensitivity to complement lysis and is susceptible to a broad array of antibiotics. The safety profile of EcN delivered intranasally should be similar. The vaccine of the present invention contains no live virus, and is delivered locally, so has the potential for a safety advantage over attenuated or recombinant viral and DNA vaccine approaches. Prevention of cell division using auxotrophies is engineered to avoid any uncontrolled bacterial growth in the body or the environment. The prototype, SYNB1891 injected intratumorally is now being evaluated in a clinical trial NCT04167137, providing additional human safety data.

Manufacturability: 6 million doses of vaccine can be produced in a single batch (at a dose level of 1×10⁹ live cells/dose).

Stability: SYNB lyophilized cells have room temperature stability for stockpiling. The vaccine would have to be lyophilized for adequate stockpiling and long term stability. There is a risk that the outer membrane of the bacteria is damaged during the lyophilization process, which could have the effect of damaging or denaturing surface-displayed components.

Therefore, the advancement over current anti-viral vaccine approaches is the integration of multiple desired features into a single organism to produce a vaccine with enhanced safety and specific immunity to viral proteins. Additionally, the technology is scalable to manufacture large quantities of vaccine.

### Technical Section:

The existing strain, SYNB1891, is engineered to express the conformationally stable spike protein (S-protein, receptor binding domain) of SARS-CoV2, a critical means of entry of the virus into respiratory cells and a target for other coronavirus vaccine initiatives (Du L., et al, 2019 Exp Mol Med 51, 1-13; Wan Y., et al., 2009 Nat Rev Microbiol. 7(3): 226-236; Wan Y., et al., 2020 J Virol 94: e00127-20; Chen W., et al., 2020. Current Tropical Medicine Reports. https://doi.org/10.1007/s40475-020-00201-6; Kirchdoerfer R, et al., 2018. Scientific Reports; 8:15701). The strain is designed for the local intranasal delivery to enhance mucosal immunity in the respiratory tract where it will mimic natural entry of SARS-CoV2.

In other embodiments, the existing strain, SYNB1891, is engineered to express a epitope which induces a CTL response. In one embodiment, the epitope is in the viral nucleocapsid (N) and/or M protein. Such proteins and epitopes are well known in the art and described at least in Liu et al., Antiviral Research 137 (2017), 82-92; Huang et al., Vaccine 25 (2007):6981-6991; Ahmed et al., Viruses (2020) 12:254; Grifoni et al., Cell Host & Microbiome (2020) 27:1-10; and Chen et al., J. Immunol (2005) 175:591-598, the entire contents of each of which are expressly incorporated by reference herein in their entireties.

A clinical candidate strain that produces a STING Agonist has been engineered. Specifically, a strain of EcN, called SYNB1891, was engineered to produce the STING agonist, c-di-AMP, in the microenvironment by expressing the dacA gene from Listeria monocytogenes under the control of an inducible promoter. SYNB1891 serves as the background strain for further COVID19 vaccine development.

Biologically active proteins can be displayed on the EcN cell surface. The display of proteins on the E. coli surface has been previously described in the literature (Van Bloois E, et al, 2011. Trends Biotechnol. 29(2):79-86).

SYNB1891 has been demonstrated to induce innate and adaptive immune responses. SYNB1891 mechanisms of action include upregulation of 2 innate immune axes: [1] direct STING activation by c-di-AMP and [2] activation of other pattern recognition receptors (including TLR4) by the bacterial chassis itself. SYNB1891 was able to induce Type I IFNs and proinflammatory cytokines from mouse and human dendritic cells and locally in the tumor. The ability of SYNB1891 to induce Type I IFNs in addition to proinflammatory cytokines led to the development of functional anti-tumor CD8+ T cells and immunological memory. These data demonstrate that SYNB1891 triggers relevant innate immune pathways that lead to antigen-specific activation of CD8+ T cell response. Since SYNB1891 mechanisms of action are similarly important for the development of protective anti-viral immunity, these data validate the use of SYNB1891 as a strain to express the SARS-CoV2 protein.

Safety has been engineered directly applicable to a potential vaccine candidate. From a safety and regulatory perspective, biocontainment controls are critical elements of a bacterial-based live therapeutic designed for clinical use. The EcN chassis itself shows serum sensitivity (Grozdanov L, et al., 2002, J Bacteriol; 184:5912-25), antibiotic sensitivity and unable to colonize human gut. Yet, engineered Thymidine (thy) and Diaminopimelic acid (dap) auxotrophies, implemented in SYNB1891 led to inability of this bacterial strain to colonize and proliferate even in immuno-privileged tumor environment. SYNB1891-specific qPCR showed low or absent bacterial biodistribution outside of site of injection.

Bacterial vaccines are not a new concept. There are approved live bacterial vaccines (i.e. for cholera) as well as vaccines being explored in clinical trials and preclinically (Ming Zeng, et al., 2015. Lancet; 386: 1457-64; Thorstensson R, et al., 2014. PLoS ONE 9(1): e83449; Pei-Feng Liu, et al. 2017. Nat Sci. 3(2): e317; Nathalie Mielcarek et al. 2001. Advanced Drug Delivery Reviews 51: 55-69; Adilson José da Silva, et al. 2014. Brazilian Journal of Microbiology 45, 4, 1117-1129). However, induction of an immune response to SARS-CoV2 by any vaccine could generate antibodies that may potentiate immunopathology during infection. This present application also include studies to evaluate both efficacy and safety of the vaccine in at least 2 species (rodent and non-rodent) in the context of a live viral infection with SARS-CoV2.

### Ability to Transition Technology and Expand Use

Probiotic EcN strains have been engineered for the treatment of metabolic diseases, immunologic diseases, and cancer, and have been tested in Phase 1/2 clinical trials, in healthy volunteers as well as in patients. Multiple doses of vaccine under cGMP can be manufactured for human use. The manufacturing capabilities currently allow for cGMP production of batch sizes of up to 300L, in both liquid and solid presentations. Numerous batches are ran throughout the year to support high level of demands. These core competencies of genetic engineering, clinical development and manufacturing provide the ability to deploy a validated platform for the development and production of a COVID-19 vaccine. Additionally, the technology developed here for a COVID-19 vaccine could be readily deployed for other respiratory viruses.

Task 1. Engineering: The current SYNB1891 strain (expressing STING agonist c-di-AMP, double auxotrophy) is engineered to express the SARS-CoV2 Spike-protein Receptor Binding Domain (S-protein RBD).

Steps:
1. Design, build and transform plasmids containing expression cassettes for S-protein with various arrangements (e.g. RBD region only, tandem design, or fusion proteins) targeted for EcN surface display.
2. Demonstrate expression of protein on the surface of EcN in vitro, in a biologically active conformation.
3. Demonstrate the production of c-di-AMP along with protein display on the surface of EcN SYNB1891 strain carrying plasmids described above.
4. Integrate key genetic elements into the chromosome of EcN SYNB1891 and demonstrate the production of c-di-AMP and surface display of protein in final integrated strain in vitro.

An EcN strain with genetic circuits designed for the production of c-di-AMP production as well as surface display of protein of S-protein (or variants thereof) in a biologically relevant conformation. The strain will also be engineered to contain a dual auxotrophy for diaminopimelic acid and thymidine, to inhibit replication in vivo and for biocontainment.

Task 2. Initial in vivo characterization: Characterize engineered SARS-CoV2-S protein expressing strains delivered intranasally to mice by evaluating initial tolerability, residence time and generation of S-antigen specific immune responses. Additionally explore oral route of vaccine delivery.

Steps:
1. Develop all necessary assays to evaluate in vivo antibody and T cell responses.
2. Demonstrate strain viability and residence time at the target mucosal surfaces.
3. Evaluate distribution of live strain in upper respiratory tract, lungs, GI tract and blood. Assess initial mouse tolerability to the treatment/route of administration.
4. Demonstrate generation of protein-specific antibodies in the lungs (target organ), GI tract and blood of Balb/c and C57BL/6 mice after immunization. Characterize type of antibody response, e.g., IgA, IgGs, IgE.
5. Demonstrate generation and characterize protein-specific T cell responses, e.g., CD4+ Th1/Th2 ratio, CD8+ T cell activation.

Demonstrate generation of protein-specific antiviral T cell responses (mostly protective CD4+Th1 and CD8+ T cells without overactivation/skewing to Th2− cell response). Demonstrate anti-viral antibody production (mucosal and systemic) in the immunized mice. Select engineered SARS-CoV2-S protein expressing strain and route of vaccine administration for further evaluation.

Task 3. Efficacy: Test development of protective immunity and neutralizing antibody responses. This work will require collaboration with a BSL3 laboratory capable of infecting a sensitive mouse strain with SARS-CoV2.

Steps:
1. Viral neutralization: Test ability of serum and mucosal antibody to neutralize and prevent infection of human lung epithelial cells with SARS-CoV2.
2. Anti-viral CTL response: Test ability of CD8+ T cells to kill mouse hACE2+ lung epithelial cells infected with SARS-CoV2 or mouse epithelial cells expressing viral S protein.
3. Demonstrate generation of protein-specific IgA/IgG antibodies in the lungs (target organ) and blood after immunization of K18-hACE2 transgenic mouse model (18) (or another mouse model susceptible to SARS CoV2) adopted for COVID-19 research. Additional models like ferrets and NHPs will also be considered.
4. Demonstrate generation of protective immune response and survival of immunized K18-hACE2 transgenic mouse (or other SARS-CoV2 model) after infection with a lethal dose of SARs-CoV2.

Task 4. Safety: Evaluate safety of engineered SARS-CoV2-S protein expressing strain in vivo. Part of this work will require collaboration with a BSL3 laboratory capable of infecting a sensitive mouse strain with SARS-CoV2.

Steps:
1. Toxicology studies to test bacterial spread in blood and multiple organs (especially lungs and brain) by sensitive strain-specific qPCR.
2. Examine systemic pro-inflammatory cytokine release e.g. IL-6, TNFα etc. after immunization.
3. Evaluate undesired antibody-dependent enhancement of immunopathogenesis: increase of viral uptake through opsonizing antibody and overactivation of macrophages, B cells and DCs, resulting in disease enhancement and viral dissemination.
4. Evaluate occurrence of Th2-type eosinophilic lung inflammation in immunized animals following SARS-CoV2 challenge.

1. A recombinant microorganism capable of displaying a displayed protein on its cell surface, wherein the microorganism expresses a display protein comprising an anchor domain, a linker, and the displayed protein.

2. The recombinant microorganism of claim 1, wherein the anchor domain is selected from the group containing, PelB-PAL, PAL, Intimin, YiaT, LppOmpA, BAN, OmsY, Invasin, IgA, PgsA, NGIgAsig-NGIgAb, and Ice nucleation protein.

3. The recombinant microorganism of claim 1, wherein the linker domain is selected from the group consisting of GGGGS (SEQ ID NO: 1477), (GGGGS)x2 (SEQ ID NO: 1478), (GGGGS)x3 (SEQ ID NO: 1479), EAAAK (SEQ ID NO: 1480), (EAAAK)x2 (SEQ ID NO: 1481), and (EAAAK)x3 (SEQ ID NO: 1482).

4. The recombinant microorganism of claim 1, wherein the displayed protein is a bacterial protein, a viral protein, a fungal protein, or a cancer protein.

5. The recombinant microorganism of claim 1, wherein the displayed protein is nanobody A4 or epidermal growth factor receptor (EGFR).

6. The recombinant microorganism of claim 1, wherein the recombinant bacterium comprises a gene encoding the display protein.

7. The recombinant microorganism of claim 6, wherein the gene encoding the display protein comprises a sequence encoding the anchor domain, a linker sequence, and a sequence encoding the displayed protein.

8. The recombinant microorganism of claim 1, wherein the displayed protein can induce an immune response in a subject.

9. A pharmaceutically acceptable composition comprising the recombinant microorganism of claim 1, and a pharmaceutically acceptable carrier.

10. A method of inducing and sustaining an immune response in a subject, the method comprising administering to the subject the pharmaceutically acceptable composition of claim 9, thereby inducing and sustaining the immune response in the subject.

11. A method of preventing and/or treating an infection in a subject, the method comprising administering to the subject the pharmaceutically acceptable composition of claim 9, thereby preventing and/or treating the infection in the subject.

12. A method of inducing and sustaining an immune response in a subject, the method comprising
administering a first recombinant microorganism to the subject, wherein the first recombinant microorganism is capable of displaying a displayed protein on its cell surface; and
administering a second recombinant microorganism to the subject, wherein the second recombinant microorganism is capable of producing an immune modulator,
thereby inducing and sustaining the immune response in the subject.

13. A method of preventing and/or treating a microbial infection in a subject, the method comprising
administering a first recombinant microorganism to the subject, wherein the first recombinant microorganism is capable of displaying a displayed protein on its cell surface; and
administering a second recombinant microorganism to the subject, wherein the second recombinant microorganism is capable of producing an immune modulator,
thereby preventing and/or treating the microbial infection in the subject.

14. A method of inducing and sustaining an immune response in a subject, the method comprising
administering a recombinant microorganism to the subject, wherein the recombinant microorganism is capable of producing a protein and an immune modulator,
thereby inducing and sustaining the immune response in the subject.
